SEQUENCE LISTING (96 sequences)

SEQ ID NO: 1 - DNA, 57 bp, Saccharomyces cerevisiae
ggactagtctgcaggaggagttttaatgtcattaccgttcttaacttctgcaccggg

SEQ ID NO: 2 - DNA, 96 bp, Saccharomyces cerevisiae
ttctcgagcttaagagtagcaatatttaccggagcagttacactagcagtatatacagtcattaaaactcctcctgtgaagtccatggtaaattcg

SEQ ID NO: 3 - DNA, 56 bp, Saccharomyces cerevisiae
tagcggccgcaggaggagttcatatgtcagagttgagagccttcagtgccccaggg

SEQ ID NO: 4 - DNA, 36 bp, Saccharomyces cerevisiae
tttctgcagtttatcaagataagtttccggatcttt

SEQ ID NO: 5 - DNA, 41 bp, Saccharomyces cerevisiae
ggaattcatgaccgtttacacagcatccgttaccgcacccg

SEQ ID NO: 6 - DNA, 45 bp, Saccharomyces cerevisiae
ggctcgagttaaaactcctcttcctttggtagaccagtctttgcg

SEQ ID NO: 7 - DNA, 68 bp, Arabidopsis thaliana
gctctagatgcgcaggaggcacatatggcgaagaacgttgggattttggctatggatatctatttccc

SEQ ID NO: 8 - DNA, 61 bp, Arabidopsis thaliana
cgctcgagtcgacggatcctcagtgtccattggctacagatccatcttcacctttcttgcc

SEQ ID NO: 9 - DNA, 72 bp, Arabidopsis thaliana
ccgctcgagcacgtggaggcacatatgcaatgctgtgagatgcctgttggatacattcagattcctgttggg

SEQ ID NO: 10 - DNA, 71 bp, Arabidopsis thaliana
ggggtacctgcggccggatcccgggtcatgttgttgttgttgtcgttgtcgttgctccagagatgtctcgg

SEQ ID NO: 11 - DNA, 74 bp, Saccharomyces cerevisiae
acaacaccgcggcggccgcgtcgacgccggcggaggcacatatgtctcagaacgtttacattgtatcgactgcc

SEQ ID NO: 12 - DNA, 53 bp, Saccharomyces cerevisiae
gctctagaggatcctcatatcttttcaatgacaatagaggaagcaccaccacc

SEQ ID NO: 13 - DNA, 65 bp, Saccharomyces cerevisiae
gctctagatacgtaggaggcacatatgagtgagcttatacccgcctgggttggtgacagactggc

SEQ ID NO: 14 - DNA, 61 bp, Artificial
  Oligonucleotide containing A. thaliana and S. cerevisiae DNA
cgctcgagcccgggggatcctcagccgcgcaggatcgatccgaaaatccggtcaagatggc

SEQ ID NO: 15 - DNA, 72 bp, Saccharomyces cerevisiae
gctctagatacgtaggaggcacatatgagttcccaacaagagaaaaaggattatgatgaagaacaattaagg

SEQ ID NO: 16 - DNA, 59 bp, Saccharomyces cerevisiae
cgctcgagcccgggggatccttagcaacgatgaattaaggtatcttggaattttgacgc

SEQ ID NO: 17 - DNA, 6215 bp, Artificial
  Vector pBSNT27 containing Nicotiana tabacum DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcttcttggctgttattcaaaaggtccaacaatgtatatatattggacattttgaggcaattatagatcctggaaggcaattctgattggtcaataaaaatcgatttcaatgctattttttttttgttttttatgagtttagccaatttatcatgaaaggtaaaaggggataaaggaaccgtgtgttgattgtcctgtaaatataagttgtcttcctccatatgtaaaaagggaataaataaatcaattaaatttcgggatgcttcatgaagtgcttctttcggagttaaacttccgtttgtccatatttcgagaaaaagtatctcttgtttttcattcccattcccataagaatgaatactatgattcgcgtttcgaacaggcatgaatacagcatctataggataacttccatcttgaaagttatgtggcgtttttataagatatccacgatttctctctatttgtaatccaatacaaaaatcaattggttccgttaaactggctatatgttgtgtattatcaacgatttctacataaggcggcaagatgatatcttgggcagttacagatccaggacccttgacacaaatagatgcgtcagaagttccatatagattacttcttaatataatttctttcaaattcattaaaatttcatgtaccgattcttgaatgcccgttatggtagaatattcatgtgggactttctcagattttacacgtgtgatacatgttccttctatttctccaagtaaagctcttcgcatcgcaatgcctattgtgtcggcttggcctttcataagtggagacagaataaagcgtccataataaaggcgtttactgtctgttcttgattcaacacacttccactgtagtgtccgagtagatactgttactttctctcgaaccatagtactattatttgattagatcatcgaatcttttatttctcttgagatttcttcaatgttcagttctacacacgtctttttttcggaggtctacagccattatgtggcataggagttacatcccgtacgaaagttaatagtataccacttcgacgaatagctcgtaatgctgcatctcttccgagaccgggaccttttatcatgacttctgctcgttgcataccttgatccactactgtacggatagcgtttgctgctgcggtttgagcagcaaacggtgttcctcttctcgtacctttgaatccagaagtaccggcggaggaccaagaaactactcgaccccgtacatctgtaacagtgacaatggtattattgaaacttgcttgaacatgaataactccctttggtattctacgtgcacccttacgtgaaccaatacgtccattcctacgcgaactaattttcggtatagcttttgccatattttatcatctcgtaaatatgagtcagagatatatggatatatccatttcatgtcaaaacagattctttatttgtacatcggctcttctggcaagtctgattatccctgtctttgtttatgtctcgggttggaacaaattactataattcgtccccgcctacggattagtcgacatttttcacaaattttacgaacggaagctcttattttcatatttctcattccttaccttaattctgaatctatttcttggaagaaaataagtttcttgaaatttttcatctcgaattgtattcccacgaaaggaatggtgaagttgaaaaacgaatccttcaaatctttgttgtggagtcgataaattatacgccctttggttgaatcataaggacttacttcaattttgactctatctcctggcagtatccgtataaaactatgccggatctttcctgaaacataatttataatcagatctaaacaaacccggaacagaccgttgggaagcgattcagtaattaaagcttcatgactcctttttggttcttaaagtccctttgaggtatcaactaataagaaagatattagacaaccccccttttttctttttcacaaataggaagtttcgaatccaatttggatattaaaaggattaccagatataacacaaaatctctccacctattccttctagtcgagcctctcggtctgtcattatacctcgagaagtagaaagaattacaatccccattccacctaaaattcgcggaattcgttgataattagaatagattcgtagaccaggtcgactgattcgttttaaatttaaaatatttctatagggtcttttcctattccttctatgtcgcagggttaaaaccaaaaaatatttgtttttttctcgatgttttctcacgttttcgataaaaccttctcgtaaaagtatttgaacaatattttcggtaatattagtagatgctattcgaaccaccctttttcgatccatatcagcatttcgtatagaagttattatctcagcaatagtgtccctacccatgatgaactaaaattattggggcctccaaatttgatataatcaacgtgttttttacttattttttttttgaatatgatatgaattattaaagatatatgcgtgagacacaatctactaattaatctatttctttcaaataccccactagaaacagatcacaatttcattttataatacctcgggagctaatgaaactattttagtaaaatttaattctctcaattcccgggcgattgcaccaaaaattcgagttccttttgatttccttccttcttgatcaataacaactgcagcattgtcatcatatcgtattatcatcccgttgtcacgtttgagttctttacaggtccgcacaattacagctctgactacttctgatctttctaggggcatatttggtacggcttctttgatcacagcaacaataacgtcaccaatatgagcatatcgacgattgctagctcctatgattcgaatacacatcaattctcgagccccgctgttatccgctacatttaaatgggtctgaggttgaatcatttttttaatccgttctttgaatgcaaagggcgaagaaaaaaaagaaatatttttgtccaaaaaaaaagaaacatgcggtttcgtttcatatctaagagccctttccgcatttttttctattacattacgaaataatgaattgagttcgtataggcattttagatgctgctagtgaaatagcccttctggctatattttctgttactccacccatttcataaagtattcgacccggtttaacaacagctacccaatattcaggggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 18 - DNA, 1332 bp, Artificial
  Oligonucleotide containing N. tabacum and S. cerevisiae DNA
atgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcataa

SEQ ID NO: 19 - DNA, 1191 bp, Artificial
  Oligonucleotide containing N. tabacum and A. thaliana DNA
atgaccgtttacacagcatccgttaccgcacccgtcaacatcgcaacccttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataa

SEQ ID NO: 20 - DNA, 1197 bp, Rhodobacter capsulatus
atgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatga

SEQ ID NO: 21 - DNA, 1386 bp, Rhodobacter capsulatus
atggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactga

SEQ ID NO: 22 - DNA, 1779 bp, Schizosaccharomyces pombe
atggatctccgtcggaggcctcctaaaccaccggttaccaacaacaacaactccaacggatctttccgttcttatcagcctcgcacttccgatgacgatcatcgtcgccgggctacaacaattgctcctccaccgaaagcatccgacgcgcttcctcttccgttatatctcacaaacgccgttttcttcacgctcttcttctccgtcgcgtattacctcctccaccggtggcgtgacaagatccgttacaatacgcctcttcacgtcgtcactatcacagaactcggcgccattattgctctcatcgcttcgtttatctatctcctagggttttttggtattgactttgttcagtcatttatctcacgtgcctctggtgatgcttgggatctcgccgatacgatcgatgatgatgaccaccgccttgtcacgtgctctccaccgactccgatcgtttccgttgctaaattacctaatccggaacctattgttaccgaatcgcttcctgaggaagacgaggagattgtgaaatcggttatcgacggagttattccatcgtactcgcttgaatctcgtctcggtgattgcaaaagagcggcgtcgattcgtcgtgaggcgttgcagagagtcaccgggagatcgattgaagggttaccgttggatggatttgattatgaatcgattttggggcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatga

SEQ ID NO: 23 - DNA, 684 bp, Schizosaccharomyces pombe
atgagttcccaacaagagaaaaaggattatgatgaagaacaattaaggttgatggaagaagtttgtatcgttgtagatgaaaatgatgtccctttaagatatggaacgaaaaaggagtgtcatttgatggaaaatataaataaaggtcttttgcatagagcattctctatgttcatctttgatgagcaaaatcgccttttacttcagcagcgtgcagaagagaaaattacatttccatccttatggacgaatacatgttgctcccacccattggatgttgctggtgaacgtggtaatactttacctgaagctgttgaaggtgttaagaatgcagctcaacgcaagctgttccatgaattgggtattcaagccaagtatattcccaaagacaaatttcagtttcttacacgaatccattaccttgctcctagtactggtgcttggggagagcatgaaattgactacattcttttcttcaaaggtaaagttgagctggatatcaatcccaatgaagttcaagcctataagtatgttactatggaagagttaaaagagatgttttccgatcctcaatatggattcacaccatggttcaaacttatttgtgagcattttatgtttaaatggtggcaggatgtagatcatgcgtcaaaattccaagataccttaattcatcgttgctaa

SEQ ID NO: 24 - DNA, 531 bp, Artificial
  PCR primer containing Streptomyces sp CL190 DNA
atgagtgagcttatacccgcctgggttggtgacagactggctccggtggacaagttggaggtgcatttgaaagggctccgccacaaggcggtgtctgttttcgtcatggatggcgaaaacgtgctgatccagcgccgctcggaggagaaatatcactctcccgggctttgggcgaacacctgctgcacccatccgggctggaccgaacgccccgaggaatgcgcggtgcggcggctgcgcgaggagctggggatcaccgggctttatcccgcccatgccgaccggctggaatatcgcgccgatgtcggcggcggcatgatcgagcatgaggtggtcgacatctatctggcctatgccaaaccgcatatgcggatcacccccgatccgcgcgaagtggccgaggtgcgctggatcggcctttacgatctggcggccgaggccggtcggcatcccgagcggttctcgaaatggctcaacatctatctgtcgagccatcttgaccggattttcggatcgatcctgcgcggctga

SEQ ID NO: 25 - DNA, 65 bp, Artificial
  PCR primer containing Streptomyces sp CL190 DNA
ggggtaccgcggccgcacgcgtctatgcaccaacctttgcggtcttgttgtcgcgttccagctgg

SEQ ID NO: 26 - DNA, 60 bp, Saccharomyces cerevisiae
gagctccaccgcggcggccgcgtcgactacggccgcaggaggagttcatatgtcagagtt

SEQ ID NO: 27 - DNA, 60 bp, Saccharomyces cerevisiae
tctaccaaaggaagaggagttttaactcgagtaggaggcacatatgtctcagaacgttta

SEQ ID NO: 28 - DNA, 60 bp, Artificial
  Oligonucleotide containing Streptomyces sp CL190 and R. capsulatus DNA
caagaccgcaaaggttggtgcatagacgcggtaaggaggcacatatgagtgagcttatac

SEQ ID NO: 29 - DNA, 60 bp, Rhodobacter capsulatus
cctgcgcggctgagcggccgcggatccgatcgcgtgcggccgcggtacccaattcgccct

SEQ ID NO: 30 - DNA, 60 bp, Artificial
  Oligonucleotide containing Streptomyces sp CL190 and S. cerevisiae DNA
tgtcattgaaaagatatgaggatcctctaggtacttccctggcgtgtgcagcggttgacg

SEQ ID NO: 31 - DNA, 60 bp, Artificial
  Oligonucleotide containing Streptomyces sp CL190 DNA
cgattccgcattatcggtacgggtgcctacctagaactagtggatcccccgggctgcagg

SEQ ID NO: 32 - DNA, 60 bp, Artificial
  Oligonucleotide containing N. tabacum and S. cerevisiae DNA
ctttcctgaaacataatttataatcagatcggccgcaggaggagttcatatgtcagagtt

SEQ ID NO: 33 - DNA, 60 bp, Artificial
  Oligonucleotide containing N. tabacum and R. capsulatus DNA
ttcggatcgatcctgcgcggctgagcggccgatctaaacaaacccggaacagaccgttgg

SEQ ID NO: 34 - DNA, 59 bp, Artificial
  Oligonucleotide containing N. tabacum and S. cerevisiae DNA
ctttcctgaaacataatttataatcagatcggccgcaggaggagttcatatgtcagagt

SEQ ID NO: 35 - DNA, 60 bp, Artificial
  Oligonucleotide containing N. tabacum and S. pombe DNA
tcgttgctaaggatcccccgggatccggccgatctaaacaaacccggaacagaccgttgg

SEQ ID NO: 36 - DNA, 13 bp, Artificial
  Oligonucleotide containing NotI restriction site
catggcggccgcg

SEQ ID NO: 37 - DNA, 13 bp, Artificial
  Oligonucleotide containing NotI restriction site
gatccgcggccgc

SEQ ID NO: 38 - DNA, 60 bp, Saccharomyces cerevisiae
ttaaataaggaggaataaaccatggcggccgcaggaggagttcatatgtcagagttgaga

SEQ ID NO: 39 - DNA, 60 bp, Arabidopsis thaliana
aacaacaacaacatgacccgggatccggccgcgatccgagctcgagatctgcagctggta

SEQ ID NO: 40 - DNA, 60 bp, Saccharomyces cerevisiae
tcgattaaataaggaggaataaaccatggcggccgcaggaggagttcatatgtcagagtt

SEQ ID NO: 41 - DNA, 60 bp, Rhodobacter capsulatus
gattttcggatcgatcctgcgcggctgagcggccgcgatccgagctcgagatctgcagct

SEQ ID NO: 42 - DNA, 60 bp, Saccharomyces cerevisiae; identical to SEQ ID NO: 40

SEQ ID NO: 43 - DNA, 60 bp, Schizosaccharomyces pombe
ttcatcgttgctaaggatcccccgggatccggccgcgatccgagctcgagatctgcagct

SEQ ID NO: 44 - DNA, 61 bp, Rhodobacter capsulatus
ttaaataaggaggaataaaccatggcggccgtaaggaggcacatatgagtgagcttatact

SEQ ID NO: 45 - DNA, 61 bp, Rhodobacter capsulatus
gcctgcgcggctgagcggccgcggatccgatggccgcgatccgagctcgagatctgcagct

SEQ ID NO: 46 - DNA, 60 bp, Schizosaccharomyces pombe
ttaaataaggaggaataaaccatggcggccgtaggaggcacatatgagttcccaacaaga

SEQ ID NO: 47 - DNA, 60 bp, Schizosaccharomyces pombe
accttaattcatcgttgctaaggatcccccggccgcgatccgagctcgagatctgcagct

SEQ ID NO: 48 - DNA, 1356 bp, Saccharomyces cerevisiae
atgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaataa

SEQ ID NO: 49 - DNA, 1332 bp, Saccharomyces cerevisiae; identical to SEQ ID NO: 18

SEQ ID NO: 50 - DNA, 1191 bp, Saccharomyces cerevisiae; identical to SEQ ID NO: 19

SEQ ID NO: 51 - DNA, 1197 bp, Saccharomyces cerevisiae; identical to SEQ ID NO: 20

SEQ ID NO: 52 - DNA, 1386 bp, Arabidopsis thaliana; identical to SEQ ID NO: 21

SEQ ID NO: 53 - DNA, 1779 bp, Arabidopsis thaliana; identical to SEQ ID NO: 22

SEQ ID NO: 54 - DNA, 684 bp, Artificial; identical to SEQ ID NO: 23
  Schizosaccharomyces pombe IDI1 (IPP isomerase)

SEQ ID NO: 55 - DNA, 531 bp, Artificial; identical to SEQ ID NO: 24
  Rhodobacter capsulatus idiB (IPP isomerase)

SEQ ID NO: 56 - DNA, 1059 bp, Streptomyces sp.
atgacggaaacgcacgccatagccggggtcccgatgaggtgggtgggaccccttcgtatttccgggaacgtcgccgagaccgagacccaggtcccgctcgccacgtacgagtcgccgctgtggccgtcggtgggccgcggggcgaaggtctcccggctgacggagaagggcatcgtcgccaccctcgtcgacgagcggatgacccgctcggtgatcgtcgaggcgacggacgcgcagaccgcgtacatggccgcgcagaccatccacgcccgcatcgacgagctgcgcgaggtggtgcgcggctgcagccggttcgcccagctgatcaacatcaagcacgagatcaacgcgaacctgctgttcatccggttcgagttcaccaccggtgacgcctccggccacaacatggccacgctcgcctccgatgtgctcctggggcacctgctggagacgatccctggcatctcctacggctcgatctccggcaactactgcacggacaagaaggccaccgcgatcaacggcatcctcggccgcggcaagaacgtgatcaccgagctgctggtgccgcgggacgtcgtcgagaacaacctgcacaccacggctgccaagatcgtcgagctgaacatccgcaagaacctgctcggcaccctgctcgccggcggcatccgctcggccaacgcccacttcgcgaacatgctgctcggcttctacctggccaccggccaggacgccgccaacatcgtcgagggctcgcagggcgtcgtcatggccgaggaccgcgacggcgacctctacttcgcctgcaccctgccgaacctgatcgtcggcacggtcggcaacggcaagggtctcggcttcgtggagacgaacctcgcccggctcggctgccgagccgaccgcgaacccggggagaacgcccgccgcctcgccgtcatcgcggcagcgaccgtgctgtgcggtgaactctcgctgctcgcggcacagacgaacccgggcgaactcatgcgcgcgcacgtccagctggaacgcgacaacaagaccgcaaaggttggtgca

SEQ ID NO: 57 - DNA, 6798 bp, Artificial
  Streptomyces sp CL190 gene cluster containing mevalonate pathway and IPP isomerase orfs
tacgtacttccctggcgtgtgcagcggttgacgcgccgtgccctcgctgcgagcggcgcgcacatctgacgtcctgctttattgctttctcagaactcgggacgaagcgatcccatgatcacgcgatctccatgcagaaaagacaaagggagctgagtgcgttgacactaccgacctcggctgagggggtatcagaaagccaccgggcccgctcggtcggcatcggtcgcgcccacgccaaggccatcctgctgggagagcatgcggtcgtctacggagcgccggcactcgctctgccgattccgcagctcacggtcacggccagcgtcggctggtcgtccgaggcctccgacagtgcgggtggcctgtcctacacgatgaccggtacgccgtcgcgggcactggtgacgcaggcctccgacggcctgcaccggctcaccgcggaattcatggcgcggatgggcgtgacgaacgcgccgcacctcgacgtgatcctggacggcgcgatcccgcacggccggggtctcggctccagcgcggccggctcacgcgcgatcgccttggccctcgccgacctcttcggccacgaactggccgagcacacggcgtacgaactggtgcagacggccgagaacatggcgcacggccgggccagcggcgtggacgcgatgacggtcggcgcgtcccggccgctgctgttccagcagggccgcaccgagcgactggccatcggctgcgacagcctgttcatcgtcgccgacagcggcgtcccgggcagcaccaaggaagcggtcgagatgctgcgggagggattcacccgcagcgccggaacacaggagcggttcgtcggccgggcgacggaactgaccgaggccgcccggcaggccctcgccgacggccggcccgaggagctgggctcgcagctgacgtactaccacgagctgctccatgaggcccgcctgagcaccgacggcatcgatgcgctggtcgaggccgcgctgaaggcaggcagcctcggagccaagatcaccggcggtggtctgggcggctgcatgatcgcacaggcccggcccgaacaggcccgggaggtcacccggcagctccacgaggccggtgccgtacagacctgggtcgtaccgctgaaagggctcgacaaccatgcgcagtgaacacccgaccacgaccgtgctccagtcgcgggagcagggcagcgcggccggcgccaccgcggtcgcgcacccaaacatcgcgctgatcaagtactggggcaagcgcgacgagcggctgatcctgccctgcaccaccagcctgtcgatgacgctggacgtcttccccacgaccaccgaggtccggctcgaccccgccgccgagcacgacacggccgccctcaacggcgaggtggccacgggcgagacgctgcgccgcatcagcgccttcctctccctggtgcgggaggtggcgggcagcgaccagcgggccgtggtggacacccgcaacaccgtgcccaccggggcgggcctggcgtcctccgccagcgggttcgccgccctcgccgtcgcggccgcggccgcctacgggctcgaactcgacgaccgcgggctgtcccggctggcccgacgtggatccggctccgcctcgcggtcgatcttcggcggcttcgccgtctggcacgccggccccgacggcacggccacggaagcggacctcggctcctacgccgagccggtgcccgcggccgacctcgacccggcgctggtcatcgccgtggtcaacgccggccccaagcccgtctccagccgcgaggccatgcgccgcaccgtcgacacctcgccgctgtaccggccgtgggccgactccagtaaggacgacctggacgagatgcgctcggcgctgctgcgcggcgacctcgaggccgtgggcgagatcgcggagcgcaacgcgctcggcatgcacgccaccatgctggccgcccgccccgcggtgcggtacctgtcgccggccacggtcaccgtgctcgacagcgtgctccagctccgcaaggacggtgtcctggcctacgcgaccatggacgccggtcccaacgtgaaggtgctgtgccggcgggcggacgccgagcgggtggccgacgtcgtacgcgccgccgcgtccggcggtcaggtcctcgtcgccgggccgggagacggtgcccgcctgctgagcgagggcgcatgacgacaggtcagcgcacgatcgtccggcacgcgccgggcaagctgttcgtcgcgggcgagtacgcggtcgtggatccgggcaacccggcgatcctggtagcggtcgaccggcacatcagcgtcaccgtgtccgacgccgacgcggacaccggggccgccgacgtcgtgatctcctccgacctcggtccgcaggcggtcggctggcgctggcacgacggccggctcgtcgtccgcgacccggacgacgggcagcaggcgcgcagcgccctggcccacgtggtgtcggcgatcgagaccgtgggccggctgctgggcgaacgcggacagaaggtccccgctctcaccctctccgtcagcagccgcctgcacgaggacggccggaagttcggcctgggctccagcggcgcggtgaccgtggcgaccgtagccgccgtcgccgcgttctgcggactcgaactgtccaccgacgaacggttccggctggccatgctcgccaccgcggaactcgaccccaagggctccggcggggacctcgccgccagcacctggggcggctggatcgcctaccaggcgcccgaccgggcctttgtgctcgacctggcccggcgcgtgggagtcgaccggacactgaaggcgccctggccggggcactcggtgcgccgactgccggcgcccaagggcctcaccctggaggtcggctggaccggagagcccgcctccaccgcgtccctggtgtccgatctgcaccgccgcacctggcggggcagcgcctcccaccagaggttcgtcgagaccacgaccgactgtgtccgctccgcggtcaccgccctggagtccggcgacgacacgagcctgctgcacgagatccgccgggcccgccaggagctggcccgcctggacgacgaggtcggcctcggcatcttcacacccaagctgacggcgctgtgcgacgccgccgaagccgtcggcggcgcggccaagccctccggggcaggcggcggcgactgcggcatcgccctgctggacgccgaggcgtcgcgggacatcacacatgtacggcaacggtgggagacagccggggtgctgcccctgcccctgactcctgccctggaagggatctaagaatgaccagcgcccaacgcaaggacgaccacgtacggctcgccatcgagcagcacaacgcccacagcggacgcaaccagttcgacgacgtgtcgttcgtccaccacgccctggccggcatcgaccggccggacgtgtccctggccacgtccttcgccgggatctcctggcaggtgccgatctacatcaacgcgatgaccggcggcagcgagaagaccggcctcatcaaccgggacctggccaccgccgcccgcgagaccggcgtccccatcgcgtccgggtccatgaacgcgtacatcaaggacccctcctgcgccgacacgttccgtgtgctgcgcgacgagaaccccaacgggttcgtcatcgcgaacatcaacgccaccacgacggtcgacaacgcgcagcgcgcgatcgacctgatcgaggcgaacgccctgcagatccacatcaacacggcgcaggagacgccgatgccggagggcgaccggtcgttcgcgtcctgggtcccgcagatcgagaagatcgcggcggccgtcgacatccccgtgatcgtcaaggaggtcggcaacggcctgagccggcagaccatcctgctgctcgccgacctcggcgtgcaggcggcggacgtcagcggccgcggcggcacggacttcgcccgcatcgagaacggccgccgggagctcggcgactacgcgttcctgcacggctgggggcagtccaccgccgcctgcctgctggacgcccaggacatctccctgcccgtcctcgcctccggcggtgtgcgtcacccgctcgacgtggtccgcgccctcgcgctcggcgcccgcgccgtcggctcctccgccggcttcctgcgcaccctgatggacgacggcgtcgacgcgctgatcacgaagctcacgacctggctggaccagctggcggcgctgcagaccatgctcggcgcgcgcaccccggccgacctcacccgctgcgacgtgctgctccacggcgagctgcgtgacttctgcgccgaccggggcatcgacacgcgccgcctcgcccagcgctccagctccatcgaggccctccagacgacgggaagcacacgatgacggaaacgcacgccatagccggggtcccgatgaggtgggtgggaccccttcgtatttccgggaacgtcgccgagaccgagacccaggtcccgctcgccacgtacgagtcgccgctgtggccgtcggtgggccgcggggcgaaggtctcccggctgacggagaagggcatcgtcgccaccctcgtcgacgagcggatgacccgctcggtgatcgtcgaggcgacggacgcgcagaccgcgtacatggccgcgcagaccatccacgcccgcatcgacgagctgcgcgaggtggtgcgcggctgcagccggttcgcccagctgatcaacatcaagcacgagatcaacgcgaacctgctgttcatccggttcgagttcaccaccggtgacgcctccggccacaacatggccacgctcgcctccgatgtgctcctggggcacctgctggagacgatccctggcatctcctacggctcgatctccggcaactactgcacggacaagaaggccaccgcgatcaacggcatcctcggccgcggcaagaacgtgatcaccgagctgctggtgccgcgggacgtcgtcgagaacaacctgcacaccacggctgccaagatcgtcgagctgaacatccgcaagaacctgctcggcaccctgctcgccggcggcatccgctcggccaacgcccacttcgcgaacatgctgctcggcttctacctggccaccggccaggacgccgccaacatcgtcgagggctcgcagggcgtcgtcatggccgaggaccgcgacggcgacctctacttcgcctgcaccctgccgaacctgatcgtcggcacggtcggcaacggcaagggtctcggcttcgtggagacgaacctcgcccggctcggctgccgagccgaccgcgaacccggggagaacgcccgccgcctcgccgtcatcgcggcagcgaccgtgctgtgcggtgaactctcgctgctcgcggcacagacgaacccgggcgaactcatgcgcgcgcacgtccagctggaacgcgacaacaagaccgcaaaggttggtgcatagggcatgtccatctccataggcattcacgacctgtcgttcgccacaaccgagttcgtcctgccgcacacggcgctcgccgagtacaacggcaccgagatcggcaagtaccacgtcggcatcggccagcagtcgatgagcgtgccggccgccgacgaggacatcgtgaccatggccgcgaccgcggcgcggcccatcatcgagcgcaacggcaagagccggatccgcacggtcgtgttcgccacggagtcgtcgatcgaccaggcgaaggcgggcggcgtgtacgtgcactccctgctggggctggagtcggcctgccgggtcgtcgagctgaagcaggcctgctacggggccaccgccgcccttcagttcgccatcggcctggtgcggcgcgaccccgcccagcaggtcctggtcatcgccagtgacgtctccaagtacgagctggacagccccggcgaggcgacccagggcgcggccgcggtggccatgctggtcggcgccgacccggccctgctgcgtatcgaggagccgtcgggcctgttcaccgccgacgtcatggacttctggcggcccaactacctcaccaccgctctggtcgacggccaggagtccatcaacgcctacctgcaggccgtcgagggcgcctggaaggactacgcggagcaggacggccggtcgctggaggagttcgcggcgttcgtctaccaccagccgttcacgaagatggcctacaaggcgcaccgccacctgctgaacttcaacggctacgacaccgacaaggacgccatcgagggcgccctcggccagacgacggcgtacaacaacgtcatcggcaacagctacaccgcgtcggtgtacctgggcctggccgccctgctcgaccaggcggacgacctgacgggccgttccatcggcttcctgagctacggctcgggcagcgtcgccgagttcttctcgggcaccgtcgtcgccgggtaccgcgagcgtctgcgcaccgaggcgaaccaggaggcgatcgcccggcgcaagagcgtcgactacgccacctaccgcgagctgcacgagtacacgctcccgtccgacggcggcgaccacgccaccccggtgcagaccaccggccccttccggctggccgggatcaacgaccacaagcgcatctacgaggcgcgctagcgacacccctcggcaacggggtgcgccactgttcggcgcaccccgtgccgggctttcgcacagctattcacgaccatttgaggggcgggcagccgcatgaccgacgtccgattccgcattatcggtacgggtgcctacgta

SEQ ID NO: 58 - DNA, 7693 bp, Artificial
  Operon containing A. thaliana and S. cerevisiae DNA
ggccgcgtcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggtcctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaattgcgaaaccgttttatgatgctaaagtgcaaccaacgactttaataccaaaggaagtcggtaacatgtacactgcttctctctacgctgcatttgcttccctcatccacaagaaacacaatgatttggcgggaaagcgggtggttatgttctcttatggaagtggctcaaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgagcacgtggaggcacatatgcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatgacccgggatccggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcacaggaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaataact

SEQ ID NO: 59 - DNA, 7695 bp, Artificial
  Operon B containing A. thaliana and S. cerevisiae DNA
ggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgagcacgtggaggcacatatgcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatgacccgggatccg

SEQ ID NO: 60 - DNA, 8235 bp, Artificial
  Operon C containing A. thaliana, S. cerevisiae, and R. caosulatus DNA
ggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgagcacgtggaggcacatatgcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatgacccgtaaggaggcacatatgagtgagcttatacccgcctgggttggtgacagactggctccggtggacaagttggaggtgcatttgaaagggctccgccacaaggcggtgtctgttttcgtcatggatggcgaaaacgtgctgatccagcgccgctcggaggagaaatatcactctcccgggctttgggcgaacacctgctgcacccatccgggctggaccgaacgccccgaggaatgcgcggtgcggcggctgcgcgaggagctggggatcaccgggctttatcccgcccatgccgaccggctggaatatcgcgccgatgtcggcggcggcatgatcgagcatgaggtggtcgacatctatctggcctatgccaaaccgcatatgcggatcacccccgatccgcgcgaagtggccgaggtgcgctggatcggcctttacgatctggcggccgaggccggtcggcatcccgagcggttctcgaaatggctcaacatctatctgtcgagccatcttgaccggattttcggatcgatcctgcgcggctgagcg

SEQ ID NO: 61 - DNA, 7681 bp, Artificial
  Operon C containing A. thaliana, S. cerevisiae, and Streptomyces sp CL190 DNA, and R. capsulatus DNA
ggccgcgtcgactacggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgagtaggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgactcgagcacgtgaggaggcacatatgacggaaacgcacgccatagccggggtcccgatgaggtgggtgggaccccttcgtatttccgggaacgtcgccgagaccgagacccaggtcccgctcgccacgtacgagtcgccgctgtggccgtcggtgggccgcggggcgaaggtctcccggctgacggagaagggcatcgtcgccaccctcgtcgacgagcggatgacccgctcggtgatcgtcgaggcgacggacgcgcagaccgcgtacatggccgcgcagaccatccacgcccgcatcgacgagctgcgcgaggtggtgcgcggctgcagccggttcgcccagctgatcaacatcaagcacgagatcaacgcgaacctgctgttcatccggttcgagttcaccaccggtgacgcctccggccacaacatggccacgctcgcctccgatgtgctcctggggcacctgctggagacgatccctggcatctcctacggctcgatctccggcaactactgcacggacaagaaggccaccgcgatcaacggcatcctcggccgcggcaagaacgtgatcaccgagctgctggtgccgcgggacgtcgtcgagaacaacctgcacaccacggctgccaagatcgtcgagctgaacatccgcaagaacctgctcggcaccctgctcgccggcggcatccgctcggccaacgcccacttcgcgaacatgctgctcggcttctacctggccaccggccaggacgccgccaacatcgtcgagggctcgcagggcgtcgtcatggccgaggaccgcgacggcgacctctacttcgcctgcaccctgccgaacctgatcgtcggcacggtcggcaacggcaagggtctcggcttcgtggagacgaacctcgcccggctcggctgccgagccgaccgcgaacccggggagaacgcccgccgcctcgccgtcatcgcggcagcgaccgtgctgtgcggtgaactctcgctgctcgcggcacagacgaacccgggcgaactcatgcgcgcgcacgtccagctggaacgcgacaacaagaccgcaaaggttggtgcatagacgcgtgcg

SEQ ID NO: 62 - DNA, 8224 bp, Artificial
  Operon E containing A. thaliana, S. cerevesiae, Steptomyces sp CL190 DNA, and R. capsulatus
ggccgcgtcgactacggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgagtaggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgactcgagcacgtgaggaggcacatatgacggaaacgcacgccatagccggggtcccgatgaggtgggtgggaccccttcgtatttccgggaacgtcgccgagaccgagacccaggtcccgctcgccacgtacgagtcgccgctgtggccgtcggtgggccgcggggcgaaggtctcccggctgacggagaagggcatcgtcgccaccctcgtcgacgagcggatgacccgctcggtgatcgtcgaggcgacggacgcgcagaccgcgtacatggccgcgcagaccatccacgcccgcatcgacgagctgcgcgaggtggtgcgcggctgcagccggttcgcccagctgatcaacatcaagcacgagatcaacgcgaacctgctgttcatccggttcgagttcaccaccggtgacgcctccggccacaacatggccacgctcgcctccgatgtgctcctggggcacctgctggagacgatccctggcatctcctacggctcgatctccggcaactactgcacggacaagaaggccaccgcgatcaacggcatcctcggccgcggcaagaacgtgatcaccgagctgctggtgccgcgggacgtcgtcgagaacaacctgcacaccacggctgccaagatcgtcgagctgaacatccgcaagaacctgctcggcaccctgctcgccggcggcatccgctcggccaacgcccacttcgcgaacatgctgctcggcttctacctggccaccggccaggacgccgccaacatcgtcgagggctcgcagggcgtcgtcatggccgaggaccgcgacggcgacctctacttcgcctgcaccctgccgaacctgatcgtcggcacggtcggcaacggcaagggtctcggcttcgtggagacgaacctcgcccggctcggctgccgagccgaccgcgaacccggggagaacgcccgccgcctcgccgtcatcgcggcagcgaccgtgctgtgcggtgaactctcgctgctcgcggcacagacgaacccgggcgaactcatgcgcgcgcacgtccagctggaacgcgacaacaagaccgcaaaggttggtgcatagacgcggtaaggaggcacatatgagtgagcttatacccgcctgggttggtgacagactggctccggtggacaagttggaggtgcatttgaaagggctccgccacaaggcggtgtctgttttcgtcatggatggcgaaaacgtgctgatccagcgccgctcggaggagaaatatcactctcccgggctttgggcgaacacctgctgcacccatccgggctggaccgaacgccccgaggaatgcgcggtgcggcggctgcgcgaggagctggggatcaccgggctttatcccgcccatgccgaccggctggaatatcgcgccgatgtcggcggcggcatgatcgagcatgaggtggtcgacatctatctggcctatgccaaaccgcatatgcggatcacccccgatccgcgcgaagtggccgaggtgcgctggatcggcctttacgatctggcggccgaggccggtcggcatcccgagcggttctcgaaatggctcaacatctatctgtcgagccatcttgaccggattttcggatcgatcctgcgcggctgagc

SEQ ID NO: 63 - DNA, 8077 bp, Artificial
  Operon F containing A. thaliana, S. cerevisiae, and Streptomyces sp CL190 DNA
ccaccgcggcggccgcgtcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctaggtacttccctggcgtgtgcagcggttgacgcgccgtgccctcgctgcgagcggcgcgcacatctgacgtcctgctttattgctttctcagaactcgggacgaagcgatcccatgatcacgcgatctccatgcagaaaagacaaagggagctgagtgcgttgacactaccgacctcggctgagggggtatcagaaagccaccgggcccgctcggtcggcatcggtcgcgcccacgccaaggccatcctgctgggagagcatgcggtcgtctacggagcgccggcactcgctctgccgattccgcagctcacggtcacggccagcgtcggctggtcgtccgaggcctccgacagtgcgggtggcctgtcctacacgatgaccggtacgccgtcgcgggcactggtgacgcaggcctccgacggcctgcaccggctcaccgcggaattcatggcgcggatgggcgtgacgaacgcgccgcacctcgacgtgatcctggacggcgcgatcccgcacggccggggtctcggctccagcgcggccggctcacgcgcgatcgccttggccctcgccgacctcttcggccacgaactggccgagcacacggcgtacgaactggtgcagacggccgagaacatggcgcacggccgggccagcggcgtggacgcgatgacggtcggcgcgtcccggccgctgctgttccagcagggccgcaccgagcgactggccatcggctgcgacagcctgttcatcgtcgccgacagcggcgtcccgggcagcaccaaggaagcggtcgagatgctgcgggagggattcacccgcagcgccggaacacaggagcggttcgtcggccgggcgacggaactgaccgaggccgcccggcaggccctcgccgacggccggcccgaggagctgggctcgcagctgacgtactaccacgagctgctccatgaggcccgcctgagcaccgacggcatcgatgcgctggtcgaggccgcgctgaaggcaggcagcctcggagccaagatcaccggcggtggtctgggcggctgcatgatcgcacaggcccggcccgaacaggcccgggaggtcacccggcagctccacgaggccggtgccgtacagacctgggtcgtaccgctgaaagggctcgacaaccatgcgcagtgaacacccgaccacgaccgtgctccagtcgcgggagcagggcagcgcggccggcgccaccgcggtcgcgcacccaaacatcgcgctgatcaagtactggggcaagcgcgacgagcggctgatcctgccctgcaccaccagcctgtcgatgacgctggacgtcttccccacgaccaccgaggtccggctcgaccccgccgccgagcacgacacggccgccctcaacggcgaggtggccacgggcgagacgctgcgccgcatcagcgccttcctctccctggtgcgggaggtggcgggcagcgaccagcgggccgtggtggacacccgcaacaccgtgcccaccggggcgggcctggcgtcctccgccagcgggttcgccgccctcgccgtcgcggccgcggccgcctacgggctcgaactcgacgaccgcgggctgtcccggctggcccgacgtggatccggctccgcctcgcggtcgatcttcggcggcttcgccgtctggcacgccggccccgacggcacggccacggaagcggacctcggctcctacgccgagccggtgcccgcggccgacctcgacccggcgctggtcatcgccgtggtcaacgccggccccaagcccgtctccagccgcgaggccatgcgccgcaccgtcgacacctcgccgctgtaccggccgtgggccgactccagtaaggacgacctggacgagatgcgctcggcgctgctgcgcggcgacctcgaggccgtgggcgagatcgcggagcgcaacgcgctcggcatgcacgccaccatgctggccgcccgccccgcggtgcggtacctgtcgccggccacggtcaccgtgctcgacagcgtgctccagctccgcaaggacggtgtcctggcctacgcgaccatggacgccggtcccaacgtgaaggtgctgtgccggcgggcggacgccgagcgggtggccgacgtcgtacgcgccgccgcgtccggcggtcaggtcctcgtcgccgggccgggagacggtgcccgcctgctgagcgagggcgcatgacgacaggtcagcgcacgatcgtccggcacgcgccgggcaagctgttcgtcgcgggcgagtacgcggtcgtggatccgggcaacccggcgatcctggtagcggtcgaccggcacatcagcgtcaccgtgtccgacgccgacgcggacaccggggccgccgacgtcgtgatctcctccgacctcggtccgcaggcggtcggctggcgctggcacgacggccggctcgtcgtccgcgacccggacgacgggcagcaggcgcgcagcgccctggcccacgtggtgtcggcgatcgagaccgtgggccggctgctgggcgaacgcggacagaaggtccccgctctcaccctctccgtcagcagccgcctgcacgaggacggccggaagttcggcctgggctccagcggcgcggtgaccgtggcgaccgtagccgccgtcgccgcgttctgcggactcgaactgtccaccgacgaacggttccggctggccatgctcgccaccgcggaactcgaccccaagggctccggcggggacctcgccgccagcacctggggcggctggatcgcctaccaggcgcccgaccgggcctttgtgctcgacctggcccggcgcgtgggagtcgaccggacactgaaggcgccctggccggggcactcggtgcgccgactgccggcgcccaagggcctcaccctggaggtcggctggaccggagagcccgcctccaccgcgtccctggtgtccgatctgcaccgccgcacctggcggggcagcgcctcccaccagaggttcgtcgagaccacgaccgactgtgtccgctccgcggtcaccgccctggagtccggcgacgacacgagcctgctgcacgagatccgccgggcccgccaggagctggcccgcctggacgacgaggtcggcctcggcatcttcacacccaagctgacggcgctgtgcgacgccgccgaagccgtcggcggcgcggccaagccctccggggcaggcggcggcgactgcggcatcgccctgctggacgccgaggcgtcgcgggacatcacacatgtacggcaacggtgggagacagccggggtgctgcccctgcccctgactcctgccctggaagggatctaagaatgaccagcgcccaacgcaaggacgaccacgtacggctcgccatcgagcagcacaacgcccacagcggacgcaaccagttcgacgacgtgtcgttcgtccaccacgccctggccggcatcgaccggccggacgtgtccctggccacgtccttcgccgggatctcctggcaggtgccgatctacatcaacgcgatgaccggcggcagcgagaagaccggcctcatcaaccgggacctggccaccgccgcccgcgagaccggcgtccccatcgcgtccgggtccatgaacgcgtacatcaaggacccctcctgcgccgacacgttccgtgtgctgcgcgacgagaaccccaacgggttcgtcatcgcgaacatcaacgccaccacgacggtcgacaacgcgcagcgcgcgatcgacctgatcgaggcgaacgccctgcagatccacatcaacacggcgcaggagacgccgatgccggagggcgaccggtcgttcgcgtcctgggtcccgcagatcgagaagatcgcggcggccgtcgacatccccgtgatcgtcaaggaggtcggcaacggcctgagccggcagaccatcctgctgctcgccgacctcggcgtgcaggcggcggacgtcagcggccgcggcggcacggacttcgcccgcatcgagaacggccgccgggagctcggcgactacgcgttcctgcacggctgggggcagtccaccgccgcctgcctgctggacgcccaggacatctccctgcccgtcctcgcctccggcggtgtgcgtcacccgctcgacgtggtccgcgccctcgcgctcggcgcccgcgccgtcggctcctccgccggcttcctgcgcaccctgatggacgacggcgtcgacgcgctgatcacgaagctcacgacctggctggaccagctggcggcgctgcagaccatgctcggcgcgcgcaccccggccgacctcacccgctgcgacgtgctgctccacggcgagctgcgtgacttctgcgccgaccggggcatcgacacgcgccgcctcgcccagcgctccagctccatcgaggccctccagacgacgggaagcacacgatgacggaaacgcacgccatagccggggtcccgatgaggtgggtgggaccccttcgtatttccgggaacgtcgccgagaccgagacccaggtcccgctcgccacgtacgagtcgccgctgtggccgtcggtgggccgcggggcgaaggtctcccggctgacggagaagggcatcgtcgccaccctcgtcgacgagcggatgacccgctcggtgatcgtcgaggcgacggacgcgcagaccgcgtacatggccgcgcagaccatccacgcccgcatcgacgagctgcgcgaggtggtgcgcggctgcagccggttcgcccagctgatcaacatcaagcacgagatcaacgcgaacctgctgttcatccggttcgagttcaccaccggtgacgcctccggccacaacatggccacgctcgcctccgatgtgctcctggggcacctgctggagacgatccctggcatctcctacggctcgatctccggcaactactgcacggacaagaaggccaccgcgatcaacggcatcctcggccgcggcaagaacgtgatcaccgagctgctggtgccgcgggacgtcgtcgagaacaacctgcacaccacggctgccaagatcgtcgagctgaacatccgcaagaacctgctcggcaccctgctcgccggcggcatccgctcggccaacgcccacttcgcgaacatgctgctcggcttctacctggccaccggccaggacgccgccaacatcgtcgagggctcgcagggcgtcgtcatggccgaggaccgcgacggcgacctctacttcgcctgcaccctgccgaacctgatcgtcggcacggtcggcaacggcaagggtctcggcttcgtggagacgaacctcgcccggctcggctgccgagccgaccgcgaacccggggagaacgcccgccgcctcgccgtcatcgcggcagcgaccgtgctgtgcggtgaactctcgctgctcgcggcacagacgaacccgggcgaactcatgcgcgcgcacgtccagctggaacgcgacaacaagaccgcaaaggttggtgcatagggcatgtccatctccataggcattcacgacctgtcgttcgccacaaccgagttcgtcctgccgcacacggcgctcgccgagtacaacggcaccgagatcggcaagtaccacgtcggcatcggccagcagtcgatgagcgtgccggccgccgacgaggacatcgtgaccatggccgcgaccgcggcgcggcccatcatcgagcgcaacggcaagagccggatccgcacggtcgtgttcgccacggagtcgtcgatcgaccaggcgaaggcgggcggcgtgtacgtgcactccctgctggggctggagtcggcctgccgggtcgtcgagctgaagcaggcctgctacggggccaccgccgcccttcagttcgccatcggcctggtgcggcgcgaccccgcccagcaggtcctggtcatcgccagtgacgtctccaagtacgagctggacagccccggcgaggcgacccagggcgcggccgcggtggccatgctggtcggcgccgacccggccctgctgcgtatcgaggagccgtcgggcctgttcaccgccgacgtcatggacttctggcggcccaactacctcaccaccgctctggtcgacggccaggagtccatcaacgcctacctgcaggccgtcgagggcgcctggaaggactacgcggagcaggacggccggtcgctggaggagttcgcggcgttcgtctaccaccagccgttcacgaagatggcctacaaggcgcaccgccacctgctgaacttcaacggctacgacaccgacaaggacgccatcgagggcgccctcggccagacgacggcgtacaacaacgtcatcggcaacagctacaccgcgtcggtgtacctgggcctggccgccctgctcgaccaggcggacgacctgacgggccgttccatcggcttcctgagctacggctcgggcagcgtcgccgagttcttctcgggcaccgtcgtcgccgggtaccgcgagcgtctgcgcaccgaggcgaaccaggaggcgatcgcccggcgcaagagcgtcgactacgccacctaccgcgagctgcacgagtacacgctcccgtccgacggcggcgaccacgccaccccggtgcagaccaccggccccttccggctggccgggatcaacgaccacaagcgcatctacgaggcgcgctagcgacacccctcggcaacggggtgcgccactgttcggcgcaccccgtgccgggctttcgcacagctattcacgaccatttgaggggcgggcagccgcatgaccgacgtccgattccgcattatcggtacgggtgcctacctagaactagtggatcccccgggctgcaggaattcgata

SEQ ID NO: 64 - DNA, 8400 bp, Artificial
  Operon G containing A. thaliana, S. cerevisiae, and S. pombe DNA
ggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgagcacgtggaggcacatatgcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatgacccgtaggaggcacatatgagttcccaacaagagaaaaaggattatgatgaagaacaattaaggttgatggaagaagtttgtatcgttgtagatgaaaatgatgtccctttaagatatggaacgaaaaaggagtgtcatttgatggaaaatataaataaaggtcttttgcatagagcattctctatgttcatctttgatgagcaaaatcgccttttacttcagcagcgtgcagaagagaaaattacatttccatccttatggacgaatacatgttgctcccacccattggatgttgctggtgaacgtggtaatactttacctgaagctgttgaaggtgttaagaatgcagctcaacgcaagctgttccatgaattgggtattcaagccaagtatattcccaaagacaaatttcagtttcttacacgaatccattaccttgctcctagtactggtgcttggggagagcatgaaattgactacattcttttcttcaaaggtaaagttgagctggatatcaatcccaatgaagttcaagcctataagtatgttactatggaagagttaaaagagatgttttccgatcctcaatatggattcacaccatggttcaaacttatttgtgagcattttatgtttaaatggtggcaggatgtagatcatgcgtcaaaattccaagataccttaattcatcgttgctaaggatcccccgggatcc

SEQ ID NO: 65 - DNA, 55 bp, Rhodobacter capsulatus
gcgatatcggatccaggaggaccatatgatcgccgaagcggatatggaggtctgc

SEQ ID NO: 66 - DNA, 50 bp, Rhodobacter capsulatus
gcgatatcaagcttggatcctcaatccatcgccaggccgcggtcgcgcgc

SEQ ID NO: 67 - DNA, 60 bp, Artificial
  Oligonucleotide containing N. tabacum and R. caopsulatus DNA
ctttcctgaaacataatttataatcagatccaggaggaccatatgatcgccgaagcggat

SEQ ID NO: 68 - DNA, 60 bp, Artificial
  Oligonucleotide containing N. tabacum and R. capsulatus DNA
cgaccgcggcctggcgatggattgaggatctaaacaaacccggaacagaccgttgggaag

SEQ ID NO: 69 - DNA, 60 bp, Artificial
  Oligonucleotide containing N. tabacum and R. capsulatus DNA
atttttcatctcgaattgtattcccacgaaggccgcgtcgactacggccgcaggaggagt

SEQ ID NO: 70 - DNA, 60 bp, Artificial
  Oligonucleotide containing N. tabacum and R. capsulatus DNA
ttcggatcgatcctgcgcggctgagcggccggaatggtgaagttgaaaaacgaatccttc

SEQ ID NO: 71 - DNA, 1020 bp, Rhodobacter capsulatus
atgatcgccgaagcggatatggaggtctgccgggagctgatccgcaccggcagctactccttccatgcggcgtccagagttctgccggcgcgggtccgtgaccccgcgctggcgctttacgccttttgccgcgtcgccgatgacgaagtcgacgaggttggcgcgccgcgcgacaaggctgcggcggttttgaaacttggcgaccggctggaggacatctatgccggtcgtccgcgcaatgcgccctcggatcgggctttcgcggcggtggtcgaggaattcgagatgccgcgcgaattgcccgaggcgctgctggagggcttcgcctgggatgccgaggggcggtggtatcacacgctttcggacgtgcaggcctattcggcgcgggtggcggccgccgtcggcgcgatgatgtgcgtgctgatgcgggtgcgcaaccccgatgcgctggcgcgggcctgcgatctcggtcttgccatgcagatgtcgaacatcgcccgcgacgtgggcgaggatgcccgggcggggcggcttttcctgccgaccgactggatggtcgaggaggggatcgatccgcaggcgttcctggccgatccgcagcccaccaagggcatccgccgggtcaccgagcggttgctgaaccgcgccgaccggctttactggcgggcggcgacgggggtgcggcttttgccctttgactgccgaccggggatcatggccgcgggcaagatctatgccgcgatcggggccgaggtggcgaaggcgaaatacgacaacatcacccggcgtgcccacacgaccaagggccgcaagctgtggctggtggcgaattccgcgatgtcggcgacggcgacctcgatgctgccgctctcgccgcgggtgcatgccaagcccgagcccgaagtggcgcatctggtcgatgccgccgcgcatcgcaacctgcatcccgaacggtccgaggtgctgatctcggcgctgatggcgctgaaggcgcgcgaccgcggcctggcgatggattga

SEQ ID NO: 72 - DNA, 13917 bp, Artificial
  Plastid transformation vector pHKO4, containing Operon B, containing A. thaliana and S. cerevisiae DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcttcttggctgttattcaaaaggtccaacaatgtatatatattggacattttgaggcaattatagatcctggaaggcaattctgattggtcaataaaaatcgatttcaatgctattttttttttgttttttatgagtttagccaatttatcatgaaaggtaaaaggggataaaggaaccgtgtgttgattgtcctgtaaatataagttgtcttcctccatatgtaaaaagggaataaataaatcaattaaatttcgggatgcttcatgaagtgcttctttcggagttaaacttccgtttgtccatatttcgagaaaaagtatctcttgtttttcattcccattcccataagaatgaatactatgattcgcgtttcgaacaggcatgaatacagcatctataggataacttccatcttgaaagttatgtggcgtttttataagatatccacgatttctctctatttgtaatccaatacaaaaatcaattggttccgttaaactggctatatgttgtgtattatcaacgatttctacataaggcggcaagatgatatcttgggcagttacagatccaggacccttgacacaaatagatgcgtcagaagttccatatagattacttcttaatataatttctttcaaattcattaaaatttcatgtaccgattcttgaatgcccgttatggtagaatattcatgtgggactttctcagattttacacgtgtgatacatgttccttctatttctccaagtaaagctcttcgcatcgcaatgcctattgtgtcggcttggcctttcataagtggagacagaataaagcgtccataataaaggcgtttactgtctgttcttgattcaacacacttccactgtagtgtccgagtagatactgttactttctctcgaaccatagtactattatttgattagatcatcgaatcttttatttctcttgagatttcttcaatgttcagttctacacacgtctttttttcggaggtctacagccattatgtggcataggagttacatcccgtacgaaagttaatagtataccacttcgacgaatagctcgtaatgctgcatctcttccgagaccgggaccttttatcatgacttctgctcgttgcataccttgatccactactgtacggatagcgtttgctgctgcggtttgagcagcaaacggtgttcctcttctcgtacctttgaatccagaagtaccggcggaggaccaagaaactactcgaccccgtacatctgtaacagtgacaatggtattattgaaacttgcttgaacatgaataactccctttggtattctacgtgcacccttacgtgaaccaatacgtccattcctacgcgaactaattttcggtatagcttttgccatattttatcatctcgtaaatatgagtcagagatatatggatatatccatttcatgtcaaaacagattctttatttgtacatcggctcttctggcaagtctgattatccctgtctttgtttatgtctcgggttggaacaaattactataattcgtccccgcctacggattagtcgacatttttcacaaattttacgaacggaagctcttattttcatatttctcattccttaccttaattctgaatctatttcttggaagaaaataagtttcttgaaatttttcatctcgaattgtattcccacgaaaggaatggtgaagttgaaaaacgaatccttcaaatctttgttgtggagtcgataaattatacgccctttggttgaatcataaggacttacttcaattttgactctatctcctggcagtatccgtataaaactatgccggatctttcctgaaacataatttataatcagatcggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgagcacgtggaggcacatatgcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatgacccgggatccggccgatctaaacaaacccggaacagaccgttgggaagcgattcagtaattaaagcttcatgactcctttttggttcttaaagtccctttgaggtatcaactaataagaaagatattagacaaccccccttttttctttttcacaaataggaagtttcgaatccaatttggatattaaaaggattaccagatataacacaaaatctctccacctattccttctagtcgagcctctcggtctgtcattatacctcgagaagtagaaagaattacaatccccattccacctaaaattcgcggaattcgttgataattagaatagattcgtagaccaggtcgactgattcgttttaaatttaaaatatttctatagggtcttttcctattccttctatgtcgcagggttaaaaccaaaaaatatttgtttttttctcgatgttttctcacgttttcgataaaaccttctcgtaaaagtatttgaacaatattttcggtaatattagtagatgctattcgaaccaccctttttcgatccatatcagcatttcgtatagaagttattatctcagcaatagtgtccctacccatgatgaactaaaattattggggcctccaaatttgatataatcaacgtgttttttacttattttttttttgaatatgatatgaattattaaagatatatgcgtgagacacaatctactaattaatctatttctttcaaataccccactagaaacagatcacaatttcattttataatacctcgggagctaatgaaactattttagtaaaatttaattctctcaattcccgggcgattgcaccaaaaattcgagttccttttgatttccttccttcttgatcaataacaactgcagcattgtcatcatatcgtattatcatcccgttgtcacgtttgagttctttacaggtccgcacaattacagctctgactacttctgatctttctaggggcatatttggtacggcttctttgatcacagcaacaataacgtcaccaatatgagcatatcgacgattgctagctcctatgattcgaatacacatcaattctcgagccccgctgttatccgctacatttaaatgggtctgaggttgaatcatttttttaatccgttctttgaatgcaaagggcgaagaaaaaaaagaaatatttttgtccaaaaaaaaagaaacatgcggtttcgtttcatatctaagagccctttccgcatttttttctattacattacgaaataatgaattgagttcgtataggcattttagatgctgctagtgaaatagcccttctggctatattttctgttactccacccatttcataaagtattcgacccggtttaacaacagctacccaatattcaggggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 73 - DNA, 7252 bp, Artificial
  Plastid transformation vector pHKO7, containing Operon C, containing A. thaliana, S. cerevisiae, and R. capsulatus DNA
gcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggccgctctagaactagtggatcttcttggctgttattcaaaaggtccaacaatgtatatatattggacattttgaggcaattatagatcctggaaggcaattctgattggtcaataaaaatcgatttcaatgctattttttttttgttttttatgagtttagccaatttatcatgaaaggtaaaaggggataaaggaaccgtgtgttgattgtcctgtaaatataagttgtcttcctccatatgtaaaaagggaataaataaatcaattaaatttcgggatgcttcatgaagtgcttctttcggagttaaacttccgtttgtccatatttcgagaaaaagtatctcttgtttttcattcccattcccataagaatgaatactatgattcgcgtttcgaacaggcatgaatacagcatctataggataacttccatcttgaaagttatgtggcgtttttataagatatccacgatttctctctatttgtaatccaatacaaaaatcaattggttccgttaaactggctatatgttgtgtattatcaacgatttctacataaggcggcaagatgatatcttgggcagttacagatccaggacccttgacacaaatagatgcgtcagaagttccatatagattacttcttaatataatttctttcaaattcattaaaatttcatgtaccgattcttgaatgcccgttatggtagaatattcatgtgggactttctcagattttacacgtgtgatacatgttccttctatttctccaagtaaagctcttcgcatcgcaatgcctattgtgtcggcttggcctttcataagtggagacagaataaagcgtccataataaaggcgtttactgtctgttcttgattcaacacacttccactgtagtgtccgagtagatactgttactttctctcgaaccatagtactattatttgattagatcatcgaatcttttatttctcttgagatttcttcaatgttcagttctacacacgtctttttttcggaggtctacagccattatgtggcataggagttacatcccgtacgaaagttaatagtataccacttcgacgaatagctcgtaatgctgcatctcttccgagaccgggaccttttatcatgacttctgctcgttgcataccttgatccactactgtacggatagcgtttgctgctgcggtttgagcagcaaacggtgttcctcttctcgtacctttgaatccagaagtaccggcggaggaccaagaaactactcgaccccgtacatctgtaacagtgacaatggtattattgaaacttgcttgaacatgaataactccctttggtattctacgtgcacccttacgtgaaccaatacgtccattcctacgcgaactaattttcggtatagcttttgccatattttatcatctcgtaaatatgagtcagagatatatggatatatccatttcatgtcaaaacagattctttatttgtacatcggctcttctggcaagtctgattatccctgtctttgtttatgtctcgggttggaacaaattactataattcgtccccgcctacggattagtcgacatttttcacaaattttacgaacggaagctcttattttcatatttctcattccttaccttaattctgaatctatttcttggaagaaaataagtttcttgaaatttttcatctcgaattgtattcccacgaaaggaatggtgaagttgaaaaacgaatccttcaaatctttgttgtggagtcgataaattatacgccctttggttgaatcataaggacttacttcaattttgactctatctcctggcagtatccgtataaaactatgccggatctttcctgaaacataatttataatcagatccaggaggaccatatgatcgccgaagcggatatggaggtctgccgggagctgatccgcaccggcagctactccttccatgcggcgtccagagttctgccggcgcgggtccgtgaccccgcgctggcgctttacgccttttgccgcgtcgccgatgacgaagtcgacgaggttggcgcgccgcgcgacaaggctgcggcggttttgaaacttggcgaccggctggaggacatctatgccggtcgtccgcgcaatgcgccctcggatcgggctttcgcggcggtggtcgaggaattcgagatgccgcgcgaattgcccgaggcgctgctggagggcttcgcctgggatgccgaggggcggtggtatcacacgctttcggacgtgcaggcctattcggcgcgggtggcggccgccgtcggcgcgatgatgtgcgtgctgatgcgggtgcgcaaccccgatgcgctggcgcgggcctgcgatctcggtcttgccatgcagatgtcgaacatcgcccgcgacgtgggcgaggatgcccgggcggggcggcttttcctgccgaccgactggatggtcgaggaggggatcgatccgcaggcgttcctggccgatccgcagcccaccaagggcatccgccgggtcaccgagcggttgctgaaccgcgccgaccggctttactggcgggcggcgacgggggtgcggcttttgccctttgactgccgaccggggatcatggccgcgggcaagatctatgccgcgatcggggccgaggtggcgaaggcgaaatacgacaacatcacccggcgtgcccacacgaccaagggccgcaagctgtggctggtggcgaattccgcgatgtcggcgacggcgacctcgatgctgccgctctcgccgcgggtgcatgccaagcccgagcccgaagtggcgcatctggtcgatgccgccgcgcatcgcaacctgcatcccgaacggtccgaggtgctgatctcggcgctgatggcgctgaaggcgcgcgaccgcggcctggcgatggattgaggatctaaacaaacccggaacagaccgttgggaagcgattcagtaattaaagcttcatgactcctttttggttcttaaagtccctttgaggtatcaactaataagaaagatattagacaaccccccttttttctttttcacaaataggaagtttcgaatccaatttggatattaaaaggattaccagatataacacaaaatctctccacctattccttctagtcgagcctctcggtctgtcattatacctcgagaagtagaaagaattacaatccccattccacctaaaattcgcggaattcgttgataattagaatagattcgtagaccaggtcgactgattcgttttaaatttaaaatatttctatagggtcttttcctattccttctatgtcgcagggttaaaaccaaaaaatatttgtttttttctcgatgttttctcacgttttcgataaaaccttctcgtaaaagtatttgaacaatattttcggtaatattagtagatgctattcgaaccaccctttttcgatccatatcagcatttcgtatagaagttattatctcagcaatagtgtccctacccatgatgaactaaaattattggggcctccaaatttgatataatcaacgtgttttttacttattttttttttgaatatgatatgaattattaaagatatatgcgtgagacacaatctactaattaatctatttctttcaaataccccactagaaacagatcacaatttcattttataatacctcgggagctaatgaaactattttagtaaaatttaattctctcaattcccgggcgattgcaccaaaaattcgagttccttttgatttccttccttcttgatcaataacaactgcagcattgtcatcatatcgtattatcatcccgttgtcacgtttgagttctttacaggtccgcacaattacagctctgactacttctgatctttctaggggcatatttggtacggcttctttgatcacagcaacaataacgtcaccaatatgagcatatcgacgattgctagctcctatgattcgaatacacatcaattctcgagccccgctgttatccgctacatttaaatgggtctgaggttgaatcatttttttaatccgttctttgaatgcaaagggcgaagaaaaaaaagaaatatttttgtccaaaaaaaaagaaacatgcggtttcgtttcatatctaagagccctttccgcatttttttctattacattacgaaataatgaattgagttcgtataggcattttagatgctgctagtgaaatagcccttctggctatattttctgttactccacccatttcataaagtattcgacccggtttaacaacagctacccaatattcaggggatcccccgggctgcaggaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtg

SEQ ID NO: 74 - DNA, 14623 bp, Artificial
  Plastid transformation vector pHKO8, containing Operon G, containing A. thaliana, S. cerevisiae, and S. pombe DNA
cacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattcctgcagcccgggggatcttcttggctgttattcaaaaggtccaacaatgtatatatattggacattttgaggcaattatagatcctggaaggcaattctgattggtcaataaaaatcgatttcaatgctattttttttttgttttttatgagtttagccaatttatcatgaaaggtaaaaggggataaaggaaccgtgtgttgattgtcctgtaaatataagttgtcttcctccatatgtaaaaagggaataaataaatcaattaaatttcgggatgcttcatgaagtgcttctttcggagttaaacttccgtttgtccatatttcgagaaaaagtatctcttgtttttcattcccattcccataagaatgaatactatgattcgcgtttcgaacaggcatgaatacagcatctataggataacttccatcttgaaagttatgtggcgtttttataagatatccacgatttctctctatttgtaatccaatacaaaaatcaattggttccgttaaactggctatatgttgtgtattatcaacgatttctacataaggcggcaagatgatatcttgggcagttacagatccaggacccttgacacaaatagatgcgtcagaagttccatatagattacttcttaatataatttctttcaaattcattaaaatttcatgtaccgattcttgaatgcccgttatggtagaatattcatgtgggactttctcagattttacacgtgtgatacatgttccttctatttctccaagtaaagctcttcgcatcgcaatgcctattgtgtcggcttggcctttcataagtggagacagaataaagcgtccataataaaggcgtttactgtctgttcttgattcaacacacttccactgtagtgtccgagtagatactgttactttctctcgaaccatagtactattatttgattagatcatcgaatcttttatttctcttgagatttcttcaatgttcagttctacacacgtctttttttcggaggtctacagccattatgtggcataggagttacatcccgtacgaaagttaatagtataccacttcgacgaatagctcgtaatgctgcatctcttccgagaccgggaccttttatcatgacttctgctcgttgcataccttgatccactactgtacggatagcgtttgctgctgcggtttgagcagcaaacggtgttcctcttctcgtacctttgaatccagaagtaccggcggaggaccaagaaactactcgaccccgtacatctgtaacagtgacaatggtattattgaaacttgcttgaacatgaataactccctttggtattctacgtgcacccttacgtgaaccaatacgtccattcctacgcgaactaattttcggtatagcttttgccatattttatcatctcgtaaatatgagtcagagatatatggatatatccatttcatgtcaaaacagattctttatttgtacatcggctcttctggcaagtctgattatccctgtctttgtttatgtctcgggttggaacaaattactataattcgtccccgcctacggattagtcgacatttttcacaaattttacgaacggaagctcttattttcatatttctcattccttaccttaattctgaatctatttcttggaagaaaataagtttcttgaaatttttcatctcgaattgtattcccacgaaaggaatggtgaagttgaaaaacgaatccttcaaatctttgttgtggagtcgataaattatacgccctttggttgaatcataaggacttacttcaattttgactctatctcctggcagtatccgtataaaactatgccggatctttcctgaaacataatttataatcagatcggccgcaggaggagttcatatgtcagagttgagagccttcagtgccccagggaaagcgttactagctggtggatatttagttttagatacaaaatatgaagcatttgtagtcggattatcggcaagaatgcatgctgtagcccatccttacggttcattgcaagggtctgataagtttgaagtgcgtgtgaaaagtaaacaatttaaagatggggagtggctgtaccatataagtcctaaaagtggcttcattcctgtttcgataggcggatctaagaaccctttcattgaaaaagttatcgctaacgtatttagctactttaaacctaacatggacgactactgcaatagaaacttgttcgttattgatattttctctgatgatgcctaccattctcaggaggatagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttagcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttgatgaagaagactggaatattacgattaaaagtaaccatttaccttcgggattaactttatggatgggcgatattaagaatggttcagaaacagtaaaactggtccagaaggtaaaaaattggtatgattcgcatatgccagaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgaaatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattactaagcaagatgttgatcttagggctcaaaccgctaatgacaaaagattttctaaggttcaatggctggatgtaactcaggctgactggggtgttaggaaagaaaaagatccggaaacttatcttgataaactgcaggaggagttttaatgtcattaccgttcttaacttctgcaccgggaaaggttattatttttggtgaacactctgctgtgtacaacaagcctgccgtcgctgctagtgtgtctgcgttgagaacctacctgctaataagcgagtcatctgcaccagatactattgaattggacttcccggacattagctttaatcataagtggtccatcaatgatttcaatgccatcaccgaggatcaagtaaactcccaaaaattggccaaggctcaacaagccaccgatggcttgtctcaggaactcgttagtcttttggatccgttgttagctcaactatccgaatccttccactaccatgcagcgttttgtttcctgtatatgtttgtttgcctatgcccccatgccaagaatattaagttttctttaaagtctactttacccatcggtgctgggttgggctcaagcgcctctatttctgtatcactggccttagctatggcctacttgggggggttaataggatctaatgacttggaaaagctgtcagaaaacgataagcatatagtgaatcaatgggccttcataggtgaaaagtgtattcacggtaccccttcaggaatagataacgctgtggccacttatggtaatgccctgctatttgaaaaagactcacataatggaacaataaacacaaacaattttaagttcttagatgatttcccagccattccaatgatcctaacctatactagaattccaaggtctacaaaagatcttgttgctcgcgttcgtgtgttggtcaccgagaaatttcctgaagttatgaagccaattctagatgccatgggtgaatgtgccctacaaggcttagagatcatgactaagttaagtaaatgtaaaggcaccgatgacgaggctgtagaaactaataatgaactgtatgaacaactattggaattgataagaataaatcatggactgcttgtctcaatcggtgtttctcatcctggattagaacttattaaaaatctgagcgatgatttgagaattggctccacaaaacttaccggtgctggtggcggcggttgctctttgactttgttacgaagagacattactcaagagcaaattgacagcttcaaaaagaaattgcaagatgattttagttacgagacatttgaaacagacttgggtgggactggctgctgtttgttaagcgcaaaaaatttgaataaagatcttaaaatcaaatccctagtattccaattatttgaaaataaaactaccacaaagcaacaaattgacgatctattattgccaggaaacacgaatttaccatggacttcagacgaggagttttaatgactgtatatactgctagtgtaactgctccggtaaatattgctactcttaagtattgggggaaaagggacacgaagttgaatctgcccaccaattcgtccatatcagtgactttatcgcaagatgacctcagaacgttgacctctgcggctactgcacctgagtttgaacgcgacactttgtggttaaatggagaaccacacagcatcgacaatgaaagaactcaaaattgtctgcgcgacctacgccaattaagaaaggaaatggaatcgaaggacgcctcattgcccacattatctcaatggaaactccacattgtctccgaaaataactttcctacagcagctggtttagcttcctccgctgctggctttgctgcattggtctctgcaattgctaagttataccaattaccacagtcaacttcagaaatatctagaatagcaagaaaggggtctggttcagcttgtagatcgttgtttggcggatacgtggcctgggaaatgggaaaagctgaagatggtcatgattccatggcagtacaaatcgcagacagctctgactggcctcagatgaaagcttgtgtcctagttgtcagcgatattaaaaaggatgtgagttccactcagggtatgcaattgaccgtggcaacctccgaactatttaaagaaagaattgaacatgtcgtaccaaagagatttgaagtcatgcgtaaagccattgttgaaaaagatttcgccacctttgcaaaggaaacaatgatggattccaactctttccatgccacatgtttggactctttccctccaatattctacatgaatgacacttccaagcgtatcatcagttggtgccacaccattaatcagttttacggagaaacaatcgttgcatacacgtttgatgcaggtccaaatgctgtgttgtactacttagctgaaaatgagtcgaaactctttgcatttatctataaattgtttggctctgttcctggatgggacaagaaatttactactgagcagcttgaggctttcaaccatcaatttgaatcatctaactttactgcacgtgaattggatcttgagttgcaaaaggatgttgccagagtgattttaactcaagtcggttcaggcccacaagaaacaaacgaatctttgattgacgcaaagactggtctaccaaaggaagaggagttttaactcgacgccggcggaggcacatatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgaggatcctctagatgcgcaggaggcacatatggcgaagaacgttgggattttggctatggatatctatttccctcccacctgtgttcaacaggaagctttggaagcacatgatggagcaagtaaagggaaatacactattggacttggccaagattgtttagctttttgcactgagcttgaagatgttatctctatgagtttcaatgcggtgacatcactttttgagaagtataagattgaccctaaccaaatcgggcgtcttgaagtaggaagtgagactgttattgacaaaagcaagtccatcaagaccttcttgatgcagctctttgagaaatgtggaaacactgatgtcgaaggtgttgactcgaccaatgcttgctatggtggaactgcagctttgttaaactgtgtcaattgggttgagagtaactcttgggatggacgttatggcctcgtcatttgtactgacagcgcggtttatgcagaaggacccgcaaggcccactggaggagctgcagcgattgctatgttgataggacctgatgctcctatcgttttcgaaagcaaattgagagcaagccacatggctcatgtctatgacttttacaagcccaatcttgctagcgagtacccggttgttgatggtaagctttcacagacttgctacctcatggctcttgactcctgctataaacatttatgcaacaagttcgagaagatcgagggcaaagagttctccataaatgatgctgattacattgttttccattctccatacaataaacttgtacagaaaagctttgctcgtctcttgtacaacgacttcttgagaaacgcaagctccattgacgaggctgccaaagaaaagttcaccccttattcatctttgacccttgacgagagttaccaaagccgtgatcttgaaaaggtgtcacaacaaatttcgaaaccgttttatgatgctaaagtgcaaccaacgactttaataccaaaggaagtcggtaacatgtacactgcttctctctacgctgcatttgcttccctcatccacaataaacacaatgatttggcgggaaagcgggtggttatgttctcttatggaagtggctccaccgcaacaatgttctcattacgcctcaacgacaataagcctcctttcagcatttcaaacattgcatctgtaatggatgttggcggtaaattgaaagctagacatgagtatgcacctgagaagtttgtggagacaatgaagctaatggaacataggtatggagcaaaggactttgtgacaaccaaggagggtattatagatcttttggcaccgggaacttattatctgaaagaggttgattccttgtaccggagattctatggcaagaaaggtgaagatggatctgtagccaatggacactgaggatccgtcgagcacgtggaggcacatatgcaatgctgtgagatgcctgttggatacattcagattcctgttgggattgctggtccattgttgcttgatggttatgagtactctgttcctatggctacaaccgaaggttgtttggttgctagcactaacagaggctgcaaggctatgtttatctctggtggcgccaccagtaccgttcttaaggacggtatgacccgagcacctgttgttcggttcgcttcggcgagacgagcttcggagcttaagtttttcttggagaatccagagaactttgatactttggcagtagtcttcaacaggtcgagtagatttgcaagactgcaaagtgttaaatgcacaatcgcggggaagaatgcttatgtaaggttctgttgtagtactggtgatgctatggggatgaatatggtttctaaaggtgtgcagaatgttcttgagtatcttaccgatgatttccctgacatggatgtgattggaatctctggtaacttctgttcggacaagaaacctgctgctgtgaactggattgagggacgtggtaaatcagttgtttgcgaggctgtaatcagaggagagatcgtgaacaaggtcttgaaaacgagcgtggctgctttagtcgagctcaacatgctcaagaacctagctggctctgctgttgcaggctctctaggtggattcaacgctcatgccagtaacatagtgtctgctgtattcatagctactggccaagatccagctcaaaacgtggagagttctcaatgcatcaccatgatggaagctattaatgacggcaaagatatccatatctcagtcactatgccatctatcgaggtggggacagtgggaggaggaacacagcttgcatctcaatcagcgtgtttaaacctgctcggagttaaaggagcaagcacagagtcgccgggaatgaacgcaaggaggctagcgacgatcgtagccggagcagttttagctggagagttatctttaatgtcagcaattgcagctggacagcttgtgagaagtcacatgaaatacaatagatccagccgagacatctctggagcaacgacaacgacaacaacaacaacatgacccgtaggaggcacatatgagttcccaacaagagaaaaaggattatgatgaagaacaattaaggttgatggaagaagtttgtatcgttgtagatgaaaatgatgtccctttaagatatggaacgaaaaaggagtgtcatttgatggaaaatataaataaaggtcttttgcatagagcattctctatgttcatctttgatgagcaaaatcgccttttacttcagcagcgtgcagaagagaaaattacatttccatccttatggacgaatacatgttgctcccacccattggatgttgctggtgaacgtggtaatactttacctgaagctgttgaaggtgttaagaatgcagctcaacgcaagctgttccatgaattgggtattcaagccaagtatattcccaaagacaaatttcagtttcttacacgaatccattaccttgctcctagtactggtgcttggggagagcatgaaattgactacattcttttcttcaaaggtaaagttgagctggatatcaatcccaatgaagttcaagcctataagtatgttactatggaagagttaaaagagatgttttccgatcctcaatatggattcacaccatggttcaaacttatttgtgagcattttatgtttaaatggtggcaggatgtagatcatgcgtcaaaattccaagataccttaattcatcgttgctaaggatcccccgggatccggccgatctaaacaaacccggaacagaccgttgggaagcgattcagtaattaaagcttcatgactcctttttggttcttaaagtccctttgaggtatcaactaataagaaagatattagacaaccccccttttttctttttcacaaataggaagtttcgaatccaatttggatattaaaaggattaccagatataacacaaaatctctccacctattccttctagtcgagcctctcggtctgtcattatacctcgagaagtagaaagaattacaatccccattccacctaaaattcgcggaattcgttgataattagaatagattcgtagaccaggtcgactgattcgttttaaatttaaaatatttctatagggtcttttcctattccttctatgtcgcagggttaaaaccaaaaaatatttgtttttttctcgatgttttctcacgttttcgataaaaccttctcgtaaaagtatttgaacaatattttcggtaatattagtagatgctattcgaaccaccctttttcgatccatatcagcatttcgtatagaagttattatctcagcaatagtgtccctacccatgatgaactaaaattattggggcctccaaatttgatataatcaacgtgttttttacttattttttttttgaatatgatatgaattattaaagatatatgcgtgagacacaatctactaattaatctatttctttcaaataccccactagaaacagatcacaatttcattttataatacctcgggagctaatgaaactattttagtaaaatttaattctctcaattcccgggcgattgcaccaaaaattcgagttccttttgatttccttccttcttgatcaataacaactgcagcattgtcatcatatcgtattatcatcccgttgtcacgtttgagttctttacaggtccgcacaattacagctctgactacttctgatctttctaggggcatatttggtacggcttctttgatcacagcaacaataacgtcaccaatatgagcatatcgacgattgctagctcctatgattcgaatacacatcaattctcgagccccgctgttatccgctacatttaaatgggtctgaggttgaatcatttttttaatccgttctttgaatgcaaagggcgaagaaaaaaaagaaatatttttgtccaaaaaaaaagaaacatgcggtttcgtttcatatctaagagccctttccgcatttttttctattacattacgaaataatgaattgagttcgtataggcattttagatgctgctagtgaaatagcccttctggctatattttctgttactccacccatttcataaagtattcgacccggtttaacaacagctacccaatattcaggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgc

SEQ ID NO: 75 - DNA, 7252 bp, Artificial; identical to SEQ ID NO: 73
  Plastid transformation vector pFHO5 containing R. capsulatus DNA encoding phytoene synthase

SEQ ID NO: 76 - DNA, 14623 bp, Artificial; identical to SEQ ID NO: 74
  Plastid transformation vector pFHO6, containing Operon E, containing A. thaliana, S. cerevisiae, Streptomycs sp CL190 DNA, and R.capsulatus DNA

SEQ ID NO: 77 - DNA, 24 bp, Artificial
  PCR primer containing Rhodobacter capsulatus DNA
gctcgatcatgccgttgttcacat

SEQ ID NO: 78 - DNA, 24 bp, Artificial
  PCR primer containing Rhodobacter capsulatus DNA
atctggcgatttcggtcttcgtga

SEQ ID NO: 79 - DNA, 31 bp, Artificial
  PCR primer containing Synechocystis DNA
ggttaattaatgcccccaccttaatcctggg

SEQ ID NO: 80 - DNA, 33 bp, Artificial
  PCR primer containing Synechocystis DNA
ctggcgcgccgattattaaaggaaaactgtggc

SEQ ID NO: 81 - DNA, 24 bp, Artificial
  PCR primer containing Escherichia coli DNA
gcagcattacgagctattcgtcga

SEQ ID NO: 82 - DNA, 20 bp, Artificial
  PCR primer containing Escherichia coli DNA
aggggataaaggaaccgtgt

SEQ ID NO: 83 - DNA, 22 bp, Artificial
  PCR primer containing Rhodobacter capsulatus DNA
tctcgagcctaggctagctcta

SEQ ID NO: 84 - DNA, 22 bp, Artificial
  PCR primer containing Rhodobacter capsulatus DNA
gtcaccggcggaaagatcatgt

SEQ ID NO: 85 - DNA, 22 bp, Artificial
  PCR primer containing Synechocystis DNA
attaatgcccccaccttaatcc

SEQ ID NO: 86 - DNA, 22 bp, Artificial
  PCR primer containing Synechocystis DNA
cggcgcgccgattattaaagga

SEQ ID NO: 87 - DNA, 30 bp, Artificial
  PCR primer containing Acinetobacter baumanii DNA
ggccggccgtttaaacctacatccgcttta

SEQ ID NO: 88 - DNA, 27 bp, Artificial
  PCR primer containing Acinetobacter baumanii DNA
cttaattaacctaggttgaatttataa

SEQ ID NO: 89 - DNA, 50 bp, Artificial
  PCR primer for detecting cDNA of Hantaan virus nucleoprotein
gtttaaacggcgcgcctcaatggtgatggtgatgatggagtttcaaaggc

SEQ ID NO: 90 - DNA, 46 bp, Artificial
  PCR primer for detecting cDNA of Hantaan virus nucleoprotein
gtttaaacttaattaaggagaggataaaatatggcaactatggagg

SEQ ID NO: 91 - DNA, 42 bp, Artificial
  PCR primer for detecting cDNA of Andes virus nucleoprotein
gtttaaacggcgcgcctcaatggtgatggtgatgatgaccgg

SEQ ID NO: 92 - DNA, 46 bp, Artificial
  PCR primer for detecting cDNA of Andes virus nucleoprotein
gtttaaacttaattaaggagaggataaaatatgagcaccctccaag

SEQ ID NO: 93 - DNA, 2858 bp, Rhodomonas salina
ctatatagctgatacaaataaagttagaatctctttactaaaagtttcggaggctttcgatcgatatttattagggttaatgacaattgataaagttctcttaatagtaatgttttcaattttaactttatgtaatattcctaattcaagctctttagcaattgcggacaatgaaacaaaagcggcacctaagcctgattgcacagcatttttaatagcttcgacagagtttaattccatttctatcttaaatcggccactatcaattccatttgttttcaacacttcgtcgataacttttctgattgttgattgtctatctaaagctataaaacgtagacgatataggtcttctttttgtatactatctaacttagcgaaaggatgtaatttgggtaaaattaagacgagttcatcttcagcataagaagttactttaagaatatttttcaattctggcggcacttgccctccgattatagcaaggtttatttgtccattggcaacactccaagaaattctccttgtcgaatgtacttgaagttggacagccacttgagggtatttttggcgaaataaacctattaatctgggcattaaataggttccagttgtttggctcgctcctactatgagagtgcctccctgtaaatttctgagatcttcaatagctctacatgtctcatcacacaaagacaaaattctatttccatatcttagtaaaatttcaccttcttctgtaaacataactcttctggttcctctatcaaagataggaacattaagttgtttttccaaattttggacatgtaaactaactgcgggttgtgatatatataaactttcagcagctttcttaaagctgccttctgcggaaattgctttgagaattcttaattgatcaagtgtaaaaggtaaatcagtcatatttttagataaaaaaatatatataataagtgcaatgaatgttagggatgtattggtataatgagttaatgcataattaacatttagtacaaatgaatgtaaaaaattaattagtaaaaaactaaatgtactaatttaatttttataaatacagaatatacagtgattagttataatatttaagaaagactacaagagggaacctgtaatggatatgcgacttttaatagttctttttcctgttctagctgcagcgtcttgggctggttttaatattgccagaatagccctaagacaattaagccgccttggatcaagataaagatcacgtattgtattagtactatagaaaagggtaaaaagaaaaattttctttttacccttttcgagtaatgtaaaaaatcttccttactctgttaatttatatgtataataaagtttgcgaatagtatatataattcaaaattacattcttcattattacaaaactactaaagataaaataaaataagaaattaataatatggcagtacctaagaaacgaacatctaagatgaaatctagatcacgacgctctgtatggatgaataaaagtaatatccaagctcaaagagctctttctttagctaaatctttagcaacaaacaacaacacaacatttgtttattcttcaagtaatagtgatgttgctgaagaataataagttttaaaaatcttacgtttgtaagatttttaaaacttatctattcaattgataaattattggttagcatatgtttataaagtaactgatcaaaggcaaaaagttactgatagggcaaaaatggtattggacaatctacaacaagttgtaacataaatattgttctactaaaaaaaaggctaaccatactgcaaataagattatgataatatcaacatttcttttttatttctataggttgtgaataaatgaaaaagcaaagcttaacacgactaaaatatgtagactgataatacaatctaaagcatttatgctggtatggattaagaacattggtacagtatttaatagcatttccaaaatgttagaggaacaagaagatcaaatttaggacaatcttaaagattatttagggatcactttcaacaaatctgtactatttatggagggataaaataccagaagcctgtttaacataacttttaactcgtttttggatgactttgaaaaagtttgcagattataacacaagcaaactgtatcacccctcaaaactaaaattattagataagattgttttcgatatagcgcttcctgtgataaccaaacagaaagaattaaacactatcagtctggtttagtggtcgcacatgtaaataacacagaagctcgaattattaatgctcaatagtccctagagtttatatttgcacagatctaagtttttacatattgcggtgatattttagaattgtaacaagacttgttagaaggcgcaaccactcgctaagaggaagtatggaaatctcatataagttaaaagtctggttatatcgtaatttaataagaaaaaattttgaagtatttatagatagattgtaatggctgtctaataaatacttcaattgtgagtaaaagtgaattttttcatcggagctaaagtttataactcttcattttgtaaaattcgacgaattggtaaattcgcaatcaaggcagtcatcctgctatcagtttttatcgagaattctaaactatcagtgtctaattcaacatatttagaaacaactactaatatttctgtgcgcatcttctctaaagtagaggcatttaatgttgagcgatcatgtgctaagactaatttaagtcttcgctttacattatctcgactgccagtattatttacaaacaaacgttcaaaaaattctgtaatcatacaatatttcca

SEQ ID NO: 94 - DNA, 5680 bp, Artificial
  Cryptomonas paramecium
caacactataggttaaataaaatttaacttcaggcatttatactttaatcatatttacctaaaaattcctcaaaaaactaaaaatgaaacaaatagccttgtcagacttattagaagcaggagtccactttggacaccaaactaacaaatggaacccaaaaatgctaccatacatctacgcagaaaaaaatgggacacatataataaatggtatagaatcagcaaaactattagctaaagcttgcaactttgtgaaagaagcttcgcaaaaagggaaaaaattcctatttataggaacaaaaaggcaagcaaccaatattgttgccagtgaagctgagaaatgtggcgcatactttatcaaccaacggtggctcggaggaacactaacaaactggcctattataaaatccagagttgaagcactaaaaagattagaaaaaaattccataggcccttccttagataaggtctcgaaaaaagaaataaccgcacacaaaaaaaatctagaaaaactaaataaaaaccttaaaggaataaaaaatatgaaacaactaccagatctagcaataattatagatcaacaaaaggaaatcacagcagttaaagaatgcataaaaatgaaaataccagtaatatgcttactagacacaaactgcgacccaagcctagcagacataccaataccagcaaatgatgactctgcaaaatctataaaactcatactagaaaaactatccgaatccatagtagaagggaaaaatagctatgcgaaaaaataactaaatcttcaatataaaaataattaaaaaaccataaaaatatgaacgagaaaacaactcctacagaaaaaattaaagaactaagaaatttatcatgcgccggaattatggactgcaagaaagcgctagcagacgcaaatgggcaactacagaaagctctagagatacttcaacaaaaaggattaacttccgcagacaagaaaacgacacgattggctaaagaaggattaatagagtgctatctacatacaggtggaaagcttggtgcaatcattgaaataaactgcgaaacagatttcgtcgcaaaacaacagaaattccaagacctagcaaaaaatatagcaatgcaaatagctgcatgcccggcagttaaatatattcgcacagaagatataccaaacgaagttataaatctagaaaaaaatattgagagctcaaaagacgacttaaacaaaaaaaccgaagatataaaagaaaaaataattacaaataggacgaaaaaaaaactagcagaattaaccttattaaaccaagcatacatcagagacccaagcataacagttgaggagcttattaaacaacacatagcaatcctaggagaaaatatacaaataagaaggtttcaacgttttgaattaggagaaacacaaaaacacaattaaaaactatacctatgcacgaaaaaaaaatagccgctgcaaccctattctgcaaagcccatgaaataaaagtgggaaatcatattccttgggaaatctttggacttcatatgaacggagaaacaatactaacttctttaatagttacagtacttatcatctctttctgcttattgactataaaaaatccaaaatatatccctggtaagatacaaaattttatagaatatatatatgaagcattagaagacatttccaacaaccaaattgggaaaaaagacagtgaaaaatgggtcccatacataagcaccttatgtatattcatctttgtttccaactggataggaatactaataccactaaaactaatcccggtacaaggagtcgagctaggatcaccaacaaatgacataaacactaccacagccctagcactcttaacttctgcaagctatttttacgcaggcctaaaaaaaaagggatggcgctactactacgggtattttagtcccaacccagcaatcttccccataaaaattctggaagactttacaaaacccttatcactcaattttcgcctcttcggaaacattctggcagacgagctagtagtatcagtcttttgtcttttagcgccaattttaataccgttacctgtaaccgccttagcattgttcgcaggctctattcaagctttaatattctcaacgctctcggcagcttatatatctgaagctatagaaaatcacaaaattgattaaaaacttggtattataataaacacaaataactcacaacacaaaaaaactaaaaaaatgaacccaatcgtatgtgcagcatctgtaatagcttctggccttgccattggactagcagcaataggcccaggcataggacaaggatctgcttcggcacaagctttagaaagtatcgcccgccagccagaggcagaaggaaagattcgtggtactctattattgtcattggcctttatggaatccttaactatatacggattagttattgcactatcacttctgtttgcaaacccattcacttcctaagcgaatgaaatgcaacaaatattcacctaaaatccaatgctctttacatcaatctacaggacaaaaaaaccacgtgccgaaaagataagcagccatttattttacttcaatgcaacgctaccaattgtagcaatccaagtgttggttttaatgtctatcctcaataaggttttttacaagccagtaaaagaagcactgcaaaacagaaaattaacgctacacaaaacgcaactacacatatctgaattaatgtctcaagcaaactacatatctaaacaaaaccaacaacaacttaacaataaaaaaagagaagctcaccaaaaagaaaatacagcacaagagagaataaaacaaagagtcagcaacgaagtaaaaaaagcccaaacaaaaattgaacaattcttaaagagccataaagccaaccttctattagaaaaagacaaaaatttacaattacttaacgcagaagcaaataaccttgctataaatgtagaagaaaaattactacccatgacaaaccccataaaaataccacgtacaccctaacccaatggaaaataaaatttgaaacatgtttttacttaataacaatattttacagtcaaatattatcaatatatgcattttaataccaatattaataaaattatggatagaccttgcaactccaaaactaactgatcgccataaatacatccttcgcgagatacaaaacagtgaaaaagaactacaacaagctagccttaacctaataaccgaagaaaatcaaacgaaaaaactaaaaacacacctacaaacaataaaaataatacaaaaataaccatcagaaatattgaactaaacgcaatacatcaggggaaagaaaaaattgaaatgctaacacaagaagagaaaaaatgcgcagatatggaaacactagccacaaaaaaacaaatctatcaacatacggccaatcttgttatccaaatttctgaaaaaacttaaagaaaatactaacctgcaaaaaacaatcagaattaataaatataaatatcaatcaactagatactctaaaatgacctttaaacatgcaagaatcagcaatacatacgcaaaaggattgttaatgggacgctctaaagaaaacttacaaagatttatagccaatacacacttatgccacagcatcctaaagcaatcaatatccctaaaaaaaacgttatttaatccactcgtagaaaggaataaaaaaaagctcatcctaaaaaaaatcctagatggaaaagtagaaaaagattttctaaatttcttatttattgtatgcgaccacaaaaaaagcaacttattacctgatattttgaataaaactatacaactagcatacaagcatcaaaaaacgcaatatgcaactatcgaatctactataccactaacccaaacccagaaacaatctttacaaaataaactgaaaattctaacaggggcacacgaaattcaactccaaccaataataaaccaggaactaataggaggaataatcatacacacaaccttaaaaacaatagattttagtataaaaacacaactacaaaagctttcaaaatcacttgcacccaagttccaaaattaaccaccctaaatataacaatatagattatgataggaattcaacctgatgaaataagctccctcataagagagcaaattaacaactacgatcaaacagtatgtacttcgcacataggaacagttgtacaaagcgcagacggaatggctcgtgcttatgggttagaccaagtcatgatgggagagctagtagaattcgaagatagaaccctaggcattgtactaaatttagaagtgaacaacgtagggattatgatcttaggaaacagccgcaccatcatcgaaggcacatcggtaaaagctacagggaagattgcccatataccagtcggaaaaaattacctaggaaggatcatagacgctctagggaaccctgtggatggcaaaggagatatttataccgaggatactagcttaatcgaaccaccggcgcctggaattatctctcgtgcccccataaccgaaccaatacatacaggaataattaccatagattctatgataccaataggaagagggcaaagagaactaattataggagacagacaaacaggaaaaacttccctcgctatcgacacaatactaaaccaaaaggacgaaggcgtaatatgcatatacatagccataggacaaaaggccagctccgtacgctcaatcgcacacatattacaagaaaaaggagcattggagtatacaaccatactttctgcctcatcagaagaatcagcagctttacaatacattgctccctatacgggaacagcaataggagaatactttatgcaccaaggacaatcggtacttatcatctacgatgatttatccaaacatgcaacagcctatagacaactatctctaatattaagaagaccacctggacgagaagcatacccaggggacgtattttatgctcactctaaactccttgagagaagcgcaaaacttaatccaaagctaggagggggaagtattacagccatcccaataatcgaaacccaagcgagtgatgtatctgcctacattcctacaaacgttatatccataacagacgggcaaatatttctatcaggagatttatttaactctggagtaagacctgcaataaacattggaatttctgtatctagagtagggtcgtccgcacaaactaaagcaataaaaaaaatatctggaaaacttaagcttgagctctcccaattttatgaactagaaactttttcccaattcacttccgacctcgataggtctacaaaaaaccaactagcccgaggccaaaggctacgtgaaattttaaagcaaccacaatattcgccaatacccgttcatgaacaagtagcaatcatttacgctgcaactaacaattttttagatgatataccaatcaaaaatatcaaacagttcattgaatacttaaaaacaactataaaaaataggggaactaaatacacaaagcttattatgaagtctaaagacctagaagaaaaaacagagcaagcactaaaagagcttataatagaagcgaaggagagcttcgcaactcactcaacataaaattatttaacctatagtgttg

SEQ ID NO: 95 - DNA, 4589 bp, Artificial
  Arthrospira platensis NIES-39
cgccgccggtcctcgactgaatcagagagcgatacaccccaaaccctgatcaacccaaaatcccacccatcactaacgcataccaaagggccagacggattctgctatggcgaaacagttgatagctattcatcgcgtcgggcggggtgaactaccccaaagagccttctgacgaggatggggcttcgggattcaacttaaaacgatagccctgaccgcgaatagtttcaatggggcatacaagcgccataactcgctagtttgcgccgtaataagcgcatttgcgccgccaccacattactgatgggttcttcctccccctaccaaagttgatagcggattttactccccggaataatgcgatcgggattttgcattaaataggcaaaaatctgaaattctttgaccgttaagggaatcattgagtgaggttgggtgagtcgattacagatagtattgttcgtcgcgccgatgcgcataaatgctcatcaagtcagggtttggggttggagggtgggcgatcgccgcggtccgtgcgcgcaacccttgccaataattccgccatcacaaaaggtttcaccaaataatcgccgcgtaggtgctacctacgtccagtaattcgatgatccggttgtcccagagccgttaacatcaaaatgggtagagggtgcgggttcgcgcggatgcgttgacataactccaaacccgacaactccggcacaagccaatcggccaatcgccaccgtgtaatccgtccactgactttcgatcgcaatgccaggcctgcactcggccgcgtcacccaatcgacaatatatttctcattgaccagaacttgcgcggtccgcctatcctaattccgcttcatcttcgaccaacagaattcgcatcgtgtgcaagggggagacgactgcgatcgaggttaacagactgcgagaaaaaaggggtgagatttcacctagatttcatctcctctttggcacactggggaagatttggcagaaaaccccatgtttttgtgaggaggtctaatgaacacaacttctatgattggctcaatgttcgctctgggcttaattttcggcacacccaccctaaccctagcccatgtgggacacggccccgggagtttcaagccgaaggcgggattgaccgagttcccgtcaacccagaaaccgatcccctcctcgggacccgtcgggctcccatgcaagtgcgcctttgatggcagttcccgcgttcttgttcatagcaagcgccttagtcgatgcgtaggggaaacagttagcctttgtcttgtatggtagtttttatgaaccctccggagtgcaaatcggagcaacaaacggggaatgggtcgaagtcacaaacgggttatccgttggggaacaactcgtgaggtggggcagtttatccctctatgctgaatcccgcaaaacccaaaccgcaacaacggtaccgggtccagaaaccgccaccactcccaccgatgaagtttccccccaaaaccagacacccatgaggacaatacagagcgcaatgtaccttacaagccactcagttttccctaatgaacactagccgccgcccgtggaggagttttgctcttctgtggtgggattttcttctggatccggaagtcgtcggaacaaaaatctcttctctgacaaataagggaggttgatagcccgtcgatgtttaacaccattcaaaatccgccgctaaaaaattccattgctcaacggtggttaatggtgattggatctctggtcatcaccatctgggggttggtgacaattactcaaatgcccgagtatgttttccctccctttgctccccctcaagtagatatccaaactgaggcggttggtttggctccggagtaagtagaggctcagattacggttcagggtgaaaccaggcaaccgtgaatggcttacccagtgtcaccgttgtctggttcgttcgtcctccaattggggactctctatggttcatgttgtctttgaccaagaggctgatatttaccaagcgcggcaggctgcagttggaccgactccaacagaatccgaaccggagtcaactgccccaagggattcatccgccggaaatttcccccttagtctctccgttgggaacgattctgcaatatgcctttacggtcaatggtcagggagaaacctctctgatggatttacgccgtttggtggatgttaccctcagaaatcagattctccgggtgccaggtgtgtctcaggtgactatttatgggggggatgaacgggaaaaccaggcaaccgtgaatccacaactgttgcgatcgcgccaagtctccctcaatgaagtcactgccgccgcacggtccgccccttccaacgccccgggggggttcggcgaggggggggtcaagagttactcgtccgagggatcggacagatgaacaccgacgccgacttacagcagtcagtggcggccgttcaagacggcgaacctccaaagctccaagatgtggcggaagtcaaaaccggagcagcacttaaactcttagatgccagcttcaacggacagccggctgttgtattgatgattaacaagcaaccggatgtagatacccccaccgtcacccaagcggtggaagcggtgatgaactccctacagtccacctttcccgccgatgtccgggttcaacgcaccttccgccagtcaaactttattgattctgctattggcaacgttagcggctccctcattcaaggcatcatcatcgtgtcggtaattatgttgctctttttaatgaattggcgcacggctatcattaccctgagtgccattcccttatctctcttgattggcttaatgttcatgaaagcctttggtttagggattaataccatgaccttggggggcttagtggtggcgattgggtcagttgtcgatgatgcccccccaggatatggaaaactgctatcgcggtttacggcggaatcaaacccaagaaaaccccaaccccccctttctcggttgtctggtttatgaaacttcggtcatggtggcgttggctgtcattttttccacggttattatcatcgtcgtctttgcccccatttttactttaacgggggtagaagggcggatttttgcccccatgggttgggcttatttgctctccattgtcgcttctaccttggcctggggaccctttcggccgccctctgtgctattttattggctcatcatcaactcccccaagaaggcacatttatttcacgtggggcatccgcgtttgtatcgcccacggcgagacttgtctggtccggactccccaactgattttaggattagccttagcctcctacgttgtctacatagcgccgttgtgccttccttgggacgggttttctgcgcctgatttcaggaaaaatctatggtgaactctatggttttatttcagtcggtctctttggagatgaccaatcgggcgggaatggctctggctcaatccctacaagatcatcccctttatgaatgggtacaagttcgtgctggacgcgctccgggggatgctgatggggcgggggtgaacatggcccatgtggatgtggagtctcgttttcttgccctacaagaccgagaggcggctgttcaacaactggcttgcgaagtttctgcaattgcccggtgttgcgcctaatattggcggttttatttctcaccgcatggatgaagttttatcgggggtcagaagtgcgatacgtgctacgcacacgctacgcgaacgctgtcaaaatttttggccccgatttagtccagttgcgtcaagtcgcttaacaagtccgagatgctataaaacctattgcgggagcccttcgacttacaattagaacctcaattacctatccgcggggttcaaattcattatgaccgccagttgcagtctggttatggtttaaccatggctgccatttctgaggtggtggaaaccgctctcaatggtcaagtggtgtcccaagtcccggaaaatcaacaactgattaatattaccgtcggttgcacttcgcctcacgcaataatttagatgccattggggcgattcccctctctacccccacgggacaaatgattcccctagcgtgctgtggctagagtctcgtcaaggtatgggggctaatgtggtagaacccgaacttgaaaatgtgtctcgtttaattgtagtgtccgcgctacaaacgcaaacgcgagactgagtccgtgtggtgagtgacattcaggctgctatccgccaaaatgtccagttacccaatggctactttatccaatatggcggacagtttgaagcggaacaaaaagccaccaataatctggtctggtatagcatcctagccgcgattataattgccattttgatgttcttcgctggcgcgtgtctctcaggcgcgactgtggccattatgctcaatcttcccttggccttggtggggggtattatctcgattgtcctaacgggggggggtgatttctatcgcttctctaatcggttttattacgctgtttggggtggctgtccgcaatggtttactgttggtggataa

SEQ ID NO: 96 - DNA, 2766 bp, Sulfolobus acidocaldarius
ttagctcacctctttaggtgattttttactaaaatattcttccctcatcttcctcttcctctcaattgcatccattctctctttatcacctgtgtctagttttgttataatgactttagaagcatgaacccatacatatactggagtaccgtctgctttctttttcgttaacccttctatagctacccttccccttttcctatctacctctaccactttaccttcataaccaacttgagaaccacgcataagcttaacactatcgccttttcttattgatattcttttaataccgtattgcttctccaaatcttcgcttagtcttgctgtaagtaacttatacctcaaatgattaggcaaattgtaaactaatttcctttgttttgacggcttagaggatatcattttcatatcaccatagttgctacactggctattttaggccatctctcagcagcttccctagctataggacctctaacctcagttcctttaggagtaccgtcaggatttataatcacaactgcattatcctcaaatgatatccacgtaccatctggcctcctaaagggcattctttgtcttactattacagccttaaacttctgttttctaacatcaggagaacccttccttactgataccatcactaaatctgctatattagcaaaaggaactctcctgagaacacccctatacccataaataccaataattacagcctctttagctccactattatcagcaacaactacactactattatgctgtagagcaggggtaaatcctttcctgctccctaaaacattcattttttctggcatcttacctcttcacccctaatataacaaatgaaatagactttgcaataggtctactttcacctattataacattatcgccttctttcacttctaaacatgaaggaatatgaacgtgtatacgactcctacgtctctcatatctcttatacttagaatcatagaaaatgtactctctttcaactgttccagtcctatttgccctagctctaattaactttccttctaatactataccccttacctttaaactgccgtgataaggacaatactcatcatcacaggtcttcgatggagctgaaacaccctctattcccacatttttaaccaattgaccctttttacccaaacttatatcccctcttttcaggtttacctattagcttagtgccaggtacaacaaaaattttaccttttgtgctaatttgaaataatccattttgcttaataactagaattttccttgaattggaaccaatcataaatgcgttctctgtgtcttctattaccaatccttccttcccaactagtgttggatcagtgtgattcaagatcctcacccatgaacctataaaatccaacttacttctttgcctccttcttactcttctcgccaattacagttaagatacgtgctatatccttcctagtattttttattgatgctgtattcttcaatgtacccattctagattcaactctcaattttaatagttgcatctctaactcattcaatctctcctttaactgtttaagatccattttcctcaattcatcaacttccagaggcatttgacacccctcctgaagacttattttcctctataaagctaacattggtaaccgttaccattccaccttgctcagttttttctaccttctcctttaactctatcttatctatacttctagtgggtttcgttattactacttcaactccataaactccaggcttaagcattgcaatacctattgcacggtctataattttctctaactgttgaccactcttatacactgtgccttcttttaatttttcatatttcgctctttccgatgtaagtttaccactaactatcacttctgcacctactgcaccagagctcattattctcctaatggttataaacgcggctcgcctgaaatgatatcctttctctaaagctatagcaagcctaaatgccattactctagcgtttaactccgggttctctgctgctgtaactgtaatctgcgggttttctaaaccaaagaacttttccaatacttgggctaactgtttaatagttttacctcccttccctattattaacggaggtctaccggcatatattatgaccctagttcctattggtgtcttaactatctctactcctgcatactccgcattgtaaaattgcttagctaagtattcatcgaccattacttttattgcagccttttgtaaaaagtgcctctttataagaaccatatcttacacctcacccagaataacctcaatatgtgataaatacttgtattttcttgtagatcttccaaaagctcttgtcatatatctctttaacactaatcctttatgagcagctatatggactatttttaaattgtctatatcaagacccttactgtttgcattattttcagcattatccaacagcttaagaacatatttaatagctttcttaggatatctgccactcttcactttccactttctggaaatgttagacctatgagatgttccgtgagaatatcgccagaacggtaaagcttccttttcttctaggacgttgttaagaaattccctagcttcctttaaattcatacctcttattgccttacatacgttgtataaatccctaacagacgcatcaatatttttgattacagccttagctaacttgtcttcaggaatagataaaattggatatacccatttgctcat

(19) United States
(12) Patent Application Publication
(10) Pub. No.: US 2012/0094385 A1
(43) Pub. Date: Apr. 19, 2012

(54) GENE POSITIONING SYSTEM FOR PLASTIDIC TRANSFORMATION AND PRODUCTS THEREOF

(75) Inventors: Adelheid R. Kuehnle, Honolulu, HI (US); Michele Champagne, Encinitas, CA (US)

(73) Assignee: KUEHNLE AGROSYSTEMS, INC., HONOLULU, HI (US)

(21) Appl. No.: 13/077,406

(22) Filed: Mar. 31, 2011

Related U.S. Application Data
(63) Continuation-in-part of application No. 12/026,316, filed on Feb. 5, 2008, now abandoned.
Application No. 12/026,316 is a continuation of application No. 11/489,050, filed on Jul. 18, 2006, now abandoned.
Application No. 11/489,050 is a division of application No. 11/053,541, filed on Feb. 8, 2005, now Pat. No. 7,618,819.
Application No. 11/053,541 is a division of application No. 10/835,516, filed on Apr. 28, 2004, now Pat. No. 7,129,392.
Application No. 10/835,516 is a division of application No. 09/918,740, filed on Jul. 31, 2001, now abandoned.
(60) Provisional application No. 60/221,703, filed on Jul. 31, 2000.

(51) Int. Cl. C12N 15/82; C12N 15/63

(52) U.S. Cl. 435/468; 435/471

(57) ABSTRACT

The present invention is directed to Gene Positioning technology for biosynthesis of one or more products of interest via plastid transformation of plants or algae, such as for example tobacco, Lemna, Rhodomonas and Cryptomonas, with pseudogene vectors containing polynucleotides encoding one or more products of interest. The present invention is also directed to transgenic plants or algae, containing pseudogene vectors integrated into a desired locus in the plastid genome, allowing simultaneous expression of multiple transgenes.

### CROSS-REFERENCE TO RELATED APPLICATIONS

This application is a continuation-in-part of U.S. application Ser. No. 12/026,316, filed Feb. 5, 2008, which is a continuation of U.S. application Ser. No. 11/489,050, filed Jul. 18, 2006, now abandoned, which is a divisional of U.S. application Ser. No. 11/053,541, filed Feb. 8, 2005, now allowed, which is a divisional of U.S. application Ser. No. 10/835,516, filed Apr. 28, 2004, now allowed, which is a divisional of U.S. application Ser. No. 09/918,740, filed Jul. 31, 2001, now abandoned, and claims the benefit of U.S. Provisional Application No. 60/221,703, filed Jul. 31, 2000, all of which are hereby incorporated by reference herein in their entirety.

The Sequence Listing for this application is labeled “Seq-List.txt” which was created on Mar. 31, 2011 and is 225 KB. The entire contents of the sequence listing is incorporated herein by reference in its entirety.

### FIELD OF THE INVENTION

This invention relates to the fields of biotechnology and genetic engineering, in particular to agricultural and aquacultural biotechnology. More specifically, the invention relates to transgenic plants and microalgae, in particular to transplastomic plants and microalgae and means for insertion of genetic material into plastids.

### BACKGROUND OF THE INVENTION

The ubiquitous isoprenoid biosynthetic pathway is responsible for the formation of the most chemically diverse family of metabolites found in nature (Hahn et al., J. Bacteriol. 178:619-624, 1996) including sterols (Popjak, Biochemical symposium no. 29 (T. W. Goodwin, ed.) Academic Press, New York, pp 17-37, 1970), carotenoids (Goodwin, Biochem. J. 123:293-329, 1971), dolichols (Matsuoka et al., J. Biol. Chem. 266:3464-3468, 1991), ubiquinones (Ashby and Edwards, J. Biol. Chem. 265:13157-13164, 1990), and prenylated proteins (Clarke, Annu. Rev. Biochem. 61:355-386, 1992). Biosynthesis of isopentenyl diphosphate (IPP), the essential 5-carbon isoprenoid precursor, occurs by two distinct compartmentalized routes in plants (Lange and Croteau, Proc. Natl. Acad. Sci. USA 96:13714-13719, 1999). In the plant cytoplasm, IPP is assembled from three molecules of acetyl coenzyme A by the well-characterized mevalonate pathway (Lange and Croteau, Proc. Natl. Acad. Sci. USA 96:13714-13719, 1999). However, a recently discovered mevalonate-independent pathway is responsible for the synthesis of IPP in plant chloroplasts (Lichtenthaler et al. FEBS Letters 400:271-274, 1997).

Following the synthesis of IPP via the mevalonate route, the carbon-carbon double bond must be isomerized to create the potent electrophile dimethylally diphosphate (DMAPP). This essential activation step, carried out by IPP isomerase, insures the existence of the two 5-carbon isomers, IPP and DMAPP, which must join together in the first of a series of head to tail condensation reactions to create the essential allylic diphosphates of the isoprenoid pathway (Hahn and Poulter, J. Biol. Chem. 270:11298-11303, 1995). Recently, it was reported that IPP isomerase activity was not essential in E. coli, one of many eubacteria containing only the non-mevalonate pathway for the synthesis of both 5-carbon isomers, suggesting the existence of two separate mevalonate-independent routes to IPP and DMAPP (Hahn et al., J. Bacteriol. 181:4499-4504, 1999). Thus, it is unclear whether an IPP isomerase is essential for the synthesis of isoprenoids in plant plastids as well. Regardless of whether IPP isomerase activity is present in plant plastids, the separation by compartmentalization of the two different biosynthetic routes, the mevalonate and deoxyxylulose phosphate pathways (or “non-mevalonate”), for IPP and DMAPP biosynthesis in plants is the fundamental tenet upon which the subject inventions are based.

The synthesis of IPP by the mevalonate pathway (Eisenreich et al., Chemistry and Biology 5:R221-R233, 1998) is cytoplasm based and occurs as follows: The condensation of two acetyl CoA molecules to yield acetoacetyl CoA is catalyzed by acetoacetyl CoA thiolase (EC 2.3.1.9). The addition of another molecule of acetyl CoA to acetoacetyl CoA is catalyzed by 3-hydroxy-3-methylglutaryl-coenzyme A (HMG-CoA) synthase (EC 4.1.3.5) to yield HMG-CoA, which is reduced in the subsequent step to mevalonate by HMG-CoA reductase (EC 1.1.1.34). Mevalonate is phosphorylated by mevalonate kinase (EC 2.7.1.36) to yield phosphomevalonate, which is phosphorylated, by phosphomevalonate kinase (EC 2.7.4.2) to form mevalonate diphosphate. The conversion of mevalonate diphosphate to IPP with the concomitant release of CO2 is catalyzed by mevalonate diphosphate decarboxylase (EC 4.1.1.33).

In organisms utilizing the deoxyxylulose phosphate pathway (aka “non-mevalonate pathway”, “methylerythritol phosphate (MEP) pathway”, and “Rohmer pathway”), the five carbon atoms in the basic isoprenoid unit are derived from pyruvate and D-glyceraldehyde phosphate (GAP) (Eisenreich et al., 1998). Thus, synthesis of IPP and/or DMAPP by the non-mevalonate route, which occurs in plastids, is as follows: Pyruvate and GAP are condensed to give 1-deoxy-D-xylulose 5-phosphate (DXP) by DXP synthase (Sprenger et al., Proc. Natl. Acad. Sci. USA 94:12857-12862, 1997). The rearrangement and reduction of DXP to form 2-C-methylerythritol 4-phosphate (MEP), the first committed intermediate in the non-mevalonate pathway for biosynthesis of isoprenoids is catalyzed by DXP reductoisomerase (Kuzuyama et al., Tetrahedron Lett. 39:4509-4512, 1998). MEP is then appended to CTP to form 4-(cytidine 5′-diphospho)-2-C-methyl-D-erythritol (Rohdich et al., Proc. Natl. Acad. Sci. USA 96:11758-11763, 1999), followed by phosphorylation of the C2 hydroxyl group (Lüttgen et al., Proc. Natl. Acad. Sci. USA 97:1062-1067, 2000) and elimination of CMP, to form a 2,4-cyclic diphosphate (Herz et al., Proc. Natl. Acad. Sci. USA 97:2486-2490, 2000). Interestingly, Herz et al. reported the possible existence of bifunctional proteins with both YgbP and YgbB activities. Once the remaining steps to the fundamental five-carbon isoprenoid building blocks, IPP and DMAPP, in the non-mevalonate pathway are discovered, they will serve as additional targets for inhibitors with antibiotic and herbicidal activity.

Since the non-mevalonate pathway is ultimately responsible for the biosynthesis of compounds critical for photosynthesis such as the prenyl side-chain of chlorophylls, which serve as lipophillic anchors for the photoreceptors and the photoprotective carotenoid pigments, any enzyme, gene, or regulatory sequence involved in the biosynthesis of IPP and/or DMAPP can be a potential target for herbicides. For example, the antibiotic fosmidomycin, a specific inhibitor of the enzyme DXP reductoisomerase (Kuzuyama et al., Tetrahedron Lett. 39:7913-7916, 1998) has been shown to have significant herbicidal activity, especially in combination with other herbicides (Kamuro et al. “Herbicide” U.S. Pat. No. 4,846,872; issued Jul. 11, 1989). The report of an Arabidopsis thaliana albino mutant being characterized as a disruption of the CLA1 gene, later revealed as encoding DXP synthase by Rohmer et al. (Lois et al., Proc. Natl. Acad. Sci. USA 95:2105-2110, 1998), also illustrates the potential of non-mevalonate pathway enzymes as targets for compounds with herbicidal activity. Accordingly, one of ordinary skill in the art can readily understand that as additional compounds are discovered exhibiting herbicidal activity based on their effects on the non-mevalonate pathway, those compounds could be used in accord with the teachings herein.

The synthesis of carotenoids from IPP and DMAPP takes place in plant plastids by a genetically- and enzymatically-defined pathway (Cunningham and Gantt, Ann. Rev. Plant Mol. Biol. 39:475-502, 1998). Enhanced production of carotenoids such as lycopene and β-carotene in plants is highly desirable due to the reported health benefits of their consumption (Kajiwara et al., Biochem. J. 324:421-426, 1997). Enhanced carotenoid production in plants can also have a dramatic effect on their coloration and be highly desirable to the growers of ornamentals, for example. The IPP isomerase reaction is considered to be a rate-limiting step for isoprenoid biosynthesis (Ramos-Valdivia et al., Nat. Prod. Rep. 6:591-603, 1997). Kajiwara et al. reported that the expression of heterologous IPP isomerase genes in a strain of E. coli specifically engineered to produce carotenoids resulted in over a 2-fold increase in β-carotene formation. Recently, it has been reported that expression of an additional gene for DXP synthase in an E. coli strain specifically engineered to produce carotenoids also increased the level of lycopene substantially (Harker and Bramley, FEBS Letters 448:115-119, 1999). Increased isoprenoid production also has been shown in bacteria by combining carotenogenic genes from bacteria with an orf encoding IPP isomerase; and was even further enhanced when additionally combined with the dxs gene from the MEP pathway to supply the precursors IPP and DMAPP (Albrecht et al. Nature Biotechnology 18: 843-846, 2000).

Accumulation of one specific isoprenoid, such as beta-carotene (yellow-orange) or astaxanthin (red-orange), can serve to enhance flower color or nutriceutical composition depending if the host is cultivated as an ornamental or as an output crop; and if the product accumulates in the tissue of interest (i.e. flower parts or harvestable tissue). In plants, tissue with intrinsic carotenoid enzymes can accumulate ketocarotenoids such as astaxanthin in chromoplasts of reproductive tissues of tobacco by addition of the biosynthetic enzyme beta-carotene ketolase (Mann et al., Nature Biotechnology 18: 888-892, 2000). Astaxanthin is the main carotenoid pigment found in aquatic animals; in microalgae it accumulates in the Chlorophyta such as in species of Haematococcus and Chlamydomonas. Thus, an increase in the essential 5-carbon precursors, IPP and DMAPP, by expression of orfs encoding IPP isomerase and orfs upstream thereof, can feed into the production output of such valuable isoprenoids in organisms other than bacteria.

As a further example of utility, Petunia flower color is usually due to the presence of modified cyanidin and delphinidin anthocyanin pigments to produce shades in red to blue groupings. Recently produced yellow seed-propagated multiflora and grandiflora petunias obtain their coloration from the presence of beta-carotene, lutein and zeaxanthin carotenoid pigments in combination with colorless flavonols (Nielsen and Bloor, Scienia Hort. 71: 257-266, 1997). Industry still lacks bright yellow and orange clonally propagated trailing petunias. Metabolic engineering of the carotenoid pathway is desired to introduce these colors in this popular potted and bedding plant.

Plant genetic engineering has evolved since the 1980s from arbitrarily located monocistronic insertions into a nuclear chromosome, often subject to multiple copies, rearrangements and methylation, to predetermined sites for defined multicistronic or multigenic operon insertions into a plastid chromosome (plastome), which thus far is thought impervious to typical nuclear gene inactivation. While breeding of crop plants by nuclear genome engineering is nevertheless a proven technology for major agronomic crops and for traits such as herbicide resistance, introgression of genes into the plastome is a highly promising breeding approach for several reasons as described by Bock and Hagemann (Bock and Hagemann, Prog. Bot. 61:76-90, 2000). Of note is the containment of transgenes in the transplastomic plant: Plastids are inherited through the maternal parent in most plant species and thus plastid-encoded transgenes are unable to spread in pollen to non-target species. Therefore plastid engineering can minimize negative impacts of genetically engineered plants. A report on potential transfer by pollen of herbicide resistance into weedy relatives of cultivated crops (Keeler et al., Herbicide Resistant Crops: Agricultural, Economic, Environmental, Regulatory and Technological Aspects, pp. 303-330, 1996) underscores the value of using plastid engineering rather than nuclear engineering for critical production traits such as herbicide resistance. Daniell et al. have recently demonstrated herbicide resistance through genetic engineering of the chloroplast genome (Daniell et al., Nat. Biotechnol., 16:345-348, 1998).

Moreover, plastids are the site of essential biosynthetic activity. Although most associate photosynthesis as the primary function of the chloroplast, studies document that the chloroplast is the center of activity for functions involving carbon metabolism, nitrogen metabolism, sulfur metabolism, biochemical regulation, and various essential biosynthetic pathways including amino acid, vitamin, and phytohormone biosynthesis. Crop traits of interest such as nutritional enhancement require genetic manipulations that impact plastid biosynthetic pathways such as carotenoid production. While nuclear-encoded gene products can be exported from the engineered nucleus into the plastid for such manipulations, the biosynthetic genes themselves can be inserted into the plastid for expression and activity. As we begin to pyramid multiple genes often required for pathway manipulations (such as the aforementioned carotenoid biosynthesis) the repeated use of selection markers is expected to lead to unstable crops through homology-dependent gene silencing (Meyer and Saedler, Ann. Rev. Plant. Physiol. Mol. Biol. 47:23-48, 1996). In addition, the requirement for higher expression levels of transgenes for effective phenotypes such as vitamin levels and herbicide and pest resistance levels often falls short in nuclear transformations. These deficiencies are overcome through plastid transformation or combining plastid with nuclear transformations: The plastid recognizes strings of genes linked together in multicistronic operons and, due to the high copy number of genes within a plastid and within plastids in a cell, can produce a hundred- to thousand-fold the amount of transgene product. Accordingly, there is a continuing need for improved methods of producing plants having transformed plastids (transpiastomic plants).

Golden rice is one example for which plastid engineering can complement nuclear engineering of pathways that reside in the plastid, yet have met with limited success. The metabolic pathway for beta-carotene (pro-vitamin A) was assembled in rice plastids by introduction into the nuclear genome of four separate genes, three encoding plastid-targeted proteins using three distinct promoters, plus a fourth selectable marker gene using a repeated promoter (Ye et al. Science 287:303-305, 2000). The wild-type rice endosperm is free of carotenoids but it does produce geranylgeranyl diphosphate; combining phytoene synthase, phytoene desaturase, and lycopene-beta cyclase resulted in accumulation of beta-carotene to make “golden rice.” However, the quantity produced was lower than the minimum desired for addressing vitamin A deficiency. An increased supply of precursors for increasing intermediates, such as geranylgeranyl diphosphate, is predicted to significantly increase isoprenoid production. Insertion of an operon encoding the entire mevalonate pathway into the rice plastome of the “golden rice” genotype, using for example the methods as described in Khan and Maliga, Nature Biotechnology 17: 910-914, 1999, can provide a means for making improvements in metabolic engineering of this important monocot crop.

Proplastid and chloroplast genetic engineering have been shown to varying degrees of homoplasmy for several major agronomic crops including potato, rice, maize, soybean, grape, sweet potato, and tobacco including starting from non-green tissues. Non-lethal selection on antibiotics is used to proliferate cells containing plastids with antibiotic resistance genes. Plastid transformation methods use two plastid-DNA flanking sequences that recombine with plastid sequences to insert chimeric DNA into the spacer regions between functional genes of the plastome, as is established in the field (see Bock and Hagemann, Prog. Bot. 61:76-90, 2000, and Guda et al., Plant Cell Reports 19:257-262, 2000, and references therein).

Antibiotics such as spectinomycin, streptomycin, and kanamycin can shut down gene expression in chloroplasts by ribosome inactivation. These antibiotics bleach leaves and form white callus when tissue is put onto regeneration medium in their presence. The bacterial genes aadA and neo encode the enzymes aminoglycoside-3′-adenyltransferase and neomycin phosphotransferase, which inactivate these antibiotics, and can be used for positive selection of plastids engineered to express these genes. Polynucleotides of interest can be linked to the selectable genes and thus can be enriched by selection during the sorting out of engineered and non-engineered plastids. Consequently, cells with plastids engineered to contain genes for these enzymes (and linkages thereto) can overcome the effects of inhibitors in the plant cell culture medium and can proliferate, while cells lacking engineered plastids cannot proliferate. Similarly, plastids engineered with polynucleotides encoding enzymes from the mevalonate pathway to produce IPP from acetyl CoA in the presence of inhibitors of the non-mevalonate pathway can overcome otherwise inhibitory culture conditions. By utilizing the polynucleotides disclosed herein in accord with this invention, an inhibitor targeting the non-mevalonate pathway and its components can be used for selection purposes of transplastomic plants produced through currently available methods, or any future methods which become known for production of transplastomic plants, to contain and express said polynucleotides and any linked coding sequences of interest.

This selection process of the subject invention is unique in that it is the first selectable trait that acts by pathway complementation to overcome inhibitors. This is distinguished from the state of the art of selection by other antibiotics to which resistance is conferred by inactivation of the antibiotic itself, e.g. compound inactivation as for the aminoglyoside 3′-adenyltransferase gene or neo gene. This method avoids the occurrence of resistant escapes due to random insertion of the resistance gene into the nuclear genome or by spontaneous mutation of the ribosomal target of the antibiotic, as is known to occur in the state of the art. Moreover, this method requires the presence of an entire functioning mevalonate pathway in plastids. For example, if one of the enzyme activities of the mevalonate pathway is not present in the plastid, resistance will not be conferred.

There is strong evidence indicating that the origin of plastids within the cell occurred via endosymbiosis and that plastids are derived from cyanobacteria. As such, the genetic organization of the plastid is prokaryotic in nature (as opposed to the eukaryotic nuclear genome of the plant cell). The plastid chromosome ranges from roughly 110 to 150 Kb in size (196 for the green alga Chlamydomonas), much smaller than that of most cyanobacteria. However, many of the bacterium genes have either been lost because their function was no longer necessary for survival, or were transferred to the chromosomes of the nuclear genome. Most, but not all, of the genes remaining on the plastid chromosome function in either carbon metabolism or plastid genetics. However, many genes involved in these functions, as well as the many other functions and pathways intrinsic to plastid function, are also nuclear encoded, and the translated products are transported from the cytoplasm to the plastid. Studies have documented nuclear encoded genes with known activity in the plastid that are genetically more similar to homologous genes in bacteria rather than genes of the same organism with the same function but activity in the cytoplasm as reviewed for example in Martin et al. (1998) Nature 393:162-165 and references therein.

The process whereby genes are transported from the plastid to the nucleus has been addressed. Evidence indicates that copies of many plastid genes are found among nuclear chromosomes. For some of these, promoter regions and transit peptides (small stretches of DNA encoding peptides that direct polypeptides to the plastid) become associated with the gene that allows it to be transcribed, and the translated polypeptide relocated back into the plastid. Once this genetic apparatus has become established, the genes present in the plastid chromosome may begin to degrade until they are no longer functional, i.e., any such gene becomes a pseudogene.

As is common in prokaryotic systems, many genes that have a common function are organized into an operon. An operon is a cluster of contiguous genes transcribed from one promoter to give rise to a polycistron mRNA. Proteins from each gene in the polycistron are then translated. There are 18 operons in the plastid chromosome of tobacco (Nicotiana tabacum). Although many of these involve as few as two genes, some are large and include many genes. Evolutionary studies indicate that gene loss—as pseudogenes or completely missing sequences—occurs as individuals rather than as blocks of genes or transcriptional units. Thus other genes surrounding a pseudogene in a polycistronic operon remain functional.

The rpl23 operon consists of genes whose products are involved in protein translation. Most of these genes are ribosomal proteins functioning in either the large or small ribosomal subunit. One particular gene of note, infA, encodes an initiation factor protein that is important in initiating protein translation. Although this gene is functional in many plants, it is a pseudogene in tobacco and all other members of that family (Solanaceae), including the horticulturally valuable tomato, petunia, and potato crops. A recent survey of plant groups has indicated that there have been numerous loses of functionality of infA (Millen et al., Plant Cell 13: 645-658, 2001). This as well as other pseudogenes are identified in species whose chloroplast genomes have not yet been fully sequenced.

Pseudogenes such as infA become potential target sequences for insertion of intact orfs. Inserted orfs are controlled by regulatory upstream and downstream elements of the polycistron and are promoterless themselves. Pseudogenes are known for a multiplicity of crops and algae with chloroplast genomes that are already fully sequenced. Crops include grains such as rice and trees such as Pinus. Of note in the latter are the eleven ndh genes; all may serve as potential targets for transgene insertion.

Transplastomic solanaceous crops are highly desirable in order to eliminate the potential for gene transfer from engineered lines to wild species, as demonstrated in Lycopersicon (Dale, P. J. 1992. Spread of engineered genes to wild relatives. Plant Physiol. 100:13-15.). A method for plastid engineering that enables altered pigmentation, for improved nutrition in tomato or improved flower color in Petunia and ornamental tobacco as examples, is desirable for solanaceous crops. The infA gene is widely lost among rosids and some asterids; among the latter, infA is a pseudogene in all solanaceous species examined (representing 16 genera). The solanaceous infA DNA sequences show high similarity, with all nucleotide changes within infA being documented. Thus one set of flanking sequences of reasonable length as known in the art should serve for directed insertion of an individual or multiple orfs into the infA sites of the solanaceous species. It is documented in a solanaceous species that flanking sequences for genes to be inserted into the plastome are not required to be specific for the target species, as incompletely homologous plastid sequences are integrated at comparable frequencies (Kavanagh et al., Genetics 152:1111-1122, 1999).

The upstream 5′ region, often referred to as the 5′ UTR, is important on the expression level of a transcript as it is translated. Knowing the translation products of surrounding genes in a polycistron allows one to select a pseudogene site that is affiliated with a strong 5′ UTR for optimizing plastid expression in a particular tissue. The plastid genome in many plant species can have multiple pseudogenes that are located in different polycistronic sites. So, if one has a choice, one can select a site based on whether it is actively transcribed in green vs non-green plastid; and then if the polycistron has high or low relative expression in that plastid type. Moreover, monocistronic mRNA of ndhD was detected in developed leaves but not in greening or expanding leaves of barley (Hordeum vulgare), despite this gene being part of a polycistronic unit as reported by del Campo et al. (1997) Plant Physiol 114:748. Thus, one can time transgene product production by treating an inactive gene, based on developmental expression, as a pseudogene for targetting and integration purposes using the invention disclosed herein.

Algal species are becoming increasingly exploited as sources of nutraceuticals, pharmaceuticals, and lend themselves to aquaculture. Mass production of the isoprenoid compound astaxanthin produced by the green microalga Haemotcoccus is one successful example of the above. Metabolic engineering that would increase product yields and composition in microalgae would significantly benefit the industry. The development of organellar transformation for the unicellular green alga Chlamydomonas reinhardtii, with its single large chloroplast, opens the door for conducting studies on genetic manipulation of the isoprenoid pathway. Filamentous or multicellular algae are also of interest as untapped biofactories, as are other nongreen algae whose pathways for producing unique fatty acids, amino acids, and pigments can be ameliorated for commercial benefit.

The biolistic DNA delivery method is a general means with which to transform the chloroplast of algae (Boynton and Gillham, Methods Enzymol. 217:510-536, 1993). In addition to gene gun bombardments, polyethyleneglycol-mediated protoplast transformation and other gene transfer methods such as electroporation can be carried out according to methods known in the art, as an alternative means to produce transgenic plants.

Sequencing of at least six plastomes from algae should facilitate transformation systems by confirming insertion sites, including pseudogene sites, and the regulatory elements directing heterologous gene expression. What is required is a dominant marker for selection of stable transformants to which natural resistance is absent (Stevens and Purton, J. Phycol 33: 713-722, 1997). For Chlamydomonas, chloroplasts can be engineered using markers that confer spectinomycin resistance following their integration into the plastome via homologous recombination. By utilizing the polynucleotides disclosed herein in accord with this invention, an inhibitor targeting the non-mevalonate pathway and its components can be used for selection purposes of transplastomic algae produced through currently available methods, or any future methods which become known for production of transplastomic algae, to contain and express said polynucleotides and any linked coding sequences of interest. This is a novel selection vehicle for transplastomic algae. Moreover, elevating the supply of essential precursors for isoprenoid production in algae as described above is enabled by this invention. Hantaviruses are worldwide endemic pathogens of concern because they cause high morbidity. Hantaviruses belong to the family Bunyaviridae and cause two rodent borne viral zoonoses. They have three negative-sense genome segments, designated as S (small), M (medium) and L (large) which encode the nucleocapsid protein (NP), two envelope glycoproteins (G1 & G2) and viral polymerase respectively. In Asia, Europe and Scandinavia, at least four hantaviruses, Hantaan (HTNV), Seoul (SEOV), Puumala (PUUV), and Dobrava (DOBV), are responsible for hemorrhagic fever with renal syndrome (HFRS) and an average mortality rate of 5-10%. Fatality rates range from <1% for milder Nephropathia epidemica caused by Puumala strain, 6-15% for hemorrhagic fever with renal syndrom (HFRS) caused by the Debrova-Belgrade and Hantaan strains, to >35% for hemorrhagic fever with pulmonary syndrome (HPS) caused by New World strains of hantavirus such as Andes (ANDV) and Sin Nombre (SNV). To date, there is no WHO approved hantavirus vaccine available although the discovery of hantavirus dates back to almost 30 years ago. Three different inactivated vaccines are developed and used locally in Korea and/or China.

Handling of hantavirus is very hazardous and requires a Biosafety Level 3 containment laboratory, complicating the production and use of inactivated whole virus vaccines. This issue can be circumvented by using recombinant subunit vaccines derived from the antigenic nucleocapsid or the glycoproteins Gn and Gc. The nucleocapsid protein plays a key role in virus replication and is one of the major antigens invoking humoral immune responses in humans and mice. The hantavirus nucleoprotein (N protein) has been established as a useful antigen for in vitro detection of human IgG and IgM and antibodies in sera of acute phase infected patients (Meng et al. 2003; Schmidt et al. 2005a, 2005b; Tischler et al. 2005). It is the preferred antigen for detecting nephropathic hantavirus infections in humans because high levels of IgM anti-N antibodies can be detected in most patients during the initial presentation of symptoms (Elgh et al. 1998; Kallio Kokko et al. 1998). Serological cross reactivity between the different hantaviruses does occur (Hujakka et al. 2003; Schmidt et al., 2005a), but for optimal diagnostic sensitivity, strain-specific antigens may be required (Hujakka et al. 2003).

To date, Hantavirus nucleoproteins have been expressed in E. coli, Saccharomyces, Vaccinia and Baculo viruses, tobacco and potato (nuclear transformation), and silkworm larvae systems. E. coli-expressed proteins are suboptimal for diagnostic purposes due to the need for extensive purification to remove bacterial components that cross react with human sera. Recombinant hantaviral antigens expressed in Vaccinia for vaccine development have a disadvantage of causing complications such as those observed when used for smallpox vaccine. Although these and other recombinant hantavirus proteins have demonstrated sufficient antigenicity, protein yields appear insufficient for large scale testing purposes, attaining, on average 0.1-2% of total soluble protein. Thus, alternative methods for production of therapeutic proteins such as Hantavirus nucleoproteins are urgently needed.

### SUMMARY OF THE INVENTION

This invention relates to the presence of enzymatic activities necessary to form IPP from acetyl CoA, generally known as the mevalonate pathway, within plant and microalgae plastids. This invention may also require the presence of IPP isomerase activity within plastids resulting from the insertion into said plants and microalgae of a polynucleotide encoding a polypeptide with IPP isomerase activity. This invention may be achieved by the use of any polynucleotide, be it a DNA molecule or molecules, or any hybrid DNA/RNA molecule or molecules, containing at least one open reading frame that when expressed provides a polypeptide(s) exhibiting said activities within plastids. These open reading frames may be identical to their wild type progenitors, or alternatively may be altered in any manner (for example, with plastid-optimized codon usage), may be isolated from the host organism to be modified, may originate from another organism or organisms, or may be any combination of origin so long as the encoded proteins are able to provide the desired enzymatic activity within the target plastids. The described open reading frames may be inserted directly into plastids using established methodology or any methodology yet to be discovered. Alternatively, plastid localization of the desired activities may be achieved by modifying genes already residing in the cell nucleus, inserting foreign polynucleotides for nuclear residence, or inserting polynucleotides contained on exogenous, autonomous plasmids into the cell cytoplasm so that in all cases their encoded proteins are transported into the plastid. For example, a chloroplast transit (targeting) peptide can be fused to a protein of interest. Any combination of the above methods for realizing said activities in plant and microalgae plastids can be utilized. By causing the complete mevalonate pathway enzymatic activity to occur in plastids normally possessing only the non-mevalonate pathway, the presence of said activities within the chloroplasts of a specific plant or microalgae will endow it with resistance to a compound, molecule, etc. that targets a component of the non-mevalonate pathway, be it an enzyme, gene, regulatory sequence, etc., thereby also providing a useful selection system based on circumvention of the inhibition of the non-mevalonate pathway in transplastomic plants and microalgae.

In addition, this invention relates to the use of open reading frames encoding polypeptides with enzymatic activities able to convert acetyl CoA to IPP, generally known as the mevalonate pathway, and a polypeptide with IPP isomerase activity as a method for increasing the production of IPP, DMAPP, and isoprenoid pathway derived products whose level within plant and microalgae plastids is dependent on the level of IPP and/or DMAPP present within the plastids. The presence of exogenous genes encoding 1-deoxy-D-xylulose-5-phosphate synthase and IPP isomerase have been shown to increase the production of carotenoids in eubacteria, presumably due to an increased production of IPP and/or DMAPP. Thus, insertion of the entire mevalonate pathway, solely or coupled with an additional IPP isomerase, into plastids will increase the level of IPP and/or DMAPP, resulting in an increased level of carotenoids and other yet to be determined isoprenoid pathway derived products within plant and microalgae plastids. This invention can utilize an open reading frame encoding the enzymatic activity for IPP isomerase independently or in addition to said open reading frames comprising the entire mevalonate pathway to obtain the increased level of isoprenoid pathway derived products within plant and microalgae plastids. This invention may be achieved by the use of any DNA molecule or molecules, or any hybrid DNA/RNA molecule or molecules, containing open reading frames able to provide said activities within plant and microalgae plastids. These open reading frames may be identical to their wild type progenitors, may be altered in any manner, may be isolated from the plant to be modified, may originate from another organism or organisms, or may be any combination of origin so long as the encoded proteins are able to provide said activities within plastids. The described open reading frames may be inserted directly into plant and microalgae plastids using established methodology or any methodology yet to be discovered. Alternatively, plastid localization of the desired activities may be achieved by modifying genes already residing in the nucleus, inserting foreign genes for nuclear residence, or inserting genes contained on exogenous, autonomous plasmids into the cytoplasm so that in all cases their encoded proteins are transported into the plastid. Any combination of the above methods for realizing said activities in plastids can be utilized.

Further, this invention also relates to the direct insertion of any foreign gene into a plant or microalgae chloroplast by coupling it to the open reading frames encoding polypeptides with enzymatic activities able to convert acetyl CoA to IPP, thus comprising the entire mevalonate pathway. By utilizing a compound, molecule, etc. that targets a component of the non-mevalonate pathway be it an enzyme, gene, regulatory sequence, etc., a method of selection analogous to the use of kanamycin and spectinomycin resistance for the transformation event is achieved. As inhibition of the non-mevalonate pathway in a plant or microalgae results in the impairment of photosynthesis, the presence of the mevalonate pathway biosynthetic capability is apparent, thus enabling the facile screening of concomitant incorporation into plastids of a foreign gene coupled to the open reading frames comprising the entire mevalonate pathway. The use of a polynucleotide comprising an open reading frame encoding a polypeptide with IPP isomerase activity in addition to the open reading frames encoding the mevalonate pathway is a particularly preferred embodiment, which provides all enzymatic activities necessary to synthesize both IPP and DMAPP and overcome the effect(s) of inhibition of the non-mevalonate pathway.

Further, this invention is unique and novel in that the transforming DNA, that is integrated by two or more homologous/heterologous recombination events, is purposefully targeted into inactive gene sites selected based on prior knowledge of transcription in plastid type, developmental expression including post-transcriptional editing, and post-transcriptional stability. Additionally, this invention uses the regulatory elements of known inactive genes (pseudogenes) to drive production of a complete transforming gene unrelated to the inserted gene site. Thus, by utilizing the transgene insertion method disclosed herein in accord with this invention, any foreign gene can be targeted to an inactive gene site (the pseudogene) through currently available methods of gene transfer, or any future methods which become known for production of transgenic and transplastomic plants, to contain and express said foreign gene and any linked coding sequences of interest. This gene insertion process of the subject invention is unique in that it is the first method specifically acting by pseudogene insertion to overcome the need for promoters and other regulatory elements normally associated with a transforming DNA vector while permitting site-specific recombination in organellar genomes. The use of the infA pseudogene insertion site in the solanaceous crops in particular is a preferred embodiment for the transformation of plastids using the open reading frames for the mevalonate pathway as well as for providing the necessary precursors for modified output traits in plants.

Further, this invention provides the Gene Positioning technology for biosynthesis of products of interest via plastid transformation of plants, algae, cyanobacteria and Archaea, such as for example, tobacco, Lemna, Rhodomonas, Cryptomonas, Arthrospira and Sulfolobus, with pseudogene vectors containing polynucleotides encoding one or more products of interest. The transformants can be successfully selected and recovered. In addition, tissues of transformants are repeatedly regenerated to promote homogeneity of the transformed chloroplasts (homoplasmy). Further, the pseudogene vector can be integrated into the desired locus in the plastid genome, allowing simultaneous expression of multiple transgenes.

### BRIEF DESCRIPTION OF THE DRAWINGS

FIG. 1 is a map of cloning vector pFCO1 containing S. cerevisiae orfs encoding phosphomevalonate kinase (PMK), mevalonate kinase (MVK), and mevalonate diphosphate decarboxylase (MDD).

FIG. 2 is a map of expression vector pFCO2 containing S. cerevisiae orfs encoding phosphomevalonate kinase (PMK), mevalonate kinase (MVK), and mevalonate diphosphate decarboxylase (MDD).

FIG. 3 is a map of cloning vector pHKO1 containing S. cerevisiae orf encoding acetoacetyl thiolase (AACT); A. thaliana orfs encoding HMG-CoA synthase (HMGS), HMG-CoA reductase (HMGRt).

FIG. 4 is a map of expression vector pHKO2 containing S. cerevisiae orfs encoding phosphomevalonate kinase (PMK), mevalonate kinase (MVK), mevalonate diphosphate decarboxylase (MDD), and acetoacetyl thiolase (AACT); A. thaliana orfs encoding HMG-CoA synthase (HMGS), HMG-CoA reductase (HMGRt) which in their summation are designated Operon A, encoding the entire mevalonate pathway.

FIG. 5 is a map of cloning vector pHKO3 containing S. cerevisiae orfs encoding phosphomevalonate kinase (PMK), mevalonate kinase (MVK), mevalonate diphosphate decarboxylase (MDD), and acetoacetyl thiolase (AACT); A. thaliana orfs encoding HMG-CoA synthase (HMGS), HMG-CoA reductase (HMGRt) which in their summation are designated Operon B, encoding the entire mevalonate pathway.

FIG. 6 is an iIllustration of how the mevalonate (MEV) pathway, by providing an alternative biosynthetic route to IPP, circumvents blocks in the MEP pathway due to a mutation in the gene for deoxyxylulose phosphate synthase (dxs) and due to inhibition by fosmidomycin of deoxyxylulose phosphate reductoisomerase (dxr).

FIG. 7 is a map of vector pBSNT27 containing N. tabacum chloroplast DNA (cpDNA) and the N. tabacum infA pseudogene and pBSNT27 sequence (SEQ ID NO: 17)

FIG. 8 is a map of plastid transformation vector pHKO4 containing N. tabacum chloroplast DNA (cpDNA) flanking the insertion of Operon B into the infA pseudogene.

FIG. 9 is a map of cloning vector pHKO5 containing S. cerevisiae orfs encoding phosphomevalonate kinase (PMK), mevalonate kinase (MVK), and mevalonate diphosphate decarboxylase (MDD), and acetoacetyl thiolase (AACT); A. thaliana orfs encoding HMG-CoA synthase (HMGS), HMG-CoA reductase (HMGRt); R. capsulatus orf encoding IPP isomerase (IPPI) which in their summation are designated Operon C, encoding the entire mevalonate pathway and IPP isomerase.

FIG. 10 is a map of cloning vector pFHO1 containing S. cerevisiae orf encoding acetoacetyl thiolase (AACT); A. thaliana orf encoding HMG-CoA synthase (HMGS); Streptomyces sp CL190 orf encoding HMG-CoA reductase (HMGR).

FIG. 11 is a map of cloning vector pFHO2 containing S. cerevisiae orfs encoding phosphomevalonate kinase (PMK), mevalonate kinase (MVK), and mevalonate diphosphate decarboxylase (MDD), and acetoacetyl thiolase (AACT); A. thaliana orf encoding HMG-CoA synthase (HMGS); Streptomyces sp CL190 orf encoding HMG-CoA reductase (HMGR) which in their summation are designated Operon D, encoding the entire mevalonate pathway.

FIG. 12 is a map of cloning vector pFHO3 containing S. cerevisiae orfs encoding phosphomevalonate kinase (PMK), mevalonate kinase (MVK), and mevalonate diphosphate decarboxylase (MDD), and acetoacetyl thiolase (AACT); A. thaliana orf encoding HMG-CoA synthase (HMGS); Streptomyces sp CL190 orf encoding HMG-CoA reductase (HMGR); R. capsulatus orf encoding IPP isomerase (IPPI) which in their summation are designated Operon E, encoding the entire mevalonate pathway and IPP isomerase.

FIG. 13 is a map of cloning vector pFHO4 containing a S. cerevisiae orf encoding acetoacetyl thiolase (AACT) coupled to the Streptomyces sp CL190 gene cluster which in their summation are designated Operon F, encoding the entire mevalonate pathway and IPP isomerase.

FIG. 14 is a plastid transformation vector pHKO7 containing N. tabacum chloroplast DNA (cpDNA) flanking the insertion of Operon C into the infA pseudogene.

FIG. 15 is a map of expression vector pHKO9 containing Operon B.

FIG. 16 is a map of expression vector pHK10 containing Operon C.

FIG. 17 is a map of plastid transformation vector pFHO6 containing N. tabacum chloroplast DNA (cpDNA) flanking the insertion of both Operon E and the R. capsulatus orf encoding phytoene synthase (PHS) into the infA pseudogene.

FIG. 18 are maps of expression vectors pKAS3, pKAS5 and pKAS6.

FIG. 19 is a map of expression vector pKAS6-aphA6.

FIG. 20 shows Southern blot analysis of transgenic tobacco plants transformed with pKAS5, showing four independently-generated homoplasmic lines. Five micrograms of total gDNA are digested with BtgI and examined. Five nanograms of plasmid controls are loaded. The digoxigenin-labeled probe is a DNA fragment containing the aadA gene plus a small fragment of chloroplast DNA from the desired locus of integration. 1) positive control plasmid, 2) pKAS5 plasmid, 3) and 5) S10 line 1, 4) and 12) S8 line 1, 6) S8 line 5, 7) WT tobacco, 8) positive control transgenic plant, 9) S8 line 4, 10) S8 line 3, 11) S8 line 2.

FIG. 21 shows transplastomic seedlings tested for inheritance of transgene and resistance to multiple antibiotics. The sample identity is the same for both photographs. Top row, from left to right: WT, spontanteous mutant line S8-5, transplastomic line S10-1; bottom row, from left to right, S8-1, S8-3, S8-4.

FIG. 22 shows Southern blot analysis of G-POS-transformed tobacco leaf DNA after restriction enzyme digestion with BglII and probing for the aphA6 gene. M: 1 kb ladder; 1:BglII-digested pKAS6aphA6 plasmid; 2: linearized pKAS6aphA6 plasmid; 3: WT DNA; 4: S24 line 8a-1; 5: S24 line 8a-2; 6: S24 line 8b-1; 7: S24 line 8b-2; 8: S25 line 6b-1; 9: S25 line 6b-2.

FIG. 23 shows Southern blot analysis of tobacco lines recovered from experiment S27 after co-transformation with vector pJS95 and G-POS vector pKAS5. Lanes: M: 1 KB molecular marker; 1: Wild type tobacco DNA; 2: pKAS5 plasmid; 3: pJS95 plasmid; 4 to 6: pKAS5 transgenic lines; 7: pJS95 transgenic line; 8: spontaneous mutant line.

FIG. 24 is a map of baseline cloning vector pLGPOS1, containing 2.5 kbp of the rpl23 operon of Lemna chloroplast.

FIG. 25 are maps of Lemna chloroplast vectors pLGPOS2, pLGPOS3, and pLGPOS4 containing antibiotic resistance genes cloned into the rpl23 operon. These are ready for introduction of partner protein genes as polycistronic constructs.

FIG. 26 are maps of Lemna G-POS chloroplast transformation vectors. Each vector is designed to integrate at the infA locus of the chloroplast genome and allow selection of transplastomic plants via resistance to aminoglycoside antibiotics such as kanamycin.

FIG. 27 shows tobacco G-POS vector pKAS6-aphA6-RcIPPI. The photograph on the left shows the results of restriction digestion of potential clones with AvrII. Lane 6 shows the desired 6711 and 1340 bp fragments produced by clone 7a. The plasmid diagram shows the Rhodobacter capsulatus IPPI gene cloned into the infA pseudogene locus of the tobacco chloroplast genome. Selection of transgenic plants is achieved by resistance to kanamycin provided by the aphA6 gene.

FIG. 28 is a diagram of cloning strategy for tobacco vector development that includes the Synechocystis IPPI gene.

FIG. 29 shows agarose gel electrophoresis of genomic DNA extracted from Synechocystis. M: 1 kb ladder; Lanes 1 and 3: 1 μl of genomic DNA; Lanes 2 and 4: 5 μl of genomic DNA. Bands are faint due to poor DNA yields.

FIG. 30 shows amplification of Synechocystis 16S rRNA gene fragment (686 bp) using the extracted genomic DNA. M: 1 kb ladder; Lane 1 and 2: genomic DNA from Synechocystis; Lane 3: negative control (no DNA).

FIG. 31 shows amplification of the Synechocystis IPPI gene using extracted genomic DNA. M: 1 kb ladder; Lanes 1 & 2: BspHI/SphI—(I) primers; Lanes 3 & 4: Pme1 primers; Lanes 5 & 6: BspHI/Sph1-(II) primers; Lanes 7 & 8: PacI/AscI primers; Lane 9: negative control (no DNA).

FIG. 32 are diagrams of vector constructs containing the RhIPPI gene (A), the SyIPPI gene (B) and tobacco chloroplast genome of nontransformed control (C) with predicted BtgI restriction enzyme sites used for genomic DNA digestion to predict insertion of the transgene in the chloroplast genome. Primer annealing sites in the rpoA gene used as probe for Southern analysis are shown.

FIG. 33 shows that PCR analysis of tobacco transformed with chloroplast transformation vector pKAS6-aphA6-RhIPPI yield a 242 bp PCR product (A) and transformation vector pKAS6-aphA6-SyIPPI yield a 1.2 kb PCR product (B). Lane M: Marker 1 kb (NEB); Lane 1: Nontransformed tobacco; Lanes 2-8 (A) and Lanes 2-7 (B): transformed tobacco; Lane 9 (A) and Lane 8 (B): plasmid positive control; Lane 10 (A) and Lane 9 (B): dH₂O control.

FIG. 34 shows Southern blot analysis of tobacco transformed with pKAS6-aphA6-RhIPPI (A) and pKAS6-aphA6-SyIPPI (B), digested with BtgI and hybridized with rpoA probe. P: plasmid positive control; M: Digoxigenin labeled marker; WT: nontransformed control; Lanes 1-7 (A) and Lanes 1-6 (B): genomic DNA of transgenic tobacco.

FIG. 35 shows Southern blot analysis of tobacco transformed with pKAS-aphA6-RhIPPI after three cycles of regeneration. P: plasmid positive control; E: empty lane; M: Dixoginen labeled marker; WT: nontransformed control; 1: 1^{st} cycle of regeneration; 2: 2^{nd} cycle; 3: 3^{rd} cycle.

FIG. 36 shows that RT-PCR analysis of tobacco transformed with chloroplast transformation vector pKAS6-aphA6-RhIPPI yields a 564 bp PCR product (A) and transformation vector pKAS6-aphA6-SyIPPI yield a 1.2 kb PCR product (B). Lane M: Marker 1 kb (NEB); Lane 1: Nontransformed tobacco; Lanes 2-8 (A) and Lanes 2-7 (B): transformed tobacco; Lane 9 (A) and Lane 8 (B): plasmid positive control; Lane 10 (A) and Lane 9 (B): dH₂O control.

FIG. 37 (A) shows RT-PCR amplification of RNA from transformed tobacco using primers designed for qRT-PCR. M: Marker, 100 bp (NEB); Lane 1: actin Tac9; Lane 2: actin Tob66; Lane 3: Glyceraldehyde-3-phosphate dehydrogenase; Lane 4: α-tubulin. (B) shows determination of the exponential range of amplification for internal controls actin Tac9 and α-tubulin and genes of interest: tobacco IPPI(1), tobacco IPPI(2), RhIPPI and SyIPPI.

FIG. 38 (A) shows a melt-curve analysis with the first derivative of the change in fluorescence intensity as a function temperature plotted, and (B) shows a sample standard curve using serial dilutions of nontransformed tobacco cDNA template as template.

FIG. 39 shows relative expression levels of the IPPI transgenes in transformed tobacco expressing the RhIPPI gene (A) or SyIPPI gene (B). The relative expression levels of transgene are calculated by using the comparative ^{ΔΔ}CT method. Nontransformed tobacco (WildType) is chosen as the calibrator. All samples are assayed in triplicate.

FIG. 40 shows relative expression levels of the IPPI transgenes in transformed tobacco expressing the RhIPPI gene (A) or SyIPPI gene (B) after 1^{st}, 2^{nd}, and 3^{rd} cycle of regeneration. The relative expression levels of transgene are calculated by using the comparative ^{ΔΔ}CT method. Nontransformed tobacco (WildType) is chosen as the calibrator. All samples are performed in triplicates.

FIG. 41 shows relative expression levels of the endogenous tobacco IPPI(1) gene in transgenic tobacco expressing the RhIPPI gene (A) or SyIPPI gene (B). The relative expression levels of the gene of interest are calculated by using the comparative ^{ΔΔ}CT method. Nontransformed tobacco (wild-type) is chosen as the calibrator. All samples are assayed in triplicate.

FIG. 42 shows relative mRNA levels of the endogenous IPPI(2) gene in transformed tobacco expressing the RhIPPI gene (A) or SyIPPI gene (B). The relative expression levels of the gene of interest are calculated by using the comparative ^{ΔΔ}CT method. Nontransformed tobacco (WildType) is chosen as the calibrator. All samples are assayed in triplicate.

FIG. 43 shows relative expression levels of all IPPI genes in transformed tobacco expressing the RhIPPI transgene after 1^{st}, 2^{nd}, and 3^{rd} cycle of regeneration under high light conditions: (A) RhIPPI, (B) tobacco IPPI(1), and (C) tobacco IPPI(2). The relative expression levels of transgene are calculated by using the comparative ^{ΔΔ}CT method. Nontransformed tobacco (WildType) is chosen as the calibrator. All samples are performed in triplicates.

FIG. 44 shows isoprenoid levels (ng pigment/mg tobacco leaf dry weight) in SyIPPI: S45(1) & S48(1), and RhIPPI: S48(7) & S48(8), tobacco transformants under normal growth conditions (150 μE/m²/sec).

FIG. 45 shows isoprenoid levels in RhIPPI clone S42(1) in tobacco transformants after 1^{st}, 2^{nd}, and 3^{rd} cycle of regeneration grown under high light conditions (300 μE/m²/sec) for one month.

FIG. 46 shows morphology of tobacco plants. (A) Leaves from a nontransformed tobacco, and RhIPPI and SyIPPI tobacco transformants. (B) Whole tobacco RhIPPI transformed plant showing changes in chlorophyll content. (C) Rooted tobacco plants.

FIG. 47 is a map of tobacco chloroplast vector pK6aphA6/HTNV-N.

FIG. 48 is a physical map of the targeted integration locus of the chloroplast genome. The predicted DNA fragments in resulting after insertion of transgenes are shown for both PCR (grey lines) and Southern blot (black bar) analyses. All genes shown are native to the tobacco chloroplast except for the contiguous DNA represented by psbA-aphA6-rbcL-Gene of Interest.

FIG. 49 PCR shows analysis of kanamycin resistant tobacco plants. (A) aphA6 PCR. Lane M: Molecular weight marker (NEB); Lane 1: Negative control with no DNA; Lane 2: wild type DNA; Lanes 3&8: Positive control plasmid DNA (pK6apha6HTNV and pK6apha6ANDV-N resply); Lanes 4-7: S24 lines (8a-1, 8a-2, 8b-1, 8b-2); Lanes 9,10: S25 lines (6b-1 & 6b-2). (B) HTNV-N PCR. Lane M: Molecular weight marker; Lane1: Negative control with no DNA; Lane2: Negative control wild type DNA; Lane3: positive control plasmid DNA pK6apha6HTNV; Lanes 4-7: S24 lines (8a-1, 8a-2, 8b-1, 8b-2) amplified at the expected size of 1.353 kb. (C) ANDV-N PCR. Lane M: Molecular marker; Lane 1: Negative control with no DNA; Lane2: Negative control wild type DNA; Lane 3: Positive control plasmid DNA from pK6apha6ANDV-N; Lane4: S25 line (6b-1) amplified at an expected size of 1422 bp.

FIG. 50 shows Southern analysis total genomic DNA isolated from S24 (Hantaan) and S25 (Andes) tobacco transformants. Arrow marks indicate non-specific or undigested fragments.

(A) BglII digested DNA/aph6 probe. Lane 1: pKAS6Apha6/Andv-N plasmid DNA (6966 bp and 1907 bp); Lane 2: pKAS6 Apha6/Htnv-N plasmid DNA (8804 bp); Lane 3: WT DNA (2054 bp); Lane 4: S24 (8a-1); Lane 5: S24 (8A-2); Lane 6: S24 (8B-1); Lane 7: S24 (8B-2); Lane 8: S25 (6B-1); Lane 9: S25 (6B-2). A 2667 bp product is expected for Lanes 5 to 9.

(B) BtgI digested S24 leaf DNA/infA/HTNV-N probe. Lane 1: Wild type DNA (11.9 kb); Lane 2: pKAS6 Apha6/HTNV-N plasmid DNA (linear; 8.8 kb); Lane 3: pKAS6 Apha6/HTNV-N plasmid DNA (4.49 kb, 4.31 kb); All transformants (Lanes 4 to 7) should have a band at 10.05 kb corresponding to HTNV-N cDNA.

(C) BtgI digested S25 leaf DNA/ANDV-N probe. Lane 1: Wild type DNA (11.9 kb); Lane 2: pKAS6 Apha6/ANDV-N plasmid DNA (linear; 8.87 kb); Lane 3: pKAS6 Apha6/ANDV-N plasmid DNA (cut with BtgI; 2.86 kb, 1.69 kb); Transformants (lanes 4 & 5) should have a band at 8.43 kb corresponding to Andy-N cDNA.

FIG. 51 (1) shows that transplastomic plants (B, C) are transferred to the soil at the same time as the wild type (A). All plants mature with no obvious phenotype. (2) shows confirmation of maternal inheritance: T1 generation seeds from S24, S25 and wild type soil grown plants are plated on Murashige and Skoog (MS) medium containing 50 mg/l Kanamycin. Panel A & B are wild type seeds on MS+Kanamycin (50 mg/l) and MS only respectively; Panel C and D are S24 (8b-1) and S25 (6b-2) on MS+Kanamycin (50 mg/l) respectively.

FIG. 52 shows protein analyses. (A) Coomassie stained 12.0% SDS-PAGE gel of His-Pur affinity purified Andes nucleoprotein. Lane M: Protein marker (BioRad); Lane 1: Flowthrough; Lane 2: Wash-1; Lane 3: Wash-2; Lane 4: Wash-3; Lane 5: Elution-1; Lane 6: Elution-2. (B) Western blot analysis of His-Pur affinity purified protein probed with Histidine antibody. 25 ug of purified protein is loaded in each lane. The wild type does not give any non-specific bands and S24 (Hantaan) gives the expected ˜49 kDa band along with a higher band which could be a dimer. S25 (Andes) samples show the desired ˜51 kDa band and no dimers or non-specific bands.

FIG. 53 shows immunoprecipitation of Hantaan and Andes affinity purified protein probed with Andes convalescent serum. Lane M: Protein Marker; Lane 1: Hantaan nucleoprotein precipitated with convalescent serum; Lane 2: Hantaan nucleoprotein precipitated with Mouse monoclonal Hantaan antibody; Lane 3: Andes nucleoprotein precipitated with Mouse monoclonal Andes antibody.

FIG. 54 is a schematic representation of the specificity of the antigen to either convalescent serum or their monoclonal antibodies by ELISA with 5 ug/ml protein concentration of wt: wild-type plant sample that goes through Hispur cobalt columns; S24: Histag purified Hantaan protein expressed in chloroplast; S25: Histag purified Andes protein expressed in chloroplast; E. coli: Histag purified Andes protein expressed in E. coli.

FIG. 55 shows nucleic acid sequences of 5′ psbY-rpl32-chlL-chlN 3′ (GenBank: EF508371.1 gene).

FIG. 56 shows nucleic acid sequences of Cryptomonas paramecium ATP Synthase operon 5′-rps2-tsf-atpl-atpH-atpG-atpF-atpD-atpA-3′.

FIG. 57 shows nucleic acid sequences of Arthrospira platensis NIES-39.

FIG. 58 shows pseudogenes of Arthrospira platensis (Spirulina platensis) nrs operon.

FIG. 59 shows open reading frame organization of the metal-resistance gene cluster from Synechocystis.

FIG. 60 shows nucleic acid sequences of GenBank: CP000077.1 Sulfolobus acidocaldarius DSM 639, complete genome (pl24-rpl14-rps7-conserved protein-rpl29-rps3-rpl22).

### BRIEF DESCRIPTION OF THE SEQUENCES

SEQ ID NO: 1) is a PCR primer containing Saccharomyces cerevisiae DNA.

SEQ ID NO: 2) is a PCR primer containing S. cerevisiae DNA.

SEQ ID NO: 3) is a PCR primer containing S. cerevisiae DNA.

SEQ ID NO: 4) is a PCR primer containing S. cerevisiae DNA.

SEQ ID NO: 5) is a PCR primer containing S. cerevisiae DNA.

SEQ ID NO: 6) is a PCR primer containing S. cerevisiae DNA.

SEQ ID NO: 7) is a PCR primer containing Arabidopsis thaliana DNA.

SEQ ID NO: 8) is a PCR primer containing A. thaliana DNA.

SEQ ID NO: 9) is a PCR primer containing A. thaliana DNA.

SEQ ID NO: 10) is a PCR primer containing A. thaliana DNA.

SEQ ID NO: 11) is a PCR primer containing S. cerevisiae DNA.

SEQ ID NO: 12) is a PCR primer containing S. cerevisiae DNA.

SEQ ID NO: 13) is an Oligonucleotide containing S. cerevisiae DNA.

SEQ ID NO: 14) is an Oligonucleotide containing A. thaliana and S. cerevisiae DNA.

SEQ ID NO: 15) is an Oligonucleotide containing S. cerevisiae DNA.

SEQ ID NO: 16) is an Oligonucleotide containing S. cerevisiae DNA.

SEQ ID NO: 17) is a Vector pBSNT27 containing Nicotiana tabacum DNA.

SEQ ID NO: 18) is an Oligonucleotide containing N. tabacum and S. cerevisiae DNA.

SEQ ID NO: 19) is an Oligonucleotide containing N. tabacum and A. thaliana DNA.

SEQ ID NO: 20) is a PCR primer containing Rhodobacter capsulatus DNA.

SEQ ID NO: 21) is a PCR is a primer containing R. capsulatus DNA.

SEQ ID NO: 22) is a PCR primer containing Schizosaccharomyces pombe DNA.

SEQ ID NO: 23) is a PCR primer containing S. pombe DNA.

SEQ ID NO: 24) is a PCR primer containing Streptomyces sp CL190 DNA.

SEQ ID NO: 25) is a PCR primer containing Streptomyces sp CL190 DNA.

SEQ ID NO: 26) is an Oligonucleotide containing S. cerevisiae DNA.

SEQ ID NO: 27) is an Oligonucleotide containing S. cerevisiae DNA.

SEQ ID NO: 28) is an Oligonucleotide containing Streptomyces sp CL190 and R. capsulatus DNA.

SEQ ID NO: 29) is an Oligonucleotide containing R. capsulatus DNA.

SEQ ID NO: 30) is an Oligonucleotide containing Streptomyces sp CL190 and S. cerevisiae DNA.

SEQ ID NO: 31) is an Oligonucleotide containing Streptomyces sp CL190 DNA.

SEQ ID NO: 32) is an Oligonucleotide containing N. tabacum and S. cerevisiae DNA.

SEQ ID NO: 33) is an Oligonucleotide containing N. tabacum and R. capsulatus DNA.

SEQ ID NO: 34) is an Oligonucleotide containing N. tabacum and S. cerevisiae DNA.

SEQ ID NO: 35) is an Oligonucleotide containing N. tabacum and S. pombe DNA.

SEQ ID NO: 36) is an Oligonucleotide containing NotI restriction site.

SEQ ID NO: 37) is an Oligonucleotide containing NotI restriction site.

SEQ ID NO: 38) is an Oligonucleotide containing S. cerevisiae DNA.

SEQ ID NO: 39) is an Oligonucleotide containing A. thaliana DNA.

SEQ ID NO: 40) is an Oligonucleotide containing S. cerevisiae DNA.

SEQ ID NO: 41) is an Oligonucleotide containing R. capsulatus DNA.

SEQ ID NO: 42) is an Oligonucleotide containing S. cerevisiae DNA.

SEQ ID NO: 43) is an Oligonucleotide containing S. pombe DNA.

SEQ ID NO: 44) is an Oligonucleotide containing R. capsulatus DNA.

SEQ ID NO: 45) is an Oligonucleotide containing R. capsulatus DNA.

SEQ ID NO: 46) is an Oligonucleotide containing S. pombe DNA.

SEQ ID NO: 47) is an Oligonucleotide containing S. pombe DNA.

SEQ ID NO: 48) is Saccharomyces cerevisiae orf for phosphomevalonate kinase (ERG8).

SEQ ID NO: 49) Saccharomyces cerevisiae orf for mevalonate kinase (ERG12).

SEQ ID NO: 50) Saccharomyces cerevisiae orf for mevalonate diphosphate decarboxylase (ERG19).

SEQ ID NO: 51) Saccharomyces cerevisiae orf for acetoacetyl thiolase.

SEQ ID NO: 52) Arabidopsis thaliana orf for 3-hydroxy-3-methylglutaryl-coenzyme A (HMG-CoA) synthase.

SEQ ID NO: 53) Arabidopsis thaliana orf for HMG-CoA reductase.

SEQ ID NO: 54) Schizosaccharomyces pombe IDI1 (IPP isomerase).

SEQ ID NO: 55) Rhodobacter capsulatus idiB (IPP isomerase).

SEQ ID NO: 56) Streptomyces sp CL190 orf encoding HMG-CoA reductase.

SEQ ID NO: 57) Streptomyces sp CL190 gene cluster containing mevalonate pathway and IPP isomerase orfs.

SEQ ID NO: 58) is Operon A containing A. thaliana and S. cerevisiae DNA

SEQ ID NO: 59) is Operon B containing A. thaliana and S. cerevisiae DNA.

SEQ ID NO: 60) is Operon C containing A. thaliana, S. cerevisiae, and R. capsulatus DNA.

SEQ ID NO: 61) is Operon D containing A. thaliana, S. cerevisiae, and Streptomyces sp CL190 DNA.

SEQ ID NO: 62) is Operon E containing A. thaliana, S. cerevisiae, Streptomyces sp CL190 DNA, and R. capsulatus DNA.

SEQ ID NO: 63) is Operon F containing S. cerevisiae and Streptomyces sp CL190 DNA.

SEQ ID NO: 64) is Operon G containing A. thaliana, S. cerevisiae and S. pombe DNA.

SEQ ID NO: 65) is PCR primer containing R. capsulatus DNA.

SEQ ID NO: 66) is PCR primer containing R. capsulatus DNA.

SEQ ID NO: 67) is an Oligonucleotide containing N. tabacum and R. capsulatus DNA.

SEQ ID NO: 68) is an Oligonucleotide containing N. tabacum and R. capsulatus DNA.

SEQ ID NO: 69) is an Oligonucleotide containing N. tabacum and S. cerevisiae DNA.

SEQ ID NO: 70) is an Oligonucleotide containing N. tabacum and R. capsulatus DNA.

SEQ ID NO: 71) is Rhodobacter capsulatus orf encoding phytoene synthase (crtB).

SEQ ID NO: 72) is plastid transformation vector pHKO4, containing Operon B, containing A. thaliana and S. cerevisiae DNA.

SEQ ID NO: 73) is plastid transformation vector pHKO7, containing Operon C, containing A. thaliana, S. cerevisiae, and R. capsulatus DNA.

SEQ ID NO: 74) is plastid transformation vector pHKO8, containing Operon G, containing A. thaliana, S. cerevisiae, and S. pombe DNA.

SEQ ID NO: 75) is plastid transformation vector pFHO5 containing R. capsulatus DNA encoding phytoene synthase.

SEQ ID NO: 76) is plastid transformation vector pFHO6, containing Operon E, containing A. thaliana, S. cerevisiae, Streptomyces sp CL190 DNA, and R. capsulatus DNA.

SEQ ID NO: 77) is a primer for PCR amplification of RhIPPI DNA.

SEQ ID NO: 78) is a primer for PCR amplification of RhIPPI DNA.

SEQ ID NO: 79) is a primer for PCR amplification of SyIPPI DNA.

SEQ ID NO: 80) is a primer for PCR amplification of SyIPPI DNA.

SEQ ID NO: 81) is a primer for PCR amplification of rpoA DNA.

SEQ ID NO: 82) is a primer for PCR amplification of rpoA DNA.

SEQ ID NO: 83) is a primer for RT-PCR amplification of RhIPPI DNA.

SEQ ID NO: 84) is a primer for RT-PCR amplification of RhIPPI DNA.

SEQ ID NO: 85) is a primer for RT-PCR amplification of SyIPPI DNA.

SEQ ID NO: 86) is a primer for RT-PCR amplification of SyIPPI DNA.

SEQ ID NO: 87) is a primer for PCR amplification of aphA6 DNA.

SEQ ID NO: 889) is a primer for PCR amplification of aphA6 DNA.

SEQ ID NO: 899) is a primer for PCR amplification of Hantaan nucleoprotein cDNA.

SEQ ID NO: 909) is a primer for PCR amplification of Hantaan nucleoprotein cDNA.

SEQ ID NO: 919) is a primer for PCR amplification of Andes nucleoprotein cDNA.

SEQ ID NO: 92) is a primer for PCR amplification of Andes nucleoprotein cDNA.

SEQ ID NO: 93) is a DNA sequence of Rhodomonas salina CCMP1319 operon.

SEQ ID NO: 94) is a DNA sequence of Cryptomonas paramecium operon.

SEQ ID NO: 95) is a DNA sequence of Arthrospira platensis NIES-39 operon.

SEQ ID NO: 96) is a DNA sequence of Sulfolobus acidocaldarius DSM 639 operon.

### DETAILED DESCRIPTION

In the description that follows, a number of terms used in genetic engineering are utilized. In order to provide a clear and consistent understanding of the specification and claims, including the scope to be given such terms, the following definitions are provided.

A protein is considered an isolated protein if it is a protein isolated from a host cell in which it is naturally produced. It can be purified or it can simply be free of other proteins and biological materials with which it is associated in nature, for example, if it is recombinantly produced.

An isolated nucleic acid is a nucleic acid the structure of which is not identical to that of any naturally occurring nucleic acid or to that of any fragment of a naturally occurring genomic nucleic acid spanning more than three separate genes. The term therefore covers, for example, (a) a DNA which has the sequence of part of a naturally occurring genomic DNA molecule, but is not flanked by both of the coding or noncoding sequences that flank that part of the molecule in the genome of the organism in which it naturally occurs; (b) a nucleic acid incorporated into a vector or into the genomic or plastomic DNA of a prokaryote or eukaryote in a manner such that the resulting molecule is not identical to any naturally occurring vector or genomic or plastomic DNA; (c) a separate molecule such as a cDNA, a genomic or plastomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library.

One DNA portion or sequence is downstream of second DNA portion or sequence when it is located 3′ of the second sequence. One DNA portion or sequence is upstream of a second DNA portion or sequence when it is located 5′ of that sequence.

One DNA molecule or sequence and another are heterologous to one another if the two are not derived from the same ultimate natural source, or are not naturally contiguous to each other. The sequences may be natural sequences, or at least one sequence can be derived from two different species or one sequence can be produced by chemical synthesis provided that the nucleotide sequence of the synthesized portion was not derived from the same organism as the other sequence.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

A nucleotide sequence is operably linked when it is placed into a functional relationship with another nucleotide sequence. For instance, a promoter is operably linked to a coding sequence if the promoter effects its transcription or expression. Generally, operably linked means that the sequences being linked are contiguous and, where necessary to join two protein coding regions, contiguous and in reading frame. However, it is well known that certain genetic elements, such as enhancers, may be operably linked even at a distance, i.e., even if not contiguous.

In a plastome, sequences are physically linked by virtue of the chromosome configuration, but they are not necessarily operably linked due to differential expression for example. Transgenes can be physically linked prior to transformation, or can become physically linked once they insert into a plastome. Transgenes can become operably linked if they share regulatory sequences upon insertion into a plastome.

The term recombinant polynucleotide refers to a polynucleotide which is made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

The polynucleotides may also be produced by chemical synthesis, e.g., by the phosphoramidite method described by Beaucage and Caruthers (1981) Tetra. Letts., 22:1859-1862 or the triester method according to Matteuci et al. (1981) J. Am. Chem. Soc., 103: 3185, and may be performed on commercial automated oligonucleotide synthesizers. A double-stranded fragment may be obtained from the single stranded product of chemical synthesis either by synthesizing the complementary strand and annealing the strands together under appropriate conditions or by adding the complementary strand using DNA polymerase with an appropriate primer sequence.

Polynucleotide constructs prepared for introduction into a prokaryotic or eukaryotic host will typically, but not always, comprise a replication system (i.e. vector) recognized by the host, including the intended polynucleotide fragment encoding the desired polypeptide, and will preferably, but not necessarily, also include transcription and translational initiation regulatory sequences operably linked to the polypeptide-encoding segment. Expression systems (expression vectors) may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides may also be included where appropriate, preferably from secreted polypeptides of the same or related species, which allow the protein to cross and/or lodge in cell membranes or be secreted from the cell.

Variants or sequences having substantial identity or homology with the polynucleotides encoding enzymes of the mevalonate pathway may be utilized in the practice of the invention. Such sequences can be referred to as variants or modified sequences. That is, a polynucleotide sequence may be modified yet still retain the ability to encode a polypeptide exhibiting the desired activity. Such variants or modified sequences are thus equivalents. Generally, the variant or modified sequence will comprise at least about 40%-60%, preferably about 60%-80%, more preferably about 80%-90%, and even more preferably about 90%-95% sequence identity with the native sequence.

Sequence relationships between two or more nucleic acids or polynucleotides are generally defined as sequence identity, percentage of sequence identity, and substantial identity. In determining sequence identity, a “reference sequence” is used as a basis for sequence comparison. The reference may be a subset or the entirety of a specified sequence. That is, the reference sequence may be a full-length gene sequence or a segment of the gene sequence.

Methods for alignment of sequences for comparison are well known in the art. See, for example, Smith et al. (1981) Adv. Appl. Math. 2:482; Needleman et al. (1970) J. Mol. Biol. 48:443; Pearson et al. (1988) Proc. Natl. Acad. Sci. 85:2444; CLUSTAL in the PC/Gene Program by Intelligenetics, Mountain View, Calif.; GAP, BESTFIT, BLAST, FASTA, and TFASTA in the Wisconsin Genetics Software Package, Genetics Computer Group (GCG), 575 Science Drive, Madison, Wis., USA. Preferred computer alignment methods also include the BLASTP, BLASTN, and BLASTX algorithms. See, Altschul et al. (1990) J. Mol. Biol. 215:403-410.

“Sequence identity” or “identity” in the context of nucleic acid or polypeptide sequences refers to the nucleic acid bases or residues in the two sequences that are the same when aligned for maximum correspondence over a specified comparison window. “Percentage of sequence identity” refers to the value determined by comparing two optimally aligned sequences over a comparison window, wherein the portion of the polynucleotide sequence in the comparison window may comprise additions or deletions as compared to the reference window for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Polynucleotide sequences having “substantial identity” are those sequences having at least about 50%-60% sequence identity, generally at least 70% sequence identity, preferably at least 80%, more preferably at least 90%, and most preferably at least 95%, compared to a reference sequence using one of the alignment programs described above. Preferably sequence identity is determined using the default parameters determined by the program. Substantial identity of amino acid sequence generally means sequence identity of at least 50%, more preferably at least 70%, 80%, 90%, and most preferably at least 95%.

Nucleotide sequences are generally substantially identical if the two molecules hybridize to each other under stringent conditions. Generally, stringent conditions are selected to be about 5° C. lower than the thermal melting point for the specific sequence at a defined ionic strength and pH. Nucleic acid molecules that do not hybridize to each other under stringent conditions may still be substantially identical if the polypeptides they encode are substantially identical. This may occur, for example, when a copy of a nucleic acid is created using the maximum codon degeneracy permitted by the genetic code.

As noted, hybridization of sequences may be carried out under stringent conditions. By “stringent conditions” is intended conditions under which a probe will hybridize to its target sequence to a detectably greater degree than to other sequences. Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary stringent conditions include hybridization with a buffer solution of 30 to 35% formamide, 1.0 M NaCl, 1% SDS (sodium dodecyl sulphate) at 37° C., and a wash in 1× to 2×SSC (20×SSC=3.0 M NaCl/0.3 M trisodium citrate) at 50 to 55° C. It is recognized that the temperature, salt, and wash conditions may be altered to increase or decrease stringency conditions. For the post-hybridization washes, the critical factors are the ionic strength and temperature of the final wash solution. See, Meinkoth and Wahl (1984) Anal. Biochem. 138:267-284.

As indicated, fragments and variants of the nucleotide sequences of the invention are encompassed herein. By “fragment” is intended a portion of the nucleotide sequence. Fragments of the polynucleotide sequence will generally encode polypeptides which retain the biological/enzymatic activity of the native protein. Those of skill in the art routinely generate fragments of polynucleotides of interest through use of commercially available restriction enzymes; synthetic construction of desired polynucleotides based on known sequences; or use of “erase-a-base” technologies such as Bal 31 exonuclease, by which the skilled artisan can generate hundreds of fragments of a known polynucleotide sequence from along the entire length of the molecule by time-controlled, limited digestion. Fragments that retain at least one biological or enzymatic activity of the native protein are equivalents of the native protein for that activity.

By “variants” is intended substantially similar sequences. For example, for nucleotide sequences, conservative variants include those sequences that, because of the degeneracy of the genetic code, encode the amino acid sequence of an enzyme of the mevalonate pathway. Variant nucleotide sequences include synthetically derived sequences, such as those generated for example, using site-directed mutagenesis. Generally, nucleotide sequence variants of the invention will have at least 40%, 50%, 60%, 70%, generally 80%, preferably 85%, 90%, up to 95% sequence identity to its respective native nucleotide sequence. Activity of polypeptides encoded by fragments or variants of polynucleotides can be confirmed by assays disclosed herein.

“Variant” in the context of proteins is intended to mean a protein derived from the native protein by deletion or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or human manipulation. Conservative amino acid substitutions will generally result in variants that retain biological function. Such variants are equivalents of the native protein. Variant proteins that retain a desired biological activity are encompassed within the subject invention. Variant proteins of the invention may include those that are altered in various ways including amino acid substitutions, deletions, truncations, and insertions. Methods for such manipulation are generally known in the art. See, for example, Kunkel (1985) Proc. Natl. Acad. Sci. USA 82:488-492; Kunkel et al. (1987) Methods and Enzymol; 154:367-382; and the references cited therein.

An expression cassette may contain at least one polynucleotide of interest to be cotransformed into the organism. Such an expression cassette is preferably provided with a plurality of restriction sites for insertion of the sequences of the invention to be under the transcriptional regulation of the regulatory regions. The expression cassette may additionally contain selectable marker genes.

The cassette may include 5′ and 3′ regulatory sequences operably linked to a polynucleotide of interest. By “operably linked” is intended, for example, a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. When a polynucleotide comprises a plurality of coding regions that are operably linked such that they are under the control of a single promoter, the polynucleotide may be referred to as an “operon”.

The expression cassette will optionally include in the 5′-3′ direction of transcription, a transcriptional and translational initiation region, a polynucleotide sequence of interest and a transcriptional and translational termination region functional in plants or microalgae. The transcriptional initiation region, the promoter, is optional, but may be native or analogous, or foreign or heterologous, to the intended host. Additionally, the promoter may be the natural sequence or alternatively a synthetic sequence. By “foreign” is intended that the transcriptional initiation region is not found in the native organism into which the transcriptional initiation region is introduced. As used herein, a chimeric gene comprises a coding sequence operably linked to a transcriptional initiation region that is heterologous to the coding sequence.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the polynucleotides of interest may be optimized for expression in the transformed organism. That is, the genes can be synthesized using plant or algae plastid-preferred codons corresponding to the plastids of the plant or algae of interest. Methods are available in the art for synthesizing such codon optimized polynucleotides. See, for example, U.S. Pat. Nos. 5,380,831 and 5,436,391, and Murray et al. (1989) Nucleic Acids Res. 17:477-498, herein incorporated by reference. Of course, the skilled artisan will appreciate that for the transplastomic purposes described herein, sequence optimization should be conducted with plastid codon usage frequency in mind, rather than the plant or algae genome codon usage exemplified in these references.

It is now well known in the art that when synthesizing a polynucleotide of interest for improved expression in a host cell it is desirable to design the gene such that its frequency of codon usage approaches the frequency of codon usage of the host cell. It is also well known that plastome codon usage may vary from that of the host plant or microalgae genome. For purposes of the subject invention, “frequency of preferred codon usage” refers to the preference exhibited by a specific host cell plastid in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a plastid can be calculated by averaging frequency of preferred codon usage in a number of genes expressed by the plastid. It usually is preferable that this analysis be limited to genes that are among those more highly expressed by the plastid. Alternatively, the polynucleotide of interest may be synthesized to have a greater number of the host plastid's most preferred codon for each amino acid, or to reduce the number of codons that are rarely used by the host.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region), Elroy-Stein et al. (1989) PNAS USA 86:6126-6130; potyvirus leaders, for example, TEV leader (Tobacco Etch Virus), Allison et al. (1986); MDMV Leader (Maize Dwarf Mosaic Virus) Virology 154:9-20; and human immunoglobulin heavy-chain binding protein (BiP), Macejak et al. (1991) Nature 353:90-94; untranslated leader from the coat protein mRNA of alfalfa mosaic virus (AMV RNA 4), Jobling et al. (1987) Nature 325:622-625; tobacco mosaic virus leader (TMV), Gallie et al. (1989) in Molecular Biology of RNA, ed. Cech (Liss, New York), pp. 237-256; and maize chlorotic mottle virus leader (MCMV), Lommel et al. (1991) Virology 81:382-385. See also, Della-Cioppa et al. (1987) Plant Physiol. 84:965-968. Other methods known to enhance translation can also be utilized, for example, introns, and the like.

In preparing an expression cassette, the various polynucleotide fragments may be manipulated, so as to provide for the polynucleotide sequences in the proper orientation and, as appropriate, in the proper reading frame. Toward this end, adapters or linkers may be employed to join the polynucleotide fragments or other manipulations may be involved to provide for convenient restriction sites, removal of superfluous nucleotides, removal of restriction sites, or the like. For this purpose, in vitro mutagenesis, primer repair, restriction, annealing, resubstitutions, e.g., transitions and transversions, may be involved.

In addition, expressed gene products may be localized to specific organelles in the target cell by ligating DNA or RNA coded for peptide leader sequences to the polynucleotide of interest. Such leader sequences can be obtained from several genes of either plant or other sources. These genes encode cytoplasmically-synthesized proteins directed to, for example, mitochondria (the F1-ATPase beta subunit from yeast or tobacco, cytochrome c1 from yeast), chloroplasts (cytochrome oxidase subunit Va from yeast, small subunit of rubisco from pea), endoplasmic reticulum lumen (protein disulfide isomerase), vacuole (carboxypeptidase Y and proteinase A from yeast, phytohemagglutinin from French bean), peroxisomes (D-aminoacid oxidase, uricase) and lysosomes (hydrolases).

Following transformation, a plant may be regenerated, e.g., from single cells, callus tissue, or leaf discs, as is standard in the art. Almost any plant can be entirely regenerated from cells, tissues, and organs of the plant. Available techniques are reviewed in Vasil et al. (1984) in Cell Culture and Somatic Cell Genetics of Plants, Vols. I, II, and III, Laboratory Procedures and Their Applications (Academic press); and Weissbach et al. (1989) Methods for Plant Mol. Biol.

The transformed plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited, and then seeds harvested to ensure expression of the desired phenotypic characteristic has been achieved.

The particular choice of a transformation technology will be determined by its efficiency to transform certain target species, as well as the experience and preference of the person practicing the invention with a particular methodology of choice. It will be apparent to the skilled person that the particular choice of a transformation system to introduce nucleic acid into plant or microalgae plastids is not essential to or a limitation of the invention, nor is the choice of technique for plant regeneration.

Plants may be transformed using biolistic introduction of DNA-coated gold particles using the particle inflow gun (PIG), where plant tissues are bombared with gold particles coated with pseudogene vectors, allowing biolistic delivery of particles into cells. The transformation efficiency can be optimized by modifying parameters such as, for example, gold particle sizes, the amount of gold used per shot, biolistic helium pressure, osmotic pressure prior to and subsequent of particle bombardment, antibiotic selection levels, tissue types, and post-bombardment conditions. In addition, polyethyleneglycol-mediated protoplast transformation can be used to generate transgenic plants, as is described in De santis-Maciossek et al., 1999; Kofer et al., 1998; Koop et al., 1996.

Also according to the invention, there is provided a plant or microalgae cell having the constructs of the invention. A further aspect of the present invention provides a method of making such a plant cell involving introduction of a vector including the construct into a plant cell. For integration of the construct into the plastid genome (the plastome), such introduction will be followed by recombination between the vector and the plastome genome to introduce the operon sequence of nucleotides into the plastome. RNA encoded by the introduced nucleic acid construct (operon) may then be transcribed in the cell and descendants thereof, including cells in plants regenerated from transformed material. A gene stably incorporated into the plastome of a plant or microalgae is passed from generation to generation to descendants of the plant or microalgae, so such descendants should show the desired phenotype.

The present invention also provides a plant or microalgae culture comprising a plant cell as disclosed. Transformed seeds and plant parts are also encompassed. As used herein, the expressions “cell,” “cell line,” and “cell culture” are used interchangeably and all such designations include progeny, meaning descendants, not limited to the immediate generation of descendants but including all generations of descendants. Thus, the words “transformants” and “transformed cells” include the primary subject cell and cultures derived therefrom without regard for the number of transfers. It is also understood that all progeny may not be precisely identical in DNA content, due to naturally occurring, deliberate, or inadvertent caused mutations. Mutant progeny that have the same function or biological activity as screened for in the originally transformed cell are included. Where distinct designations are intended, it will be clear from the context.

In addition to a plant or microalgae, the present invention provides any clone of such a plant or microalgae, seed, selfed or hybrid or mated descendants, and any part of any of these, such as cuttings or seed for plants. The invention provides any plant propagule, that is any part which may be used in reproduction or propagation, sexual or asexual, including cuttings, seed, and so on. Also encompassed by the invention is a plant or microalgae which is a sexually or asexually propagated off-spring, clone, or descendant of such a plant or microalgae, or any part or propagule of said plant, off-spring, clone, or descendant. Plant or microalgae extracts and derivatives are also provided.

The present invention may be used for transformation of any plant species, including, but not limited to, corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium hirsutum), sweet potato (Ipomoea batatus), cassaya (Manihot esculenta), coffee (Cofea ssp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidental), macadamia (Macadamia integrifolia), almond (Prunus amygdalus), sugar beets (Beta vulgaris), oats, barley, vegetables, ornamentals, and conifers.

Preferably, plants of the present invention are crop plants (for example, cereals and pulses, maize, wheat, potatoes, tapioca, rice, sorghum, millet, cassaya, barley, pea, and other root, tuber, or seed crops. Important seed crops are oil-seed rape, sugar beet, maize, sunflower, soybean, and sorghum. Horticultural plants to which the present invention may be applied may include lettuce; endive; and vegetable brassicas including cabbage, broccoli, and cauliflower; and carnations and geraniums. The present invention may be applied to tobacco, cucurbits, carrot, strawberry, sunflower, tomato, pepper, chrysanthemum, petunia, rose, poplar, eucalyptus, and pine.

Grain plants that provide seeds of interest include oil-seed plants and leguminous plants. Seeds of interest include grain seeds, such as corn, wheat, barley, rice, sorghum, rye, etc. Oil seed plants include cotton, soybean, safflower, sunflower, Brassica, maize, alfalfa, palm, coconut, etc. Leguminous plants include beans and peas. Beans including guar, locust bean, fenugreek, soybean, garden beans, cowpea, mungbean, lima bean, fava bean, lentils, chickpea, etc.

Microalgae include but are not limited to the Chlorophyta and the Rhodophyta and may be such organisms as Chlamydomonas, Haematococcus, and Ouneliella.

Further, this invention provides the Gene Positioning technology for biosynthesis of products of interest via plastid transformation of plants, algae, cyanobacteria and Archaea with pseudogene vectors containing polynucleotides encoding one or more products of interest. The transformants can be successfully selected and recovered. In addition, tissues of transformants are repeatedly regenerated to promote homogeneity of the transformed chloroplasts (homoplasmy). Further, the pseudogene vector can be integrated into the desired locus in the plastid genome, allowing simultaneous expression of multiple transgenes.

In certain embodiments, this invention provides a method for biosynthesis of proteins of interest, for example, enzymes of mevalonate pathway, optionally including but not limited to, Rhodobacter capsulatis IPPI and Synechocystis IPPI; and viral proteins such as, for example, Hankaan or Andes nucleoprotein antigens, via plastid transformation of transgenic tobacco, lemna, and Chlamydomonas using the Gene Positioning System (G-POS).

Two types of IPP isomerase are known to date. Type I enzymes, such as the one found in Rhodobacter, function as monomeric enzymes in the MVA pathway. They have been found in humans, archebacteria, Saccharomyces, and the cytoplasm of higher plants. A second type of IPP isomerase that functions in the DXP pathway was identified in Streptomyces (Kaneda et al., 2001) and was determined to function as a tetramer in a flavin mononucleotide- and NADPH-dependent manner. Type II enzymes are found in plant chloroplasts, eubacteria such as E. coli, B. subtilis, S. aureus, and cyanobacteria such as Synechocystis sp. Comparison of the kinetic constants for Type II IPP isomerases (Barkley et al., 2004) demonstrated that the enzyme found in Synechocystis sp. PCC 6803 has a relatively high enzymatic efficiency as measured by kcat/Km (4.4×10³ versus 1.6×10³ for Streptomyces). The highest efficiency has been found for Staphylococcus aureus (6.8×10⁴); however, one obstacle in utilizing Staphylococcus aureus as a source of IPPI gene is the pathogenicity of the organism. Nevertheless, Synechocystis is one example of a preferred source of Type II IPP isomerase.

One embodiment of the present invention is a tobacco-chloroplast-specific vector containing IPPI gene. In certain embodiments, tobacco chloroplast-specific vectors containing the R. capsulatus IPPI (RhIPPI) gene or the Synechocystis IPPI (SyIPPI) gene are constructed. These vectors use the aphA6 enzyme from Acinetobacter baumanii as the selectable marker gene providing resistance to kanamycin (Herz et al, 2005; Huang et al, 2002). The Rhodobacter IPPI or Synechocystis IPPI genes are inserted downstream of the selectable marker into the infA locus. InfA gene is a pseudogene that has lost its functionality in many plants, but is part of the actively transcribed rpl23 operon (Millen et al, 2001). Transgenes inserted using the gene-positioning system (GPOS)™ technology of the present invention lack their own transcriptional promoter, but are controlled by upstream regulatory elements of the polycistron.

The expression of transgene can be quantified by quantitative RT-PCR. As exemplified herein, quantitative RT-PCR tests show that five out of seven RhIPPI transformants and four out of six SyIPPI transformants produce similar elevated levels of transgene mRNA. The lines containing lower levels of transgene mRNA are first generation plants can further be developed into lines with higher transgene expression by repeated subculturing and selection on antibiotics such as kanamycin, as is demonstrated for the RhIPPI line S42(1). In certain instances, transcript expression of transgenes, such as RhIPPI and SyIPPI as exemplified herein may reach a plateau after the 2^{nd} cycle of regeneration.

In tobacco, two IPPI cDNAs have been reported and their subcellular localizations have been determined (Nakamura et al., 2001). IPPI(1) is a Type II isomerase, like that of Synechocystis, and functions in the chloroplast. IPPI(2) is a Type I isomerase, similar to the Rhodobacter IPPI, and it is located in the cytosol. Under normal growth conditions, IPPI(1) mRNA is more abundant than IPPI(2) perhaps owing to a high constitutive requirement by plants for isoprenoid based compounds such as chlorophyll and carotenoids. Cytosolic isoprenoid production controlled by IPPI(2) are generally induced in response to salt, cold and pathogen stresses.

As exemplified herein, no significant alteration of cytosolic tobacco IPPI(2) mRNA is detected in the transgenic plants of the present invention, expressing either the RhIPPI or SyIPPI gene in the chloroplast. Effects on endogenous tobacco IPPI gene expression are confined to the chloroplast, where the transgenes are located. The endogenous tobacco IPPI gene expression exemplified herein is applicable to other plants, as the Type I and Type II enzymes and biosynthetic pathway flux in higher plants are compartmentalized.

In another embodiment, IPPI(1) mRNA expression increases significantly in transgenic tobacco expressing the RhIPPI or SyIPPI in the chloroplast under normal growth conditions. This effect on endogenous IPPI(1) transcription is even more pronounced in plants exposed to high-light, a known trigger for induction; qRT-PCR analyses show that the level of RhIPPI transgene remains the same, but the level of IPPI(1) increases dramatically from 10⁴ under normal growth conditions to 10²⁰ under high light. Although a significant increase in IPPI(1) mRNA is detected in the transformed tobacco plants exemplified herein, there is a slight reduction in chloroplast isoprenoids as measured by HPLC when compared to wild-type tobacco plants.

In addition, in the chloroplast, regulation of gene expression is primarily regulated by post-transcriptional processes, frequently via translation initiation. A direct correlation between mRNA and protein levels is uncommon. Transgenic plants appear similar to wild-type plants with the exception of reduced chlorophyll in RhIPPI plants. Also, Rhodobacter IPPI has an effect on tobacco IPPI and chlorophyll biosynthesis, possibly due to that this heterologous protein is able to bind IPP substrate, but unable to convert it effectively to DMAPP. Manganese or magnesium is believed to be a required co-factor for Type I enzymes; limitation of Mg⁺⁺ or Mn⁺⁺ in the chloroplast environment could limit the production of IPP.

For Synechocystis IPPI expression in tobacco chloroplasts, Type II proteins such as these naturally form homotetrameric complexes; a heterotetramer of SyIPPI and IPPI(1) can distort tertiary structure sufficiently well to interfere with catalysis. In addition, the lack of a visible effect of SyIPPI expression on leaf chlorophyll, similar to that observed with RhIPPI, suggests that the transgene is not significantly interfering with tobacco IPPI(1) catalytic activity, but is altering the ability of the tobacco protein to regulate its own transcription. In addition, the SyIPPI protein can be functioning in tobacco chloroplasts while masking any negative effects of its expression upon tobacco IPPI(1) protein. The degree to which IPPI(1) and IPPI(2) interact in vivo can be determined by co-immunoprecipitation experiments as is known in the art.

The effects of IPPI expression in chloroplasts can be observed in a variety of plant organs other than tobacco leaves exemplified herein. Sun et al. 1998 demonstrated an observable phenotype in Hematococcus pluvialis algae, whereby cells increased IPPI transcription and carotenoid biosynthesis in response to high light exposure. In another embodiment, this invention provides a method for biosynthesis of pharmaceuticals, diagnostic antigens and vaccines, such as for example, Hantaan nucleoprotein and Andes nucleoprotein, via plastid transformation of tobacco plants. Tobacco (Nicotiana tabacum var. ‘Petit Havana’) is particularly of interest for chloroplast engineering due to the ease of handling in tissue culture, the ease of manipulating the chloroplast genome, and the rapidity of recovery of transgenic plants. Using the Gene Positioning System (G-POS)™, a 3- to 4-fold elevated expression of Hantaan and Andes nucleoprotein antigens in plastids of tobacco to approximately 6% to 8% of total soluble protein are obtained without system optimization. G-POS-based antigens are sufficiently reactive with convalescent human antisera to function in an in vitro diagnostic assay, with reactivity similar, or superior, to that prepared in E. coli. Thus, the G-POS method can produce protein antigens at low cost and be applied to other species of chloroplast-based production platforms.

Specifically, the G-POS™ (Gene Positioning) strategy of the present invention utilizes pseudogene sites as targets for plastid transgenesis. As chloroplast genomes have evolved over time, many plastid genes have been lost because their function was no longer necessary for survival or because the genes were transferred to nuclear chromosomes (Martin et al. 1998). The original copies of such genes in the plastid chromosome may mutate until they are no longer functional, i.e., any such gene becomes a pseudogene. Pseudogenes in chloroplast genomes, identified for a multiplicity of crops and algae, become potential target sites for insertion of genes of interest using the pre-existing regulatory sequences (Hahn and Kuehnle 2003). This is in contrast to the paradigm of chloroplast transformation vectors, which contain multiple monocistronic operons, each controlled by distinct regulatory elements. Such vectors may lead to undesirable recombination events due to repeat sequences in the vectors that result in elimination of necessary transgene sequences.

In an embodiment, vectors of the present invention use rpl23 along with the pseudogene (infA) as the region of transgene insertion. The pseudogene infA has lost its functionality due to endosymbiosis. However, they possess an operon that is actively transcribed (Millen et al, 2001). It has been shown that pseudogenes occur by individual gene loss rather than loss of clusters of genes.

Using a non-optimized G-POS system, expression of Hantaan and Andes nucleoproteins at 6-8% of total soluble protein (tsp) is achieved, which demonstrates the potential for large scale production of recombinant proteins. Gene expression in chloroplasts is primarily regulated via translation, and therefore higher levels of productivity are anticipated with minor enhancements of DNA sequences in the 5′ UTR of the transgene.

In addition, the present invention also provides improved protein purification techniques, which successfully yield active proteins after purification. Elgh et al. (1996) describes an irreversible disruption of the epitopes during the purification process due to the presence of urea in the buffer. The use of purification agents such as Guanidine HCl, a strong denaturant, may prevent the protein from displaying the necessary epitopes for recognition by the antisera. It has been shown that Puumala nucleoprotein expressed in E. coli forms insoluble aggregates in which the protein may be improperly folded.

This invention provides optimized protein purification methods, where proteins produced by transgenic plants can be purified by affinity purification, using 6M Guanidine HCl, per manufacturer's instructions for HisPur affinity columns. In an embodiment, an efficient yield of concentrated recombinant protein (2 mg/ml) is obtained. The purified protein with 6 M urea is subsequently transferred into physiological buffers, allowing the protein to renature/refold into an antigenically proper conformation. For example, purified proteins in 6M Guanidine HCl are renatured by dialysis into PBS or by substituting 6M Urea for 6M Guanidine HCl during the affinity purification step. Both of these modified procedures succeeded in producing immunoreactive nucleoproteins. Comparison of chloroplast-derived proteins of the present invention with that produced in E. coli conclusively demonstrates that G-POS chloroplast system produces a product with equal or superior diagnostic utility. Hantavirus proteins produced in the tobacco chloroplast are highly reactive to human convalescent serum, an important and necessary prerequisite for employment in an in vitro diagnostic test.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description. Unless indicated otherwise, the respective contents of the documents cited herein are hereby incorporated by reference to the extent they are not inconsistent with the teachings of this specification.

Percentages and ratios given herein are by weight, and temperatures are in degrees Celsius unless otherwise indicated. The references cited within this application are herein incorporated by reference to the extent applicable. Where necessary to better exemplify the invention, percentages and ratios may be cross-combined.

### Example 1

### Isolation of Orfs Encoding Enzymes of the Mevalonate Pathway for the Construction of Vectors pFCO1 and pFCO2

In an exemplified embodiment, vectors containing open reading frames (orfs) encoding enzymes of the mevalonate pathway are constructed. Polynucleotides derived from the yeast Saccharomyces cerevisiae, the plant Arabidopsis thaliana, and the eubacterium Streptomyces sp CL190 are used for the construction of vectors, including plastid delivery vehicles, containing orfs for biosynthesis of the mevalonate pathway enzymes. Construction of the vectors is not limited to the methods described. It is routine for one skilled in the art to choose alternative restriction sites, PCR primers, etc. to create analogous plasmids containing the same orfs or other orfs encoding the enzymes of the mevalonate pathway. Many of the steps in the construction of the plasmids of the subject invention can utilize the joining of blunt-end DNA fragments by ligation. As orientation with respect to the promoter upstream (5′) of the described orfs can be critical for biosynthesis of the encoded polypeptides, restriction analysis is used to determine the orientation in all instances involving blunt-end ligations. A novel directional ligation methodology, chain reaction cloning (Pachuk et al., Gene 243:19-25, 2000), can also be used as an alternative to standard ligations in which the resultant orientation of the insert is not fixed. All PCR products are evaluated by sequence analysis as is well known in the art.

The construction of a synthetic operon comprising three yeast orfs encoding phosphomevalonate kinase, mevalonate kinase, and mevalonate diphosphate decarboxylase is described by Hahn et al. (Hahn et al., J. Bacteriol. 183:1-11, 2001). This same synthetic operon, contained within plasmid pFCO2, is able to synthesize, in vivo, polypeptides with enzymatic activities able to convert exogenously supplied mevalonate to IPP as demonstrated by the ability of the mevalonate pathway orfs to complement the temperature sensitive dxs::kanr lethal mutation in E. coli strain FH11 (Hahn et al., 2001).

Plasmids pFCO1 and pFCO2 containing a synthetic operon for the biosynthesis of IPP from mevalonate are constructed as follows: Three yeast orfs encoding mevalonate kinase, phosphomevalonate kinase, and mevalonate diphosphate decarboxylase are isolated from S. cerevisiae genomic DNA by PCR using the respective primer sets

FH0129-2:
(SEQ ID NO: 1)
5′ GGACTAGTCTGCAGGAGGAGTTTTAATGTCATTACCGTTCTTAAC
TTCTGCACCGGG-3′ (sense)
and
FH0129-1:
(SEQ ID NO: 2)
5′ TTCTCGAG [symbol] [symbol]
ATTAAAACTCCTCCTGTGAAGTCCATGGTAAATTC
G 3′ (antisense);
FH0211-1:
(SEQ ID NO: 3)
5′ TAGCGGCCGCAGGAGGAGTTCATATGTCAGAGTTGAGAGCCTTC
AGTGCCCCAGGG 3′ (sense)
and
FH0211-2:
(SEQ ID NO: 4)
5′ TTTCTGCAGTTTATCAAGATAAGTTTCCGGATCTTT 3′
(antisense);
CT0419-1:
(SEQ ID NO: 5)
5′ GGAATTCATGACCGTTTACACAGCATCCGTTACCGCACCCG 3′
(sense)
and
CT0419-2:
(SEQ ID NO: 6)
5′ GGCTCGAGTTAAAACTCCTCTTCCTTTGGTAGACCAGTCTTTGCG
3′ (antisense).

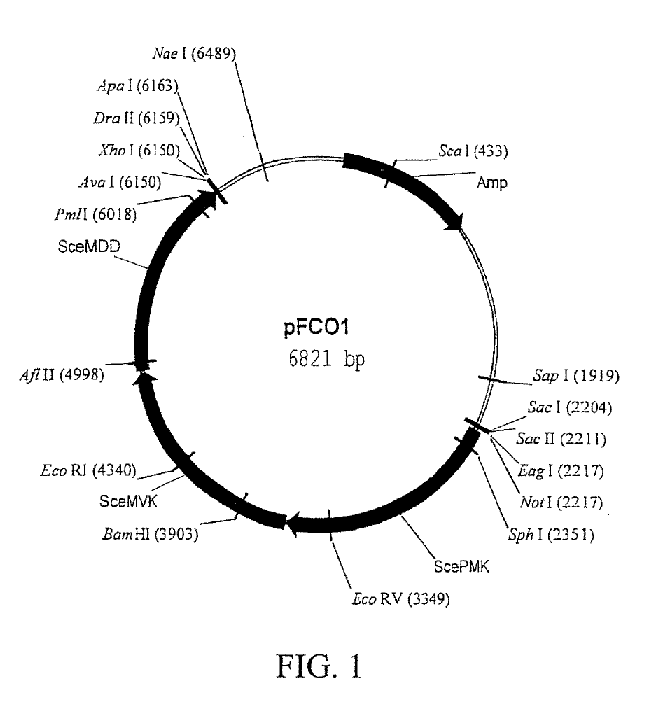

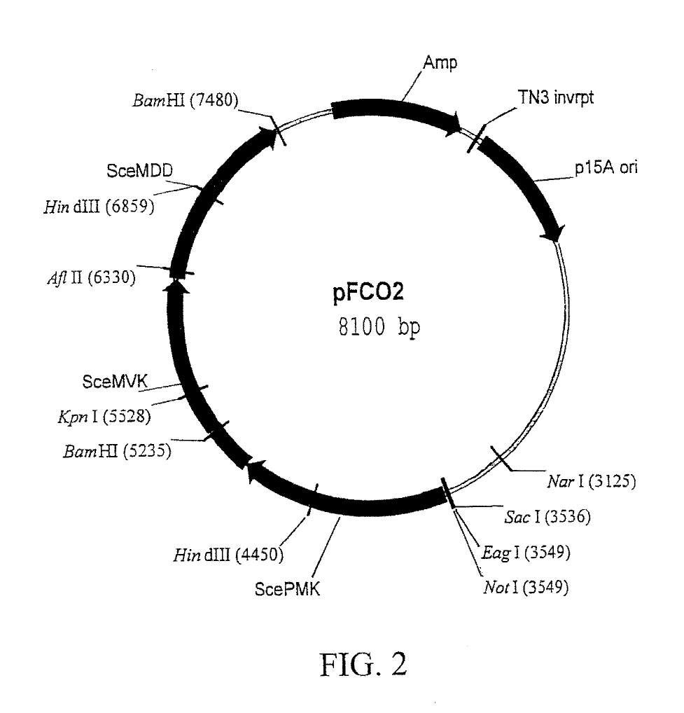

Primer FH0129-2 includes a SpeI site (underlined). Primer FH0129-1 contains an XhoI site (underlined), an AflII site (double-underlined), and 54 nucleotides (bold italics) corresponding to the 5′ end of the yeast orf for mevalonate diphosphate decarboxylase. Following PCR using primers FH0129-1 and FH0129-2, a product containing the orf encoding yeast mevalonate kinase is isolated by agarose gel electrophoresis and GeneClean purified. Following restriction with SpeI-XhoI, the PCR product is inserted into the SpeI-XhoI sites of pBluescript(SK+) (Stratagene, LaJolla, Calif.) by ligation to create pBRG12. Primers FH0211-1 and FH0211-2 contain a NotI site (underlined) and a PstI site (underlined), respectively. Following PCR using primers FH0211-1 and FH0211-2, a product containing the orf encoding yeast phosphomevalonate kinase is restricted with NotI-PstI, purified by GeneClean, and inserted into pGEM-T Easy (Promega Corp, Madison, Wis.) by ligation to create pERG8. An orf encoding yeast mevalonate diphosphate decarboxylase is isolated by PCR using primers CT0419-1 and CT0419-2 and inserted directly into pGEM-T Easy by ligation to create pERG19. Restriction of pERG8 with NotI-PstI yields a 1.4 Kb DNA fragment containing the orf for phosphomevalonate kinase. Restriction of pBRG12 with NotI-PstI is followed by the insertion of the 1.4 Kb NotI-PstI DNA fragment by ligation to create pBRG812 containing the orfs for both phosphomevalonate kinase and mevalonate kinase and the 5′ end of the orf for yeast mevalonate diphosphate decarboxylase. Restriction of pERG19 with AflII-XhoI yields a 1.2 Kb DNA fragment containing the 3′ end of the orf for yeast mevalonate diphosphate decarboxylase missing in pBRG812. Insertion of the 1.2 Kb AflII-XhoI DNA fragment into pBRG812/AflII-XhoI by ligation yields pFCO1 containing the three yeast mevalonate pathway orfs (FIG. 1). Restriction of pFCO1 with XhoI is followed by treatment with the Klenow fragment of T7 DNA polymerase and dNTPs to create blunt ends. Subsequent restriction of pFCO1/XhoI/Klenow with Sad yields a 3.9 Kb DNA fragment containing the three yeast mevalonate pathway orfs. Following agarose gel electrophoresis and GeneClean purification of the 3.9 Kb DNA fragment, it is inserted into the SmaI-SacI sites of pNGH1-amp (Garrett et al., J. Biol. Chem. 273:12457-12465, 1998) by ligation to create pFCO2 (FIG. 2).

### Example 2

### Construction of E. coli Strain FH11 (JM101/dxs::kan^{r}/pDX4)

A mutant E. coli strain containing a disruption of the chromosomal dxs gene is constructed as described by Hamilton et al. (Hamilton et al., J. Bacteriol. 171:4617-4622, 1989). The strains are grown at 30° C. or 44° C. in Luria-Bertani (LB) supplemented with the following antibiotics as necessary; ampicillin (Amp) (50 (g/ml), chloramphenicol (Cam) (30 (g/ml), and kanamycin (Kan) (25 (g/ml). Within phagemid DD92 (F. R. Blattner, University of Wisconsin, Madison, Wis.) is a 19.8 Kb EcoRI fragment of E. coli genomic DNA containing dxs, the gene for DXP synthase. Following the isolation of the phage from E. coli strain LE392, DD92 is restricted with SphI, and the resultant 6.3 Kb fragment is isolated by agarose gel electrophoresis. GeneClean purification of the SphI fragment and restriction with SmaI yields a 2.0 Kb SphI-SmaI fragment containing E. coli dxs. The 2.0 Kb fragment is purified by GeneClean and inserted by ligation into the SphI-HindIII sites of pMAK705, a plasmid containing a temperature-sensitive origin of replication (Hamilton et al., J. Bacteriol. 171:4617-4622, 1989). The resulting plasmid containing wt dxs, pDX4, is restricted with SapI, a unique site located in the middle of the dxs gene, and the 5′-overhangs are filled in with Klenow and dNTPs. The blunt-ended DNA fragment is purified by GeneClean and treated with shrimp alkaline phosphatase (SAP, USB Corp., Cleveland, Ohio) according to the manufacturer's instructions. pUC4K (Amersham Pharmacia Biotech, Piscataway, N.J.) is restricted with EcoRI, Klenow-treated, and the resulting 1.3 Kb blunt-ended DNA fragment containing the gene for Kan resistance is inserted into the filled-in SapI site of pDX4 by blunt-end ligation to create pDX5 with a disruption in E. coli dxs. Competent E. coli JM101 cells are transformed with pDX5, a pMAK705 derivative containing dxs::kanr, and grown to an optical density (A600) of 0.6 at 30° C. Approximately 10,000 cells are plated out on LB/Cam medium prewarmed to 44° C. The plates were incubated at 44° C., and several of the resulting colonies are grown at 44° C. in 4 ml of LB/Cam medium. Four 50 ml LB/Cam cultures are started with 0.5 ml from four of the 4 ml cultures and grown overnight at 30° C. Four fresh 50 ml LB/Cam cultures are started with 100 μl of the previous cultures and grown overnight at 30° C. An aliquot of one of the 50 ml cultures is serially diluted 5×105 fold, and 5 μl is plated on LB/Cam medium. Following incubation at 30° C., the resulting colonies are used to individually inoculate 3 ml of LB medium containing Cam and Kan. Twelve LB/Cam/Kan cultures are grown overnight at 30° C. and used for plasmid DNA isolation. E. coli cells where the disrupted copy of dxs is incorporated into the genome are identified by restriction analysis of the isolated plasmid DNA and verified by sequence analysis of the DNA contained in the plasmids. The E. coli JM101 derivative containing the dxs::kanr mutation is designated FH11 (Hahn et al. 2001).

### Example 3

### Assay Demonstrating Synthesis of IPP from Mevalonic Acid in E. coli

The episomal copy of dxs contained on pDX4 in E. coli strain FH11 is “turned off” at 44° C. due to a temperature sensitive origin of replication on the pMAK705 derivative (Hamilton et al., J. Bacteriol. 171:4617-4622, 1989). The inability of FH11 to grow at the restrictive temperature demonstrates that dxs is an essential single copy gene in E. coli (Hahn et al., 2001). A cassette containing three yeast mevalonate pathway orfs is removed from pFCO1 and inserted into pNGH1-Amp to form pFCO2 for testing the ability of the mevalonate pathway orfs to complement the dxs::kanr disruption when FH11 is grown at 44° C. on medium containing mevalonate. The utility of strain FH11 as a component of an assay for testing the ability of mevalonate pathway orfs to direct the synthesis of IPP is demonstrated as follows:

Colonies of E. coli strain FH11 transformed with pFCO2 or pNGH1-Amp, the expression vector without an insert, are isolated by incubation at 30° C. on LB plates containing Kan and Amp. Four ml LB/Kan/Amp cultures containing either FH11/pFCO2 or FH11/pNGH1-Amp are grown overnight at 30° C. Following a 10,000-fold dilution, 10 μl portions from the cultures are spread on LB/Kan/Amp plates that are prewarmed to 44° C. or are at rt. Approximately 1.3 mg of mevalonic acid is spread on each plate used for FH11/pFCO2. The prewarmed plates are incubated at 44° C., and the rt plates are incubated at 30° C. overnight.

FH11/pNGH1-amp cells will not grow at the restrictive temperature of 44° C. and FH11/pFCO2 cells are unable to grow at of 44° C. unless mevalonic acid (50 mg/L) is added to the growth medium thus establishing the ability of the polypeptides encoded by the mevalonate pathway orfs contained in the synthetic operon within pFCO2 to form IPP from mevalonate in vivo (Hahn et al., 2001).

### Example 4

### Isolation of Mevalonate Pathway Orfs

In a specific, exemplified embodiment, the isolation of orfs, each encoding a polypeptide with either HMG-CoA synthase enzyme activity, HMG-CoA reductase enzyme activity, or acetoacetyl-CoA thiolase enzyme activity, and construction of vectors containing these orfs is as follows: Synthesis of A. thaliana first strand cDNAs is performed utilizing PowerScript™(reverse transcriptase (Clontech Laboratories, Inc., Palo Alto, Calif.) according to the manufacturer's instructions. Specifically, a microfuge tube containing 5 μl of A. thaliana RNA (Arabidopsis Biological Resource Center, Ohio State University, Columbus, Ohio), 1.8 μl poly(dT)15 primer (0.28 μg/μl, Integrated DNA Technologies, Inc. Coralville, Iowa), and 6.2 μl DEPC-treated H₂O is heated at 70° C. for 10 min and then immediately cooled on ice. The mixture is spun down by centrifugation and 4 μl of 5× First-Strand Buffer (Clontech), 2 μ(l Advantage UltraPure PCR dNTP mix (10 mM each, Clontech) and 2 μ(l 100 mM DTT are added and the entire contents mixed by pipetting. Following the addition of 1 μ(l reverse transcriptase (Clontech) and mixing by pipetting, the contents are incubated at 42° C. for 90 min and then heated at 70° C. for 15 min to terminate the reaction.

The resulting A. thaliana first strand cDNAs are used as templates for the synthesis of an orf encoding HMG-CoA synthase and a truncated HMG-CoA reductase by PCR in a Perkin-Elmer GeneAmp PCR System 2400 thermal cycler utilizing the Advantage®-HF 2 PCR Kit (Clontech) according to the manufacturer's instructions. An A. thaliana HMG-CoA synthase orf is isolated using the following PCR primers:

(SEQ ID NO: 7)
1) 5′ GCTCTAGATGCGCAGGAGGCACATATGGCGAAGAACGTTGGGAT
TTTGGCTATGGATATCTATTTCCC 3′ (sense);
and
(SEQ ID NO: 8)
2) 5′ CG [symbol] TCGACGGATCCTCAGTGTCCATTGGCTACAGATCCA
TCTTCACCTTTCTTGCC 3′ (antisense);

containing the restriction site XbaI shown underlined, the restriction site XhoI shown in bold italic and the restriction site SalI shown double underlined. Specifically, 2 μ(l cDNA, 5 μ(l 10×HF 2 PCR Buffer (Clontech), 5 μl 10×HF 2 dNTP Mix (Clontech), 1 μl each of the primers described above, 1 μl 50× Advantage-HF 2 Polymerase Mix (Clontech), and 35 μl PCR-Grade H2O (Clontech) are combined in a 0.5 ml PCR tube. The mixture is heated at 94° C. for 15 sec then subjected to 40 PCR cycles consisting of 15 sec at 94° C. and 4 min at 68° C. After a final incubation at 68° C. for 3 min, the reaction is cooled to 4° C. Agarose gel electrophoresis is performed on a 10 μl aliquot to confirm the presence of a DNA fragment of the predicted size of 1.4 Kb. The PCR is repeated in triplicate to generate enough product for its isolation by gel excision and purification by GeneClean (Qbiogene, Inc., Carlsbad Calif.). Following restriction with XbaI-XhoI and purification by GeneClean, the 1.4 Kb PCR product is inserted into the XbaI-XhoI sites of pBluescript(SK+) by ligation to form putative pBSHMGS constructs. Sequence analysis of several of the candidate constructs is performed to identify inserts with DNA identical to the published A. thaliana orf for HMG-CoA synthase and are used for the construction of pBSHMGSR as described below.

An A. thaliana orf encoding a polypeptide with HMG-CoA reductase enzyme activity is synthesized by PCR essentially as described above using the following primers:

3) 5′ CCGCTCGAGCACGTGGAGGCACATATGCAATGCTGTGAGATGCCT GTTGGATACATTCAGATTCCTGTTGGG 3′ (sense) (SEQ ID NO: 9); and
4) 5′ GGGGTACCTGCGGCCGGATCCCGGGTCATGTTGTTGTTGTTGTCGT TGTCGTTGCTCCAGAGATGTCTCGG 3′ (antisense) (SEQ ID NO: 10); containing the restriction site XhoI shown underlined, the restriction site KpnI shown in italic, the restriction site EagI shown in bold, and the restriction site SmaI shown double underlined. The 1.1 Kb PCR product is isolated by agarose gel electrophoresis, purified by GeneClean and inserted into the pT7Blue-3 vector (Novagen, Inc., Madison, Wis.) using the Perfectly Blunt™ Cloning Kit (Novagen) according to the manufacturer's instructions. Sequence analysis is performed to identify constructs containing A. thaliana DNA encoding the desired C-terminal portion of the published HMG-CoA reductase amino acid sequence and are designated pHMGR. PCR is performed on S. cerevisiae genomic DNA (Invitrogen, Corp., Carlsbad, Calif.) by using the Advantage®-HF 2 PCR Kit (Clontech) according to the manufacturer's instructions and the following primers:

(SEQ ID NO: 11)
5) 5′ ACAACACCGCGGCGGCCGCGTCGACTACGTAGGAGGCACATATG
TCTCAGAACGTTTACATTGTATCGACTGCC 3′ (sense);
and
(SEQ ID NO: 12)
6) 5′ GC [symbol] GGATCCTCATATCTTTTCAATGACAATAGAGGAAGC
ACCACCACC 3′ (antisense);

containing the restriction site NotI shown underlined, the restriction site SacII shown in italic, the restriction site SalI shown in bold, the restriction site SnaBI shown double underlined, and the restriction site XbaI in bold italic. The 1.2 Kb PCR product is isolated by agarose gel electrophoresis, purified by GeneClean and inserted into the vector pT7Blue-3 (Novagen,) using the Perfectly Blunt™ Cloning Kit (Novagen) according to the manufacturer's instructions. Sequence analysis is performed to identify constructs containing S. cerevisiae DNA identical to the published orf encoding acetoacetyl-CoA thiolase and they are designated pAACT.

### Example 5

### Construction of pHKO1

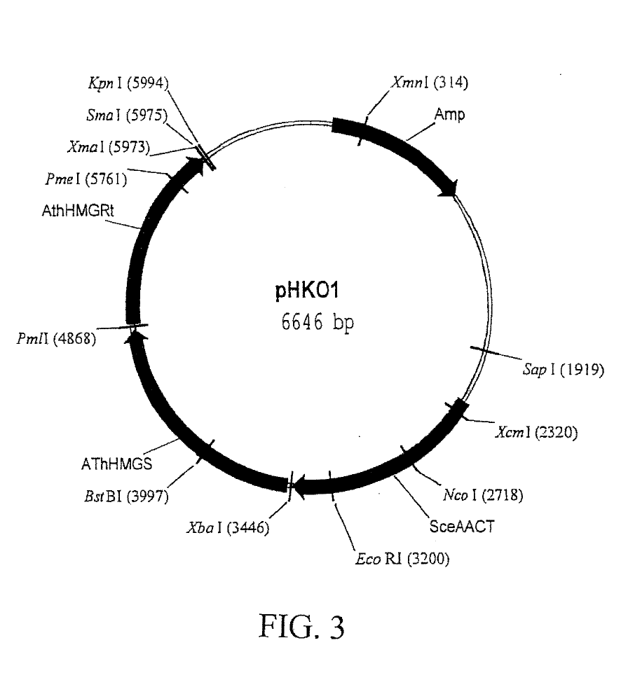

In an exemplified embodiment, a pBluescript(SK+) derivative containing an operon with orfs encoding polypeptides with enzymatic activities for HMG-CoA synthase, HMG-CoA reductase, and acetoacetyl-CoA thiolase is constructed as follows: Following restriction of pHMGR with XhoI-KpnI, isolation of the 1.1 Kb DNA fragment by agarose gel electrophoresis, and purification by GeneClean, the 1.1 Kb XhoI-KpnI DNA fragment containing the orf encoding the C-terminal portion of A. thaliana HMG-CoA reductase is inserted into the SalI-KpnI sites of pBSHMGS by ligation to create pBSHMGSR. Following restriction of pAACT with SacII-XbaI, isolation of the 1.2 Kb DNA fragment containing the orf encoding yeast acetoacetyl-CoA thiolase by agarose gel electrophoresis, and purification by GeneClean, the 1.2 Kb SacII-XbaI DNA fragment is inserted into the SacII-XbaI sites of pBSHMGSR by ligation to create pHKO1 (FIG. 3).

### Example 6

### Construction of pHKO2

In a specific, exemplified embodiment, a vector containing a synthetic operon consisting of six orfs encoding polypeptides with acetoacetyl-CoA thiolase, HMG-CoA synthase, HMG-CoA reductase, mevalonate kinase, phosphomevalonate kinase, and mevalonate diphosphate decarboxylase enzymatic activities, thus comprising the entire mevalonate pathway, is constructed as follows: Restriction of pHKO1 with Eagl yields a 3.7 Kb DNA fragment containing orfs encoding yeast acetoacetyl-CoA thiolase, A. thaliana HMG-CoA synthase, and a truncated A. thaliana HMG-CoA reductase. Following isolation of the 3.7 Kb EagI DNA fragment by agarose gel electrophoresis and purification by GeneClean, it is directionally inserted into the NotI site of pFCO2 (Hahn et al., 2001) utilizing the methodology of chain reaction cloning (Pachuk et al., 2000), thermostable Ampligase® (Epicentre Technologies, Madison. WI), and the following bridge oligonucleotide primers:

(SEQ ID NO: 13)
1) 5′ TGGAATTCGAGCTCCACCGCGGTGGCGGCCGCGTCGACGCCGGC
GGAGGCACATATGTCT 3′;
and
(SEQ ID NO: 14)
2) 5′ AACAACAACAACATGACCCGGGATCCGGCCGCAGGAGGAGTTCA
TATGTCAGAGTTGAGA 3′;

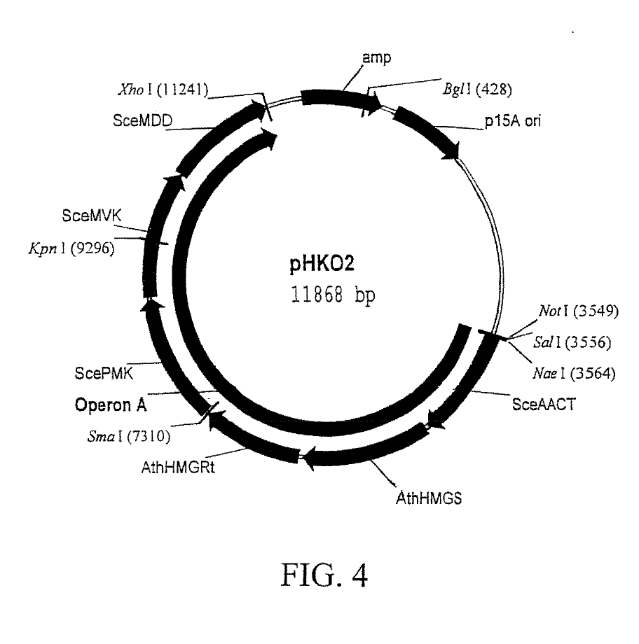

as follows: Agarose gel electrophoresis is performed on the 8.1 Kb pFCO2/NotI DNA fragment and the 3.7 Kb Eagl DNA fragment isolated from pHKO1 to visually estimate their relative concentrations. Approximately equivalent amounts of each fragment totaling 4.5 μl, 1 μl of each bridge oligo at a concentration of 200 nM, 5 μl Ampligase® 10× Reaction Buffer (Epicentre), 3 μl Ampligase® (5 U/(1) (Epicentre), and 35.5 μl PCR grade H2O are added to a 0.5 ml PCR tube. The mixture is heated at 94° C. for 2 min then subjected to 50 PCR cycles consisting of 30 sec at 94° C., 30 sec at 60° C., and 1 min at 66° C. After a final incubation at 66° C. for 5 min, the reaction is cooled to 4° C. Colonies resulting from the transformation of E. coli strain NovaBlue (Novagen) with 1 μl of the directional ligation reaction are grown in LB medium supplemented with ampicillin at a final concentration of 50 μg/ml. Restriction analysis with NaeI-KpnI of mini-prep plasmid DNA from the liquid cultures is performed to identify candidate pHKO2 constructs by the presence of both a 5.7 and a 6.2 Kb DNA fragment. Further analysis by restriction with SmaI-XhoI to generate both a 3.9 and 7.9 Kb DNA fragment confirms the successful construction of pHKO2 (FIG. 4).

### Example 7

### Assay Demonstrating the Synthesis of IPP from Acetyl-CoA in E. coli

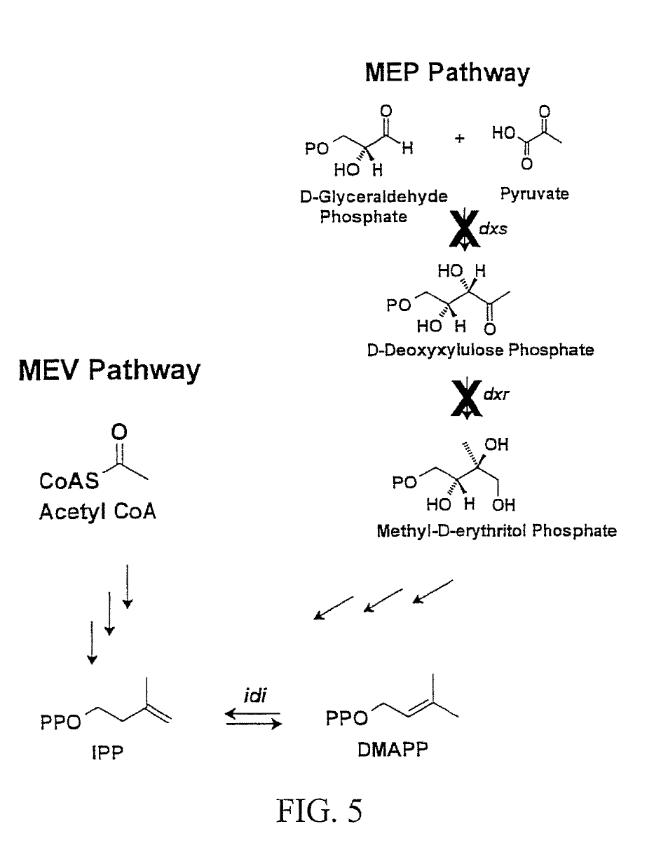

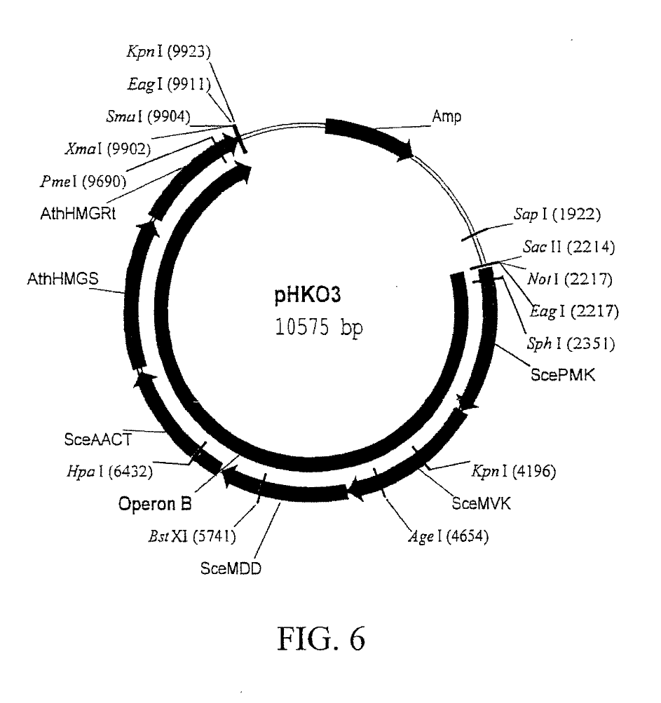

In a specific, exemplified embodiment, a derivative of pNGH1-amp (Hahn et al., 2001), containing the entire mevalonate pathway, is assayed (FIG. 5) for its ability to synthesize IPP from endogenous acetyl-CoA in E. coli strain FH11, containing the temperature sensitive dxs::kanr^{r} knockout (Hahn et al., 2001), as follows: Colonies resulting from the transformation of FH11, by pHKO2, containing orfs encoding polypeptides with enzymatic activities for acetoacetyl-CoA thiolase, HMG-CoA synthase, HMG-CoA reductase, mevalonate kinase, phosphomevalonate kinase, and mevalonate diphosphate decarboxylase, are isolated by incubation at 30° C. on LB plates containing Kan and Amp. Several 4 ml LB/Kan/amp samples are individually inoculated with single colonies from the FH11/pHKO2 transformation. Following growth at 30° C. overnight, the FH11/pHKO2 cultures are diluted 100,000-fold, and 5 μl aliquots are spread on LB/Kan/amp plates at room temperature (rt) or that are prewarmed to 44° C. The prewarmed plates are incubated at 44° C., and the rt plates are incubated at 30° C. overnight. FH11 and FH11/pNGH1 amp cells will not grow at the restrictive temperature of 44° C. (Hahn et al., 2001). FH11/pHKO2 cells are able to grow at 44° C., thus establishing the ability, of a synthetic operon comprising the entire mevalonate pathway, to form IPP from acetyl-CoA and thereby overcome the dxs::kan^{r} block to MEP pathway biosynthesis of IPP in E. coli strain FH11.

### Example 8

### Construction of pHKO3

In another exemplified embodiment, a derivative of pBluescript(SK+) containing an operon comprising orfs, which in their summation is the entire mevalonate pathway, is constructed as follows: pHKO1, containing orfs encoding acetoacetyl-CoA thiolase, HMG-CoA synthase, and an N-terminal truncated HMG-CoA reductase, is restricted with SalI-NotI and purified by GeneClean. The pBluescript(SK+) derivative pFCO1, containing the orfs encoding mevalonate kinase, phosphomevalonate kinase, and mevalonate diphosphate decarboxylase, has been described above in Example 1. Following restriction of pFCO1 with XhoI-NotI, isolation by agarose gel electrophoresis, and purification by GeneClean, the 3.9 Kb DNA fragment containing the mevalonate pathway orfs is inserted into pHKO1/SalI-NotI by directional ligation (Pachuk et al., 2000) utilizing thermostable Ampligase® (Epicentre Technologies, Madison, Wis.), and the following bridging oligonucleotides:

(SEQ ID NO: 15)
1) 5′ CTCAACTCTGACATATGAACTCCTCCTGCGGCCGCCGCGGTGGA
GCTCCAGCTTTTGTTCCC 3′;
and
(SEQ ID NO: 16)
2) 5′ GGTCTACCAAAGGAAGAGGAGTTTTAACTCGACGCCGGCGGAGG
CACATATGTCTCAGAACG 3′;

essentially as described for the construction of pHKO2. Restriction analysis is performed with KpnI to confirm the successful construction of pHKO3 (FIG. 6).

### Example 9

### Construction of Tobacco Plastid Transformation Vector pHKO4

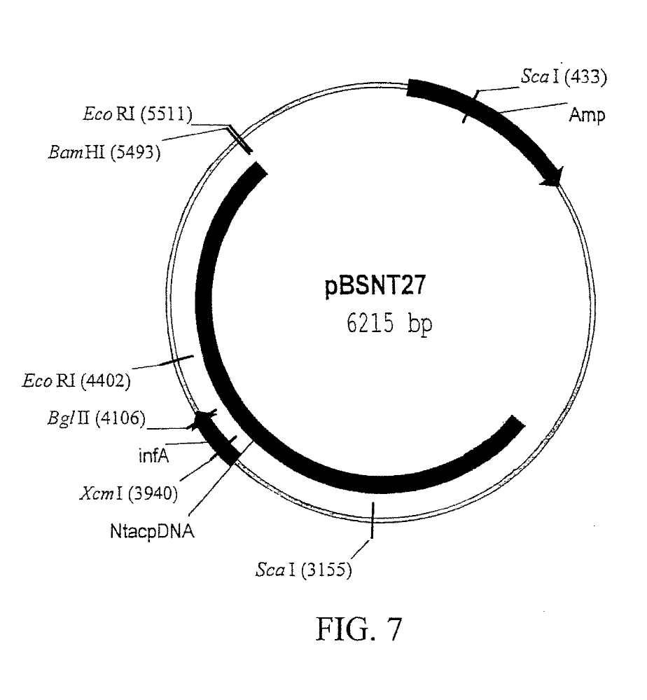

In an exemplified embodiment, a vector containing a Nicotiana tabacum plastid pseudogene is utilized to create a plastid transformation vector as follows: The pBluescript(SK+) derivative designated as pBSNT27 (FIG. 7, SEQ ID NO: 17) contains a 3.3 Kb BglII-BamHI DNA fragment of the N. tabacum chloroplast genome corresponding approximately to base-pairs 80553-83810 of the published nucleotide sequence (Sugiura, M., 1986, and Tsudsuki, T., 1998.). A unique restriction site contained within the tobacco infA pseudogene located on pBSNT27 is cleaved with BglII and the resulting 5′ overhangs are filled in with Klenow and dNTPs. The resulting 6.2 Kb blunt-ended DNA fragment is GeneClean purified. Following restriction of pHKO3 with EagI, filling in of the resulting 5′ overhangs with Klenow and dNTPs, isolation by agarose gel electrophoresis, and purification by GeneClean, the resulting 7.7 Kb blunt-ended DNA fragment, containing orfs encoding the entire mevalonate pathway, is directionally inserted into the blunt-ended BglII site of pBSNT27 utilizing chain reaction cloning (Pachuk et al., 2000.), thermostable Ampligase® (Epicentre Technologies, Madison, Wis.), and the following bridging oligonucleotides:

(SEQ ID NO: 18)
1) 5′ GATCTTTCCTGAAACATAATTTATAATCAGATCGGCCGCAGGAG
GAGTTCATATGTCAGAGTTGAG 3′;
and
(SEQ ID NO: 19)
2) GACAACAACAACAACATGACCCGGGATCCGGCCGATCTAAACAAACC
CGGAACAGACCGTTGGGAA 3′;

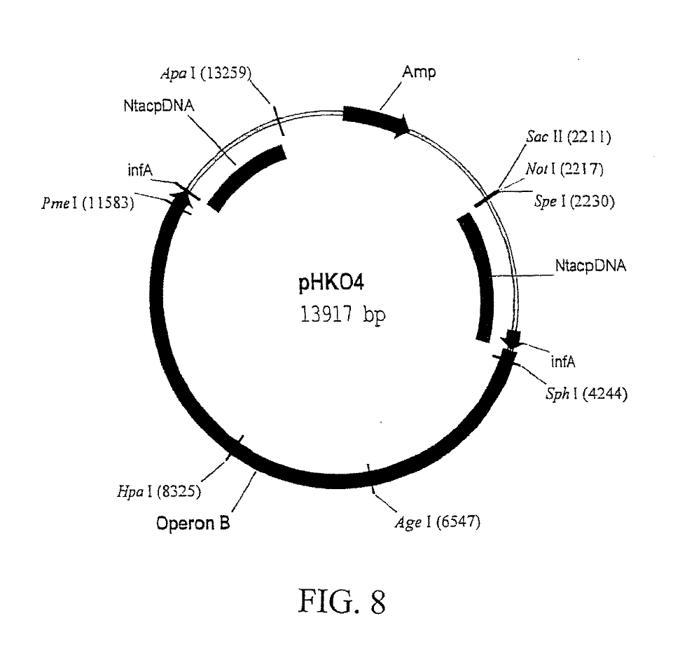

to form the tobacco plastid-specific transformation vector pHKO4 (FIG. 8).

Alternatively, other derivatives of pBSNT27 can be constructed, using skills as known in the art, that are not reliant upon an available restriction site(s) in the pseudogene. For example, although the infA pseudogene comprises basepairs 3861-4150 in pBSNT27, there are unique restriction sites in close proximity, upstream and downstream, that can be utilized to excise the entire pseudogene followed by its replacement with an orf or gene cluster comprising multiple orfs, e.g., the complete mevalonate pathway described above. Specifically, there is a unique BsrGI site at 3708 base pairs and a unique SexAI restriction site at 4433 base pairs within pBSNT27. Thus, as will be readily apparent to those skilled in the art, one can replace the infA pseudogene entirely by inserting a BsrGI- SexAI DNA fragment containing DNA, comprising orfs encoding the entire mevalonate pathway, that is flanked by the excised DNA originally flanking the infA pseudogene, i.e. DNA corresponding to 3708-3860 and 4151-4433 base pairs in pBSNT27. The resultant construct will be missing the pseudogene, but will contain the excised flanking DNA restored to its original position and now surrounding the mevalonate pathway orfs. Also, a similar strategy, that will also be apparent to those skilled in the art in view of this disclosure, can be employed that restores the intact pseudogene to a location between the DNA originally flanking it, yet linked to an orf or orfs located upstream and/or downstream of the pseudogene and adjacent to the original flanking DNA.

### Example 10

### Construction of Vectors Containing Orfs Encoding IPP Isomerase (pHKO5 and pHKO6)

In a specific, exemplified embodiment, orfs encoding IPP isomerase are isolated and vectors containing an operon comprising orfs for the entire mevalonate pathway and an additional orf for IPP isomerase are constructed as follows: A Rhodobacter capsulatus orf encoding a polypeptide with IPP isomerase activity is isolated by PCR from genomic DNA (J. E. Hearst, Lawrence Berkeley Laboratories, Berkeley, Calif.) using the following primers:

(SEQ ID NO: 20)
1) 5′ CGCTCGAGTACGTAAGGAGGCACATATGAGTGAGCTTATACCCG
CCTGGGTTGG 3′ (sense);
and
(SEQ ID NO: 21)
2) 5′ GCTCTAGAGATATCGGATCCGCGGCCGCTCAGCCGCGCAGGATC
GATCCGAAAATCC 3′ (antisense);

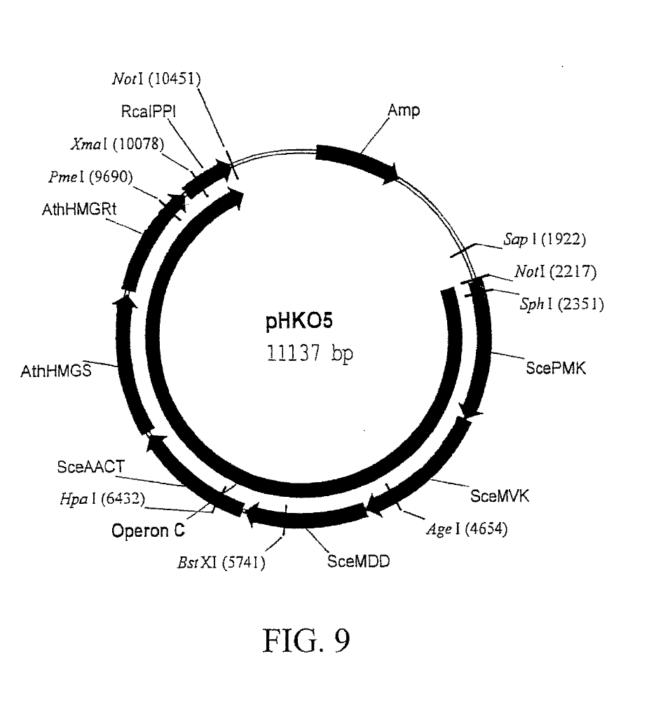

containing the restriction sites XhoI shown underlined, BsaAI shown in bold, XbaI shown in italic, EcoRV shown double underlined, and NotI shown in bold italic. The PCR product is restricted with XhoI-XbaI, isolated by agarose gel electrophoresis, purified by GeneClean, and inserted into the XhoI-XbaI sites of pBluescript(SK+) by ligation to form pBSIDI. Sequence analysis is performed to identify the plasmids containing R. capsulatus DNA identical to the complementary sequence of base pairs 34678-34148, located on contig rc04 (Rhodobacter Capsulapedia, University of Chicago, Chicago, Ill.). Following restriction of pBSIDI with BsaAI-EcoRV, agarose gel electrophoresis and GeneClean purification, the 0.5 Kb BsaAI-EcoRV DNA fragment containing the R. capsulatus orf is inserted into the dephosphorylated SmaI site of pHKO3 by blunt-end ligation to create pHKO5 (FIG. 9). This establishes the isolation of a previously unknown and unique orf encoding R. capsulatus IPP isomerase.

A Schizosaccharomyces pombe orf encoding a polypeptide with IPP isomerase activity is isolated from plasmid pBSF19 (Hahn and Poulter, J. Biol. Chem. 270:11298-11303, 1995) by PCR using the following primers

(SEQ ID NO: 22)
3) 5′ GCTCTAGATACGTAGGAGGCACATATGAGTTCCCAACAAGAGAA
AAAGGATTATGATGAAGAACAATTAAGG 3′ (sense);
and
(SEQ ID NO: 23)
4) 5′ CGCTCGAGCCCGGGGGATCCTTAGCAACGATGAATTAAGGTATC
TTGGAATTTTGACGC 3′ (antisense);

containing the restriction site BsaAI shown in bold and the restriction site SmaI shown double underlined. The 0.7 Kb PCR product is isolated by agarose gel electrophoresis, purified by GeneClean and inserted into the pT7Blue-3 vector (Novagen, Inc., Madison, Wis.) using the Perfectly Blunt™ Cloning Kit (Novagen) according to the manufacturer's instructions. Sequence analysis is performed to identify constructs containing S. pombe DNA identical to the published DNA sequence (Hahn and Poulter, 1995) and are designated pIDI. Following restriction of pIDI with BsaAI-SmaI, isolation by agarose gel electrophoresis, and purification by GeneClean, the 0.7 Kb BsaAI-SmaI DNA fragment containing the orf encoding S. pombe IPP isomerase is inserted into the dephosphorylated SmaI site of pHKO3 by blunt-end ligation to create pHKO6.

### Example 11

### Construction of Vectors Containing Alternative Orfs for Mevalonate Pathway Enzymes and IPP Isomerase

In another exemplified embodiment, vectors containing open reading frames (orfs) encoding enzymes of the mevalonate pathway and IPP isomerase other than those described above are constructed. Polynucleotides derived from the yeast Saccharomyces cerevisiae, the plant Arabidopsis thaliana, and the bacteria Rhodobacter capsulatus and Streptomyces sp strain CL190 are used for the construction of vectors, including plastid delivery vehicles, containing orfs for biosynthesis of the encoded enzymes. Construction of the vectors is not limited to the methods described. One skilled in the art may choose alternative restriction sites, PCR primers, etc. to create analogous plasmids containing the same orfs or other orfs encoding the enzymes of the mevalonate pathway and IPP isomerase.

Specifically, by way of example, genomic DNA is isolated from Streptomyces sp strain CL190 (American Type Culture Collection, Manassas, Va.) using the DNeasy Tissue Kit (Qiagen) according to the manufacturer's instructions. An orf encoding a polypeptide with HMG-CoA reductase activity (Takahashi et al., J. Bacteriol. 181:1256-1263, 1999) is isolated from the Streptomyces DNA by PCR using the following primers:

(SEQ ID NO: 24)
1) 5′ CCGCTCGAGCACGTGAGGAGGCACATATGACGGAAACGCACGCC
ATAGCCGGGGTCCCGATGAGG 3′ (sense);
and
(SEQ ID NO: 25)
2) 5′ GGGGTACCGCGGCCGCACGCGTCTATGCACCAACCTTTGCGGTC
TTGTTGTCGCGTTCCAGCTGG 3′ (antisense);

containing the restriction site XhoI shown underlined, the restriction site KpnI shown in italics, the restriction site NotI shown in bold, and the restriction site MluI shown double underlined. The 1.1 Kb PCR product is isolated by agarose gel electrophoresis, purified by GeneClean and inserted into the pT7Blue-3 vector (Novagen, Inc., Madison, Wis.) using the Perfectly Blunt™ Cloning Kit (Novagen) according to the manufacturer's instructions. Sequence analysis is performed to identify constructs containing Streptomyces sp CL190 DNA identical to the published sequence and are designated pHMGR2.

Alternatively, using skills as known in the art, an orf encoding a truncated S. cerevisiae HMG-CoA reductase (Chappel et al., U.S. Pat. No. 5,349,126 1994) can be isolated by PCR and inserted into pT7Blue-3 (Novagen, Inc., Madison, Wis.) to construct a vector for use in building a gene cluster comprising the entire mevalonate pathway, in an analogous fashion to the use of the Streptomyces sp CL190 orf encoding HMG-CoA reductase, as described herein.

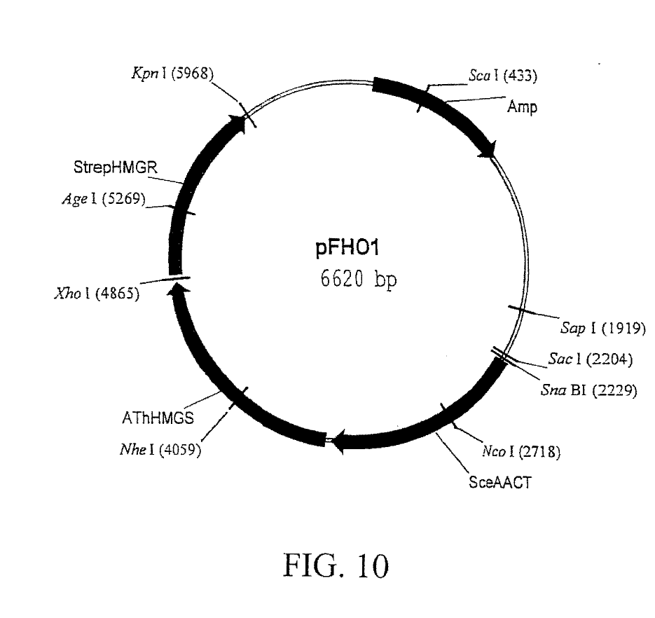

Following restriction of pAACT (see Example 4) with SacII-XbaI, isolation of the 1.2 Kb DNA fragment containing the orf encoding yeast acetoacetyl-CoA thiolase by agarose gel electrophoresis, and purification by GeneClean, the 1.2 Kb SacII-XbaI DNA fragment is inserted into the SacII-XbaI sites of pBSHMGS (see Example 4) by ligation to create pBSCTGS. Following restriction of pHMGR2 with XhoI-KpnI, isolation of the 1.1 Kb DNA fragment by agarose gel electrophoresis, and purification by GeneClean, the 1.1 Kb XhoI-KpnI DNA fragment containing the orf encoding Streptomyces sp CL190 HMG-CoA reductase is inserted into the XhoI-KpnI sites of pBSCTGS by ligation to create the pBluescript(SK+) derivative, pFHO1 (FIG. 10).

A derivative of pFHO1 containing an operon with orfs, which in their summation comprise the entire mevalonate pathway, is constructed as follows: pFHO1 is restricted with SnaBI and the resulting 6.6 Kb blunt-ended DNA fragment is purified by GeneClean. Following the restriction of pFCO1 (see Example 1) with NotI-XhoI, the resulting 3.9 Kb DNA fragment is isolated by agarose gel electrophoresis and purified by GeneClean. The 5′ overhangs of the 3.9 Kb DNA fragment are filled in with Klenow and dNTPs. Following purification by GeneClean, the blunt-ended DNA fragment containing three mevalonate pathway orfs (Hahn et al., 2001) is inserted into the SnaBI site of pFHO1 utilizing directional ligation methodology (Pachuk et al., 2000), thermostable Ampligase® (Epicentre Technologies, Madison, Wis.), and the bridging oligonucleotides:

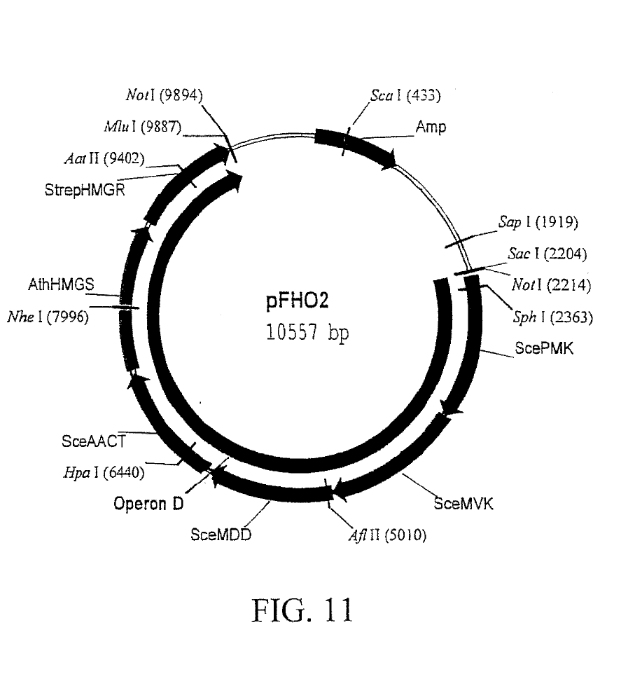

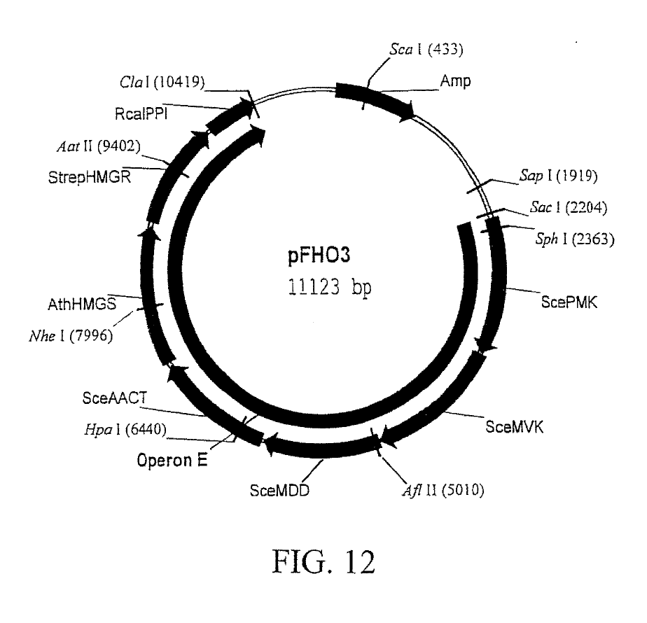

(SEQ ID NO: 26)
3) 5′ GAGCTCCACCGCGGCGGCCGCGTCGACTACGGCCGCAGGAGGAG
TTCATATGTCAGAGTT 3′;
and
(SEQ ID NO: 27)
4) 5′ TCTACCAAAGGAAGAGGAGTTTTAACTCGAGTAGGAGGCACATA
TGTCTCAGAACGTTTA 3′;
to form pFHO2 (FIG. 11).

A derivative of pFHO2 containing an operon with orfs, which in their summation comprise the entire mevalonate pathway and an orf encoding IPP isomerase is constructed as follows: pFHO2 is restricted with MluI and the resulting 5′ overhangs are filled in with Klenow and dNTPs. The 10.6 Kb blunt-ended DNA fragment is purified by GeneClean. Following restriction of pBSIDI with BsaAI-EcoRV, agarose gel electrophoresis and GeneClean purification, the resulting blunt-ended 0.5 Kb DNA fragment containing the R. capsulatus IPP isomerase orf is inserted into the filled in MluI site of pFHO2 utilizing directional ligation methodology (Pachuk et al., 2000), thermostable Ampligase® (Epicentre Technologies, Madison, Wis.), and the following bridging oligonucleotides:

(SEQ ID NO: 28)
5) 5′ CAAGACCGCAAAGGTTGGTGCATAGACGCGGTAAGGAGGCACAT
ATGAGTGAGCTTATAC 3′;
and
(SEQ ID NO: 29)
6) 5′ CCTGCGCGGCTGAGCGGCCGCGGATCCGATCGCGTGCGGCCGCG
GTACCCAATTCGCCCT 3′;
to form pFHO3 (FIG. 12).

Following the restriction of pBluescript(SK+) with SacII-XbaI and purification by GeneClean, a 1.3 Kb SacII-XbaI DNA fragment containing the orf encoding S. cerevisiae acetoacetyl-CoA thiolase, isolated from pAACT (see Example 4) by restriction and agarose gel electrophoresis, is inserted into pBluescript(SK+)/SacII-XbaI by ligation. The resulting plasmid, pBSAACT, is restricted with XbaI, treated with Klenow and dNTPs, and purified by GeneClean. Following restriction of Streptomyces sp CL190 genomic DNA with SnaBI, a blunt-ended 6.8 Kb DNA fragment, containing five (5) orfs encoding polypeptides with HMG-CoA synthase, HMG-CoA reductase, mevalonate kinase, phosphomevalonate kinase, mevalonate diphosphate decarboxylase and IPP isomerase enzymatic activities (Takagi et al., J. Bacteriol. 182:4153-4157, 2000 and Kuzuyama et al., Proc. Natl. Acad. Sci. USA 98:932-7, 2001), is isolated by agarose gel electrophoresis, purified by GeneClean and inserted into the filled in XbaI site of pBSAACT utilizing directional ligation methodology (Pachuk et al., 2000), thermostable AmpligaseD (Epicentre Technologies, Madison, Wis.), and the bridging oligonucleotides:

(SEQ ID NO: 30)
7) 5′ TGTCATTGAAAAGATATGAGGATCCTCTAGGTACTTCCCTGGCG
TGTGCAGCGGTTGACG 3′;
and
(SEQ ID NO: 31)
8) 5′ CGATTCCGCATTATCGGTACGGGTGCCTACCTAGAACTAGTGGA
TCCCCCGGGCTGCAGG 3′;

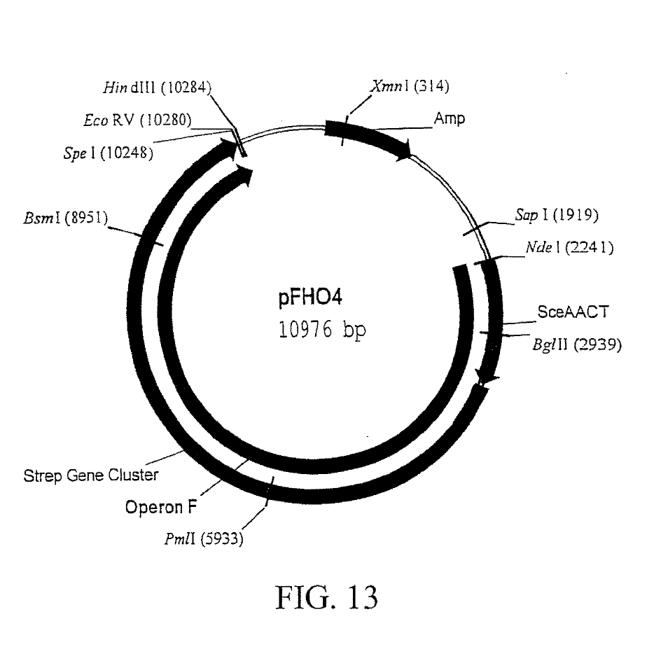

to form pFHO4 (FIG. 13). Transformation experiments to isolate pFHO4 constructs are performed with E. coli competent cells utilizing media containing ampicillin. Alternatively, media containing only fosmidomycin (20 μg/ml) as the selection agent is used for the direct isolation of pFHO4 constructs containing the Streptomyces sp CL190 gene cluster. The construction of vectors pHKO2, pHKO3, pHKO5, pHKO6, pFHO2, pFHO3, and pFHO4, illustrates the many ways of combining orfs isolated from a variety of organisms to encode polypeptides such that in their summation they comprise the entire mevalonate pathway or comprise the entire mevalonate pathway and IPP isomerase.

### Example 12

### Construction of Tobacco Plastid Transformation Vectors pHKO7 and pHKO8

In a specific, exemplified embodiment, tobacco plastid-specific transformation vectors containing orfs, which in their summation comprise the mevalonate pathway, and an additional orf encoding IPP isomerase are constructed as follows: Restriction of pHKO5 with NotI generates a DNA fragment containing six orfs comprising the entire mevalonate pathway and an additional orf encoding R. capsulatus IPP isomerase. Restriction of pHKO6 with Eagl generates a DNA fragment containing the six orfs comprising the complete mevalonate pathway and an additional orf encoding S. pombe IPP isomerase. Following isolation by agarose gel electrophoresis and purification by GeneClean, the 8.2 Kb NotI DNA fragment from pHKO5 is blunt-ended with Klenow and dNTPs and inserted into the blunt-ended BglII site of pBSNT27 utilizing chain reaction cloning (Pachuk et al., 2000), thermostable Ampligase® (Epicentre Technologies, Madison, Wis.), and the following bridging oligonucleotides:

(SEQ ID NO: 32)
1) 5′ CTTTCCTGAAACATAATTTATAATCAGATCGGCCGCAGGAGGAG
TTCATATGTCAGAGTT 3′;
and
(SEQ ID NO: 33)
2) 5′ TTCGGATCGATCCTGCGCGGCTGAGCGGCCGATCTAAACAAACC
CGGAACAGACCGTTGG 3′;

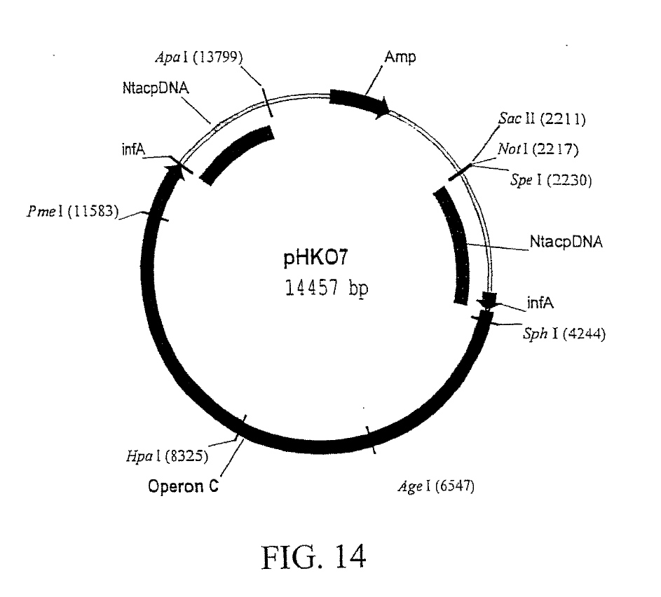

to create the plastid delivery vehicle pHKO7 (FIG. 14) containing orfs encoding the entire mevalonate pathway and an orf encoding R. capsulatus IPP isomerase. Following isolation by agarose gel electrophoresis and purification by GeneClean, the 8.4 Kb EagI DNA fragment from pHKO6 is blunt-ended with Klenow and dNTPs and inserted into the blunt-ended BglII site of pBSNT27 utilizing chain reaction cloning (Pachuk et al., 2000), thermostable Ampligase® (Epicentre Technologies, Madison, Wis.), and the following bridging oligonucleotides:

(SEQ ID NO: 34)
3) 5′ CTTTCCTGAAACATAATTTATAATCAGATCGGCCGCAGGAGGAG
TTCATATGTCAGAGT 3′;
and
(SEQ ID NO: 35)
4) 5′ TCGTTGCTAAGGATCCCCCGGGATCCGGCCGATCTAAACAAACC
CGGAACAGACCGTTGG 3′;

to create the plastid delivery vehicle pHKO8 containing orfs encoding the entire mevalonate pathway plus the S. pombe IPP isomerase orf.

Alternatively, either of the IPP isomerase orfs described above can be solely inserted, without orfs for the mevalonate pathway, directly into pBSNT27 (or into any suitable plant transformation vector, known in the art), using skills known in the art.

### Example 13

### Construction of Vectors used for Increasing Carotenoid Production (pHKO9, pHK10, pHK11, pHK12, and pHK13)

In yet another exemplified embodiment, a derivative of pTrcHisB (Invitrogen) containing a synthetic operon comprising orfs, which in their summation is the entire mevalonate pathway, is constructed as follows: A unique NotI site was inserted into pTrcHisB utilizing the following oligonucleotides:

(SEQ ID NO: 36)
1) 5′ CATGGCGGCCGCG 3′;
and
(SEQ ID NO: 37)
2) 5′ GATCCGCGGCCGC 3′;

that upon annealing, form a double-stranded DNA linker containing NotI with 5′ overhangs compatible with StyI and BamHI. Following restriction of pTrcHisB with Styl-BamHI, isolation of the resulting 4.3 Kb DNA fragment by agarose gel electrophoresis, and its purification by GeneClean, the NotI linker was inserted into pTrcHisB/Styl-BamHI by ligation. Restriction analysis with BsaAI-NotI confirms the successful construction of pTrcHisB-NotI (pTHBN1) by the presence of both 2.5 and 1.8 Kb DNA fragments. Following restriction of pHKO3 with EagI, the 7.7 Kb DNA fragment, containing the six mevalonate pathway orfs, is isolated by agarose gel electrophoresis, purified by GeneClean, and inserted into the NotI site of pTHBN1 utilizing directional ligation methodology (Pachuk et al., 2000), thermostable Ampligase® (Epicentre Technologies, Madison, Wis.), and the bridging oligonucleotides:

(SEQ ID NO: 38)
3) 5′ TTAAATAAGGAGGAATAAACCATGGCGGCCGCAGGAGGAGTT
CATATGTCAGAGTTGAGA 3′;
and
(SEQ ID NO: 39)
4) 5′ AACAACAACAACATGACCCGGGATCCGGCCGCGATCCGAGCT
CGAGATCTGCAGCTGGTA 3′;

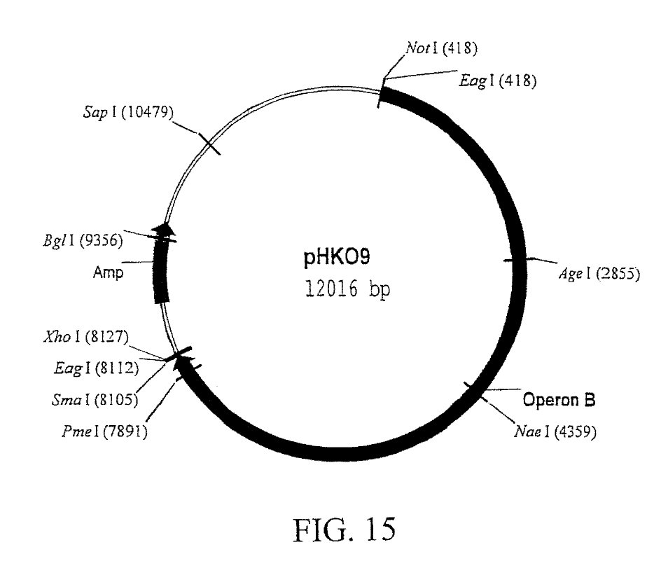

to form pHKO9 (FIG. 15).

Derivatives of pTHBN1 containing the entire mevalonate pathway plus an additional orf encoding IPP isomerase are constructed as follows: Following restriction of pHKO5 with NotI, the 8.2 Kb DNA fragment, containing the six mevalonate pathway orfs plus an orf encoding R. capsulatus IPP isomerase, is isolated by agarose gel electrophoresis, purified by GeneClean, and inserted into the NotI site of pTHBN1 utilizing directional ligation methodology (Pachuk et al., 2000), thermostable Ampligase® (Epicentre Technologies, Madison, Wis.), and the bridging oligonucleotides:

(SEQ ID NO: 40)
5) 5′ TCGATTAAATAAGGAGGAATAAACCATGGCGGCCGCAGGAGG
AGTTCATATGTCAGAGTT 3′;
and
(SEQ ID NO: 41)
6) 5′ GATTTTCGGATCGATCCTGCGCGGCTGAGCGGCCGCGATCCG
AGCTCGAGATCTGCAGCT 3′;

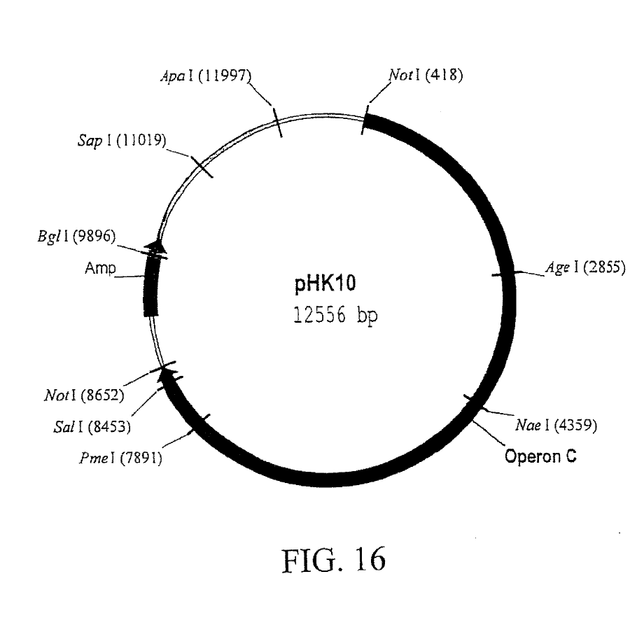

to form pHK10 (FIG. 16). Following restriction of pHKO6 with Eagl, the 8.4 Kb DNA fragment, containing the six mevalonate pathway orfs plus an orf encoding S. pombe IPP isomerase, is isolated by agarose gel electrophoresis, purified by GeneClean, and inserted into the NotI site of pTHBN1 utilizing directional ligation methodology (Pachuk et al., 2000), thermostable Ampligase® (Epicentre Technologies, Madison, Wis.), and the following bridging oligonucleotides:

(SEQ ID NO: 42)
7) 5′ TCGATTAAATAAGGAGGAATAAACCATGGCGGCCGCAGGAGG
AGTTCATATGTCAGAGTT 3′;
and
(SEQ ID NO: 43)
8) 5′ TTCATCGTTGCTAAGGATCCCCCGGGATCCGGCCGCGATCCG
AGCTCGAGATCTGCAGCT 3′;

to form pHK11.

Derivatives of pTHBN1 containing only an orf encoding IPP isomerase are constructed as follows: pTHBN1 is restricted with NotI and the resulting 5′ overhangs are filled in with Klenow and dNTPs. The 4.3 Kb pTHBN1/NotI blunt-ended DNA fragment is GeneClean purified. Following restriction of pBSIDI with BsaAI-EcoRV, agarose gel electrophoresis and GeneClean purification, the resulting blunt-ended 0.5 Kb DNA fragment containing the R. capsulatus IPP isomerase orf is inserted into the filled in NotI site of pTHBN1 utilizing chain reaction cloning (Pachuk et al., 2000), thermostable Ampligase® (Epicentre Technologies, Madison, Wis.), and the following bridging oligonucleotides:

(SEQ ID NO: 44)
9) 5′ TTAAATAAGGAGGAATAAACCATGGCGGCCGTAAGGAGGCAC
ATATGAGTGAGCTTATAC T 3′;
and
(SEQ ID NO: 45)
10) 5′ GCCTGCGCGGCTGAGCGGCCGCGGATCCGATGGCCGCGATC
CGAGCTCGAGATCTGCAGCT 3′;

to form pHK12. Following restriction of pIDI with BsaAI-SmaI, agarose gel electrophoresis and GeneClean purification, the resulting blunt-ended 0.7 Kb DNA fragment containing the S. pombe IPP isomerase orf is inserted into the filled in NotI site of pTHBN1 utilizing chain reaction cloning (Pachuk et al., 2000), thermostable Ampligase® (Epicentre Technologies, Madison, Wis.), and the bridging oligonucleotides:

(SEQ ID NO: 46)
11) 5′ TTAAATAAGGAGGAATAAACCATGGCGGCCGTAGGAGGCAC
ATATGAGTTCCCAACAAGA 3′;
and
(SEQ ID NO: 47)
12) 5′ ACCTTAATTCATCGTTGCTAAGGATCCCCCGGCCGCGATCC
GAGCTCGAGATCTGCAGCT 3′;

to form pHK13.

### Example 14

### Increased Isoprenoid Production in Cells Containing the MEP Pathway

In another exemplified embodiment, a carotenoid producing E. coli strain is utilized to demonstrate the effect of the insertion of orfs encoding the entire mevalonate pathway, or orfs encoding the entire mevalonate pathway and IPP isomerase, or an orf encoding just IPP isomerase, on production of lycopene as follows: Following the transformation of E. coli TOP10 F′ (Invitrogen) with pAC-LYC (Cunningham et al., J. Bacteriol. 182:5841-5848, 2000), transformed cells are isolated on LB/Cam (30 μg/ml) plates grown at 30° C. TOP10 F′/pAC-LYC competent cells are prepared by the CaCl₂ method (Sambrook et al., 1989) following growth in LB/Cam in darkness at 28° C. and 225 rpm to an optical density (A₆₀₀) of 0.6. Competent TOP10 F′/pAC-LYC cells are transformed with one of the following plasmids: pTrcHisB; pHKO9, a pTrcHisB derivative containing the entire mevalonate pathway; pHK10, a pTrcHisB derivative containing the entire mevalonate pathway plus the orf encoding R. capsulatus IPP isomerase; pHK11, a pTrcHisB derivative containing the entire mevalonate pathway plus the orf encoding S. pombe IPP isomerase; pHK12, a pTrcHisB derivative containing the orf encoding R. capsulatus IPP isomerase; and pHK13, a pTrcHisB derivative containing the orf encoding S. pombe IPP isomerase. The bacterial strains described above, comprising pTHBN1 derivatives containing the mevalonate pathway orfs and/or an orf encoding IPP isomerase, are designated HK1, HK2, HK3, HK4, and HK5 respectively. The resulting transformants are isolated as colonies from LB/Cam/amp plates grown at 30° C. Single colonies of TOP10 F′/pAC-LYC/pTrcHisB and HK1 (TOP10 F′/pAC-LYC/pHKO9) are used to individually inoculate 4 ml LB/Cam/amp cultures and grown overnight in the dark at 28° C. and 225 rpm. The cultures are serially diluted 10,000 to 100,000-fold, plated on LB/Cam/amp medium containing IPTG, and grown in the dark at rt for 2 to 10 days. The plates are visually examined for an increase in lycopene production as evident by a “darkening” of the light pink colored colonies that are present on the control plates corresponding to TOP10 F′/pAC-LYC/pTrcHisB. The same experiments are performed with strains HK2, HK3, HK4, and HK5 to determine, visually, the effect of the orfs contained within pHK10, pHK11, pHK12, and pHK13 on lycopene production in TOP10 F′/pAC-LYC cells. The quantification of the carotenoid lycopene in cells, identified as potential overproducers due to their darker color when compared to the color of TOP10 F′/pAC-LYC/pTHBN1 cells, is performed utilizing a spectrophotometric assay as described by Cunningham et al. (Cunningham et al., 2000). Increased production of lycopene in E. coli cells containing the entire mevalonate pathway or the entire mevalonate pathway plus an additional orf for IPP isomerase establishes that the presence in cells of an additional biosynthetic pathway for the formation of IPP or IPP and DMAPP enhances the production of isoprenoid compounds, such as carotenoids, that are derived from IPP and DMAPP.

### Example 15

### Demonstration of Antibiotic Resistance Due to the Mevalonate Pathway in MEP Pathway Dependent Cells

In still another exemplified embodiment, E. coli cells are transformed with DNA containing orfs, which in their summation comprise the entire mevalonate pathway, and the resulting cells are tested for resistance to the antibiotic fosmidomycin as follows: Following the separate transformation of E. coli TOP 10 F′ (Invitrogen) with pHKO2, pHKO3 and pHKO9, transformed cells are isolated on LB/Amp (50 μg/ml) plates grown at 30° C. Single colonies of TOP10 F′/pHKO2 (designated strain HK6), TOP10 F′/pHKO3 (designated strain HK7), and TOP 10 F′/pHKO9 (designated strain HK8), are used to individually inoculate 4 ml LB/amp cultures and grown overnight at 30° C., 225 rpm. The HK6 and HK7 cultures are serially diluted 10,000 to 100,000-fold and plated on LB containing fosmidomycin (20 μg/ml). The HK8 cultures are serially diluted 10,000 to 100,000-fold and plated on LB/IPTG containing fosmidomycin (20 μg/ml) Controls are performed with cells comprising TOP10 F′ transformed with the parent vectors of pHKO2, pHKO3 and pHKO9, by plating on the appropriate medium containing fosmidomycin establishing that E. coli control cells are unable to grow on medium containing fosmidomycin. The ability of transformed E. coli cells to grow in the presence of the antibiotic fosmidomycin establishes that the inserted DNA, comprising the entire mevalonate pathway and thus an alternative biosynthetic route to IPP, is functional and can circumvent the inhibition of an enzyme in the trunk line of the MEP pathway.

### Example 16

### Construction of Plastid Transformation Vectors

In a specific, exemplified embodiment, a plant plastid transformation vector containing a synthetic operon comprising orfs, which in their summation is the entire mevalonate pathway, is constructed as follows: Plasmid pHKO3, a pBluescript derivative containing all six mevalonate pathway orfs, is assembled by restriction of pFCO1 to yield a 3.9 Kb NotI-XhoI DNA fragments containing three mevalonate orfs and its subsequent insertion into the SalI-NotI sites of pHKO1 by directional ligation as described above in Example 8. The plastid transformation vehicle, pHK14 containing the entire mevalonate pathway is constructed as follows: Plastid vector pGS104 (Serino and Maliga, Plant J. 12:687-701, 1997) is restricted with NcoI-XbaI and the two resulting DNA fragment are separated by agarose gel electrophoresis. Following isolation of the larger DNA fragment by gel excision and its purification by GeneClean, the NcoI-XbaI 5′ overhangs are dephosphorylated using SAP and filled in with Klenow and dNTPs. The resulting blunt-ended, dephosphorylated DNA fragment derived from pGS104 is GeneClean purified. Following restriction of pHKO3 with EagI, isolation by agarose gel electrophoresis, and purification by GeneClean, the 7.7 Kb DNA fragment is treated with Klenow and dNTPs to fill in the 5′ overhangs. The resulting blunt-ended DNA fragment containing the mevalonate pathway is purified by GeneClean and inserted into the dephosphorylated, Klenow-treated NcoI-XbaI sites of pGS104 by blunt-end ligation to yield pHK14.

Derivatives of pGS104 containing the entire mevalonate pathway plus an additional orf encoding IPP isomerase are constructed as follows: Following restriction of pHKO5 with NotI and treatment with Klenow and dNTPs, the resulting 8.2 Kb blunt-ended DNA fragment, containing the six mevalonate pathway orfs plus an orf encoding R. capsulatus IPP isomerase, is isolated by agarose gel electrophoresis, purified by GeneClean, and inserted into the dephosphorylated, filled in NcoI-XbaI sites of pGS104 by blunt-end ligation to yield pHK15. Following restriction of pHKO6 with EagI and treatment with Klenow and dNTPs, the resulting 8.4 Kb blunt-ended DNA fragment, containing the six mevalonate pathway orfs plus an orf encoding S. pombe IPP isomerase, is isolated by agarose gel electrophoresis, purified by GeneClean, and inserted into the dephosphorylated, filled in NcoI-XbaI sites of pGS104 by blunt-end ligation to yield pHK16.

Derivatives of pGS104 containing only an orf encoding IPP isomerase are constructed as follows: Following restriction of pBSIDI with BsaAI-EcoRV, agarose gel electrophoresis and GeneClean purification, the resulting blunt-ended 0.5 Kb DNA fragment containing the R. capsulatus IPP isomerase orf is inserted into the dephosphorylated, filled in NcoI-XbaI sites of pGS104 by blunt-end ligation to yield pHK17. Following restriction of pIDI with BsaAI-SmaI, agarose gel electrophoresis and GeneClean purification, the resulting blunt-ended 0.7 Kb DNA fragment containing the S. pombe IPP isomerase orf is inserted into the dephosphorylated, filled in NcoI-XbaI sites of pGS104 by blunt-end ligation to yield pHK18.

### Example 17

### Construction of Transplastomic Plants Containing Orfs Encoding the Mevalonate Pathway or Orfs Encoding the Mevalonate Pathway Coupled with IPP Isomerase

In another exemplified embodiment, tobacco is engineered at the plastid level by using any of the plastid transformation vectors described above, or their equivalents, such as variants of those plastid transformation vectors as can be routinely constructed by means known in the art and containing the orfs as taught and described above. Specifically, Nicotiana tabacum var. ‘Xanthi NC’ leaf sections (1×0.5 cm strips from in vitro plants with 3 to 5 cm long leaves) are centered in the dish, top side up and bombarded with 1 μm gold micro particles (Kota et al., 1999) coated with DNA containing orfs, which in their summation comprise the entire mevalonate pathway, using a PDS1000 He device, at 1100 psi. Toxicity is evident in tobacco after three weeks of growth on medium containing the antibiotic fosmidomycin at a concentration of at least 500 micromolar. Transplastomic plants are recovered from leaf sections cultured under lights on standard RMOP shoot regeneration medium or on a Murashige-Skoog salts shoot regeneration medium with 3% sucrose, Gamborg's B5 vitamins, 2 mg/L 6-benzylamino-purine and Phytagel (2.7 g/L), containing 500 μM fosmidomycin for the direct selection of insertion of the entire mevalonate pathway into plastids. Alternatively, the regeneration medium contains an antibiotic, e.g. spectinomycin, for selection based on antibiotic resistance due to any co-transformed gene on the transforming DNA vector, as would be readily apparent to the skilled artisan. De novo green leaf tissue is visible after three weeks. Tissue is removed to undergo a second round of selection on shoot regeneration medium with 500 μM fosmidomycin to encourage homoplasmy and plants are rooted. Genomic DNA is isolated from T0 leaf tissue or T1 leaf tissue derived from in vitro germinated transplastomic seeds utilizing the DNeasy Plant Mini Kit (Qiagen Inc, Valencia, Calif.) according to the manufacturer's instructions and is subjected to analysis as is known in the art to confirm homoplasmy. The ability to select directly for a transformation event corresponding to the successful insertion of the mevalonate pathway orfs into plastids establishes the use of orfs, which in their summation comprise the entire mevalonate pathway, as a selectable marker for plastid transformation. The construction of fosmidomycin resistant plants establishes the ability of the mevalonate pathway, when functioning in plant plastids, to provide an alternate biosynthetic route to IPP, thus overcoming the effect of an inhibitor targeting an enzyme in the trunk line of the MEP pathway.

### Example 18

### Metabolic Engineering in Transplastomic Solanaceae Plants

In another exemplified embodiment, Solanaceae species are engineered at the plastid level using infA pseudogene insertion of a selectable marker and orfs for expression. Specifically, leaf sections of a genetically defined white petunia (or other petunia), are engineered, as for the Solanaceous species tobacco (see Example 16), using vectors pHK04 or pHKO7, or their equivalents, for insertion of orfs encoding the entire mevalonate pathway or orfs encoding the entire mevalonate pathway and IPP isomerase. Transplastomic Solanaceae plants containing orfs encoding the entire mevalonate pathway and IPP isomerase, and containing an additional orf encoding phytoene synthase, are created by insertion of a pBSNT27 (see Example 9) derived vector, constructed as follows:

A Rhodobacter capsulatus orf encoding a polypeptide with phytoene synthase activity is isolated by PCR from genomic DNA using the primers

(SEQ ID NO: 65)
1) 5′ GCGATATCGGATCCAGGAGGACCATATGATCGCCGAAGCGGA
TATGGAGGTCTGC 3′ (sense)
(SEQ ID NO: 66)
2) 5′ GCGATATCAAGCTTGGATCCTCAATCCATCGCCAGGCCGCGG
TCGCGCGC 3′ (antisense)

containing the restriction site BamHI shown underlined. The 1.1 Kb PCR product is isolated by agarose gel electrophoresis, purified by GeneClean and inserted into the pT7Blue-3 vector (Novagen) using the Perfectly Blunt (Cloning Kit (Novagen) according to the manufacturer's instructions. Sequence analysis is performed to identify constructs containing R. capsulatus DNA identical to the published DNA sequence (SEQ ID NO: 71) and are designated pPHS.

Following restriction of pPHS with BamHI, isolation by agarose gel electrophoresis, and purification by GeneClean, the 1.1 Kb BamHI DNA fragment containing the orf encoding R. capsulatus phytoene synthase is inserted into the BglII site of pBSNT27 utilizing chain reaction cloning (Pachuk et al., 2000), thermostable Ampligase((Epicentre Technologies, Madison, Wis.), and the bridging oligonucleotides

(SEQ ID NO: 67)
3) 5′ CTTTCCTGAAACATAATTTATAATCAGATCCAGGAGGACCAT
ATGATCGCCGAAGCGGAT 3′;
and
(SEQ ID NO: 68)
4) 5′ CGACCGCGGCCTGGCGATGGATTGAGGATCTAAACAAACCCG
GAACAGACCGTTGGGAAG 3′;

to create plastid transformation vector pFHO5. Following restriction of pFHO5 with XcmI, a unique site in the infA pseudogene, and purification by GeneClean, the resulting 3′ overhangs are removed by treatment with Mung Bean nuclease and the resulting blunt-ended DNA fragment is purified by GeneClean. Vector pFHO3 is restricted with NotI and the resulting 8.3 Kb DNA fragment, containing Operon E, is isolated by agarose gel electrophoresis and purified by GeneClean. The 5′ overhangs of the isolated DNA fragment are filled in with Klenow and dNTPs and the resulting blunt end DNA fragment, containing Operon E, is inserted into the Mung Bean nuclease treated XcmI site of pFHO5 utilizing chain reaction cloning (Pachuk et al., 2000), thermostable Ampligase((Epicentre Technologies, Madison, Wis.), and the bridging oligonucleotides

(SEQ ID NO: 69)
5) 5′ ATTTTTCATCTCGAATTGTATTCCCACGAAGGCCGCGTCGAC
TACGGCCGCAGGAGGAGT3′;
and
(SEQ ID NO: 70)
6) 5′ TTCGGATCGATCCTGCGCGGCTGAGCGGCCGGAATGGTGAAG
TTGAAAAACGAATCCTTC3′;

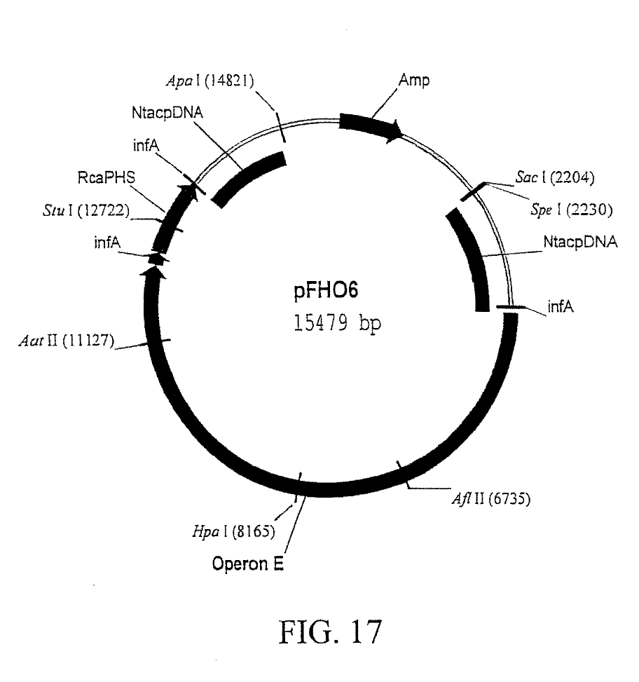

to create the plastid transformation vector pFHO6 (FIG. 17).

Alternatively, an orf encoding IPP isomerase can be inserted into the XcmI site of pFHO5, utilizing skills as known in the art, to create a plastid transformation vector containing both an orf encoding phytoene synthase and an orf encoding IPP isomerase. Another alternative uses the infA pseudogene as an insertion site for orfs, encoding phytoene synthase, and/or IPP isomerase, and/or the entire mevalonate pathway, linked with the aadA gene as is known in the art for selection of transplastomic plastids on 500 microgram per liter spectinomycin.

The BioRad PDS1000 He gene gun is used to deliver BioRad tungsten M10 (0.7 micron approx.) microspheres into petunia (Petunia hybrida ‘Mitchell’) leaves positioned top-side up. Intact leaves, or equivalent tissues of about 6-8 cm² per sample are plated onto shoot regeneration medium consisting of Murashige and Skoog basal medium, B5 vitamins, 3% sucrose, 0.7% (w/v) agar and 3 mg/l BA (6-benzylamino-purine), 0.1 mg/l IAA (Deroles and Gardner, Plant Molec. Biol. 11: 355-364, 1988) in 100×10 mm plastic Petri dishes. Leaves are centered in the target zone of the gene gun for bombardment at 1100 psi, third shelf from bottom, ˜5.6 cm gap, 28 mgHg vacuum. M10 microspheres are coated with DNA using standard procedures of CaCl₂ and spermidine precipitation, 1.5 to 2 ug DNA/bombardment. After bombardment, tissues are cultured in light in the presence of antibiotic (500 micromolar fosmidomycin). Each leaf sample is then cut into about 6 pieces and cultured on petunia shooting medium containing 500 micromolar fosmidomycin for 3 to 8 weeks, with subculture onto fresh medium every three weeks. Any green shoots are removed and leaves plated onto the same medium containing 500 micromolar fosmidomycin. Plantlets with at least four leaves and of solid green color (no bleaching on petioles or whorls) are transferred for rooting onto solidified hormone-free Murashige and Skoog salts with B5 vitamins and 2% sucrose and are grown to flowering. The dependency of increased carotenoid production in Solanacae on the combination of the orfs inserted, be it an orf encoding phytoene synthase alone; or orfs encoding the entire mevalonate pathway and phytoene synthase; or orfs encoding phytoene synthase, the entire mevalonate pathway and IPP isomerase; or orfs for phytoene synthase and IPP isomerase, establishes that the addition of the mevalonate pathway and/or IPP isomerase to plant plastids enhances the production of isoprenoid compounds that are derived from IPP and DMAPP; and the suitability of a pseudogene insertion site for creating transplastomic Petunia.

### Example 19

### Transformation of Microalgae

In a specific exemplified embodiment, chloroplast transformants are obtained by microprojectile bombardment of Chlamydomonas reinhardtii cells and subsequent selection on fosmidomycin. Specifically, a genecluster containing the complete mevalonate pathway is substituted, as a selectable marker, for the coding sequence of the aadA gene in the pUC18 derived vector containing 5-atpA:aadA:rbcL-3 (Goldschmidt-Clermont M., Nucleic Acids Res. 19:4083-4089, 1991) as follows: Plasmid pUC-atpX-AAD is restricted with NcoI, purified by GeneCleanand treated with Mung Bean nuclease to remove the resulting 5′ overhangs. Following GeneClean purification, the blunt ended DNA fragment is restricted with HindIII to remove the aadA orf and the remaining DNA fragment, containing approximately 653 base pairs of the C. reinhardtii atpA gene and approximately 437 base pairs of the C. reinhardtii rbcL gene (Goldschmidt-Clermont M., 1991), is isolated by agarose gel electrophoresis and purified by GeneClean. Plasmid pFHO4 is restricted with NdeI, purified by GeneClean, and the resulting 5 overhangs are filled in with Klenow and dNTPs. Following GeneClean purification, the blunt ended DNA fragment is restricted with HindIII and the resulting DNA fragment, containing Operon F (see FIG. 13), is isolated by agarose gel electrophoresis and purified by GeneClean. The blunt end-HindIII fragment is inserted into the blunt end HindIII sites of the DNA fragment isolated from pUC-atpX-AAD by ligation resulting in the orf encoding S. cerevisiae acetoacetylCoA thiolase, located at the beginning of Operon F, to be in frame with the ATG start codon of the SatpA DNA in pUC-atpX-AAD (Goldschmidt-Clermont M., 1991). The resulting modified yeast orf only encodes 2 extra amino acids, Met and Ser, appended to the N-terminal Met of the acetoacetylCoA thiolase polypeptide encoded by Operon F. The resulting chlamydomonas plastid transformation vector is designated pHK19. About 10,000 cells are spread on TAP plates containing 200 micromolar fosmidomycin, plates are dried, and then cells are immediately bombarded with M10 or 1 micron gold particles coated with about 2 micrograms of plasmid DNA using the PDS-1000 He gene gun, 1100 psi, fourth shelf from bottom, ˜2 cm gap, ˜28 mgHg vacuum (alternatively cells are spread over a Nytran nylon 0.45 micron membrane placed on top of TAP agar and bombarded without a drying phase). Plates are incubated in low light for two to three weeks before colonies are counted. Fosmidomycin-resistant colonies are green (vs yellowish for susceptible cells) and transformants are characterized using skills as known in the art. This demonstrates use of orfs encoding the entire mevalonate pathway as a selectable marker for green algae and by virtue of its functioning demonstrates its utility for overproduction of isoprenoid metabolites in microalgae.

### Example 20

### Metabolic Engineering in Transplastomic Grain Crops (Rice)

In another exemplified embodiment, an operon comprising orfs encoding the entire mevalonate pathway are inserted into the plastids of rice as follows: A DNA fragment isolated from pHKO3, containing the complete mevalonate pathway, or from pFHO2, containing orfs encoding the entire mevalonate pathway and IPP isomerase, is inserted into the NcoI-XbaI sites of plasmid pMSK49 to replace the gfp coding region adjacent to the coding region for streptomycin resistance, aadA; or inserted into the BstXI-NcoI digested DNA of plasmid pMSK48 using skills as is known in the art for direct selection on fosmidomycin. The resulting plasmids contain rice-specific insertion sequences of pMSK35 as described in Khan and Maliga, Nature Biotechnology 17: 910-914, 1999. Embryonic suspensions, induced as previously described (Khan and Maliga 1999), of japonica rice Oryza sativa ‘Taipei 309’ engineered with the beta-carotene pathway (Ye et al. Science 287:303-305) are plated into filter paper and bombarded with the PDS1000 He device as described in Example 17. After two days on non-selective medium and then one to two weeks in selective AA medium (Toriyama and Hinata, Plant Science 41: 179-183, 1985) tissue is transferred to agar solidified medium of MS salts, and vitamins, 100 mg/L myo-inositol, 4 mg/L 6-benzylaminopurine, 0.5 mg/L indoleacetic acid, 0.5 mg/L1-napthaleneacetic acid, 3% sucrose, 4% maltose and 100 mg/L streptomycin sulfate or 500 μM fosmidomycin. Transplastomic shoots appear following cultivation in the light after three weeks and leaf samples are analyzed for the operon by PCR.

### Example 21

### Construction of Tobacco-Specific Polycistronic Vectors

In an exemplified embodiment, a series of tobacco-specific polycistronic vectors are constructed to assess the utility of chloroplast pseudogene vectors for multiple gene expression in the tobacco model.

Vector pKAS3

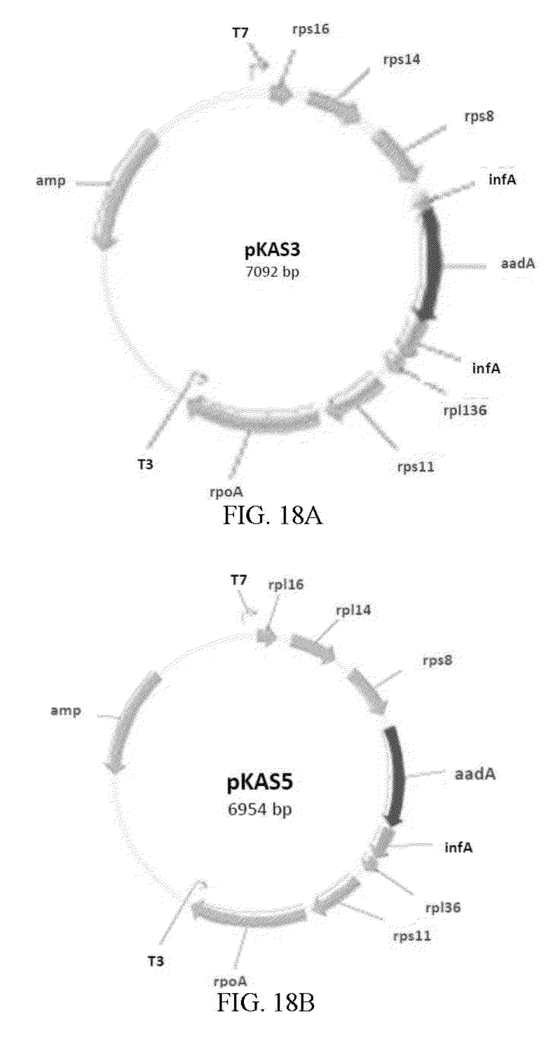

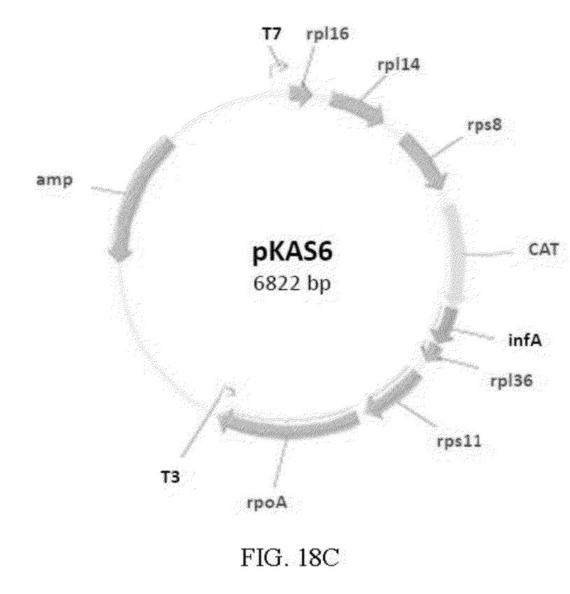

The original tobacco G-POS vector, pKAS3, is developed by cloning part of the rpl23 operon from rpl16 through rpoA genes. Using a restriction site in the middle of the infA pseudogene sequence, the bacterial aadA coding sequence is inserted into pKAS3, allowing selection of chloroplast transformants in the presence of spectinomycin (FIG. 18A).

Vector pKAS5

Another vector, pKAS5, is constructed by placing the selectable marker gene 11 bp downstream of a slightly modified ribosome binding site (RBS) (FIG. 18B). The pK5 vector has a canonical RBS “AAGGAGG” and an additional set of nucleotides to make the spacing between the RBS and +1 similar to that of Antirrhinum and Spinacia. Advantageously, pKAS5 represents a closer-to-natural gene arrangement, such as is found in other 5′ UTR positions in the tobacco chloroplast. pKAS5 can be used in parallel with pKAS3 to access utility of G-POS chloroplast pseudogene vectors in the tobacco model.

Vector pKAS6-aphA6

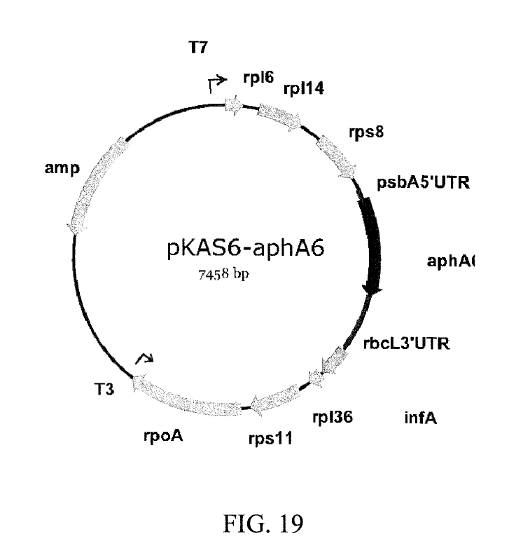

In order to optimize selection of transgenic tobacco plants and allow better recovery of regenerating shoots, pKAS6-aphA6 (FIG. 19) vector series are constructed with the Acinetobacter baumannii aminoglycoside phosphotransferase enzyme APH(3′)-VI. As shown in FIG. 19, the aphA6 gene expression is regulated by the Chlamydomonas psbA 5′UTR and rbcL 3′UTR. AphA6 has been employed successfully in recovery of transplastomic Chlamydomonas (Bateman and Purton, 2000) and tobacco (Herz et al., 2005; Huang et al., 2002).

### Example 22

### Tobacco Plastid Transformation

In an exemplified embodiment, tobacco plastid transformation is performed. Briefly, ten particle bombardment experiments are performed in tobacco plastid, using vectors pKAS3 or pKAS5 to optimize conditions for bombardment and early selection. Post-treatment, high-level exposure to antibiotic with a medium conducive to callusing, or low-level exposure to antibiotic to permit sorting out of recombinant genotypes, is employed on a medium conducive to caulogenesis. Parallel transient beta-glucuronidase gene (GUS) expression assay is performed, and the transgenic plants exhibiting excellent growth condition are selected. Specifically, experiments (S7, S8, S9 and S10) show excellent results with the controls using the transient GUS expression assay; S8 yields promising growth. Tissues of the selected plants are proliferated on antibiotic to promote homoplasmy and then tested by molecular assays for the presence of the transgenes in the chloroplast.

### Example 23

### Demonstration of Integration of the Transgene in Tobacco Chloroplast Genome

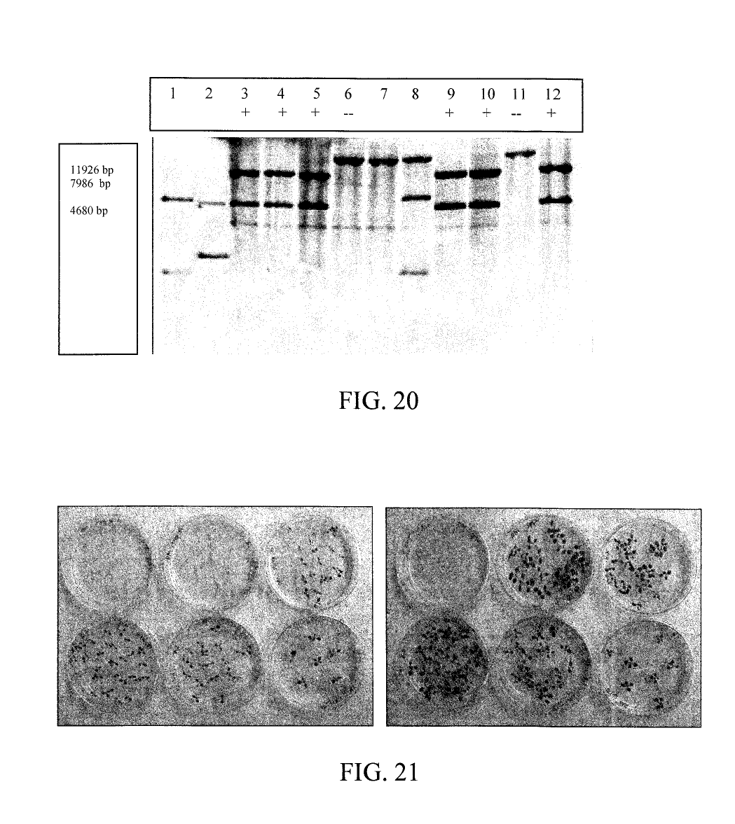

This Example demonstrates the successful integration of the transgene in tobacco chloroplast genome. Briefly, vector pKAS5 is introduced into tobacco leaves using the Particle Inflow Gun, and tobacco shoots are recovered from explants of these leaves after culture on regeneration medium containing spectinomycin. A total of 6 shoots are recovered from 46 bombarded leaves in two experiments. These shoots are repeatedly regenerated to promote homogeneity of the transformed chloroplasts (homoplasmy), and DNA is subsequently extracted from rooted plants. PCR analysis is performed to determine the presence or absence of the expected aadA adenylyltransferase transgene, and to determine whether the transgene is integrated into the desired chloroplast location (data not shown). Subsequently, Southern blot analysis is performed for determining the integration of the transgene into the chloroplast genome (FIG. 20).

The results, as shown in FIG. 20, demonstrate that the transgene can be successfully integrated at the desired locus in the chloroplast genome. Specifically, the results show that four out of the six lines exhibit the integration of the transgene at the desired locus in the chloroplast (FIG. 20, lanes marked+) by the presence of the expected 7986 and 4680 bp bands. Two lines display the WT 11926 bp band and are presumed to contain a spontaneous mutation in the 16srRNA which confers resistance to spectinomycin (FIG. 20, lanes 6 and 11), which is expected to occur at a low frequency in such experiments. The sample size of this experiment is small, however, it is of interest to note that no transgenic lines are recovered containing a standard chloroplast DNA vector commonly used in the art, despite simultaneous co-bombardment of all leaf samples with an equimolar concentration of both the pKAS5 and state of the art vectors. The two vectors integrate at different loci, and thus, plants containing both transgenes can also be recovered. Therefore, vectors of the present invention, as represented by pKAS5, are equal to and possibly superior to the state of the art chloroplast transformation vectors in use in the art.

### Example 24

### Demonstration that Phenotypes of the Transgenic Tobacco Result from the Selective Marker

This Example demonstrates that the phenotype of transgenic plants is due to the selectable marker, and not as a result of a spontaneous mutation in the chloroplast 16s rRNA. Cultivation of tissue in the presence of both spectinomycin and streptomycin allows regeneration of green shoots only when the selectable marker gene is present. Spontaneous mutations in the 16s rRNA allowing resistance to aminoglycoside antibiotics such as spectinomycin, streptomycin and kanamycin is well documented for Chlorophytes and for numerous prokaryotes. Recovery of 16s rRNA mutants after culture on kanamycin is reported for Medicago (Rosellini et al., 2004) and Chlamydomonas, where approximately 10% of recovered cells are due to such mutations (Bateman and Purton, 2000). A literature search fails to yield any information on the frequency of such spontaneous mutants of tobacco resulting from kanamycin selection.

Specifically, the results show that, in positive control, shoots regenerate as expected from WT tissue on RMOP medium lacking antibiotic; negative control fails to regenerate shoots from WT leaf pieces on RMOP medium containing both antibiotics. Four transplastomic lines are able to regenerate green calli/shoots under double selection (data not shown).

In addition, seeds harvested from the transgenic lines are similarly tested to demonstrate heritability of the transgene (FIG. 21). For homoplasmic plants, 100% of the germinating seeds are resistant to both antibiotics; heteroplasmic lines yield a mixed population of resistant and sensitive progeny. Seeds are sterilized by 10% bleach, and germinate on RM media containing either spectinomycin or streptomycin at 500 μg/ml. All WT seedlings are small and bleach as expected, due to sensitivity to the selection agent. All seedlings of transgenic lines S8-1, -3, -4 and S10-1 are green and develop true leaves as expected for transplastomic plants. Line S8-5, a spontaneous 16s rRNA mutant line, is also included in the germination experiment as a control; it is resistant to spectinomycin but sensitive to streptomycin as expected for this genotype. The spectinomycin selection level is preferred for recovery of transplastomic tobacco based upon the prior success at recovering three transgenic lines with vector pKAS5.

For the selection of pK6aphA6-treated tobacco leaves, kanamycin selection is performed at a level of 25 ug/ml. The selection of transplastomic tobacco via kanamycin may require a narrow effective concentration of the antibiotic.

### Example 25

### Physical Apparatus for Introduction of DNA into Tobacco Cells

This Example embodies physical apparatuses useful for introducing DNA into cells.

An exempliflied transformation method is the gene gun bombardment of tobacco, comprising the steps of: coating gold particles with DNA vectors; and biolistically delivering the particles into cells. In an embodiment, Bio-Rad 0.6 μm gold (Hercules, Calif.) or Seashell 0.6 μm gold (La Jolla, Calif.) can be used. Seashell gold offers an advantage due to its superior ability to be coated with DNA and its better dispersion characteristics. Preferably, Seashell gold can be delivered at a reduced amount per shot, as compared to the Bio-Rad 0.6 μm gold. For example, 60 μg gold is used per shot, which is lower than the 500 μg Bio-Rad gold per shot used in earlier experiments. Control tissue shot with a GUS vector demonstrates a high efficiency of DNA delivery with Seashell gold even at the lower quantity of gold per shot. Increasing the amount of gold to 120 μg per single shot does not appear to improve the delivery of plasmids into the cell.

### Example 26

### Analysis of the Integration of the Transgene in the Transplastomic Lines

In this Example, additional new transplastomic lines with vectors shown in FIG. 18 and FIG. 19. are obtained. Nine particle inflow gun (PIG) bombardment experiments are executed with 1 to 2 shots per experiment. A total number of 67 tobacco leaves are shot: 52 with pK6aphA6 and 15 with pKAS5.

Vector pK6aphA6

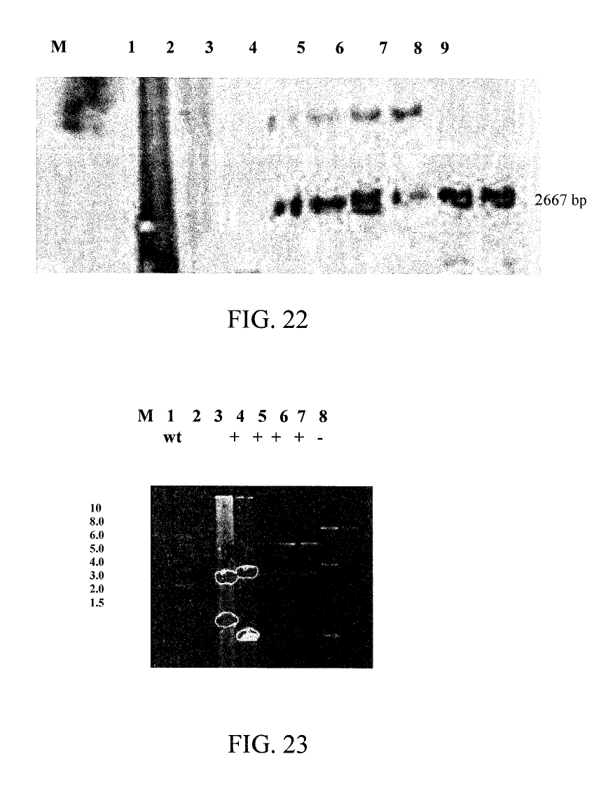

In this Example, kanamycin is applied at 25 ug/ml, and twenty-five kanamycin resistant shoots are recovered. Subsequent rounds of selection decreases the number of putative transgenic plants to six. Six of these plants are produced and are tested by PCR and Southern blot for the aphA6 transgene (FIG. 22). The data show that the transgene is integrated in the chloroplast at the desired locus. Restriction digestion of genomic DNA is performed. If the integration of the transgene occurs in the chloroplast, restriction digestion will yield a predictive product; whereas random nuclear integration will yield digestion products that cannot be predicted. As shown in FIG. 22, BglII digestion of the chloroplast-integrated aphA6 transgene yields a unique predicted product of 2667 base pairs, which is absent in wild-type genomic DNA (lane 3). This indicates that the transgene is integrated into the chloroplast genome.

Vector pKAS5

For vector pKAS5, experiments are performed and data demonstrate that recovery of transgenic tobacco chloroplasts is preferentially accomplished with the G-POS vectors of the present invention, when compared to an alternative state of the art tobacco transformation vector. Vector pKAS5 is introduced into tobacco leaves in a co-bombardment test with control vector pJS95 as is known in the art. Equal quantities of the two vectors are used to coat gold microparticles that are then introduced into tobacco leaves using the Particle Infiltration Gun (PIG).

Twelve tobacco lines are recovered in three experiments after PIG treatment of 91 leaves and selection of explant tissue on suboptimal levels of the antibiotic spectinomycin. To distinguish between true transplastomic lines versus spontaneous mutant lines, the plants are subjected to a round of co-selection on spectinomycin and streptomycin. DNA is collected from the dual-resistant tobacco plants and analyzed by PCR and Southern blot to determine which vectors are introduced into the plants, and confirm whether integration of DNA occurs in the targeted chloroplast locus or randomly in the nuclear genome.

FIG. 23 shows the Southern blot results for experiment S27. Combining the data from experiments S8, S10 and S27, a total of 11 plants with dual antibiotic resistance are observed. Seven of these plants are shown to contain the pKAS5 vector in the chloroplast (examples in FIG. 23, lanes 4-6), one plant contains vector pJS95 (FIG. 23, lane 7). Three plants (example in FIG. 23, lane 8) contain no vector DNA and therefore represent lines with a probable mutation in the chloroplast 16s rRNA, allowing them to escape dual selection.

### Example 27

### Analysis of Selection Conditions and Vector Efficiencies

This Example compares the results of the tobacco transformation experiments to determine the efficiency of the chloroplast vector and selection methods. Results demonstrate superior performance of vector pKAS5 when compared to alternative vectors. A total of 7 transgenic lines are recovered from treatment of 153 leaves with vector pKAS5. Simultaneous treatment of the same 153 leaves with a typical state of the art vector comprising a heterologous transcriptional promoter such as described in Maliga 1999 yields one transgenic event. The efficiency of the present invention is 4.6% and that of the state of the art is 0.6% in our hands. Treatment of 130 tobacco leaves with vector pKAS6 produces 6 transgenic lines and a transformation efficiency of 4.6%. pKAS5 and pKAS6 do not have a heterologous transcriptional promoter, a key feature of the present invention that distinguishes it from the state of the art. Transcription of the selectable marker gene in all pKAS vectors is regulated by the promoter of the rpl23 operon, more than 6,000 base pairs upstream. Transcript stability is provided by the operon, and thus the GPOS vector strategy of the present invention differs significantly from the prior art vector strategies by eliminating the need for a heterologous 3′UTR.

### Example 28

### Construction of Lemna-Specific Vectors

In an exemplified embodiment, a series of Lemna-specific vectors are constructed to assess the utility of chloroplast pseudogene vectors for multiple gene expression in the Lemna model.

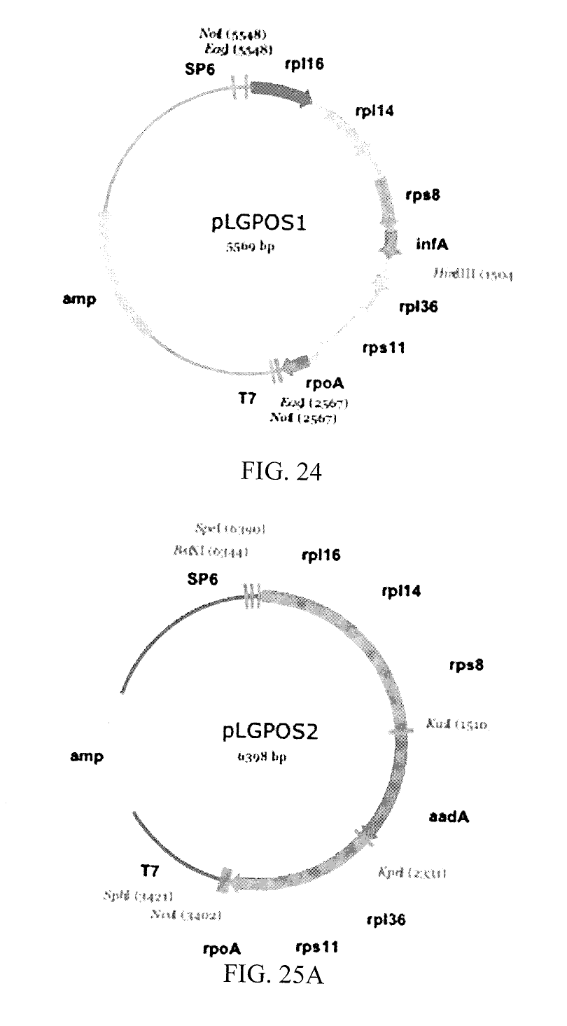

A baseline cloning vector, pLGPOS1 (FIG. 24), is first constructed by cloning a large portion of the Lemna rpl23 operon, using homology-based PCR cloning with NCBI sequence data from members of the order Liliopsidae. Variant vectors allowing reporting or selection can be constructed, as is known in the art.

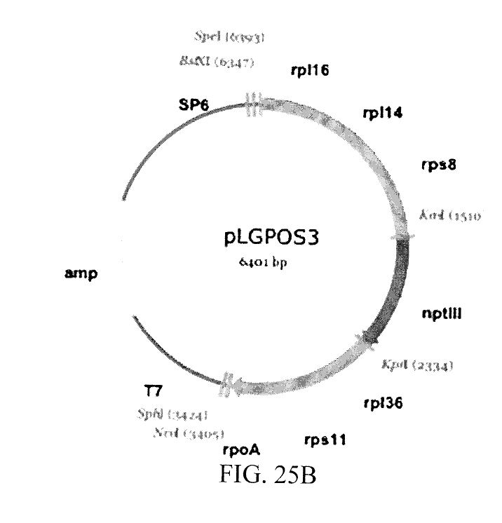

An example of a variant is as follows: The pLGPOS1 vector can be used to construct three plasmids containing genes allowing selection of transgenic Lemna chloroplasts. Vector pLGPOS2 contains a possible typical selection or reporter coding sequence or gene, the bacterial aadA gene, conferring resistance to spectinomycin; vector pLGPOS3 contains the nptIII gene conferring resistance to kanamycin; pLGPOS4 contains the aphA6 gene providing kanamycin resistance (FIG. 25). Examples of possible reporter genes include, but are not limited to, green fluorescent protein (GFP) and β-glucuronidase. Selectable marker genes are PCR amplified from donor plasmids, with the addition of a ribosome binding site at the 5′ end of the gene and useful restriction sites at both the 5′ and 3′ ends to facilitate future subcloning objectives. PCR products are digested with HindIII and ligated into the HindIII site present in the Lemna infA pseudogene of vector pLGPOS1.

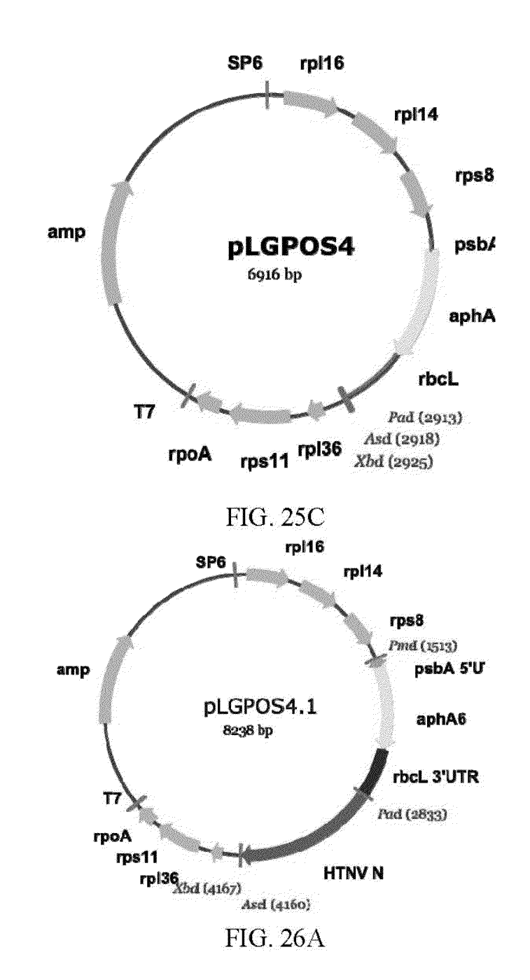

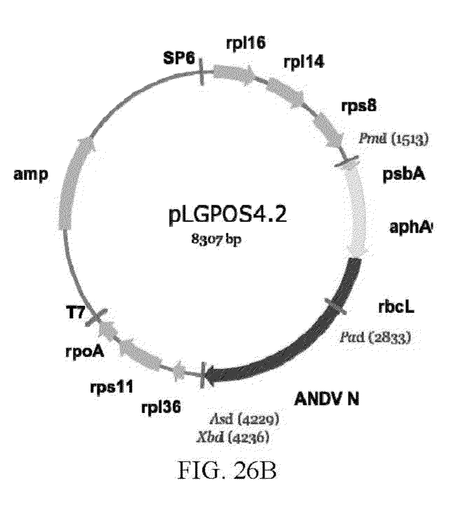

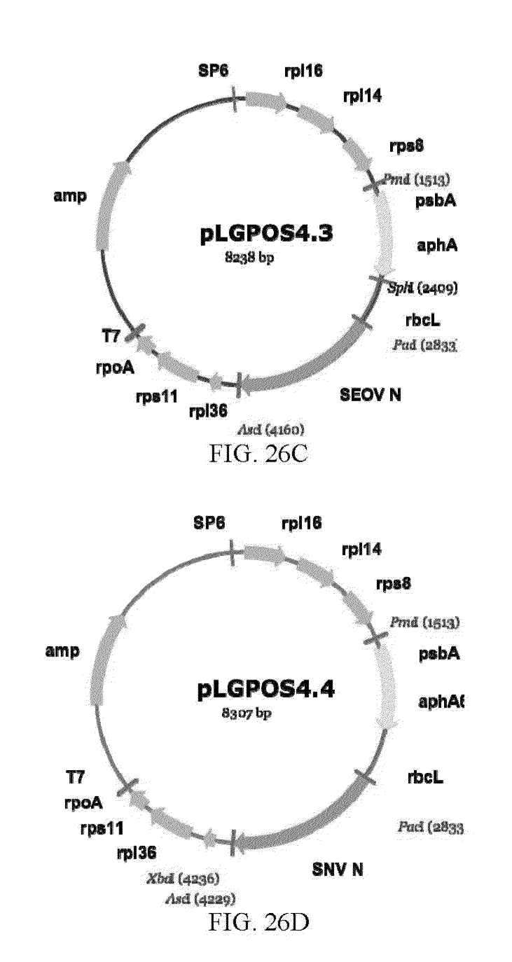

For subcloning for expression of therapeutic proteins in the chloroplast of Lemna, vector pLGPOS4 is specifically exemplified herein because the aphA6 marker has been used successfully in recovering transplastomic tobacco and Chlamydomonas. The selectable marker gene also has demonstrated functionality when the plasmid is propagated in E. coli. Similar vectors pLGPOS4.1-4.4 are also constructed (FIG. 26).

### Example 29

### Construction of Tobacco-Specific Vector Containing the Rhodobacter capsulatus IPPI Gene in the InfA Pseudogene Locus

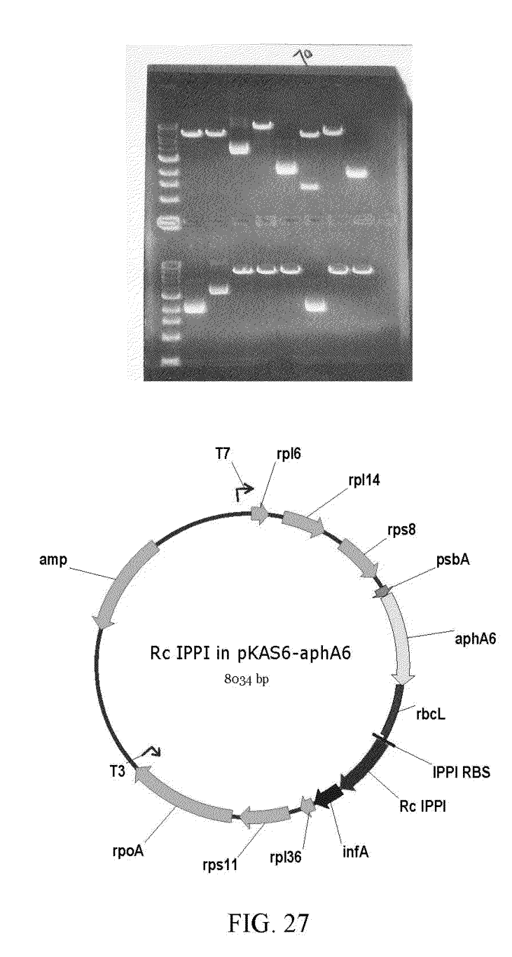

This Example illustrates the construction of tobacco-specific vector containing the Rhodobacter capsulatus IPPI gene in the infA pseudogene locus. The prokaryotic Rhodobacter capsulatus IPPI gene is isolated from proprietary vector pK3CATI by restriction digestion with the enzymes SphI and HindIII, yielding a 580 bp DNA fragment. Tobacco chloroplast vector pKAS6-aphA6-ANDV is linearized with enzymes AscI and PacI to produce a 7477 bp molecule. Both molecules are gel purified, blunted with T4 DNA polymerase, and the pKAS6 molecule is treated with Shrimp Alkaline Phosphatase to prevent recircularization. The DNA is purified using a Qiagen PCR purification column and the yield is confirmed by spectrophotometry and gel electrophoresis. Ligation of the two fragments overnight with T4 DNA ligase produces 30 colonies for subsequent PCR screening. Four clones chosen based on PCR results are cultured overnight, plasmid DNA is isolated by miniprep and is confirmed by restriction digestion with AvrII (FIG. 27). One of the four clones yields the correct fragments and is further confirmed by bidirectional DNA sequencing. The results of the restriction analysis and DNA sequencing confirm construction of the desired plasmid, and the DNA vector is stored in an E. coli host for propagation.

In addition, the results show a “C” deletion 45-base-pairs upstream of the IPPI coding region and a 20-base-pair deletion downstream of the IPPI coding region. The downstream deletion removes a PmeI restriction site from the parental vector that could be useful for subcloning purposes. Both of the deletions are attributed to incomplete processing of the ends of cloned fragments with T4 DNA polymerase prior to ligation. The “C” deletion is not predicted to affect IPPI translation because it is located in a multicloning site 28-base-pair upstream of the ribosome binding site. The downstream deletion is not expected to affect IPPI expression because of the location of the transgene in the operon; the mRNA 3′ UTR secondary structure and subsequent transcript stability are not of concern for this gene in this polycistronic context. In addition, the larger multicloning site deletion is within the infA pseudogene where its effect on translation of the inactive gene is not of concern.

### Example 30

### Construction of Tobacco Vector pKAS6-aphA6 Containing Synechocystis IPPI Gene

Two types of IPP isomerase are known to date. Type I enzymes, such as the one found in Rhodobacter, function in the mevalonate pathway of isoprenoid biosynthesis. They have been found in humans, archebacteria, Saccharomyces, and the cytoplasm of higher plants. A second type of IPP isomerase that functions in the MEP pathway of isoprenoid synthesis was identified in Streptomyces (Kaneda et al., 2000) and was determined to function as a tetramer in a flavin mononucleotide- and NADPH-dependent manner. This Type II enzyme also is found in plant chloroplasts, eubacteria such as E. coli, B. subtilis, S. aureus, and cyanobacteria such as Synechocystis sp. Comparison of the kinetic constants for type II IPP isomerases (Barkley et al., 2004) demonstrated that the enzyme found in Synechocystis sp. PCC 6803 has relatively high enzymatic efficiency as measured by k_{cat}/Kₘ (4.4×10³ versus 1.6×10³ for Streptomyces). The highest efficiency is found for Staphylococcus aureus (6.8×10⁴), but this organism is not selected as a source of IPPI gene owing to the pathogenicity of the organism.

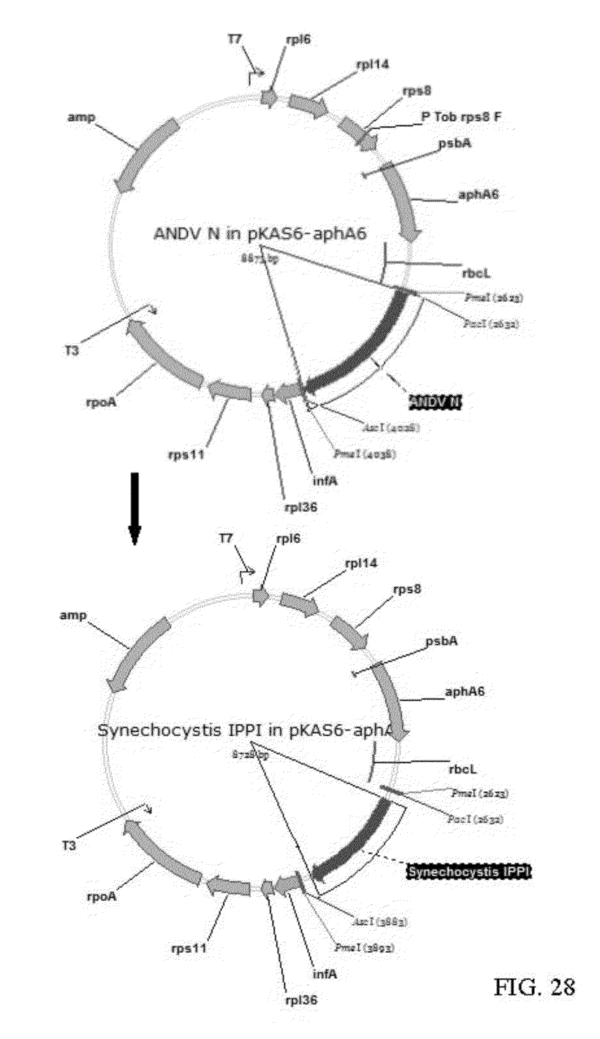

Exemplified herein is the use of Synechocystis as a source of type II IPP isomerase. Similar to the completed tobacco vector described hereinabove, vector pKAS6-aphA6-ANDV N is used as the backbone to construct the vector containing the Synechocystis IPPI gene. The ANDY N gene is removed by restriction digestion using the enzymes PacI and AscI, or the enzyme PmeI (FIG. 28. Specific primers for the Synechocystis IPPI gene are designed that include the said restriction enzymes at the ends for cloning. The PCR amplified product includes the native 5′ and 3′ UTR of the Synechocystis IPPI gene.

Genomic DNA Extraction from Synechocystis

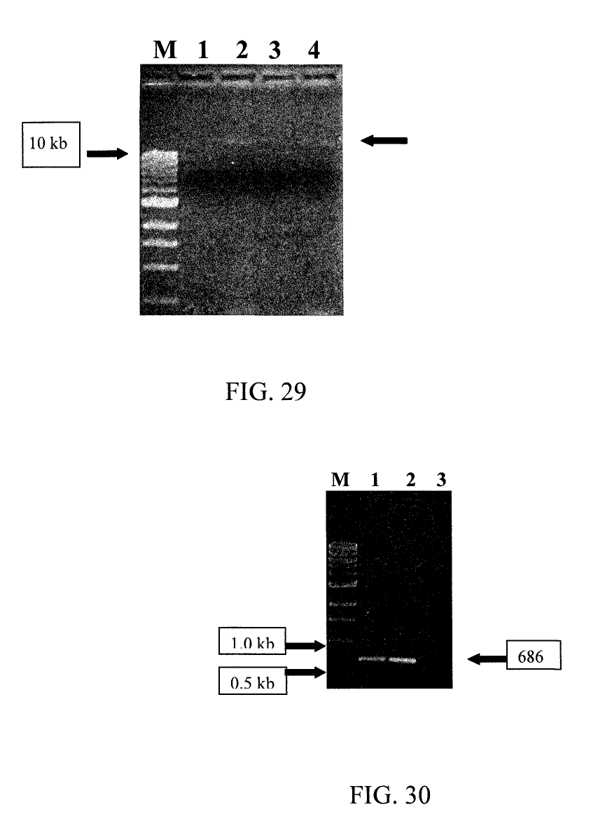

Genomic DNA can be extracted from Synechocystis using methods known in the art. FIG. 29 shows a representative agarose gel electrophoresis with genomic DNA extracted from Synechocystis (The bands are faint due to imperfect extraction process).

### Polymerase Chain Reaction (PCR) Using 16S Ribosomal RNA Specific Primers

To examine whether the genomic DNA obtained is suitable for PCR, amplification of a fragment of the gene encoding the 16S ribosomal RNA is performed. The 16S rRNA fragment (686 bp) is successfully amplified using the genomic DNA extracted (FIG. 30).

### Polymerase Chain Reaction (PCR) Using Synechocystis IPPI Gene Specific Primers

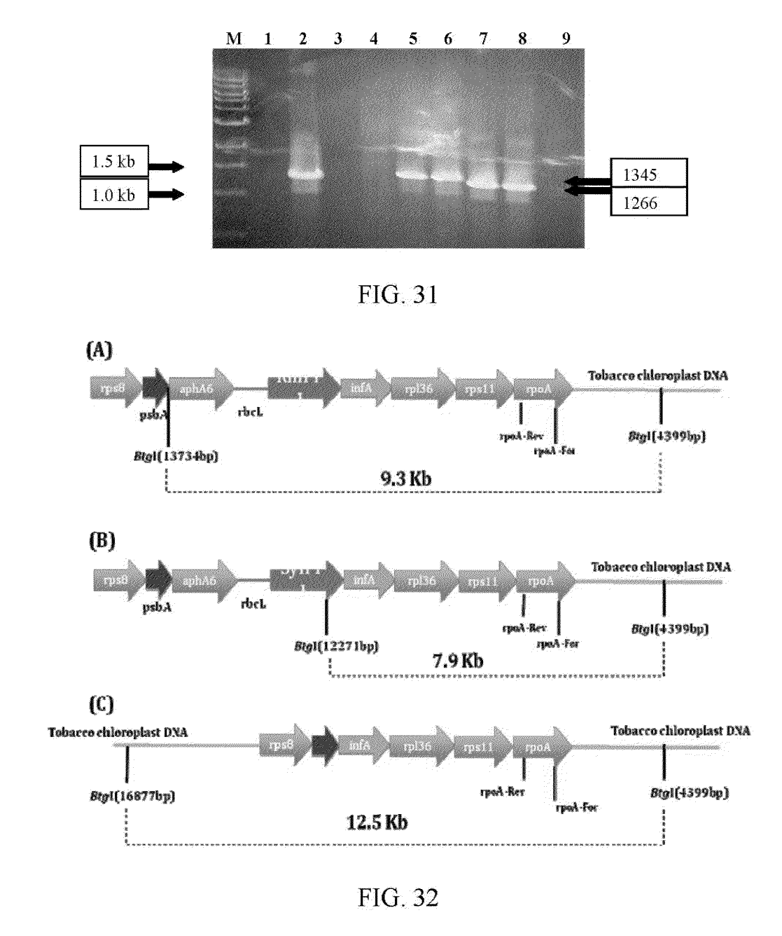

A preliminary PCR assay is performed using Synechocystis IPPI gene specific primers. As shown in FIG. 31, the Synechocystis IPPI gene is amplified using the gene specific primers that are designed with restriction enzyme sites at the ends for cloning purposes. Specific primers designed with the restriction enzyme Pad and AscI, and PmeI sites yield a PCR product size of 1266 bp. Specific primers designed with the restriction enzyme BspHI and SphI sites yield a PCR product size of 1345 bp.

The PCR amplification of Synechocystis IPPI, as shown in FIG. 31, is performed using Taq DNA polymerase (Qiagen Hot Start Taq). Although Taq DNA polymerase is a robust enzyme for optimizing PCR conditions, its lack of 3′ to 5′ processing activity results in a relatively low fidelity.

In addition to Taq DNA polymerase, a higher fidelity DNA polymerase such as Pfx (Invitrogen, Inc.) can be used. The resulting product is digested with restriction enzymes and ligated into the pKAS6-aphA6 vector backbone to produce tobacco chloroplast vector pKAS6-aphA6-SyIPPI.

### Example 31

### Production of Tobacco Plants that Express the Rhodobacter capsulatus IPPI Gene in the Chloroplast

DNA vectors shown in FIGS. 27 and 28 are introduced into tobacco chloroplasts by Particle Inflow Gun (PIG) in the present invention. Specifically, 0.2 νμ gold particles (Seashell Technologies, Inc.) are coated with 0.25 μg DNA per shot and accelerated into leaf tissue using the PIG at 70 p.s.i. helium, 30 msec. Transgenic lines are selected by regeneration of explant tissue on RMOP medium containing 25 ug/ml kanamycin. A total of 6 transformations are initiated on 60 leaves. Four kanamycin resistant shoots are recovered for subsequent selection and propagation.

### Example 32

### Production of Transplastomic Nicotiana tabacum Containing RhIPPI Gene and/or SyIPPI Gene

This Example illustrates the transformation of tobacco chloroplasts. Exemplified herein is the chloroplast transformation of Nicotiana tabacum with vectors containing RhIPPI gene and/or SyIPPI gene at the desired locus.

FIG. 32 shows diagrams of vector constructs containing the RhIPPI gene (A), the SyIPPI gene (B) and tobacco chloroplast genome of nontransformed control (C) with predicted BtgI restriction enzyme sites used for genomic DNA digestion to predict insertion of the transgene in the chloroplast genome. Primer annealing sites in the rpoA gene used as probe for Southern analysis are shown.

### Plant Material

Nicotiana tabacum plants are grown on solid medium (3% (w/v) sucrose, 4.4 g/L Murashige and Skoog salts supplemented with Gamborg vitamins, 1 mg/mL thiamine, solidified with 1.2% (w/v) agar, pH 5.8). The axenic cultures are kept in a growth room on racks equipped with Philips F32T8/Cool White Plus (32 Watt) fluorescent bulbs. The fluence rate of the fluorescent light is 20-30 μmol m⁻²s⁻¹. The photoperiod is 12/12 h with room temperature at 22-25° C.

Aseptic leaves of 2-3-month-old tobacco seedlings are stably transformed using the particle inflow gun (PIG). Prior to transformation, 10 μg of plasmid DNA carrying desired constructs (the RhIPPI gene and/or SyIPPI gene) for transformation is precipitated upon 2.5 mg of 550 nm diameter gold particles (S550d, Seashell Technology, La Jolla, Calif.) according to manufacturer's instructions. For control experiments, dH₂O is substituted for the plasmid DNA. Tobacco leaves are bombarded twice with 250 μg of DNA coated gold particles. After bombardment, the leaves are left for three days on solid medium without selection at room temperature in the dark. Homoplastomic transgenic shoots are obtained by repeated shoot regeneration on RMOP medium (MS salts with Gamborg vitamins, 3% (w/v) sucrose, 1 mg/L thiamine, 1 mg/L BAP, 0.1 mg/L NAA) containing 25 mg/L kanamycin to inhibit growth of nontransformed plant cells. The transformed cells grow into callus and differentiate into shoots via organogenesis.

### Example 33

### Dna and RNA Analysis of Transplastomic Nicotiana tabacum Containing RhIPPI Gene and/or SyIPPI Gene

Fully expanded leaves of re-generated tobacco plants produced in Example 31 are excised, immediately frozen in liquid nitrogen, and stored in a −80° C. freezer until extraction of genomic DNA. Genomic DNA is isolated using the E.Z.N.A. plant genomic DNA isolation kit (VWR) according to manufacturer's instructions. PCR amplification of the RhIPPI gene is carried out using primers

P266
(SEQ ID NO: 77)
(5′ - GCTCGATCATGCCGTTGTTCACAT - 3′);
and
P267
(SEQ ID NO: 78)
(5′ - ATCTGGCGATTTCGGTCTTCGTGA - 3′).

PCR amplification of the SyIPPI gene is carried out using primers

P274
(SEQ ID NO: 79)
(5′ - GGTTAATTAATGCCCCCACCTTAATCCTGGG - 3′);
and
P275
(SEQ ID NO: 80)
(5′ - CTGGCGCGCCGATTATTAAAGGAAAACTGTGGC - 3′).

The PCR reactions are carried out in 50 μl volume consisting of 50 ng template DNA, 0.75 μl each primer (4 μmol), 1 μl dNTPs (10 mM each), 5 μl 10×PCR buffer, 0.25 μl HotStarTaqPlus DNA Polymerase (Qiagen). The PCR reaction conditions are: 30 cycles of 30 s denaturation at 94° C., 30 s primer annealing at 55° C., and 1 min primer extension at 72° C. The amplified PCR products are separated by electrophoresis in 1.0% agarose gel.

For Southern blot analysis, plant DNA (10 μg) is digested overnight with Btgl, fractionated by electrophoresis on a 0.8% agarose gel, followed by neutral transfer to Nytran Super Charge Nylon Membrane (Whatman, USA) using the Turboblotter™ Rapid Downward Transfer System according to the manufacturer's instructions. The blots are probed with a 900-bp rpoA coding sequence that is generated by PCR using the forward primer sequence

5′-GCAGCATTACGAGCTATTCGTCGA-3′ (SEQ ID NO: 81); and the reverse primer sequence—

### 5′-AGGGGATAAAGGAACCGTGT-3′ (SEQ ID NO: 82) (FIG. 32).

The chemifluorescent probe is labeled with Digoxigenin-11-dUTP using DIG-High Prime (Roche, USA) and Southern blot hybridization is carried out according to the manufacturer's instructions.

Total RNA is obtained with the RNeasy Plant Mini Kit (Qiagen) performed with an optional on-column DNAse digestion to remove genomic DNA contamination. RT-PCR is carried out using 50 ng template, 0.75 μl each primer (4 μmmol), 2 μl dNTPs (10 mM each), 10 μl 5× OneStep RT-PCR buffer, 2.0 μl OneStep RT-PCR enzyme mix (Qiagen). RT-PCR amplification of the RhIPPI gene is carried out using primers

P299
(SEQ ID NO: 83)
(5′ - TCTCGAGCCTAGGCTAGCTCTA - 3′);
and
P300
(SEQ ID NO: 84)
(5′ - GTCACCGGCGGAAAGATCATGT - 3′).

RT-PCR amplification of the SyIPPI gene is carried out using primer

P301
(SEQ ID NO: 85)
(5′ - ATTAATGCCCCCACCTTAATCC - 3′);
and
P302
(SEQ ID NO: 86)
(5′ - CGGCGCGCCGATTATTAAAGGA - 3′).

The RT-PCR reaction conditions are: 30 cycles of 30 s denaturation at 94° C., 30 s primer annealing at 55° C., and 1 min primer extension at 72° C. The amplified RT-PCR products are separated by electrophoresis in 1.0% agarose gel.

Quantitative Real-Time-PCR (qRT-PCR) is performed on a CFX96 Real-Time PCR Detection System (Bio-Rad) using iScript One-Step RT-PCR Kit with SYBR Green. The relative levels of the amplified mRNA are evaluated according to the 2^{-ΔΔCt}method using the α-tubulin gene for normalization. A value, Ct, is calculated based on the time at which the reporter fluorescent emission increases beyond a threshold level. Specific primers are designed based on cDNA sequences from tobacco using the primer design software from IDT Technologies to amplify the different IPPI genes in the tobacco transformants.

### Example 34

### HPLC Analysis of Transplastomic Nicotiana tabacum Containing RhIPPI Gene and/or SyIPPI Gene

Samples of the transplastomic Nicotiana tabacum produced in Example 32 are extracted for 24 hr (0° C., dark). Prior to analysis, the pigment extracts are vortexed and centrifuged to remove cellular and filter debris. Samples (200 μL) of a mixture of 0.3 mL H₂O plus 1.0 mL extract are injected onto a Varian 9012 HPLC system equipped with a Varian 9300 autosampler, a Timberline column heater (26° C.), and Spherisorb 5 μm ODS2 analytical (4.6×250 mm) column and corresponding guard cartridge. Pigments are detected with a ThermoSeparation UV2000 detector (λ=436 nm). A ternary solvent system is employed for HPLC pigment analysis: eluent A (MeOH: 0.5 M ammonium acetate, 80:20), eluent B (acetonitrile:water, 85:15), and eluent C (ethyl acetate). The linear gradient used for pigment separation is a modified version of the Wright et al. (1991) method: 0.0′ (90% A, 10% B), 1.0′ (100% B), 11.0′ (78% B, 22% C), 27.5′ (10% B, 90% C), 29.0′ (100% B), and 30.0′ (100% B). Eluents A and B contain 0.01% of 2,6-di-tert-butyl-p-cresol (BHT) (Sigma Chemical Co.) to prevent the conversion of chlorophyll a into chlorophyll a allomers. HPLC grade solvents (Fisher) are used to prepare eluents A, B and C. The eluent flow rate is held constant at 1 mL min⁻¹. Pigment peaks are identified by comparison of retention times with those of pure standards and extracts prepared from wild-type tobacco.

### Example 35

### Demonstration of Integration of Transgene into Tobacco Chloroplast Genome

This Example further illustrates the transformation of tobacco plastids and plant regeneration, and demonstrates that transgene is integrated into the tobacco chloroplast genome.

### Transformation of Tobacco Plastids and Plant Regeneration

A total number of 156 tobacco leaves are used in particle inflow gun (PIG) and Bio-Rad gene gun bombardment experiments. 70 leaves are treated with pKAS6-aphA6-RhIPPI; 86 are treated with pKAS6-aphA6-SyIPPI. Bombarded tobacco leaf discs are initially cut into smaller pieces then transferred to fresh RMOP medium containing, for example, 25 μg/mL kanamycin as the selection agent every 3 to 4 weeks. Typical results, for example, are seven true transformants produced containing the RhIPPI gene, and six true transformants are produced containing the SyIPPI gene and transformation efficiencies, for example, of 10% for vector pKAS6-aphA6-RhIPPI (7 events per 70 leaves) and 6.98% for vector pKAS6-aphA6-SyIPPI (6 events per 86 leaves). These typical results indicate highly efficient performance of the invention as a baseline vector without requiring significant optimization of vector sequences.

Confirmation of Transgene Integration into Tobacco Chloroplast Genome

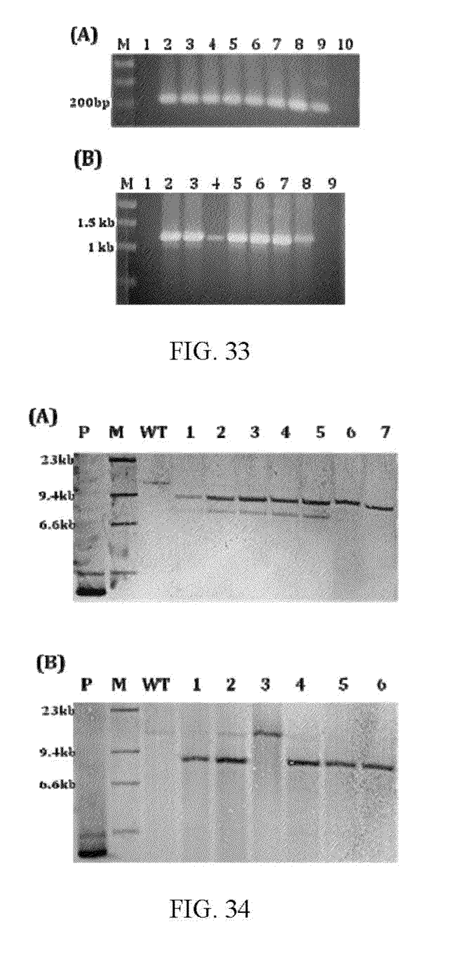

The presence of the RhIPPI and SyIPPI genes in the regenerated tobacco lines is confirmed by PCR using specific primers that amplified a region of the IPPI coding sequence. A 242 bp size PCR product is amplified using the RhIPPI primers (FIG. 33A) and a 1.2 kb PCR product is amplified using the SyIPPI primers (FIG. 33B).

Since this PCR product can be obtained in the event of nuclear integration mediated by promiscuous DNA, the possibility of nuclear integration is examined and ruled out by Southern analysis. Integration of the transgenes (RhIPPI and SyIPPI) into tobacco plastid genome and homoplasmy are confirmed. Genomic DNA from transformed and nontransformed cultures is digested with the Btgl restriction enzymes, transferred to nylon membrane and probed with Digoxigenin-11-dUTP labeled rpoA gene fragment (FIG. 32). Total genomic DNA digested with BtgI yields an expected 9.3 kb size hybridizing fragment for RhIPPI transformants and 7.9 kb size for SyIPPI transformants, thereby confirming the site-specific stable integration of the transgenes into the chloroplast genome in tobacco plants (FIG. 34). As expected, a 12.5 kb fragment is observed on the nontransformed tobacco control.

The chloroplast genome is present in tens or hundreds of copies within each chloroplast. Integration of a transgene is believed to occur at one (or very few) copies of a specific genomic locus via homologous recombination. Therefore, immediately after the recombination event, most genome copies are wild-type and a few are recombinant. Conversion from this heteroplasmic state to homoplasmy, wherein all genome copies contain the desired transgene, is believed to require consistent selective pressure over several generations of plant propagation.

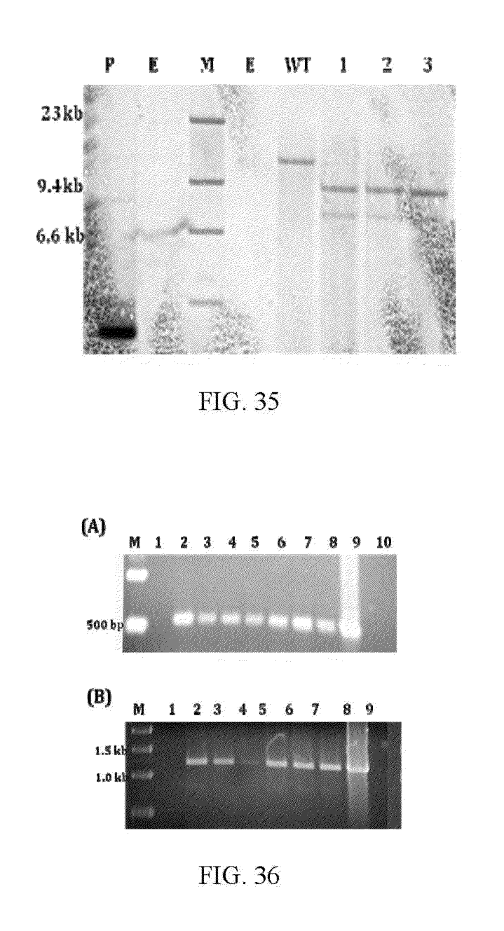

In the experiments, heteroplasmic transgenic tobacco lines yield two fragments by Southern blot testing: a 12.5 kb wild-type fragment and 9.3 kb (RhIPPI) or a 7.9 kb (SyIPPI) fragment. All of the RhIPPI transformants reach homoplasmy after just one cycle of regeneration (FIG. 34A lanes 1-7), whereas three of the six SyIPPI transformants are still heteroplasmic as shown by the presence of the two bands (FIG. 34B lanes 1, 2, 4). In addition, one SyIPPI transformant that yields a positive result from PCR amplification produces no hybridization result on Southern blot (FIG. 34B lane 3). This shows that PCR amplification is more sensitive in detecting foreign genes integrated into plants than Southern blotting. Homoplasmic transgenic tobacco lines are selected by repetitive subcultures of leaf discs. FIG. 35 shows Southern blot results from one line of tobacco transformant after three cycles of regeneration. These results further show that there is stable integration of the transgene even after three cycles of regeneration. In addition to DNA, transgene messenger RNA was readily detected using reverse transcriptase (RT)-PCR (FIG. 36).

### Example 36

### Quantitative Real-Time PCR (qRT-PCR) analyses

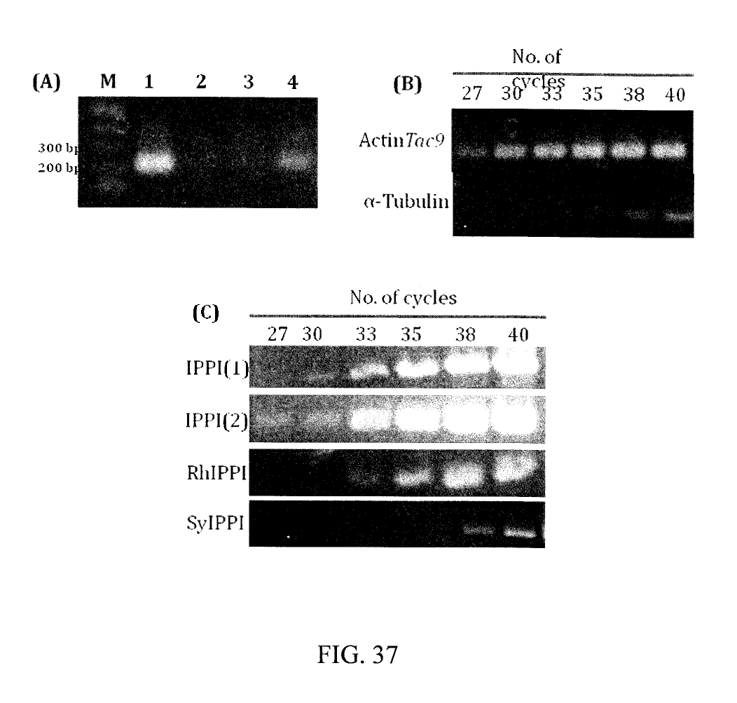

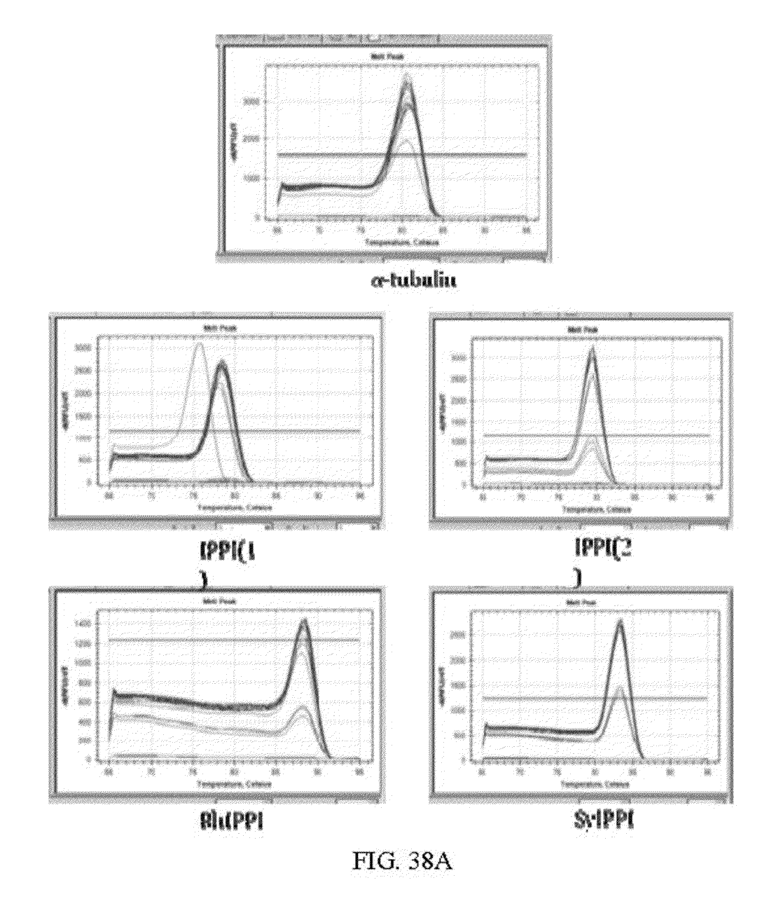

qRT-PCR represents a highly sensitive and powerful technique for the quantitation of nucleic acids. Selection of an internal control RNA for normalizing data to account for sampling error is critical in order to draw valid quantitative conclusions. Potential internal control RNAs that are evaluated included actin, G3PDH, and α-tubulin (FIG. 37A). RT-PCR results show that one set of actin primers (actinTac9) and α-tubulin primers produce a PCR product, whereas actinTob66 and G3PDH does not produce any amplification. Since these housekeeping genes are normally expressed at very high levels, their amplification kinetics reaches a plateau much earlier than the relatively low abundant RNAs produced by our transgenes. One qRT-PCR parameter that requires optimization is the number of amplification cycles to perform. Amplification is initially exponential but reaches a plateau when the activity of the enzyme declines or when any of the reaction reagents become limiting. At plateau, RNAs initially present at high levels may give products of equal intensity to low abundant RNAs.

In this Example, a number of cycles ranging from 27 to 40 (FIG. 37B) are tested. The α-tubulin PCR product first appears at 35 cycles, whereas actin appears at 27 cycles. These results demonstrate that α-tubulin is a better candidate to use as the internal control RNA for subsequent qRT-PCR analyses since it amplifies almost at the same time as our genes of interests (FIG. 37C).

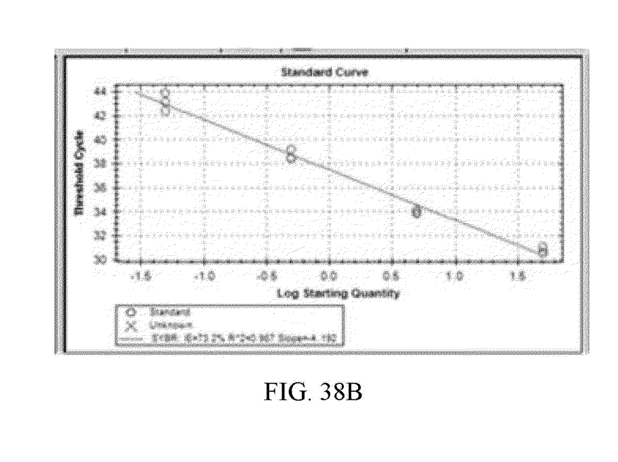

In addition to the selection of an ideal internal control, it is also important to optimize SYBR Green I reactions for each qRT-PCR assay. First, the optimal annealing temperature for each of the primers used is identified, and then a standard curve to evaluate assay performance is constructed. For each PCR primer set, a range (from 46° C.-70° C.) of annealing temperatures are tested. Then a melt-curve function is employed to check the specificity of the present qRT-PCR assay. An optimized reaction has a single peak in the melt curve as shown in FIG. 38A. The efficiency, reproducibility and dynamic range of the assay is determined by constructing a standard curve using serial dilutions of the template. FIG. 38B shows the standard curve from α-tubulin where the R² is 0.987.

### Example 37

### Quantification of Transgene Expression

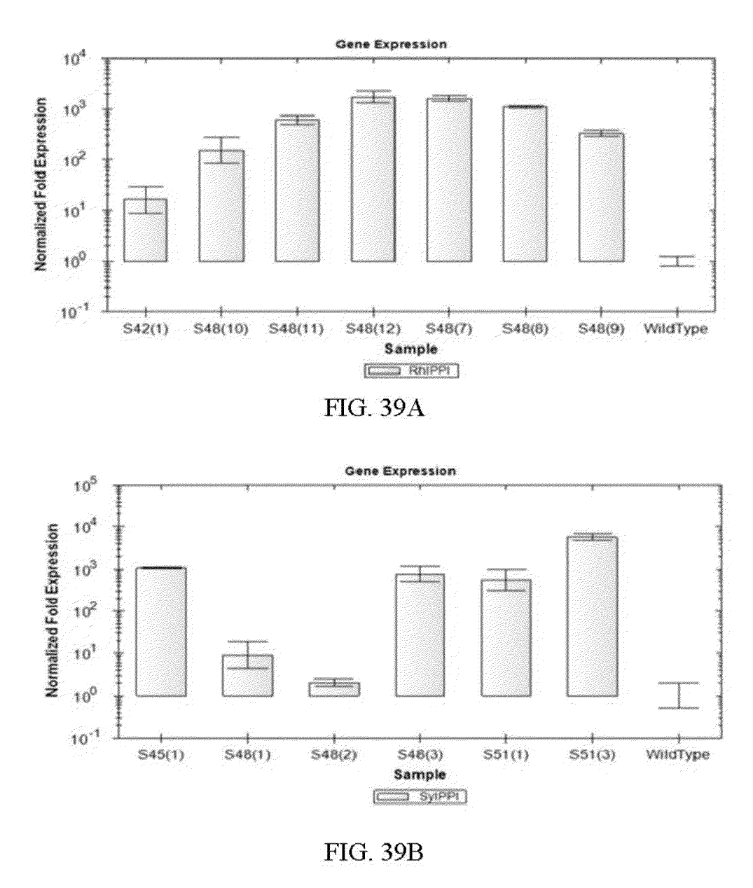

All transgenic lines containing RhIPPI or SyIPPI DNA are tested by means of the SYBR Green I assay to quantify transgene mRNA. The RhIPPI transcript is detected in all seven RhIPPI transgenic lines (FIG. 39A), and SyIPPI transcript is detected in all six SyIPPI transgenic lines (FIG. 39B). Most of the RhIPPI transformed lines have comparable RhIPPI mRNA levels except for one line, S42-1 that yields the lowest expression. Among SyIPPI transformants, two (S48-1 and S48-2) of six lines tested yield lower levels of gene expression while the remaining lines yield very similar levels of expression.

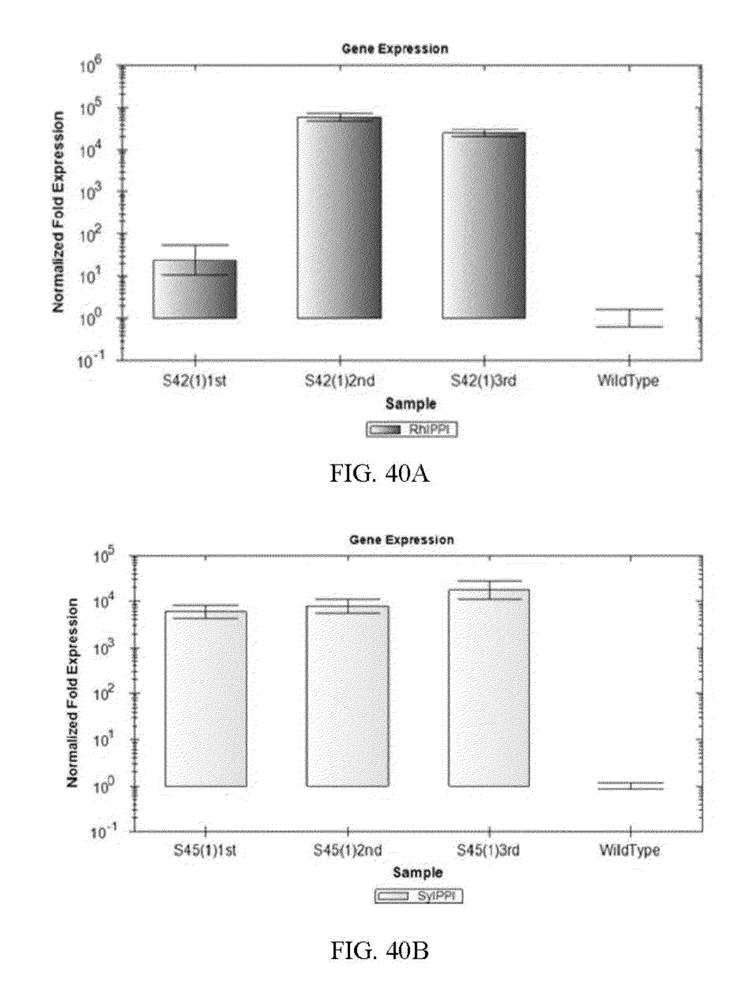

The differences of the transgene expression between plants of subsequent cycles of regeneration are determined, and mRNA levels from clones of the same transformation event at one, two and three cycles of regeneration are measured. The results show that expression of both IPPI transgenes reach a plateau after the 2^{nd} cycle of regeneration (FIG. 40). This shows that maximum level of transgene expression could be reached after the 2^{nd} cycle of regeneration.

### Example 38

### Expression of Tobacco IPPI Genes in Transformed Tobacco Plants

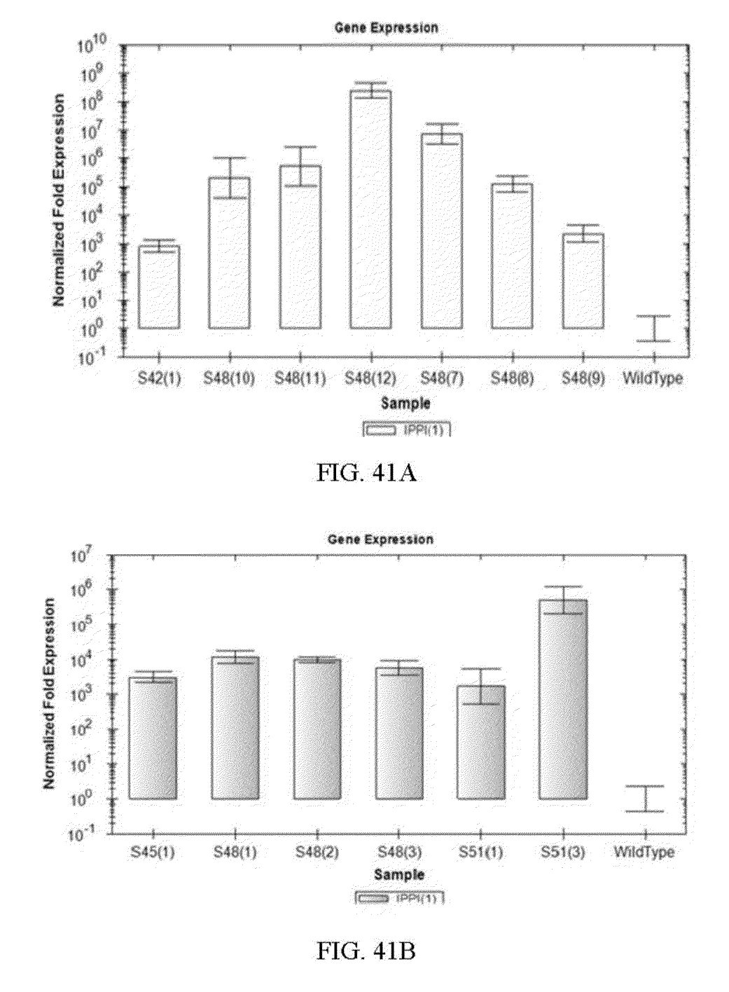

Nakamura, et al. 2001 reported the isolation and characterization of two distinct cDNA clones, IPPI(1) and IPPI(2), in tobacco. To determine if the expression of these endogenous nuclear-encoded genes is influenced by the presence of the present IPPI transgenes in the chloroplast, mRNA levels for the tobacco genes are quantified using qRT-PCR. The amount of IPPI(1) mRNA detected in RhIPPI transformants ranges from 10³- to 10⁸-fold higher when compared to IPPI(1) detected in wild-type nontransformed control tobacco (FIG. 41A); in SyIPPI transformants it varies from 10³- to 10⁶-fold higher than that found in wild-type tobacco (FIG. 41B).

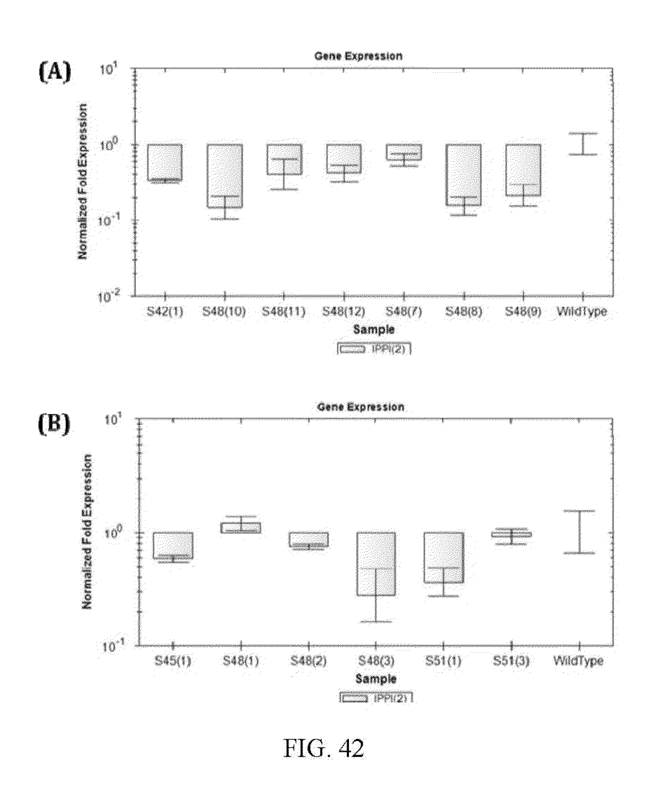

Tobacco IPPI(2) protein functions in the cytosol and its transcript is relatively less abundant and metabolically more stable under normal conditions when compared to the chloroplastic IPPI(1). qRT-PCR testing of the transgenic tobacco plants herein show that there is an approximate 10-fold reduction of IPPI(2) mRNA detected in both RhIPPI (FIG. 42A) and SyIPPI (FIG. 42B) transgenic plants when compared to wild-type plants. However, this alteration of endogenous IPPI gene transcription is not as dramatic as the 10³- to 10⁸-fold increase in IPPI(1) mRNA detected in these same plants (FIG. 41).

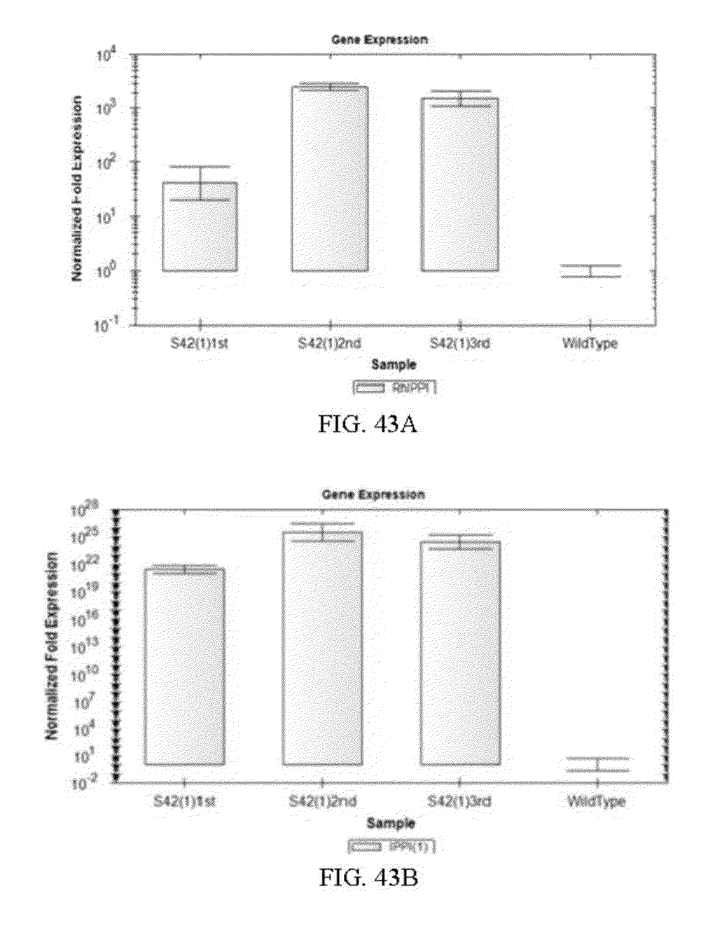

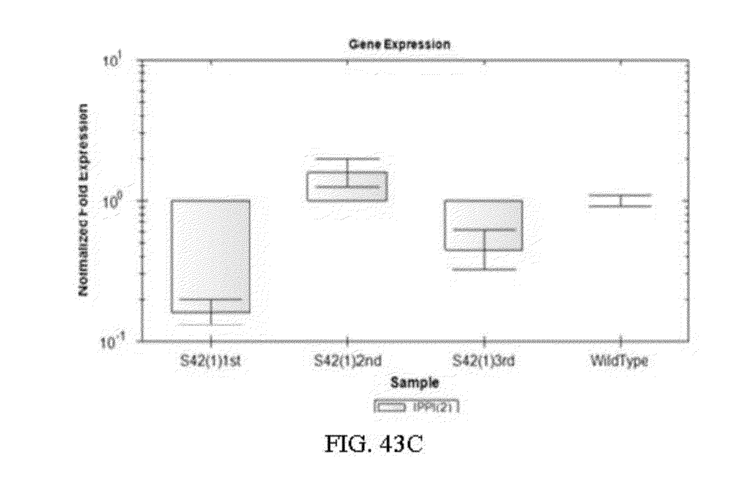

The chloroplastic tobacco IPPI(1) gene is known to be induced by high light exposure. Thus, the effect of high light on transgenic tobacco lines is also investigated to determine if an environmental stress could influence transcription of the endogenous or heterologous IPPI genes. The results show that foreign gene expression (RhIPPI) is unchanged after exposing the transgenic tobacco plants to high light stress (compare FIGS. 40A and 43A). The quantity of RhIPPI mRNA does not increase with high light exposure, because the Rhodobacter IPPI regulatory sequences included in the presently cloned vectors are unresponsive to the regulatory machinery of the tobacco chloroplast. Further, the transcription of IPPI transgenes would be regulated similarly to that of the tobacco rpl23 operon into which they are inserted. This operon encodes ribosomal proteins and RNA polymerase subunits, and as such, would maintain a constant level of housekeeping gene expression.

The results also show a significant induction of chloroplastic tobacco IPPI(1) mRNA in these same transgenic plants. When compared with wild-type tobacco plants containing no transgene but exposed to high light, IPPI(1) mRNA is detected at levels 10²²- to 10²⁵-fold higher than that of the wild type (FIG. 43B). This suggests that RhIPPI mRNA is interfering with the activity of the endogenous tobacco IPPI(1) gene in these plants. Most chloroplast genes are highly regulated through post-transcriptional processes, and the mechanisms that could account for these observations are numerous). Ultimately, the response of the transgenic plants is likely designed to increase the flux of isoprenoid biosynthesis for protection against stress in the plant. Finally, transcription of the nuclear tobacco IPPI(2) gene is unaffected (FIG. 43C).

### Example 39

### Isoprenoid Content of Tobacco Transgenic Plants

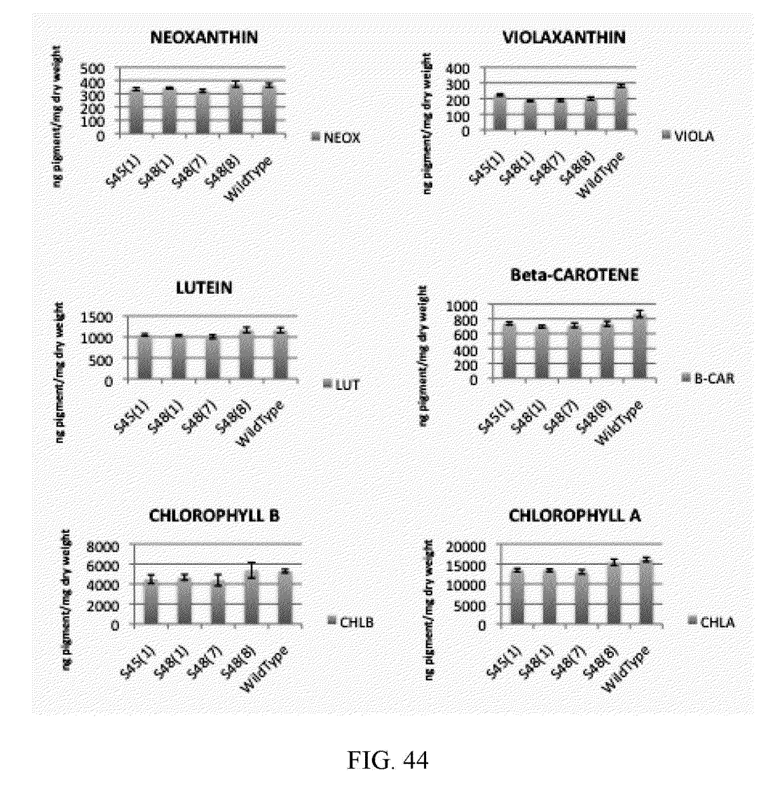

This Example demonstrates that the level of isoprenoid production in transgenic organisms can be increased by overexpressing IPPI, a key regulator of biosynthesis, in the chloroplast. HPLC analyses are performed to quantify some common isoprenoid compounds normally synthesized in the chloroplast. The data indicate that under normal growth conditions, no significant increase in isoprenoids, as detected by this particular HPLC test, is observed in the transgenic plants. Five isoprenoids are unchanged or slightly reduced (FIG. 44) when compared to the wild-type control. The levels of violaxanthin are reduced by about 30% in transgenic plants.

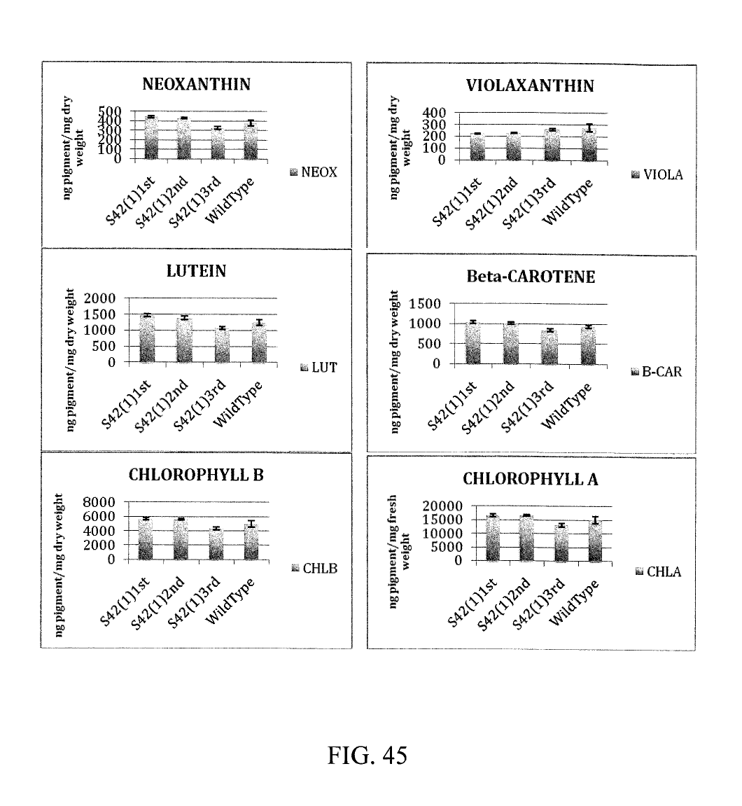

In addition, isoprenoids in transgenic plants grown under high light conditions are also quantified to determine whether the environmental stress produces an otherwise hidden effect on the heterologous transgene. The transgenic tobacco plants are treated in 300 μE/m²/sec light for one month before harvesting leaf samples for analysis. FIG. 45 summarizes the results of testing three generations of the RhIPPI transgenic tobacco line, S42. RhIPPI mRNA is detected in these plants and increases from 10¹ to 10³ from generation 1 to generation 2 and 3 (FIG. 43A). The increase in RhIPPI mRNA, however, is not accompanied by an obvious increase in transgene DNA, nor a conversion from heteroplasmy to homoplasmy, which could be detected by Southern blot tests (FIG. 35). Quantitative RT-PCR tests for the endogenous tobacco genes show a significant induction of chloroplastic IPPI(1) mRNA (10²⁰ to 10²⁵) and little change in cytosolic IPPI(2) mRNA (FIGS. 43B-C) between subsequent generations.

To summarize, the above data sets show a slight increase in RhIPPI, a significant increase in IPPI(1), and slightly elevated levels of isoprenoids that decrease to slightly lower than wild-type levels with each subsequent generation.

### Example 40

### Plant Morphology in Tobacco Transformants

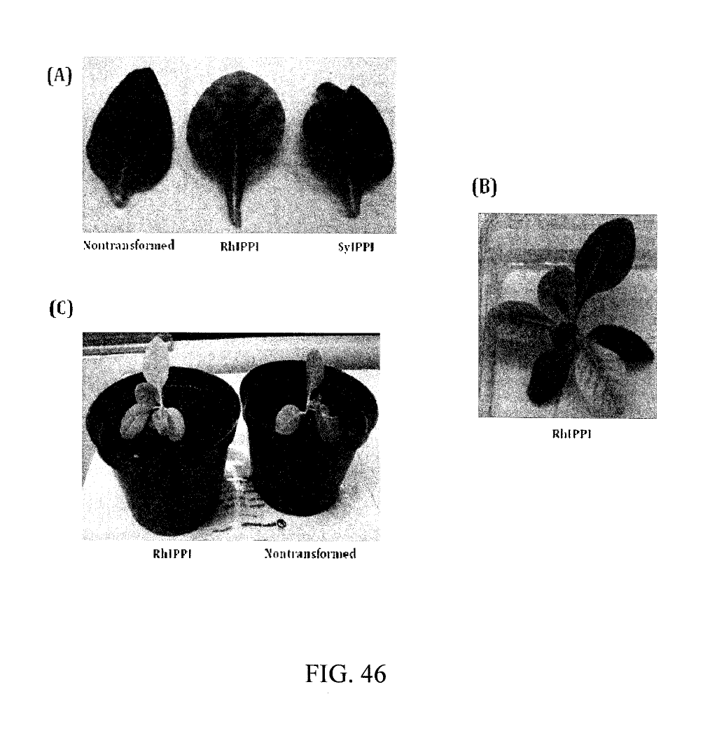

In this Example, the leaf morphology of tobacco transformants is compared to wild-type tobacco. In general, plants containing an active SyIPPI gene in the chloroplast appear similar to the wild type, whereas leaves from RhIPPI transformants displayed chlorosis, or reduced chlorophyll content, in areas remote from the veins (FIGS. 46A and B). However, once the transformed plants are transferred to soil for rooting, they start to grow and develop the same as the nontransformed tobacco control (FIG. 46C).

TABLE 1
Summary of mRNA and HPLC assays in transgenic tobacco plants

| | | Effect of heterologous transgene expression on: | | |
|---|---|---|---|---|
| IPPI | Examples | | | Chloroplastic |
| enzyme characteristics | tested in this report | IPPI(1) mRNA | IPPI(2) mRNA | isoprenoids |
| Type I | | normal light | unchanged | slight ↓ |
| MVA pathway | Rhodobacter IPPI | ↑10³ to 10⁸ | | |
| Cytoplasmic | | high light | unchanged | slight ↓ |
| Monomeric | (Tobacco IPPI-2) | ↑ 10²⁰ | | |
| Type II | | normal light | unchanged | unchanged or |
| DXP pathway | Synechocystis IPPI | ↑10³ to 10⁶ | | slight ↓ |
| Chloroplast | | high light | unchanged | slight ↓ |
| Tetrameric | (Tobacco IPPI-1) | not tested | | |

### Example 41

### Transformation of Nicotiana tabacum cv. Petit Havana Chloroplasts with Trangenes Expressing Hantavirus Antigens

This Example illustrates the transformation of tobacco chloroplasts with vectors containing trangenes expressing hantavirus antigens. Exemplified herein is the chloroplast transformation of Nicotiana tabacum cv. Petit Havana.

Hantaviruses are RNA viruses that cause significant human mortality, are endemic worldwide and are transmitted to humans by aerosolized mouse (or rodent) urine or excreta. Hantaviruses are a worldwide public health concern as infection can lead to hemorrhagic fever and renal or cardiopulmonary failure with fatality rates approaching 36%. A rapid, accurate diagnosis is crucial to reducing morbidity and mortality. To manufacture a diagnostic test, a reliable source of recombinant nucleoprotein is critical. Hantavirus nucleoproteins expressed in E. coli, Vaccinia virus, yeast, tobacco, potato and mammalian cells demonstrate antigenicity, but yield has been poor (0.1% to 2% of total soluble protein). Thus, there is a need for a higher yielding production platform.

Chloroplast genetic engineering has emerged as an attractive tool for hyper-expression of biopharmaceuticals in plants. This invention illustrates another novel method of chloroplast recombinant protein production capable of producing expression of Hantaan and Andes nucleoprotein antigens in plastids of tobacco to approximately 6% to 8% of total soluble protein without optimization. Said antigens are sufficiently reactive with convalescent human antisera to function in an in vitro diagnostic assay, with reactivity similar, or superior, to that prepared in E. coli. Further, the method of this invention can produce protein antigens at low cost and be applied to other species of chloroplast-based production platforms.

Plant Material Nicotiana tabacum cv. Petit Havana is plants grown under aseptic conditions on MS medium (Murashige and Skoog, 1962) solidified on 1.2% agar containing 30 g/L sucrose (pH 5.8). Seeds are sterilized by using 6% sodium hypochlorite (Clorox bleach) followed by washing with autoclaved deionized water and plating on MS medium. Regenerated shoots are grown on RMOP medium (MS medium with Benzlaminopurine (1 mg/l), ThiamineHCl (1 mg/l), Naphthaleneacetic acid (0.1 mg/l), and kanamycin (25 mg/L); the RMOP medium is filter-sterilized and added after autoclaving. Homoplasmic plants are grown in soil under green house conditions.

### Plastid Transformation Vectors

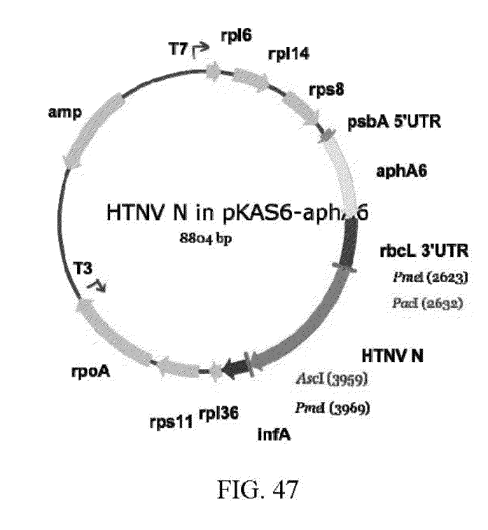

Construction of tobacco chloroplast vectors pK6aphA6/HTNV-N (FIG. 47) and pK6aphA6/ANDV-N begin with the cloning of the tobacco rpl23 operon from genes rpl16 through rpoA. A unique BglII site in the infA pseudogene locus is used to insert a multiple cloning oligomer for vector development. The Acinetobacter baumannii aminoglycoside phosphotransferase enzyme APH(3′)-VI selectable marker gene is inserted 5′ to infA, thereby allowing recovery of transplastomic tobacco plants in the presence of kanamycin.

In this Example, the expression of aphA6 is regulated by the Chlamydomonas psbA 5′UTR and rbcL 3′UTR. cDNA for Hantaan or Andes is inserted between the aphA6 selectable marker gene and the infA locus. A C-terminal hexahistidine tag is added to the nucleoprotein cDNA to facilitate purification of the recombinant protein using metal-affinity chromatography.

### Transformation of Tobacco Chloroplasts by Particle Inflow Gun (PIG)

Plastid transformation is carried out using helium-driven Particle Inflow Gun (Kiwi Scientific, Levin, New Zealand). Young tobacco leaves from aseptically grown tobacco plants are bombarded with plasmid DNA coated with 0.55 μm gold particles (Seashell Technology). The bombarded explants are selected on regeneration medium of plants (RMOP) containing kanamycin (25 mg/L). Putative transformed shoots are analyzed by PCR and the positive lines are taken through two additional cycles of regeneration to achieve homoplasmy.

Approximately 8 weeks after transformation, resistant shoots develop from wild-type tobacco leaves that have been transformed with chloroplast expression vectors pK6aphA6+His-HTNV-N or pK6aphA6+His-ANDV-N by PIG bombardment followed by selection of explants on regeneration medium containing kanamycin (25 mg/l). The explants from these lines are given additional rounds of regeneration in selection medium to achieve homoplasmy.

### Example 42

### Polymerase Chain Reaction (PCR) of the Transgenic Nicotiana tabacum Cv. Petit Havana

### PCR Analysis

Kanamycin resistant shoots from the bombarded leaf explants are analyzed by PCR. Total genomic DNA is isolated using a Qiagen mini plant DNA isolation kit. The DNA is amplified using a BioRad Icycler and a Qiagen Hot StarTaq Plus PCR kit. The aphA6 transgene is analyzed in both S24 and S25 resistant explants by using the following primer sets:

aphA6 Right:
(SEQ ID NO: 87)
5′ GGC CGG CCG TTT AAA CCT ACA TCC GCT TTA G-3′;
and
apha6 Left:
(SEQ ID NO: 88)
5′ CTT AAT TAA CCT AGG TTG AAT TTA TAA-3′.

The aphA6 positive lines are further analyzed by using hantavirus transgene-specific primers. Hantaan nucleoprotein cDNA, is detected with primers

HTN-N-Right:
(SEQ ID NO: 89)
5′GTT TAA ACG GCG CGC CTC AAT GGT GAT GGT GAT GAT
GGA GTT TCA AAG GC-3′;
and
HTN-N-Left:
(SEQ ID NO: 90)
5′ GTT TAA ACT TAA TTA AGG AGA GGA TAA AAT ATG GCA
ACT ATG GAG G-3′.

Andes nucleoprotein cDNA is detected with primers used are:

AND-N-Right:
(SEQ ID NO: 91)
5′ GTT TAA ACG GCG CGC CTC AAT GGT GAT GGT GAT GAT
GAC CGG-3′
and
AND-N-Left:
(SEQ ID NO: 92)
5′GTT TAA ACT TAA TTA AGG AGA GGA TAA AAT ATG AGC
ACC CTC CAA G-3′.

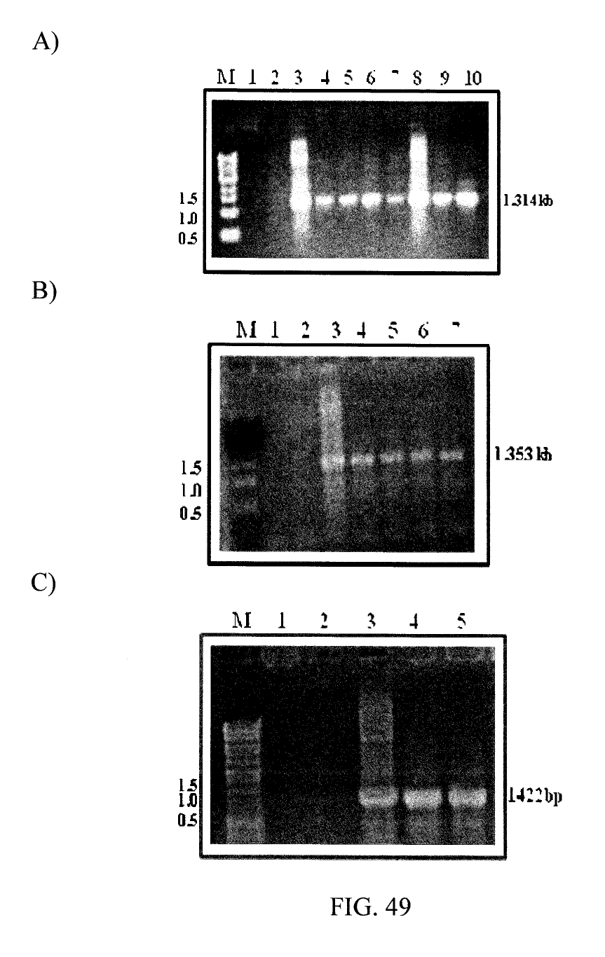

The results, as shown in FIG. 49A, reveal that PCR amplification of the resistant shoots yields a predicted 1314 bp product. No non-specific amplification is detected in wild-type tobacco or negative controls. Transgenic lines containing the aphA6 gene are further analyzed with gene-specific primer sets for either Hantaan (FIG. 49B) or Andes (FIG. 49C) cDNA. The expected products of 1353 bp and 1422 bp are detected in Hantaan and Andes transformed lines, respectively. Of the total number of kanamycin-resistant shoots recovered, 29% (4/14) and 18% (2/11) are positive for the aphA6 gene. All of the four lines from experiment S24 (lines 8a-1, 8a-2, 8b-1 & 8b-2) and both lines from experiment S25 (lines 6b-1, 6b-2) are positive for their respective Hantavirus cDNA transgenes.

### Example 43

### Demonstration of Homoplasmy of the Transgenic Nicotiana tabacum Cv. Petit Havana Chloroplast Genome

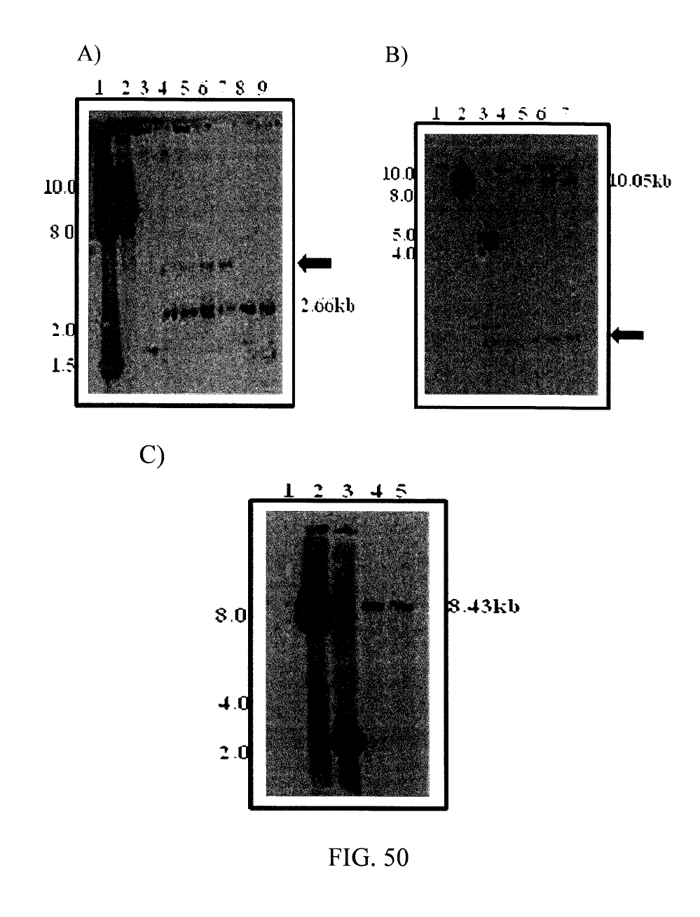

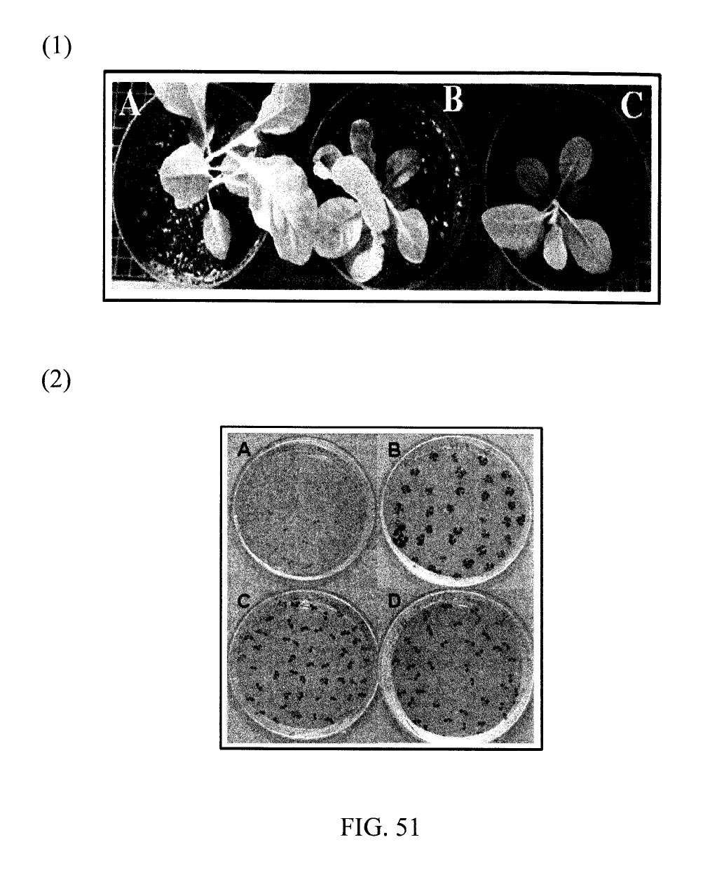

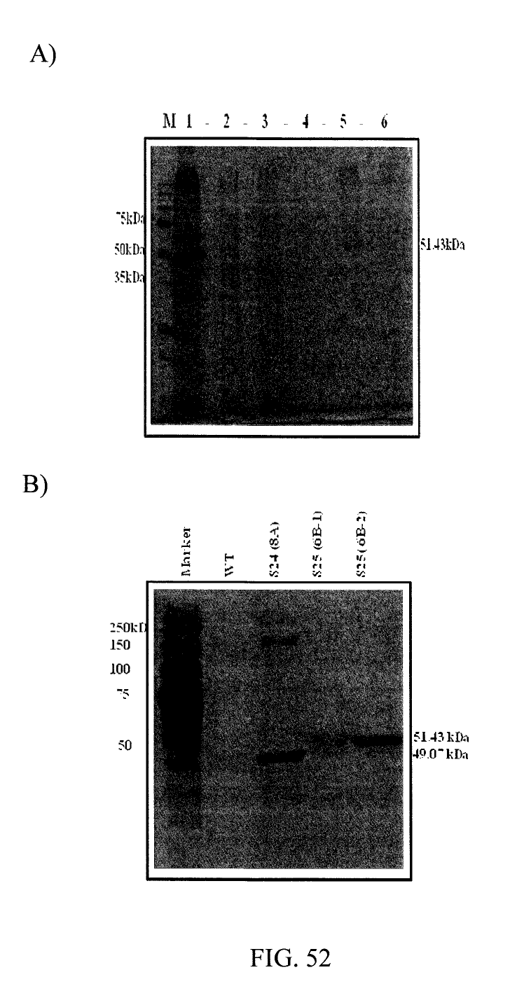

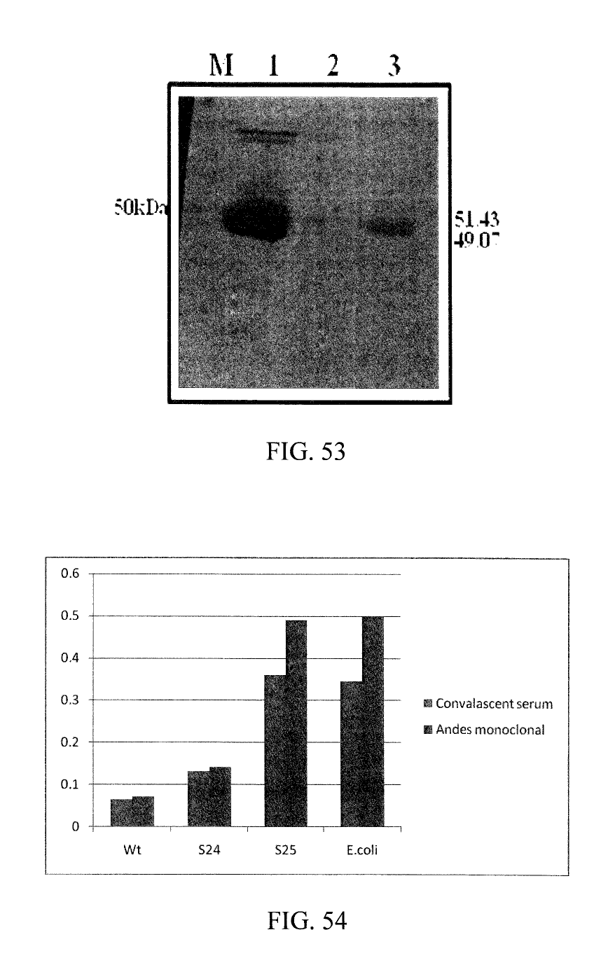

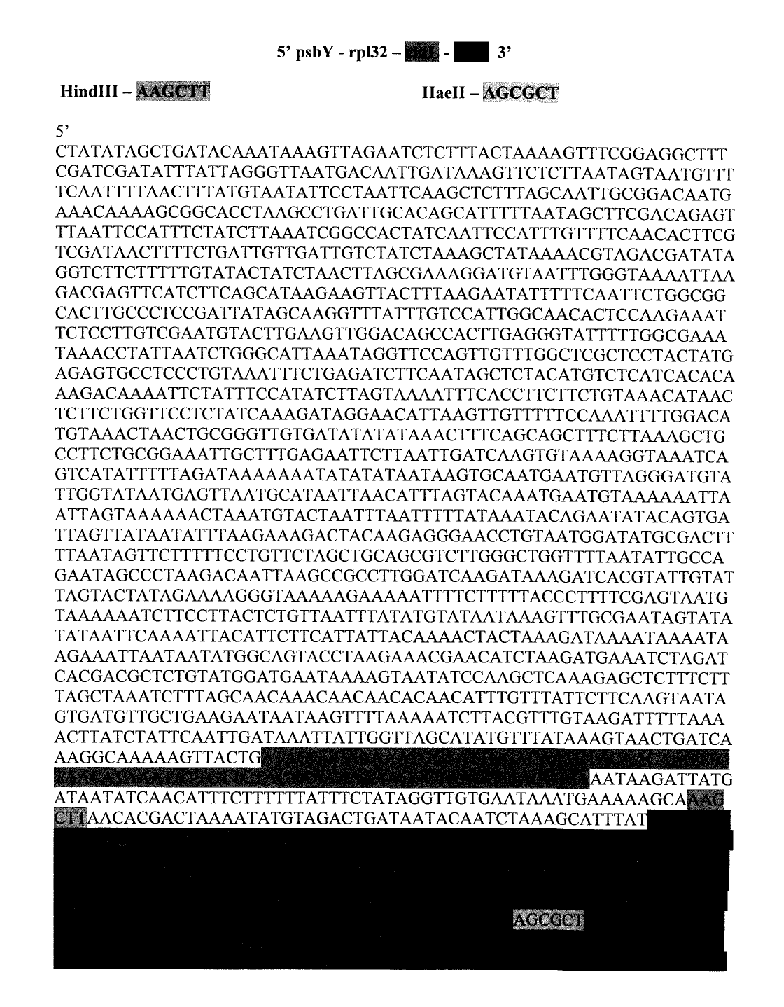

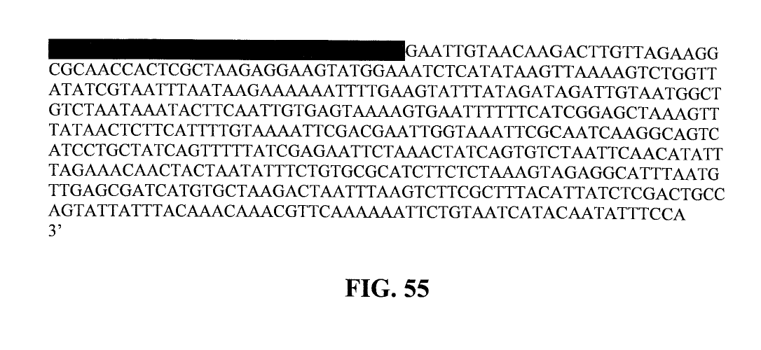

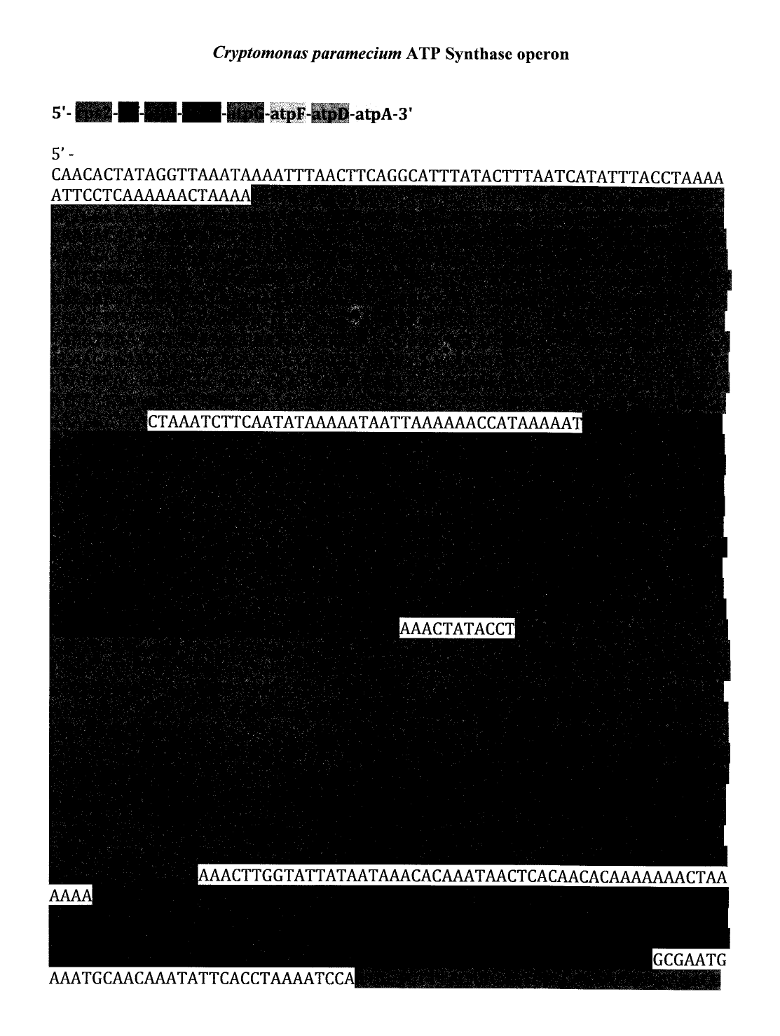

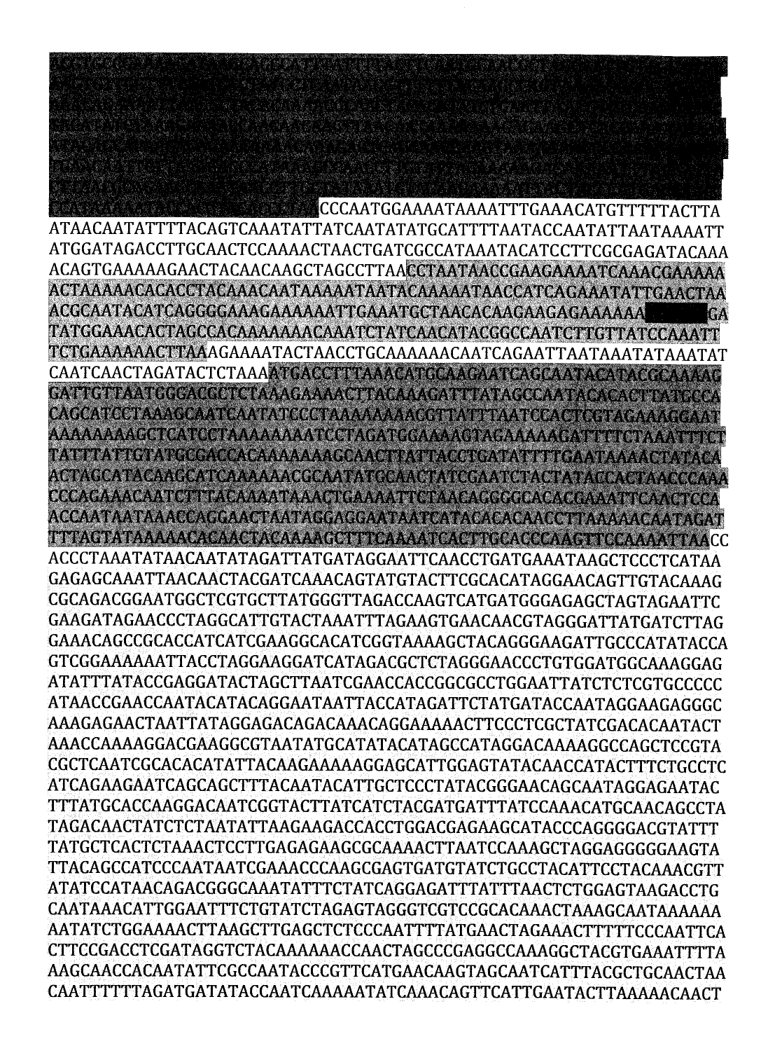

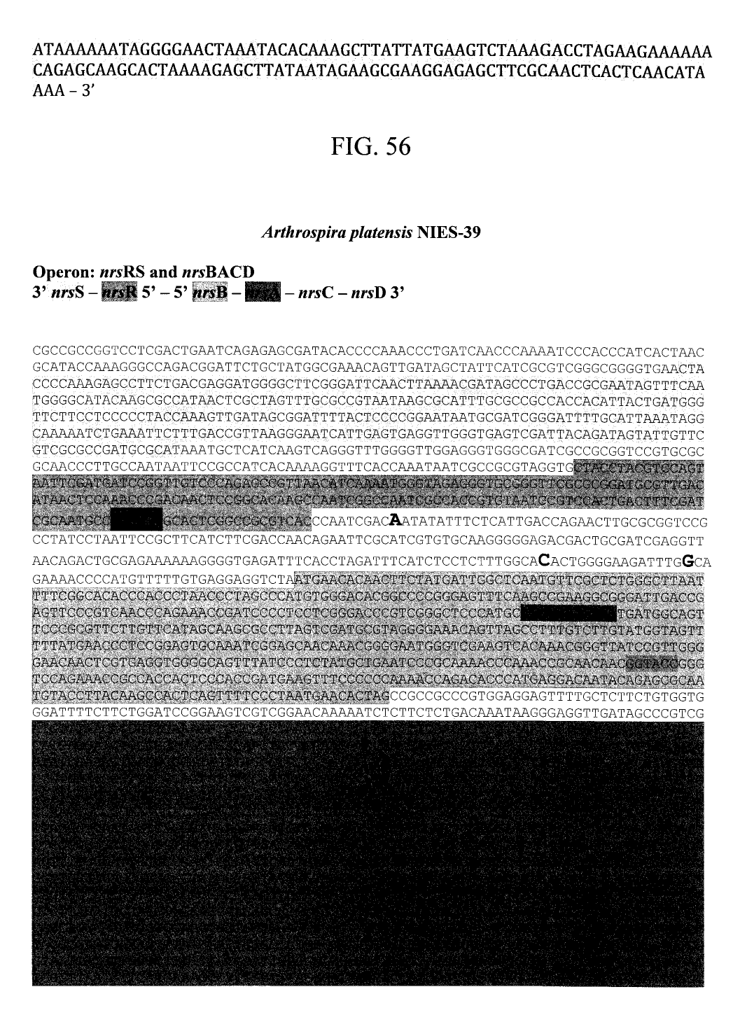

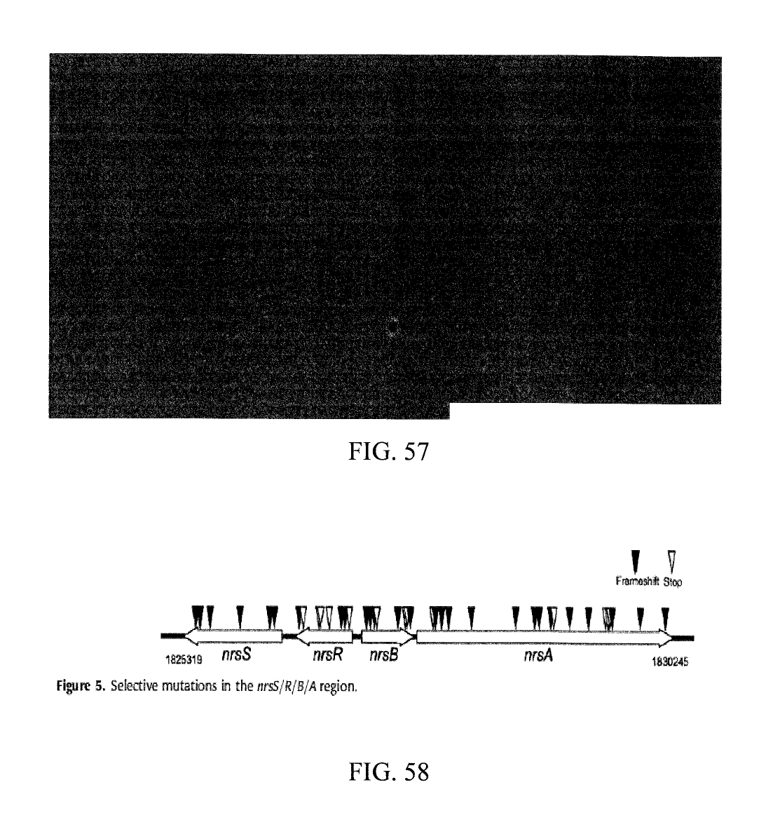

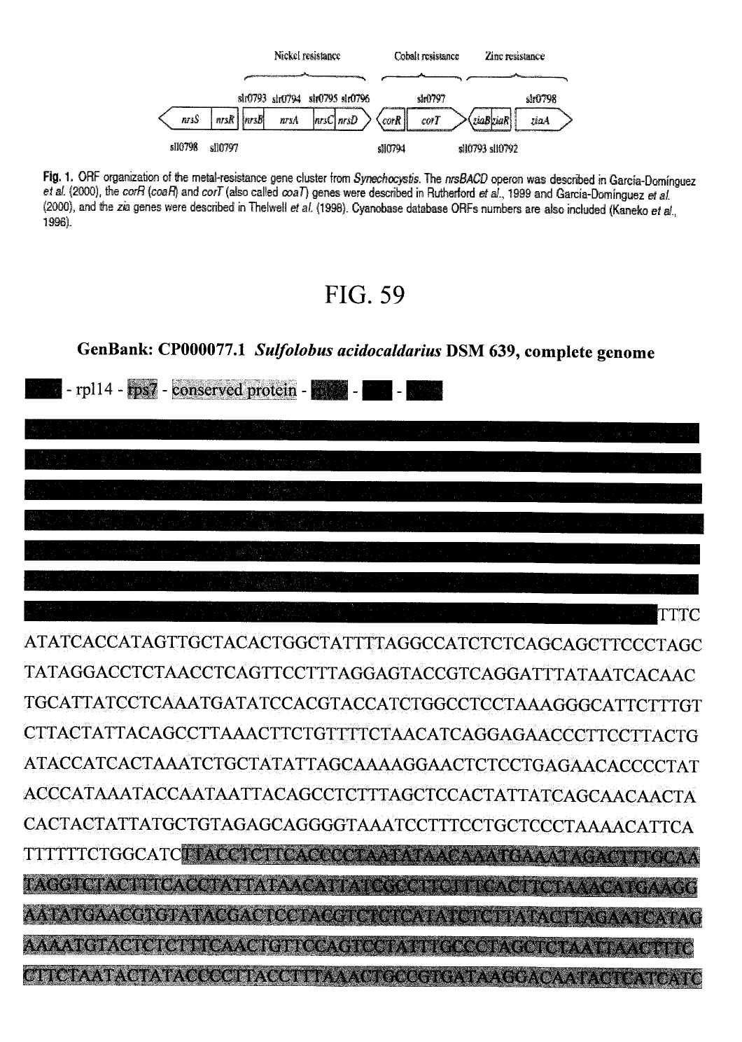

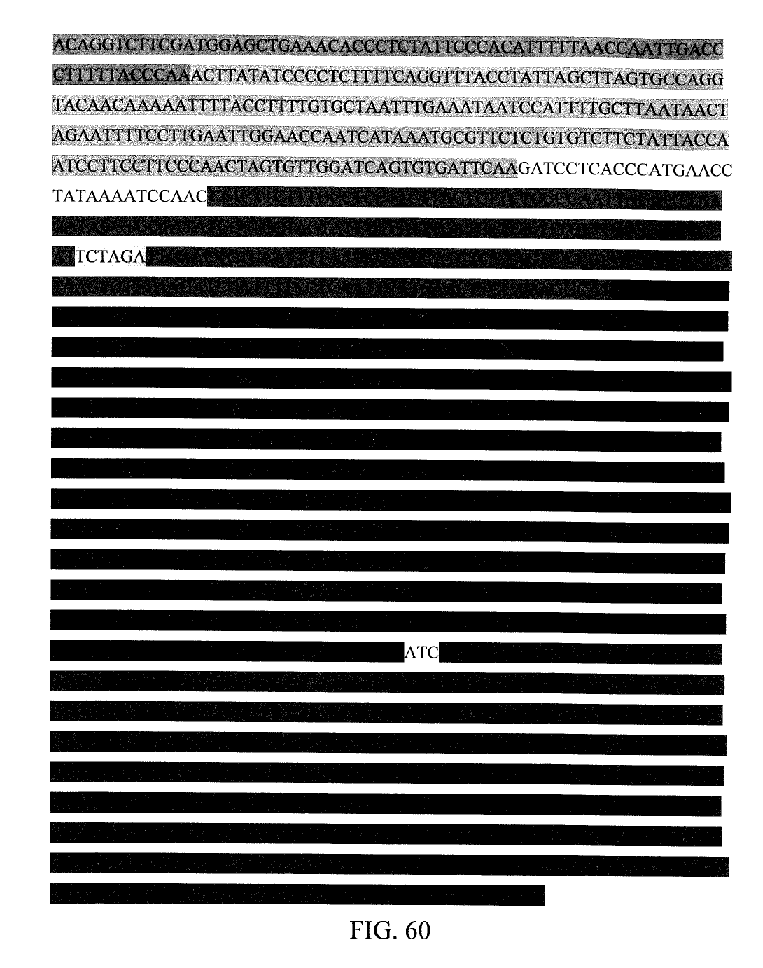

Six independently generated Nicotiana tabacum cv. Petit Havana lines are identified as transgenic based on PCR analysis. Southern blot analyses (FIGS. 50A-C) are performed to determine if all expected transgenes were present, and if so, whether integration occurred in the chloroplast or nucleus and whether chloroplast integration by homologous recombination results in a predictable restriction fragment.

### Southern Blot Analysis

Total genomic DNA is isolated from fresh tobacco leaf samples using Invitrogen Plant DNAZOL Reagent following the manufacturer's protocol. For Southern blot analysis, 3 μg of total cellular DNA is digested with BglII or Btgl restriction enzymes, separated by gel electrophoresis in 0.8% agarose gels and transferred to a Biodyne B membrane (+ charged membrane; Pall) using a Turboblotter (Schleicher and Schuell) following the instructions of the manufacturer. The probes for aphA6-rps8, HTNV-N/InfA and Andv/InfA are synthesized by PCR and extracted from gel using the QIA Gel Extracting Kit (Qiagen). 100 ng of the PCR product for each probe is labeled using the N2S Biotin Random Prime labeling kit (Pierce) following the manufacturer's protocol. The probe is hybridized to the Biodyne B membrane and the DNA. DNA hybrids are detected using a Chemiluminiscent detection substrate (Pierce).

The results, as shown in FIG. 50, reveal that the expected 2667 bp product corresponding to the aphA6 gene is observed in all transgenic lines (FIG. 50A, lanes 4 through 9). This fragment is not detected in wild-type tobacco plants nor from the digestion of the plasmid DNA used to produce the transgenic plants. A larger fragment (>2667 bp) is also detected in lanes 4 through 7, and the size of the fragment is consistent with that of incomplete digestion of the DNA by the BglII restriction enzyme. The DNA from four transgenic lines produced with vector pKAS6-aphA6-HTNV is digested with BtgI and probed for the HTNV N cDNA. The expected 10054 bp band is detected in all plants containing the aphA6 transgene (FIG. 50B, lanes 4 through 7). An additional larger band of 11,922 base pairs expected in wild-type tobacco is present in all plants (FIG. 50B, lane 1). The presence of both fragments in the transgenic plants indicates that not all copies of the multicopy chloroplast genome have been converted to the recombinant form (also referred to as heteroplasmy). Analysis of two ANDV-N transgenic lines digested with BtgI reveals the desired 8439 bp product corresponding to Andes nucleoprotein cDNA (FIG. 50C, lanes 4 and 5). The expected wild-type band of 11922 base pairs is also detected (FIG. 50C lane 1).

Thus, all transgenic plants from experiments S24 and S25 yield the predicted results on Southern blots, confirming the integration of transgenes at the desired chloroplast locus, rather than random insertion into the nuclear genome. There is also no obvious phenotypic difference (FIG. 51 (1)) between the wild type and transformant plants when grown in soil. Since the transgenic plants are under kanamycin selection prior to culture in soil, the transgenic plants initially grow more slowly when compared to the wild type which is never cultured with kanamycin; however, shortly after transfer to soil, no apparent difference is observed between the two. To confirm homoplasmy have been achieved in the transgenic lines after several rounds of repeat regeneration, a study of maternal inheritance (FIG. 51(2)) is performed. T1 generation seedlings show no Kanamycin sensitive plants, as expected for a homoplasmic, plastid-encoded trait in tobacco.

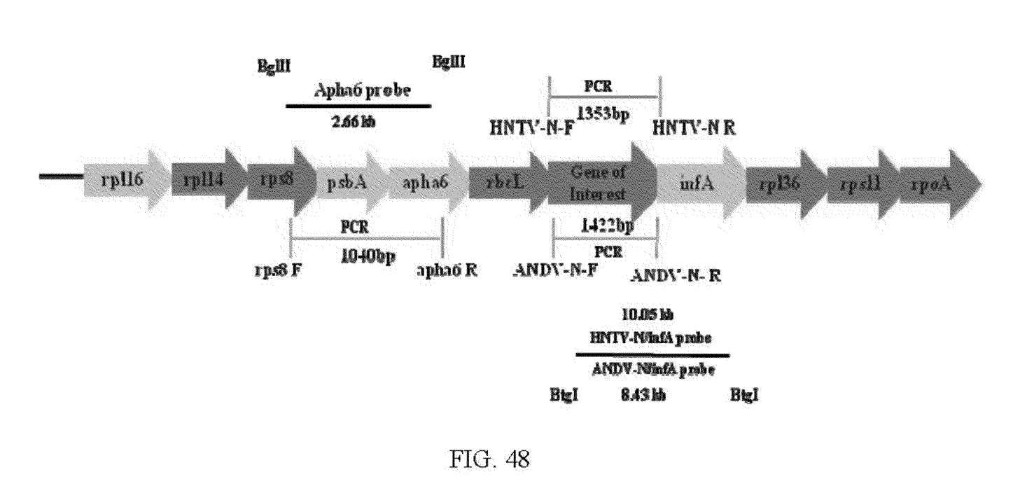

FIG. 48 shows the physical map of the targeted integration locus of the chloroplast genome. The predicted DNA fragments resulting after insertion of transgenes is shown for both PCR (grey lines) and Southern blot (black bar) analyses. All genes shown are native to the tobacco chloroplast except for the contiguous DNA represented by psbA-aphA6-rbcL-Gene of Interest.

### Example 44

### Nicotiana Tabacum cv. Petit Havana Chloroplast Protein Extraction

Total soluble protein is extracted from leaf samples homogenized in a buffer containing 15 mM Sodium carbonate, 35 mM Sodium bicarbonate, 3.08 mM sodium azide, 0.1% Tween 20 and protease inhibitor tablet (Roche Diagnostics). The protein concentration of the extracts is determined by Bradford assay (BioRad) by comparison with a bovine serum albumin (BSA) standard. Hantaan and Andes nucleoproteins are purified from total soluble protein extracts using His-Pur Cobalt columns (Pierce). The purification is performed initially under denaturing conditions using 6M Guanidine-HCl per manufacturer's instructions. Due to aggregation problems and the incompatibility of Guanidine HCl with SDS-PAGE gels, Guanidine HCl is replaced by 6M Urea and fresh urea is added at the time of purification.

### Example 45

### Production of Andes Nucleoprotein in E. coli

Andes nucleoprotein (vector pET151) is expressed in E. coli (BL21 strain). Expression is induced by adding 1 mM IPTG to a 250 ml culture in the logarithmic phase and the bacteria are harvested after 4 hrs of induction by centrifugation of the sample at 1000×g for 15 minutes. The bacterial pellet is lysed with 50 mM Potassium phosphate, pH 7.8, 400 mM NaCl, 100 mM KCl, 10% glycerol, 0.5% Triton-X-100, and 10 mM Imidazole.

The bacterial pellet is resuspended in 5 mL of lysis buffer (for culture volumes of 100 to 500 mL). The samples are frozen in dry ice+EtOH, followed by thawing at room temperature; this procedure is repeated at least 3 times. One μl of protease inhibitor is added for each 100-500 mL of initial culture volume. Samples are split into microcentrifuge tubes and centrifuged at maximum speed for 1 min to pellet debris. The supernatant containing the total soluble protein is transferred into clean tubes and stored at −20° C. The concentration of protein in the sample is determined by Bradford assay (BioRad). Andes nucleoprotein is affinity purified using His Pur cobalt columns as performed for the plant samples. The purified protein samples are confirmed by Coomassie staining and Western blot analysis.

### Example 46

### Western Blot Analysis of Hantaan and Andes Produced in Tobacco (Nicotiana Tabacum Cv. Petit Havana) and E. Coli

Protein samples are separated by electrophoresis in 12% SDS-polyacrylamide gels (BioRad), and the proteins are transferred to either PVDF or Nitrocellulose membranes (BioRad). Monoclonal antibodies for Hantaan and Andes (raised in mouse) are purchased from Abcam, Inc. Western blots are performed using 1:1000 of the primary anti-Hantaan and anti-Andes antibody and 1:2000 for the secondary anti-mouse antibody. The proteins are detected using West Pico HRP substrate. Detection of the histidine tag attached to the C-terminus of the nucleoproteins is accomplished with a Polyhistidine His-Probe HRP antibody (Pierce) at a 1:5000 dilution per manufacturer's instructions.

### Example 47

### Demonstration of the Accumulation of Hantaan and Andes Nucleoprotein Antigens in Nicotiana tabacum Cv. Petit Havana Chloroplast Genome

To demonstrate the accumulation of Hantaan and Andes nucleoproteins in transgenic Nicotiana tabacum cv. Petit Havana chloroplasts, total soluble protein (TSP) extracts are prepared from wild-type and transplastomic plants and analyzed by western blotting. Anti-histidine polyclonal antibodies detect a single ˜49.07 kDa and ˜51.43 kDa protein in Hantaan and Andes transgenic plants, respectively (data not shown). No immunoreactive protein is detected in the wild-type protein extract. Histidine tag affinity purification of nucleoprotein is performed using His-Pur cobalt columns (Pierce). Bradford assays are performed on these samples to determine the total soluble proteins present in extracts prior to and after affinity purification. In the highest yields attained, 9 mg of purified recombinant protein is obtained from 20 g of fresh tobacco leaves. The purified protein is confirmed by Coomassie (FIG. 52A) and Western blot using histidine polyclonal (FIG. 52B), Hantaan monoclonal and Andes monoclonal antibodies, respectively.

### Example 48

### Immunoprecipitation of Hantaan Nucleoprotein

To further concentrate proteins after affinity column purification, immunoprecipitation is performed. Each sample is treated differently. Hantaan nucleoprotein is immunoprecipitated with convalescent serum from Andes-infected individuals and also with Hantaan-specific monoclonal antibody. Andes antigen is immunoprecipitated with Andes convalescent serum and Andes-specific monoclonal antibody. Subsequently, Western blot analysis is performed using Andes convalescent serum.

The lysates (Histag purified proteins or total soluble proteins) are pre-cleared by incubation with normal Mouse sera (1:100 dilution) and protein G-Plus (Pierce) for 1 hour at room temperature (higher specificity). 250 μl of the protein lysates is combined with a 1:100 dilution of the mouse antibody and the total volume is adjusted to 500 ul using 1×PBS. After 1 hr of incubation, the samples are centrifuged at 2000 rpm for 7-10 min. If the lysates are cloudy, they are re-centrifuged at 3000 rpm for 7 min. After centrifugation, the supernatant is recovered into new tubes. To this is added 1:100 of primary monoclonal antibody followed by incubation for 2 hrs at 4° C. Subsequently 50 μl of Protein G-Plus is added and incubated overnight at 4° C. The resulting immune complexes are pelleted and washed four times with 1×PBS buffer. Gel loading buffer is added to each of the pellets followed by boiling for 5 min and loading on a 12% SDS-PAGE gel. After transferring the separated proteins to a PVDF membrane, the blots are probed with 1:1000 Andes convalescent human serum for an hour at 4° C. and then a secondary label with anti-human HRP. Detection of immunoreactive proteins is accomplished by DAB peroxidase substrate (Vector Laboratories Inc.).

The results, as shown in FIG. 53, reveal that monoclonal antibodies react poorly when compared with the convalescent antisera. Slight cross reactivity of Hantaan nucleoprotein with Andes convalescent sera is also observed. A higher yield of Hantaan nucleoprotein is observed (FIG. 53 lane 1) and is attributed to immunoprecipitation of large protein aggregates; Andes nucleoprotein does not aggregate during purification procedures to the same degree as is observed with Hantaan nucleoprotein (data not shown).

### Example 49

### Quantification of the Antigen Reactivity of Recombinant Protein by ELISA

ELISA assays are performed with affinity purified nucleoprotein to quantify the antigenic reactivity of recombinant protein. Chloroplast-produced nucleoprotein is highly reactive with convalescent serum, and exceeds the linear range of the detector (OD 4.0) unless diluted 1:10 (OD 2.8). Antigenic reactivity is less effective when tested with monoclonal antibodies with optical densities averaging one tenth that achieved with human sera.

Briefly, a 96-well plate is coated at 4° C. overnight with 5 μg/ml of Hantaan or Andes nucleoproteins affinity purified from E. coli or tobacco chloroplasts. Coated plates are blocked with 3% milk powder in 0.5% Tween-20 PBS. The plates are washed thrice with 0.5% Tween 20 PBS. After blocking, human convalescent serum from Andes-infected individuals (1:1000) or monoclonal mouse Andes antibody (1:1000) is added and incubated at 37° C. for an hour, followed by incubation with a secondary antibody (1:1000 for anti human and 1:2000 for anti-mouse). Immunoreactivity is detected using ABTS peroxidase substrate (1-component; KPL) and the product is measured at 450 nm. 1% SDS is used to stop the reaction.

The ELISA results demonstrate that production of recombinant nucleoprotein in tobacco chloroplasts averages 6-8% of the total soluble protein, a 3-to-4-fold increase when compared other expression systems. Similar to the immunoprecipitation results, cross-reactivity is seen between Hantaan nucleoprotein and Andes antisera. In contrast, there is no cross reactivity when Andes nucleoprotein is reacted with Hantaan monoclonal antibody.

To compare antigenic reactivity of nucleoproteins produced in E coli and tobacco chloroplasts, equal amounts (5 μg) of the affinity purified protein prepared in parallel is tested using ELISA. Convalescent serum and Andes monoclonal antibodies are used to detect recombinant proteins. The data reveal that the reactivity of the chloroplast-derived nucleoprotein (OD 0.36 with convalescent serum and 0.49 with Andes monoclonal antibody) is similar to that of protein produced in E. coli (OD 0.34 and 0.49). FIG. 54 summarizes the antigenic reactivity of chloroplast-derived and E. coli-derived Andes nucleoproteins. The results demonstrate that the chloroplast system of the present invention yields Hantavirus recombinant proteins that are equal in quality to that of E. coli.

### Example 50

### Construction of Recombinant Rhodomonas salina Multicloning Cassette Using Psby Operon

This Example illustrates construction of a multicloning cassette using Rhodomonas salina psbY operon for insertion of genes of interest. The genome sequence of Cryptophyte alga Rhodomonas salina CCMP1319 plastid is known in the art (GenBank Accession No. EF508371). The CCMP1319 operon, shown as 5′ psbY-rpl32-chlL-chlN 3′, contains pseudogenes chlL and chlN that are potential target sequences for insertion of genes of interest (FIG. 55).

SEQ ID NO: 93 is a partial DNA sequence of the CCMP1319 operon, containing pseudogenes chlL and chlN. The light-independent protochlorophyllide reductase subunit L (chlL, gene 1.8) consists of nucleic acids 1740-1823 of SEQ ID NO: 93. The light-independent protochlorophyllide reductase subunit N (chlN, gene 1 . . . 436) consists of nucleic acids 1943-2378 of SEQ ID NO: 93. The CCMP1319 operon has two unique restriction sites: HindIII (AAGCTT) and HaeII (AGCGCT). HindIII is located between the pseudogenes chlL and chlN (nucleic acids 1890-1895 of SEQ ID NO: 93). HaeII is located within the pseudogene chlN (nucleic acids 2217-2222 of SEQ ID NO: 93). These two restriction sites are useful for insertion of multicloning cassettes containing genes of interest, as illustrated in the examples for construction of tobacco- and Lemna-specific cloning cassettes. Transcription of the genes of interest is driven by the regulatory sequences of the CCMP1319 operon.

### Example 51

### Construction of Recombinant Algae Multicloning Cassette Using Cryptomonas paramecium ATP Synthase Operon

This Example illustrates construction of a multicloning cassette, for example recombinant Rhodomonas salina multicloning cassette, using Cryptomonas paramecium ATP synthase operon for insertion of genes of interest. The genome sequence of Cryptomonas paramecium CCAP977/2a plastid is known in the art (GenBank Accession No. GQ358203.1). The Cryptomonas paramecium CCAP977/2a ATP synthase operon is shown as 5′-rps2-tsf-atpI-atpH-atpG-atpF-atpD-atpA-3′ (FIG. 56).

SEQ ID NO: 94 is a partial sequence of the Cryptomonas paramecium CCAP977/2a ATP synthase operon, containing pseudogenes rps2 (nucleic acids 84-779 of SEQ ID NO: 94), tsf (nucleic acids 821-1141 of SEQ ID NO: 94), atpI (nucleic acids 1453-2199 of SEQ ID NO: 94), atpH (nucleic acids 2254-2502 of SEQ ID NO: 94), atpG (nucleic acids 2539-3048 of SEQ ID NO: 94), atpF (nucleic acids 3250-3486 of SEQ ID NO: 94), atpD (nucleic acids 3557-4111 of SEQ ID NO: 94) and atpA (nucleic acids 4121-5658 of SEQ ID NO: 94).

The atpF pseudogene lacks an obvious start codon and is truncated at its 5′ and 3′ ends relative to the Giullardia theta and Rhodomonas salina ATP synthase pseudogenes. The atpF pseudogene comprises a unique AviII (3′TGCGCAS′) (nucleic acids 3391-3405 of SEQ ID NO: 94) restriction site. This AviII restriction enzyme site is useful for insertion of a multiple cloning cassette containing genes of interest, as illustrated in the examples for construction of tobacco- and Lemna-specific cloning cassettes. Transcription of the genes of interest is driven by the regulatory sequences of the Cryptomonas paramecium ATP synthase operon.

### Example 52

### Construction of Arthrospira platensis (Spirulina platensis Cloning Cassette Using nrs Operon

This Example illustrates construction of a multicloning cassette using Arthrospira platensis (Spirulina platensis) nrs operon for insertion of genes of interest. The Arthrospira platensis (Spirulina platensis) NIES-39 (GenBank Accession No. AP011615.1, sequencing-in-progress) contains operons nrsRS and nrsBACD, 3′ nrsS-nrsR 5′-5′ nrsB-nrsA-nrsC-nrsD 3′ (FIG. 57). SEQ ID NO: 95 is a partial sequence of the Arthrospira platensis (Spirulina platensis) NIES-39 operon, containing pseudogenes nrsS (nucleic acids 152-479 of SEQ ID NO: 95), nrsR (nucleic acids 602-802 of SEQ ID NO: 95), nrsB (nucleic acids 1032-1581 of SEQ ID NO: 95), and nrsA (nucleic acids 1695-4589 of SEQ ID NO: 95). FIG. 58 shows genes 3′ nrsS-nrsR 5′-5′ nrsB-nrsA, which are likely pseudogenes encoding non-functional proteins. Fujisawa et al., DNA Research. 1-19 (2010).

The transcription start sites of the Arthrospira platensis (Spirulina platensis) NIES-39 nrsRS-nrsBA operon include nucleic acids 812, 983, and 999 of SEQ ID NO: 95, according to the start-points determined by Lopez-Maury et al., Molecular Microbiology 43(1): 247-256 (2002). The open reading frame (ORF) organization of the gene cluster from Synechocystis is shown in FIG. 59. The nrsRS-nrsBA operon contains a unique restriction site, StuI (AGGCCT), within nrsR. The StuI restriction site, consisting of nucleic acids 780-785 of SEQ ID NO: 95, is a desired location for insertion of a multicloning cassette containing genes of interest. Transcription of genes inserted into the StuI site is under the regulation of the nrsRS promoter and the transcription start site is “A” (nucleic acid 812 of SEQ ID NO: 95). The nrsRS-nrsBA operon also contains unique restriction sites KpnI (GGTACC) and Fall (AAGTGCGCCTT) within nrsB. The KpnI restriction site consists of nucleic acids 1455-1460 of SEQ ID NO: 95. The Fall restriction site consists of nucleic acids 1212-1222 of SEQ ID NO: 95. Transcription of genes inserted into the KpnI or Fall site is under the regulation of the nrsBACD promoter, and transcription start sites are “C” (nucleic acid 983 of SEQ ID NO: 95) and “G” (nucleic acid 999 of SEQ ID NO: 95), respectively.

### Example 53

### Construction of Sulfolobus acidocaldarius Cloing Cassette

This Example illustrates construction of a multicloning cassette using Sulfolobus acidocaldarius operon for insertion of genes of interest. The complete genome of Sulfolobus acidocaldarius DSM 639 is known in the art (GenBank Accession No. CP000077.1). A partial sequence of the Sulfolobus acidocaldarius DSM 639 operon (FIG. 60), rpl24 (nucleic acids 1-405 of SEQ ID NO: 96)-rpl14 (nucleic acids 408-824 of SEQ ID NO: 96)-rps7 (nucleic acids 826-1170 of SEQ ID NO: 96)-conserved protein (nucleic acids 1171-1372 of SEQ ID NO: 96)-rp129 (nucleic acids 1404-1613 of SEQ ID NO: 96)-rps3 (nucleic acids 1614-2292 of SEQ ID NO: 96)-rpl22 (nucleic acids 2296-2766 of SEQ ID NO: 96), is SEQ ID NO: 96. The complete S10 operon is about 5 kb from rpl14 to rpl3, which is illustrated as 5′ rpl14-rps7-conserved protein-rpl29-rps3-rpl22-rps19-rpl2-rpl23-rpl4-rpl3 3′. To enable homologous recombination into the operon, approximately 1 kb of flanking DNA is required on each side of the inserted gene of interest, and the required flanking DNA can be constructed by cloning DNA sequence from rpl24 to rpl22. The S10 operon contains a unique restriction site XbaI (5′ TCTAGA 3′) (nucleic acids 1509-1514 of SEQ ID NO: 96) suitable for insertion of a multicloning cassette containing genes of interest. The XbaI restriction site consists of nucleic acids 1509-1514 of SEQ ID NO: 96 and is located within the rpl29 pseudogene region. Table 2 illustrates positions of certain pseudogenes of Archaea. Van Passel et al., Archaea 2, 137-143 (2007).

TABLE 2
Positions of Certain Pseudogenes

| | | | PsiGene | Reference | |
|---|---|---|---|---|---|
| Psigene ID | Reference ID | Coordinates | Function | Function | Mechanism |
| YP_255275.1 | NP_376303.1 | 471496-471633 | 50S ribosomal | 50S ribosomal | truncated |
| | | | protein L29P | protein L29 | |

### REFERENCES CITED

U.S. Patent Documents
Adang et al., “Synthetic Insecticidal Crystal Protein Gene,” U.S. Pat. No. 5,380,831 (1995)
Chappel et al., “Process for Composition for Increasing Squalene and Sterol Accumulation in Higher Plants,” U.S. Pat. No. 5,349,126 (1994)
Fujimoto et al., “Synthetic Insecticidal Gene, Plants of the Genus Oryza Transformed with the Gene, and Production Thereof,” U.S. Pat. No. 5,436,391 (1995)
Hahn F M, Kuehnle A R. Manipulation of genes of the Mevalonate and isoprenoid patways to create novel traits in transgenic plants. U.S. Pat. No. 7,129,392.
Kamuro et al. “Herbicide” U.S. Pat. No. 4,846,872 (1989)
Other References
Albrecht et al., “Novel Hydroxycarotenoids with Improved Antioxidative Properties Produced by Gene Combination in Escherichia coli,” Nature Biotech. 18:843-846 (2000)
Allison et al., MDMV Leader (Maize Dwarf Mosaic Virus) Virology 154:9-20 (1986)
Altschul et al., J. Mol. Biol. 215:403-410 (1990)
Ashby and Edwards, “Elucidation of the Deficiency in Two Yeast Coenzyme Q Mutants: Characterization of the Structural Gene Encoding Hexaprenyl Pyrophosphate Synthetase,” J. Biol. Chem. 265:13157-13164 (1990) Ballas et al., Nucleic Acids Res. 17:7891-7903 (1989) Barkley S J, Shrivallabh D B, and Poulter C D, Type II Isopentenyl Diphosphate Isomerase from Synechocystis sp. Strain PCC 6803. J. Bacteria 186(23):8156-8158 (2004)
Bateman J M and Purton S, Tools for chloroplast transformation in Chlamydomonas: expression vectors and a new dominant selectable marker. Mol. Gen. Genet. 263:404-410 (2002)
Beaucage and Caruthers, Tetra. Letts., 22:1859-1862 (1981)
Bock and Hagemann, “Extranuclear Inheritance: Plastid Genetic: Manipulation of Plastid Genomes and Biotechnological Application,” Prog. Bot. 61:76-90 (2000)
Bock R., Transgenic plastid in basic research and plant biotechnology. J Mol. Biol. 2001; 312:425-438.
Bostik P, Winter J, Ksiazek T G, Rollin P E, Villinger F, Zaki S R, Peters C J, Ansari A A. Sin, Nombre virus (SNV) Ig isotype antibody response during acute and convalescent phases of Hantavirus pulmonary syndrome. Emerg. Infect. Dis. 2000; 2: 184-188.
Boyton and Gillham, “Chloroplast Transformation in Chlamydomonas,” Methods Enzymol. 217:510-536 (1993)
Bucht G, Brus Sjolander K, Eriksson S, Lindgren L, Lundkvist A, Elgh F. Modifying the cellular transport of DNA-based vaccines alters the immune response to hantavirus nucleocapsid protein. Vaccine 2001; 19:3820-3829.
Cho H-W, Howard C R, Lee H-W. Review of an Inactivated Vaccine against Hantaviruses. Intervirology 2002; 45:328-333.
Clarke, “Protein Isoprenylation and Methylation at Carboxy-terminal Cysteine Residues,” Annu. Rev. Biochem. 61:355-386 (1992)
Cunningham and Gantt, “Genes and Enzymes of Carotenoid Biosynthesis in Plants,” Ann. Rev.
Plant Mol. Biol. 39:475-502 (1998)
Cunningham et al., “Evidence of a Role for LytB in the Nonmevalonate Pathway of Isoprenoid Biosyhthesis,” J. Bacteriol. 182:5841-5848 (2000)
Dale, P. J., “Spread of Engineered Genes to Wild Relatives,” Plant Physiol. 100:13-15 (1992)
Daniell et al., “Containment of Herbicide Resistance Through Genetic Engineering of the Chloroplast Genome,” Nat. Biotechnol. 16:345-348 (1998)
Daniell H, Chebolu S, Kumar Sashi, Singleton M, Falconer R. Chloroplast-derived vaccine antigens and other therapeutic proteins. Vaccine. 2005; 1779-1783.
Dargeviciute A, Brus S K, Sasnauskas K, Kruger D H, Meisel H, Ulrich R, Lundqvist A. Yeast-expressed Puumala hantavirus nucleocapsid protein induces protection in a bank vole model. Vaccine. 2002; 20: 3523-3531.
del Campo et al, Plant Physiol 114:748 (1997)
Della-Cioppa et al., Plant Physiol. 84:965-968 (1987)
Deroles and Gardner, “Expression and Inheritance of Kanamycin Resistance in a large Number of Transgenic Petunias Generated by Agrobacterium-Mediated Transformation,” Plant Molec. Biol. 11: 355-364 (1988)
De Santis-Maciossek G, Kofer W, Bock A, Schoch S, Majer R, Wanner G, Rudiger W, Koop H-U, and Herrmann R G. Targeted disruption of the plastid RNA polymerase genes rpoA, B and C1: molecular biology, biochemistry and ultrastructure. The Plant 118:477-489 (1999)
Eisenreich et al., “The Deoxyxylulose Phosphate Pathway of Terpenoid Biosynthesis in Plants and Microorganisms,” Chemistry and Biology 5:R221-R233 (1998)
Elgh F, Linderholm M, Wadell G, Juto P. The clinical usefulness of a Puumalavirus recombinant nucleocapsid protein based enzyme-linked immunosorbent assay in the diagnosis of nephropathia epidemica as compared with an immunofluorescence assay. Clin Diagn Virol. 1996; 6(1) 17-26.
Elgh F, Lunkvist A, Alexeyev O, Stenlund H, Avisc-Zupanc T, Hjelle B et al. Serological diagnosis of hantavirus infections by an enzyme-linked Immunosorbent assay based on detection of immunoglobilun G and M responses to recombinant nucelocapsid proteins of five viral serotypes. J. Clin Microbiol 1997; 35 [5]: 1122-1130.
Elroy-Stein et al., PNAS USA 86:6126-6130 (1989)
Gallie et al., in Molecular Biology of RNA, ed. Cech, (Liss, New York) 237-256 (1989)
Garrett et al., “Accumulation of a Lipid A Precursor Lacking the 4′-Phosphate following Inactivation of the Escherichia coli 1pxK Gene,” J. Biol. Chem. 273:12457-12465 (1998)
Glenz K, Bouchon B, Stehle t, Wallich R, Simon M M, Warzecha H. Production of recombinant bacterial lipoprotein in higher plant chloroplasts. Nature Biotechnol. 2006; 24: 76-77.
Goldschmidt-Clermont M., “Transgenic Expression of Aminoglycoside Adenine Transferase in the Chloroplast: A Selectable Marker for Site-directed Transformation of Chlamydomonas,” Nucleic Acids Res.19:4083-4089 (1991)
Goodwin, “Biosynthesis of Carotenoids and Plant Triterpenes: the Fifth CIBA Medal Lecture,” Biochem. J. 123:293-329 (1971)
Gott P, Zoller L, Yang S, Stohwasser R, Bautz, EKF, Darai G. Antigenicity of hantavirus nucleocapsid proteins expressed in E. coli. Virus Res. 1991; 19: 11-16.
Gött P, Zöller L, Darai Z, Bautz E K. A major Antigenic domain of hantaviruses is located on the aminoproximal site of the viral nuceocapsid protein. Virus Genes 1997; 14: 31-40.
Guda et al., “Stable Expression for a Biodegradable Protein Based Polymer in Tobacco Chloroplasts,” Plant Cell Reports 19:257-262 (2000)
Guerineau et al., Mol. Gen. Genet. 262:141-144 (1991)
Hahn et al., “1-Deoxy-D-Xylulose 5-Phosphate Synthase, the Gene Product of Open Reading Frame (ORF) 2816 and ORF2895 in Rhodobacter capsulatus,” J. Bacteriol. 183:1-11 (2001)
Hahn and Poulter, “Isolation of Schizosaccharomyces pombe Isopentenyl Diphosphate Isomerase cDNA Clones by Complementation and Synthesis of the Enzyme in Escherichia coli,” J. Biol. Chem. 270:11298-11303 (1995)
Hahn et al., ‘Escherichia coli Open Reading Frame 696 Is idi, a Nonessential Gene Encoding Isopentenyl Diphosphate Isomerase,” J. Bacteriol. 181:4499-4504 (1999)
Hahn et al., “Open Reading Frame 176 in the Photosynthesis Gene Cluster of Rhodobacter capsulatus Encodes idi, a Gene for Isopentenyl Diphosphate Isomerase,” J. Bacteriol. 178:619-624 (1996)
Hamilton et al., “New Method for Generating Deletions and Gene Replacements in Escherichia coli,” J. Bacteriol. 171:4617-4622 (1989)
Harker and Bramley, “Expression of Prokaryotic 1-Deoxy-D-Xylulose 5-Phosphates in Escherichia coli Increases Carotenoid and Ubiquinone Biosynthesis,” FEBS Letters 448:115-119 (1999)
Herz et al., “Biosynthesis of Terpenoids: YgbB Protein Converts 4-Diphosphocytidyl-2C-Methyl-D-Erythritol 2-Phosphate to 2C-Methyl-D-Erythritol 2,4-Cyclodiphosphate,” Proc. Natl. Acad. Sci. USA 97:2486-2490 (2000)
Herz S, Fu M, Steiger S, and Koop H-U. Development of novel types of plastid transformation vectors and evaluation of factors controlling expression. Transgenic Res. 14:969-982 (2005).
Huang F-C, Klaus S M J, Herz S, Zou Z, Koop H-U, and Golds T J. Efficient plastid transformation in tobacco using the aphA-6 gene and kanamycin selection. Mol. Gen. Genomics 268:19-27 (2002).
Hujakka H, Koistinen V, Kuronen I, Eerikainen P, Parviainen M, Lundkvist A et al. Diagnostic rapid tests for acute hantavirus infections: specific tests for Hantaan, Dobrava and Puumala viruses versus a hantavirus combination test. J Virol Methods 2003; 108:117-122.
Jobling et al., Nature 325:622-625 (1987)
Joshi et al., Nucleic Acid Res. 15:9627-9639 (1987)
Kaneda K, Kuzuyama T, Takagi M, Hayakawa Y, and Seto H, An unusual Isopentenyl Diphosphate Isomerase Found in the Mevalonate Pathway Gene Cluster from Streptomyces sp. Strain CL 190. Proc. Natl. Acad. Sci. 98(3):932-937 (2001).
Kajiwara et al., “Expression of an Exogenous Isopentenyl Diphosphate Isomerase Gene Enhances Isoprenoid Biosynthesis in Escherichia coli,” Biochem. J. 324:421-426 (1997)
Kallio-Kakko H, Vapalahi O, Lundkvist A, Vaheri A. Evaluation of Puumala virus IgG and IgM enzyme immunoassays based on recombinant Baculovirus-expressed nucleocapsid protein for early nephropathica diagnosis. Clin. Diagn. Viro1.1998; 10: 83-90.
Kavanagh et al., “Homeologous Plastid DNA Transformation in Tobacco is Mediated by Multiple Recombination Events,” Genetics 152:1111-1122 (1999)
Keeler et al., “Movement of Crop Transgenes into Wild Plants,” in Herbicide Resistant Crops: Agricultural, Economic, Environmental, Regulatory and Technological Aspects, (S. O. Duke, ed.) CRC Press, Boca Rotan, Fla., pp 303-330 (1996)

Kehm R, Jakob J N, Welzel T M, Tobiasch E, Viczian O, Jock S et al. Expression of immunogenic puumala virus nucleocapsid protein in transgenic tobacco and potato plants. Virus Genes. 2001; 22 (1); 73-83.
Khan and Maliga, “Fluorescent Antibiotic Resistance Marker for Tracking Plastid Transformation in Higher Plants,” Nature Biotech. 17:910-914 (1999)
Khattak S, Darai G, Sule S, Rosen-Wolff A. Characterization of expression of Puumala virus nucleocapsid protein in transgenic plants. Intervirology. 2002; 45: 334-339.
Khattak S, Darai G, Rosen-Wolff A. Puumala virus nucleocapsid protein expressed in transgenic plants is not immunogenic after oral administration. Virus Genes. 2004; 29: 109-116.
Kofer W, Eibl C, Steinmuller K, and Koop H-U, PEG-mediated plastid transformation in higher plants. In Vitro Cell. Dev. Biol. 34:303-308 (1998).
Koop H-U, Steinmuller K, Wagner H, Robler C, Eibl C, Sacher L, Integration of foreign sequences into the tobacco plastome via polyethylene glycol-mediated protoplast transformation. Planta 19:193-201 (1996).
Kota et al., “Overexpression of the Bacilllus thuringiensis (Bt) Cry2Aa2 Protein in Chloroplasts Confers Resistance to Plants Against Susceptible and Bt-resistant Insects,” Proc. Natl. Acad. Sci. USA 96:1840-1845 (1999)
Koya V, Moayeri M, Leppla S H, Daniell H. Plant-based vaccine: mice immunized with chloroplast-derived anthrax protective antigen survive anthrax lethal toxin challenge. Infect Immun. 2005; 73: 8266-8274.
Kunkel, Proc. Natl. Acad. Sci. USA 82:488-492 (1985)
Kunkel et al., Methods and Enzymol; 154:367-382 (1987)
Kuzuyama et al., “Direct Formation of 2-C-Methyl-D-Erythritol 4-Phosphate by 1-Deoxy-D-Xylulose 5-Phosphate Reductoisomerase, a New Enzyme in the Non-Mevalonate Pathway to Isopentenyl Diphosphate, Tetrahedron Lett. 39:4509-4512 (1998)
Kuzuyama et al., “Fosmidomycin, a Specific Inhibitor of 1-Deoxy-D-Xylulose 5-Phosphate Reductoisomerase in the Nonmevalonate Pathway for Terpenoid Biosynthesis,” Tetrahedron Lett. 39:7913-7916 (1998)
Kuzuyama et al., “An Unusual Isopentenyl Diphosphate Isomerase Found in the Mevalonate Pathway Gene Cluster from Streptomyces sp. strain CL190,” Proc. Natl. Acad. Sci. USA 98:932-7 (2001)
Lange and Croteau, “Isopentenyl diphosphate biosynthesis via a mevalonate independent pathway: Isopentenyl monophosphate kinase catalyzes the terminal enzymatic step,” Proc. Natl. Acad. Sci. USA 96:13714-13719 (1999)
Lichtenthaler et al., “Biosynthesis of Isoprenoids in Higher Plant Chloroplasts Proceeds via a Mevalonate-Independent Pathway,” FEBS Letters 400:271-274 (1997)
Lois et al, “Cloning and Characterization of a Gene from Escherichia coli Encoding a Transketolase-Like Enzyme that Catalyzes the Synthesis of D-1-Deoxyxylulose 5-Phosphate, a Common Precursor for Isoprenoid, Thiamin, and Pyridoxol Biosynthesis,” Proc. Natl. Acad. Sci. USA 95:2105-2110 (1998)
Lommel et al., Virology 81:382-385 (1991)
Lundkvist A, Kallio-kakko H, Brus-Sjolander K, Lankinen H, Niklasson B, Vaheri A, Vapalahti O. Virology. 1996; 216:397-406.
Lüttgen et al., “Biosynthesis of Terpenoids: YchB Protein of Escherichia coli Phosphorylates the 2-Hydroxy Group of 4-Diphosphocytidyl-2-C-Methyl-D-Erythritol,” Proc. Natl. Acad. Sci. USA 97:1062-1067 (2000)
Macejak et al., Nature 353:90-94 (1991)
Maliga, Pal, DNA constructs and methods for stably transforming plastids of multicellular plants and expressing recombinant proteins therein. U.S. Pat. No. 5,877,402. Filed Jan. 31, 1994, and issued Mar. 2, 1999.
Maliga P. Plastid transformation in higher plants. Annu. Rev. Plant Biol. 2004; 55: 289-313.
Martin W, Stobe B, Goremykin V, Hansmann S, Hasegawa M, Kowallik K V. Gene transfer to the nucleus and the evolution of chloroplasts. Nature. 1998; 393:162-165.
Mann et al., “Metabolic Engineering of Astaxanthin Production in Tobacco Flowers,” Nature Biotech. 18:888-892 (2000)
Martin et al., “Gene Transfer to the Nucleus and the Evolution of Chloroplasts,” Nature 393:162-165 (1998)
Matsuoka et al., “Variable Product Specificity of Microsomal Dehydrodolichyl Diphosphate Synthase from Rat Liver,” J. Biol. Chem. 266:3464-3468 (1991)
Matteuci et al., J. Am. Chem. Soc., 103: 3185 (1981)
Meinkoth and Wahl, Anal. Biochem. 138:267-284 (1984)
Meyer and Saedler, “Homology-Dependent Gene Silencing in Plants,” Ann. Rev. Plant. Physiol. Mol. Biol. 47:23-48 (1996)
Millen et al., “Many Parallel Losses of infA from Chloroplast DNA During Angiosperm Evolution with Multiple Independent Transfers to the Nucleus,” Plant Cell 13: 645-658 (2001)
Morii M, Yoshimatsu K, Arikawa J, Zhou G, Kariwa H, Takashima I. Antigenic characterization of Hantaan and Seoul Virus Nucleocapsid proteins expressed by recombinant Baculovirus: Application of a truncated protein, lacking an antigenic region common to the two viruses, as a serotyping antigen. J Clin Microbiol 1998; 2514-2521.
Murashige T, Skoog F. A revised medium for rapid growth and bioassays with tobacco tissue culture. Physiol Plant. 1962; 15: 47-497.
Nakamura A, Shimada H, Masuda T, Ohta H, Takamiya A., Two distinct isopentenyl diphosphate isomerases in cytosol and plastid are differentially induced by environmental stresses in tobacco. FEBS Lett. 506:61-64 (2001).
Mogen et al., Plant Cell 2:1261-1272 (1990)
Munroe et al., Gene 91:151-158 (1990)
Murray et al., Nucleic Acids Res. 17:477-498 (1989)
Needleman et al., J. Mol. Biol. 48:443 (1970)
Newman et al., “Genes Galore: A Summary of Methods for Accessing Results from Large-Scale Partial Sequencing of Anonymous Arabidopsis cDNA Clones,” Plant Physiology 106:1241-1255 (1994)
Nielsen and Bloor, “Analysis and Developmental Profile of Carotenoid Pigments in Petals of Three Yellow Petunia Cultivars,” Scientia Hort. 71:257-266 (1997)
Oey M, Lohse M, Kreikemeyer B, Bock R. Exhaustion of the chloroplast protein synthesis capacity by massive expression of a highly stable protein antibiotic. Plant J. 2009; 57; 436-445
Pachuk et al., Gene 243:19-25 (2000)
Pearson et al., Proc. Natl. Acad. Sci. 85:2444 (1988)
Popjak, “Natural Substances Formed Biologically from Mevalonic Acid,” Biochemical symposium no. 29 (T. W. Goodwin, ed.) Academic Press, New York, pp 17-37 (1970)
Proudfoot, Cell 64:671-674 (1991)
Ramos-Valdivia et al., “Isopentenyl Diphosphate Isomerase: A Core Enzyme in Isoprenoid Biosynthesis: A Review of its Biochemistry and Function,” Nat. Prod. Rep. 6:591-603 (1997)
Rohdich et al., “Cytidine 5′-Triphosphate-Dependent Biosynthesis of Isoprenoids: YgbP Protein of Escherichia coli Catalyzes the Formation of 4-Diphosphocytidyl-2-C-methylerythritol,” Proc. Natl. Acad. Sci. USA 96:11758-11763 (1999)
Razanskiene A, Schmidt J, Geldmacher A, Ritzi A, Niedrig M, Lundkvist A, Kruger D H, Meisel H, Sasnauskas K, Ulrich R. High yields of stable and highly pure nucleocapsid proteins of different hantaviruses can be generated in the yeast Saccharomyces cerevisiae. 2004; 111:319-333.
Rosellini D, LaFayette P R, Barone P, Veronesi F, Parrott W A, Kanamycin-resistant alfalfa has a point mutation in the 16S plastid rRNA. Plant Cell Reports 22:774-779 (2004)
Ruf S, Karcher D, Bock R. Determining the transgene containment level provided by chloroplast transformation. Proc. Natl. Acad. Sci. USA. 2007; 104: 6998-7002.
Ruhlman T, Ahangari R, Devine A, Samsam M, Daniell H. Expression of cholera toxin B-proinsulin fusion protein in lettuce and tobacco chloroplasts—oral administration protects against development of insulitis in non-obese diabetic mice. Plant Biotechnol J. 2007; 5: 495-510
Sambrook et al., “Molecular Cloning: A Laboratory Manual,” 2nd ed., Cold Spring Harbor Laboratory Press, Cold Spring Harbor, N.Y. (1989)
Sanfacon et al., Genes Dev. 5:141-149 (1991)
Serino and Maliga, “A Negative Selection Scheme Based on the Expression of Cytosine Deaminase in Plastids,” Plant J. 12:687-701 (1997)
Schmaljohn C S, Sugiyma K, Schmaljohn A L, Bishop D H. Baculovirus expression of the small genome segment of Hantaan virus and potential use of the expressed nucleocapsid proteins as a diagnoistic antigen. J. Gen. Virol. 1988; 69: 777-786
Schmaljohn C. S., Chu Y K, Schmaljohn A L, Dalrymple J M. Antigenic subunits of Hantaan virus expressed by baculovirus and vaccinia virus recombinants. J. Virol. 1990; 64:3162-3170.
Schmidt J, Jandrig B, Klempa B, Yoshimatsu K, Arikawa J et al. Nucleocapsid protein of cell culture-adapted Seoul virus strain 80-39: analysis of its encoding sequence, expression in yeast and immuno-reactivity. Virus Genes 2005a; 30: 37-48.
Schmidt J, Meisel H, Hjelle B, Kruge D, Ulrich R. Development and evaluation of serological assays for detection of human hantavirus infections caused by Sin Nombre virus. J. Clin. Virol 2005b; 33: 247-253.
Smith et al., Adv. Appl. Math. 2:482 (1981)
Svab Z, Maliga P. Exceptional transmission of plastids and mitochondria from the transplastomic pollen parent and its impact on transgene containment. Proc. Natl. Acad. Sci. USA. 1990; 104:7003-7008.
Sun Z, Cunnningham F, and Gantt E., Differential expression of two isopentenyl pyrophosphate isomerases and enhanced carotenoid accumulation in a unicellular chlorophyte. PNAS 95(19):11482-11488 (1998)
Sprenger et al., “Identification of a Thiamin-Dependent. Synthase in Escherichia coli Required for the Formation of the 1-Deoxy-D-Xylulose 5-Phosphate Precursor to Isoprenoids, Thiamin, and Pyridoxol,” Proc. Natl. Acad. Sci. USA 94:12857-12862 (1997)
Stevens and Burton, “Genetic Engineering of Eukaryotic Algae: Progress and prospects,” J. Phycol 33:713-722 (1997)
Sugiura, M., “Direct submission to the EMBL/GenBank/DDBJ databases, bases 1-155939,” (1986)
Takagi et al., “A Gene Cluster for the Mevalonate Pathway from Streptomyces sp Strain CL190,” J. Bacteriol. 182:4153-4157 (2000)
Takahashi, et al., “Purification, Characterization, and Cloning of a Eubacterial 3-Hydroxy-3-Methylglutaryl Coenzyme A Reductase, a Key Enzyme Involved in Biosynthesis of Terpenoids,” J. Bacteriol. 181:1256-1263 (1999)
Tischler N D, Galeno H, Rosemblatt M, Valenzuela P D. Human and rodent humoral immune responses to Andes virus structural proteins. Virology 2005; 344: 319-326.
Toriyama and Hinata, “Cell Suspension and Protoplast Culture in Rice,” Plant Science 41:179-183 (1985)
Tregoning J S, Nixon P, Kuroda H, Svab Z, Clare S, Bowe F, et al. Expression of tetanus toxin fragment C in tobacco chloroplasts. Nucleic Acids Research. 2003; 31:1174-1179.
Vapalahti O, Lundkvist A, Kallio-kokko H, Paukku K, Julkunen I, Lankinen H, Vaheri A. J. Clin. Microbiol. 1996; 34 (1): 119-125.
Tsudsuki, T., “Direct submission, bases 1-155939. Data Processing Center, Aichi-Gakuin University, Aichi, Japan,” (1998)
Vain P, McMullen M D, Finer J J, Osmotic treatment enhances particle bombardment-mediated transient and stable transformation of maize. Plant Cell Reports 12:84-88 (1993)
Vapalahti O, Lundkvist A, Kallio-kokko H, Paukku K, Julkunen I, Lankinen H, Vaheri A. J. Clin. Microbiol. 1996; 34 (1): 119-125.
Watson J, Koya V, Leppla S H, Daniell H. Expression of Bacillus anthracis protective antigen in transgenic chloroplasts of tobacco, a non food/feed crop. Vaccine. 2004, 22: 4372-4384.
Vasil et al., in Cell Culture and Somatic Cell Genetics of Plants, Vols. I, II, and III, Laboratory Procedures and Their Applications (Academic press) (1984)
Weissbach et al., Methods for Plant Mol. Biol. (1989)
Wurbs D, Ruf S, Bock R. Contained metabolic engineering in tomatoes by expression of carotenoid biosynthesis genes from the plastid genome. Plant J. 2007; 49:276-288.
Yamada T, Hjelle B, Lanzi R, Morris C, Anderson B, Jenison S. Antibody responses to four corners hantavirus infections in the deer mouse (Peromyscus maniculatus): identification of an immunodominant region of the viral nuceocapsid protein. J. Virol. 1995; 69: 1939-1943.
Ye et al., Science 287:303-30 (2000)
Yoshimatsu K, Arikawa J, Yoshida R, Li H, Yoo Y C, Kariwa H et al. Production of recombinant hantavirus nucleocapsid protein expressed in silkworm larvae and its use as a diagnostic antigen in detecting antibodies in serum from infected rats. Lab Animals. Sci. 1995; 45: 641-646.
Yoshimatsu K, Arikawa J, Tamura R, Yoshida, Lundkvist A, Niklasson B, Kariwa H, Azuma I. Characterization of the nucelocapsid protein of Hantaan virus strain 76-118 using monoclonal antibodies. J. Gen. Virol. 1996; 77: 695-704.
Yusibov V, Hooper D C, Spitsin S V, Fleysh N, Kean R B, Mikheeva T et al. Expression in plants and immunogenicity of plant virus-based experimental rabies vaccine. Vaccine. 2002; 3155-3164.
Zhang Y-Z, Dong X, Li X, Ma C, Xiong H-P, Yan G-J et al. Seoul Virus and Hantavirus Disease, Sheyang, People's Republic of China. Emerg Infect Dis; 2009; 15 [2]200-206.
Zhou F, Badillo-Corona J A, Karcher D, Gonzalez-Rabade N, Piepenburg K, Borchers A-MI, Maloney A P, Kavanagh, T A, Gray J C, Bock R. High-level, expression of HIV antigens from the tobacco and tomato plastid genomes. Plant Biotechnol J. 2008.

We claim:

1. A method of providing a cell with an inserted polynucleotide sequence encoding one or more products of interest, said method comprising:
providing a plurality of target cells having an identified endogenous plastidic pseudogene site located within an operon;
providing an isolated polynucleotide lacking transcriptional regulatory sequences and comprising polynucleotide sequences of said pseudogene site flanking at least one heterologous coding sequence of interest;
introducing said isolated polynucleotide into a plurality of said target cells; and
selecting at least one target cell which contains the heterologous coding sequence of interest inserted into said pseudogene site, wherein the coding sequence of interest is operably linked to the regulatory sequences of the operon containing the pseudogene and is transcribed, and
wherein said target cell is a tobacco, Rhodomonas, Cryptomonas, Arthrospira, Archaea, Lemna or Chlamydomonas cell.

2. A method of providing a cell with an inserted polynucleotide sequence encoding one or more products of interest, said method comprising:
providing a plurality of target cells having an identified endogenous plastidic pseudogene site located within an operon;
providing an isolated polynucleotide lacking transcriptional regulatory sequences and comprising polynucleotide sequences of said pseudogene site flanking at least one heterologous coding sequence of interest;
introducing said isolated polynucleotide into a plurality of said target cells; and
selecting at least one target cell which contains the heterologous coding sequence of interest inserted into said pseudogene site, wherein the coding sequence of interest is operably linked to the regulatory sequences of the operon containing the pseudogene and is transcribed, and
wherein said coding sequence of interest comprises a portion encoding a viral protein.

3. The method according to claim 2, wherein said viral protein is a Hantaan protein or an Andes protein.

4. The method according to claim 3, wherein the Hantaan protein is Haantan nucleoprotein.

5. The method according to claim 3, wherein the Andes protein is Andes nucleoprotein.

6. The method according to claim 1, wherein the inserted coding sequence of interest is operably linked to the regulatory sequences of the rpl23 operon.

7. The method according to claim 2, wherein the inserted coding sequence of interest is operably linked to the regulatory sequences of the rpl23 operon.

8. The method according to claim 1, wherein the inserted coding sequence of interest is operably linked to the regulatory sequences of a Rhodomonas psbY operon, Cryptomonas_ATP synthase operon, Arthrospira nrs operon, or Archaea operon.

9. The method according to claim 8, wherein the Rhodomonas psbY operon is a Rhodomonas salina psbY operon.

10. The method according to claim 8, wherein the Rhodomonas psbY operon comprises a pseudogene site selected from chlL, chlN, or both.

11. The method according to claim 8, wherein Cryptomonas_ATP synthase operon is a Cryptomonas paramecium ATP synthase operon.

12. The method according to claim 8, wherein the Cryptomonas ATP synthase operon comprises a pseudogene site selected from the group consisting of rps2, tsf, atpI, atpH, atpG, atpF, atpD, and atpA.

13. The method according to claim 8, wherein Arthrospira nrs operon is an Arthrospira platensis nrs operon.

14. The method according to claim 8, wherein the Arthrospira nrs operon comprises a pseudogene site selected from the group consisting of nrsS, nrsR, nrsB, and nrsA.

15. The method according to claim 8, wherein the Archaea operon is a Sulfolobus acidocaldarius S10 operon.

16. The method according to claim 8, wherein the Archaea operon comprises a pseudogene site selected from the group consisting of rpl24, rpl14, rps7, rpl29, rps3, and rpl22.

17. The method according to claim 2, wherein said target cell is a plant cell.

18. The method according to claim 2, wherein said target cell is a microalgae cell.

19. The method according to claim 2, wherein said target cell is a tobacco, Rhodomonas, Cryptomonas, Arthrospira, Archaea, Lemna or Chlamydomonas cell.

20. The method according to claim 17, wherein said plant cell is from a solanaceous species.

21. The method according to claim 2, wherein said plant cell is selected from the group consisting of petunia, tomato, potato, and tobacco cells.
